SEQUENCE LISTING (1 sequence)

SEQ ID NO: 1 - protein, 11 aa, Artificial Sequence
  Cell-penetrating peptide
CGRKKRRQRRR

(19) United States
(12) Patent Application Publication
(10) Pub. No.: US 2023/0038373 A1
(43) Pub. Date: Feb. 9, 2023

(54) STABILE CONJUGATE

(71) Applicant: Glykos Biomedical OY, Helsinki (FI)

(72) Inventors: Tero SATOMAA, Helsinki (FI); Juhani SAARINEN, Helsinki (FI); Jari HELIN, Rajamäki (FI); Olli AITIO, Helsinki (FI); Henna PYNNÖNEN, Vantaa (FI)

(73) Assignee: Glykos Biomedical OY, Helsinki (FI)

(21) Appl. No.: 17/783,738

(22) PCT Filed: Dec. 18, 2020
(86) PCT No.: PCT/FI2020/050857

§ 371 Date: Jun. 9, 2022

(30) Foreign Application Priority Data
Dec. 18, 2019 (FI) 20196092

(51) Int. Cl. A61K 47/68; A61P 35/00

(52) U.S. Cl. CPC A61K 47/6851; A61K 47/6803; A61P 35/00

(57) ABSTRACT

A conjugate is disclosed. The conjugate may be represented by Formula I: [D-G-L]ₙ-T Formula I wherein D is a payload molecule; O is an oxygen atom of said payload molecule; T is a targeting unit capable of binding a target molecule, cell and/or tissue; and n is at least 1.

### TECHNICAL FIELD

The present disclosure relates to a conjugate.

### BACKGROUND

Various payload molecules are known in the art that would benefit from specific delivery to a target tissue for treatment of cancer or other diseases. A number of these payloads comprise a hydroxyl group. However, methods to conjugate such payloads via a relatively stabile bond to the hydroxyl group have been lacking.

### SUMMARY

A conjugate is disclosed.

The conjugate may be represented by Formula I:

[D-O-L]ₙ-T Formula I

wherein D is a payload molecule;

O is an oxygen atom of said payload molecule;

T is a targeting unit capable of binding a target molecule, cell and/or tissue; and

n is at least 1.

### BRIEF DESCRIPTION OF THE DRAWINGS

The accompanying drawings, which are included to provide a further understanding of the embodiments and constitute a part of this specification, illustrate various embodiments. In the drawings:

FIG. 1 illustrates the ¹H-NMR spectra of kifunensine (upper panel) and 6-succinyl-kifunensine ester (lower panel). x-axis shows the chemical shift in parts per million (ppm) and y-axis shows the relative signal intensity. Proton numbering is shown for kifunensine in the upper panel.

FIG. 2 shows MALDI-TOF mass spectra of kifunensine ester compounds after 5 days' incubation in buffered cell culture medium at +37° C. A. 6-(2-methyl)butanoyl-kifunensine at m/z 339.222 [M+Na]⁺. B. 6-(2,2-dimethyl)propanoyl-kifunensine at m/z 339.230 [M+Na]⁺. C. 6-butanoyl-kifunensine at m/z 325.188 [M+Na]⁺. D. 6-succinyl-kifunensine at m/z 355.188 [M+Na]⁺. The hydrolysis product kifunensine was visible in all panels A.-D. at m/z 255.1 [M+Na]⁺.

FIG. 3 shows MALDI-TOF mass spectra of kifunensine ester compounds after 4 days' incubation in mouse serum at +37° C. A. 6-(2-methyl)butanoyl-kifunensine (not detected on day 4). B. 6-(2,2-dimethyl)propanoyl-kifunensine at m/z 339.187 [M+Na]⁺. C. 6-butanoyl-kifunensine (not detected on day 4). D. 6-succinyl-kifunensine at m/z 355.163 [M+Na]⁺. The hydrolysis product kifunensine was visible in all panels A.-D. at m/z 255.1 [M+Na]⁺.

FIG. 4 shows the successful generation of kifunensine-conjugated trastuzumab, DAR=28, from azide-modified trastuzumab, 4 azides/antibody, wherein N-azidoacetylgalactosamine (GalNAz) residues were transferred to N-glycan antenna N-acetylglucosamine residues with mutant galactosyltransferase reaction after cleaving the galactose residues with R-galactosidase. The MALDI-TOF mass spectrum of the heavy chain Fc domain was recorded after isolation of the fragments by Fabricator enzyme digestion showed the expected m/z values after A. GalNAz transfer reaction, at m/z 25717.629 [M+H]⁺, and B. after conjugation of 4 DBCO-PEG4-(octakis-amino)γ-cyclodextrin-(9-(di-3-thio-propanoyl)-kifunensine)₇, DAR=28, at m/z 35046.433 [M+H]⁺.

FIG. 5 shows the successful generation of kifunensine-conjugated trastuzumab, average DAR=9, with 9-(NHS-4,4-dimethyl-4-thio-3-thiopropanoyl)-kifunensine. The MALDI-TOF mass spectrum of the heavy chain Fc domain was recorded after isolation of the fragments by Fabricator enzyme digestion showed the expected m/z values A. before the reaction, at m/z 24146.303 [M+H]⁺, and B. after conjugation, average DAR=9 calculated from the Fc domain data, at m/z 24137.338 (0 kifunensines), m/z 24586.699 (1 kifunensine), m/z 25036.322 (2 kifunensines), m/z 25484.339 (3 kifunensines) and m/z 25932.785 (4 kifunensines), all [M+H]⁺.

FIG. 6 shows effective inhibition of N-glycan processing in SK-BR-3 cells by trastuzumab-kifunensine conjugate (lysineconjugated with 9-(NHS-4,4-dimethyl-4-thio-3-thiopropanoyl)-kifunensine, average DAR=21) treatment. The cells were cultured in standard culture conditions in the presence of the ADC for three days, after which neutral N-glycans were isolated from the cells and analyzed by MALDI-TOF mass spectrometry. A. shows the neutral N-glycan profile before the treatment, with normal distribution of the high-mannose N-glycans Man₅GlcNAc₂ (H5N2), Man₆GlcNAc₂ (H6N2), Man₇GlcNAc₂ (H7N2), Man₈GlcNAc₂ (H8N2) and Man₉GlcNAc₂ (H9N2). B. shows the profile after treatment with 100 pM ADC, with increase in H9N2 and H8N2 relative to decrease in H5N2, H6N2 and H7N2. C. shows the profile after treatment with 1 nM ADC, with increase in especially H9N2, as well as H8N2, relative to decrease in H5N2, H6N2 and H7N2. IC50 for inhibition of mannosidase I activity with the ADC was about 100 pM.

FIG. 7 shows MALDI-TOF mass spectra of the heavy chain Fc domains of trastuzumab after conjugation of NHS-linker payloads to lysine side chains as amide-conjugated ADCs. A. ADC with NHS-DS-kifunensine, with 0-7 linker-payloads/Fc. B. ADC with NHS-MeMe-kifunensine, with 0-5 linker-payloads/Fc. C. ADC with NHS-DiDi-kifunensine, with 0-5 linker-payloads/Fc.

FIG. 8 shows MALDI-TOF mass spectra of the heavy chain Fc domains of the same trastuzumab-linker payload ADCs as in FIG. 7 after incubation in PBS at +37° C. for 7 days. A. ADC with NHS-DS-kifunensine shows release of kifunensine payload by linker hydrolysis (marked with asterisks *), whereas ADCs with NHS-MeMe-kifunensine (B.) and NHS-DiDi-kifunensine (C.) show no signs of payload release in the same conditions.

FIG. 9 shows MALDI-TOF mass spectra of the heavy chain Fc domains of the same trastuzumab-linker payload ADCs as in FIG. 7 and FIG. 8 after incubation in mouse serum at +37° C. for 2 days. A. ADC with NHS-DS-kifunensine shows partial release of kifunensine payload by linker hydrolysis at 2 days in PBS and B. marked release at 2 days in mouse serum (marked with asterisks *), whereas ADCs with NHS-MeMe-kifunensine (C.) and NHS-DiDi-kifunensine (D.) show no signs of payload release in the same conditions.

FIG. 10 shows successful generation of kifunensine-conjugated trastuzumab, DAR=28, via A. glycoconjugation with two GalNAz residues/antibody to antenna GlcNAc residues (Tmab-GalNAz), by B. DBCO-cyclo-MeMe-kifunensine and C. DBCO-cyclo-DiDi-kifunensine. The MALDI-TOF mass spectrum of the heavy chain Fc domain was recorded after isolation of the fragments by Fabricator enzyme digestion showed the expected m/z changes with addition of two linker-payloads/Fc, all [M+H]⁺.

FIG. 11 shows average tumor volumes of syngeneic murine B16-F10 melanoma subcutaneous tumor model in C57BL/6J mice. Treatment groups (n=7) were administered with three 10 mg/kg intravenous infusions of antibody or ADC to the tail vein in PBS on days 2, 7 and 12 after subcutaneous inoculation of 0.5 million B16-F10 cells in each mouse. Anti-PD-1 antibody (n=7) was administered by intraperitoneal injection. Control mice (n=5) received no injections. Plotting of the average tumor size was stopped when first mouse in the group was killed due to tumor growth. Kifunensine-conjugated ADCs showed increased anti-tumor efficacy compared to naked antibody treatment.

### DETAILED DESCRIPTION

### Outline of Sections

I) Definitions

II) Payload molecules

III) Linker units

IV) Stability units

V) Targeting units

VI) Stretcher units

VII) Specificity units

VIII) Spacer units

IX) Further linker units

X) Conjugates

XI) Compositions and methods

### I) Definitions

A conjugate is disclosed.

The conjugate may be represented by Formula I:

[D-O-L]ₙ-T Formula I
wherein D is a payload molecule, wherein the payload molecule is optionally a glycosylation inhibitor or a galectin inhibitor;

O is an oxygen atom of said payload molecule;

T is a targeting unit capable of binding a target molecule, cell and/or tissue; and

n is at least 1.

The conjugate may be represented by Formula I:

[D-O-L]ₙ-T Formula I

wherein D is a payload molecule comprising a sugar moiety;

O is an oxygen atom of said sugar moiety;

T is a targeting unit capable of binding a target molecule, cell and/or tissue; and

n is at least 1.

L may be a linker unit. The linker unit may link the oxygen atom to the targeting unit. The linker unit may link the oxygen atom to the targeting unit covalently. The payload molecule may thus be conjugated to the targeting unit via a relatively stabile bond.

In Formula I, L may be represented by Formula C:

—R₇-L₁-Sₚ-L₂-R₈— Formula C

wherein

R₇ is absent or a group covalently bonded to said oxygen atom (i.e. the oxygen atom of said sugar moiety);

L₁ is a spacer unit of the formula —Sₜ-L₁′-, wherein L₁′ is absent or a spacer moiety;

Sₚ is absent or a specificity unit;

L₂ is absent or a stretcher unit, wherein the stretcher unit optionally comprises a moiety represented by the formula —SₜL₂′-, wherein L₂′ is absent or a stretcher moiety;

R₈ is absent or a group covalently bonded to the targeting unit;

each Sₜ is independently absent or a moiety represented by any one of the formulas LI to LXVII set forth below;

wherein L comprises at least one Sₜ.

The moiety Sₜ, where present, may be referred to as a stability unit in the present disclosure.

In an embodiment, which may be in accordance with any one of the embodiments described above, each Sₜ is independently absent or a moiety represented by formula LI

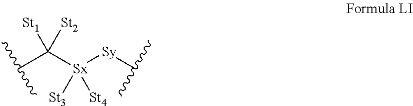

wherein St₁, St₂, St₃, and St₄ are each independently selected from H, CH₃, CH₂CH₃, unsubstituted or substituted C₁-C₆ alkyl, unsubstituted or substituted C₁-C₆ cycloalkyl, unsubstituted or substituted aryl, OH, OCH₃, OR_{O}, wherein R_{O} is either a C₁-C₆ alkyl or a C₁-C₆ substituted alkyl, and an amino acid side chain;

or wherein St₁ together with the carbon to which it is attached, with Sx and optionally with St₃ form an unsubstituted or substituted carbocyclyl or heterocyclyl group;

Sx is either C or N, wherein St₄ is absent if Sx is N;

Sy is either absent, —C(═O)O— or —(CH₂)ₘ—, wherein m is 1 to 4; and

wherein L comprises at least one Sₜ.

R₇ may be a cleavable group.

R₇ may be absent or any one of the groups a-i:
a. —C(═O)—,
b. —C(═O)NH—,
c. —C(═O)O—,
d. —NHC(═O)—,
e. —NHC(═O)O—,
f. —C(═O)NH—,
g. —NHC(═O)NH—,
h. —P(═O)(OH)—, or
i. —S(═O)₂—.

In an embodiment, R₇ may be absent or any one of the groups a, b, c, f, h, or i.

In an embodiment, R₇ may be absent or —C(═O)—.

In an embodiment, R₇ may be —C(═O)—. In such an embodiment, L may be considered to form an ester bond with D, such that the oxygen atom of the sugar moiety of the payload molecule is incorporated in the ester group (—O—C(═O)—) linking the payload molecule to L₁. The ester bond may be cleavable. For example, intercellular esterases may be capable of hydrolyzing the ester bond and thereby releasing the payload molecule where desired.

In an embodiment, R₇ is absent.

In an embodiment, R₇ is —C(═O)—.

In an embodiment, R₇ is —C(═O)NH—.

In an embodiment, R₇ is —C(═O)O—.

In an embodiment, R₇ is —NHC(═O)—.

In an embodiment, R₇ is —NHC(═O)O—.

In an embodiment, R₇ is —C(═O)NH—.

In an embodiment, R₇ is —NHC(═O)NH—.

In an embodiment, R₇ is —P(═O)(OH)—.

In an embodiment, R₇ is —S(═O)₂—.

In an embodiment, R₇ may be absent and L may be considered to form an ether bond with D, such that the oxygen atom of the sugar moiety of the payload molecule forms an ether bond to an alkylene group of L₁. The ether bond may be cleavable when it is a part of a self-immolative group in the linker L. For example, intercellular reducing conditions or peptidases may be capable of cleaving a disulfide or a peptide elsewhere in the linker L, respectively, such that the linker is capable of self-immolating, thereby releasing the payload molecule with a free hydroxyl group.

In an embodiment, R₇ may be —C(═O)O—. In such an embodiment, L may be considered to form a carbonate bond with D, such that the oxygen atom of the sugar moiety of the payload molecule is incorporated in the carbonate ester group (—O—C(═O)O—) linking the payload molecule to L₁. The carbonate bond may be cleavable or self-immolative after the linker is cleaved. For example, intercellular reducing conditions or peptidases may be capable of cleaving a disulfide or a peptide elsewhere in the linker L, respectively, such that the carbonate is capable of self-immolating, thereby releasing the payload molecule where desired.

However, while R₇ may be a cleavable group, it may be desirable for it to be cleavable only or preferably at desired conditions, in particular within a cell to which the payload molecule is to be targeted. If the payload is too unstable and relatively easily cleaved e.g. during preparation or storage in a buffer (for example, in phosphate buffered saline), or in serum, its use may be impractical, and its pharmacological properties may be less than optimal.

Likewise, the specificity unit Sₚ may be cleavable. For example, in embodiments in which the specificity unit Sₚ is a disulfide, the disulfide moiety may be cleavable within a target cell. Again, it may be undesirable for the specificity unit to be cleaved too easily and/or in conditions in which its cleavage is not appropriate.

The presence of the stability unit (Sₜ) in L₁ and/or in L₂ may significantly improve the stability of the conjugate. It may, additionally or alternatively, also significantly improve the pharmacological properties of the conjugate and/or its biological activity. For example, conjugates comprising a stability unit according to one or more embodiments described in this specification may be more stable in buffer (e.g. in phosphate buffered saline) and/or in serum. Some conjugates comprising a stability unit according to one or more embodiments described in this specification may exhibit significant improvements in efficacy (for example, 1000-fold better efficacy) than corresponding conjugates that do not comprise a stability unit.

D may be a payload molecule comprising a sugar moiety.

D may be a glycosylation inhibitor or a galectin inhibitor.

D may be a glycosylation inhibitor or a galectin inhibitor, wherein the glycosylation inhibitor or the galectin inhibitor comprises a sugar moiety.

The payload molecule may be a glycosylation inhibitor selected from the group of a metabolic inhibitor, a cellular trafficking inhibitor, tunicamycin, a plant alkaloid, a substrate analog, a glycoside primer, a specific inhibitor of glycosylation, an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a glycosaminoglycan biosynthesis inhibitor, a glycosphingolipid biosynthesis inhibitor, a sulphation inhibitor, 2-deoxyglucose, a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluorosialic acid, 3-deoxy-3ax-fluorosialic acid, 3-deoxy-3eq-fluorosialic acid, 3-deoxy-3-fluoro-Neu5Ac, 3-deoxy-3ax-fluoro-Neu5Ac, 3-deoxy-3eq-fluoroNeu5Ac, 3-deoxy-3-fluorofucose, 2-deoxy-2-fluoroglucose, 2-deoxy-2-fluoromannose, 2-deoxy-2-fluorofucose, 3-fluorosialic acid, castanospermine, australine, deoxynojirimycin, N-butyldeoxynojirimycin, deoxymannojirimycin, kifunensin, swainsonine, mannostatin A, streptozotocin, 2-acetamido-2,5-dideoxy-5-thioglucosamine, 2-acetamido-2,4-dideoxy-4-thioglucosamine, PUGNAc (0-[2-acetamido-2-deoxy-Dglucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, a glycoside primer, a β-xyloside, a β-N-acetylgalactosaminide, a β-glucoside, a β-galactoside, β-N-acetylglucosaminide, a β-N-acetyllactosaminide, a disaccharide glycoside and a trisaccharides glycosideglucosylceramide epoxide, 2-amino-2-deoxymannose, a 2-acyl-2-deoxy-glucosyl-phosphatidylinositol, Neu5Ac-2-ene (DANA), 4-amino-DANA, 4-guanidino-DANA, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, a concanamycin, concanamycin A, concanamycin B, concanamycin C, a bafilomycin, bafilomycin A1, epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, an N-acyldeoxynojirimycin, N-acetyldeoxynojirimycin, an Nacyldeoxymannojirimycin, N-acetyldeoxymannojirimycin, 3-deoxy-3-fluoro-Neu5N, 3-deoxy-3ax-fluoro-Neu5N, 3-deoxy-3eq-fluoro-Neu5N, 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, 3′-fluoro-2′,3′-dideoxycytidine, and any analogs, modifications, acylated analogs, acetylated analogs, methylated analogs, or combinations thereof.

The payload molecule may be a galectin inhibitor selected from the group of galactose, a 3-substituted galactose, a β-D-galactoside, a galactoside, a 3-substituted galactoside, a β-D-galactoside, a 3-substituted β-D-galactoside, lactose, a 3′-substituted lactose, a lactoside, a 3′-substituted lactoside, N-acetyllactosamine, a 3′-substituted N-acetyllactosamine, an N-acetyllactosaminide, a 3′-substituted N-acetyllactosaminide, N,N′-di-N-acetyllactosediamine, a 3′-substituted N,N′-di-N-acetyllactosediamine, an N,N′-di-N-acetyllactosediaminide, a 3′-substituted N,N′-di-N-acetyllactosediaminide, a taloside, a 3′-substituted taloside, a β-D-taloside, a 3′-substituted β-D-taloside, a mannoside, a 3′-substituted mannoside, a β-D-mannoside, a 3′-substituted β-D-mannoside, thiodigalactose (TDG), a 3-substituted thiodigalactose, a 3,3′-disubstituted thiodigalactose, 3,3′-dideoxy-3,3′-bis-[4-(3-fluorophenyl)-1H1,2,3-triazol-1-yl]-1,1′-sulfanediyl-di-β-D-galactopyranoside (33DFTG or TD139), 6-acyl-33DFTG, 6-succinyl-33DFTG, di-6-acyl-33DFTG, di-6-succinyl-33DFTG, a 6-substituted 33DFTG, a 6,6′-disubstituted 33DFTG, (E)-methyl-2-phenyl-4-(β-D-galactopyranosyl)-but-2-enoate, Galβ1-4Fuc, a 3′-substituted Galβ1-4Fuc, GM-CT-01, GR-MD-02, a pectin, reduced pectin, modified citrus pectin, GCS-100, a poly-N-acetyllactosaminide, lactulose, a lactuloside, a 3′-substituted lactulose, a 3′-substituted lactuloside, lactulosyl-L-leucine, a 3′-substituted lactulosyl-L-leucine, a galectin-binding molecule that inhibits galectin-galectin ligand interaction, an RNAi inhibiting galectin expression, GB1107, and any analog, modification, combination or multivalent combination thereof.

However, other payload molecules may also be contemplated, for example payload molecules containing hydroxyl groups in their unconjugated form.

In an embodiment, D is kifunensine or an analog thereof.

In an embodiment, D is castanospermine or an analog thereof.

In an embodiment, D is a tunicamycin or an analog thereof.

In an embodiment, D is 33DFTG or an analog thereof.

The term “sugar moiety” may be understood as referring to single simple sugar moieties, monosaccharides, sugar analogs or their derivatives, and/or combinations of two or more single sugar moieties or monosaccharides covalently linked to form disaccharides, oligosaccharides, and polysaccharides. A sugar moiety can be a compound or moiety that includes one or more open chain or cyclized monomer units. Thus, the monomer units can include trioses, tetroses, pentoses, hexoses, heptoses, octoses, nonoses, and mixtures thereof. One or several of the hydroxyl groups in the chemical structure can be replaced with other groups such as hydrogen, amino, amine, acylamido, acetylamido, halogen, mercapto, acyl, acetyl, phosphate or sulphate ester, and the like; and the saccharides can also comprise other functional groups such as carboxyl, carbonyl, hemiacetal, acetal and thio groups. Saccharides can include monosaccharides including, but not limited to, simple aldoses such as glyceraldehyde, erythrose, threose, ribose, arabinose, xylose, lyxose, allose, altrose, glucose, mannose, gulose, idose, galactose, talose and mannoheptulose; simple ketoses such as dihydroxyacetone, erythrulose, ribulose, xylulose, psicose, fructose, sorbose, tagatose and sedoheptulose; deoxysugars such as fucose, 2-deoxyglucose, 2-deoxyribose and rhamnose; sialic acids such as ketodeoxynonulosonic acid, N-acetylneuraminic acid and 9-O-acetyl-N-acetylneuraminic acid; uronic acids such as glucuronic acid, galacturonic acid and iduronic acid; amino sugars such as 2-amino-2-deoxygalactose and 2-amino-2-deoxyglucose; acylamino sugars such as 2-acetamido-2- deoxygalactose, 2-acetamido-2-deoxyglucose and N-glycolylneuraminic acid; phosphorylated and sulphated sugars such as 6-phosphomannose, 6-sulpho-N-acetylglucosamine and 3-sulphogalactose; and derivatives and modifications thereof. The term sugar moiety may also include non-reducing carbohydrates, such as inositols and alditols and their derivatives.

The term sugar moiety may also include sugar analogs, such as thiosugars, iminosugars and their derivatives. In this context, a thiosugar may be any analog of a sugar where a sulphur atom has replaced an oxygen atom in the structure. In this context, an iminosugar may be any analog of a sugar where a nitrogen atom has replaced the oxygen atom in the ring of the structure.

Sugar moieties disclosed in this specification may be in D- or L-configuration; in open-chain, pyranose or furanose form; α or β anomer; and/or any combination thereof.

In an embodiment, the sugar moiety is comprised as a covalently bonded part of the payload molecule. In an embodiment, the sugar moiety is a glycoside bound to the rest of the payload molecule. In an embodiment, the sugar moiety is a glycoside bound with an O-glycosidic or an N-glycosidic bond to the rest of the payload molecule.

Carbohydrate nomenclature in this context is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293), unless otherwise stated herein.

The term “oligosaccharide” may be understood as referring to sugar moieties composed of two or several monosaccharides linked together by glycosidic bonds having a degree of polymerization in the range of from 2 to about 20. The term “oligosaccharide” may be understood as referring hetero- and homopolymers that can be either branched or linear and can have a reducing end and a non-reducing end, whether or not the saccharide at the reducing end is in fact a reducing sugar. An oligosaccharide described herein may be described with the name or abbreviation for the non-reducing saccharide, followed by the configuration of the glycosidic bond (α or β), the ring bond, the ring position of the reducing saccharide involved in the bond, and then the name or abbreviation of the reducing saccharide, and so on (e.g. Galβ1-4Glc for lactose and Galα1-4Galβ1-4Glc for globotriose).

The term “disaccharide” may be understood as referring to a sugar moiety composed of two monosaccharides linked together. Examples of disaccharides include, but are not limited to, lactose, N-acetyllactosamine, galactobiose, maltose, isomaltose and cellobiose.

The term “trisaccharide” may be understood as referring to a sugar moiety composed of three monosaccharides linked together. Examples of trisaccharides include, but are not limited to, maltotriose, sialyllactose, globotriose, lacto-N-triose and gangliotriose.

The sugar moiety may, at least in some embodiments, be understood as referring to a sugar moiety or sugar analog containing a 5 to 6 membered ring structure, which may optionally contain a heteroatom (e.g. O, N, or S) as a ring member.

The sugar moiety may be reducing or non-reducing.

For example, the glycosylation inhibitors and galectin inhibitors described in this specification may be considered to contain a sugar moiety.

In an embodiment, which may be in accordance with any embodiment described above, L₁′ is either absent or any one of the groups a-h:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety, or
h. a substituted branched PEG₂₋₅₀ polyethylene glycol moiety.

In an embodiment, which may be in accordance with any embodiment described above, L₁′ is absent.

In an embodiment, which may be in accordance with any embodiment described above, L₁′ is a C₁₋₁₂ alkylene.

In an embodiment, which may be in accordance with any embodiment described above, L₁′ is a substituted C₁₋₁₂ alkylene.

In an embodiment, which may be in accordance with any embodiment described above, L₁′ is a C₅₋₂₀ arylene.

In an embodiment, which may be in accordance with any embodiment described above, L₁′ is a substituted C₅₋₂₀ arylene.

In an embodiment, which may be in accordance with any embodiment described above, L₁′ is a PEG₁₋₅₀ polyethylene glycol moiety.

In an embodiment, which may be in accordance with any embodiment described above, L₁′ is a substituted PEG₁₋₅₀ polyethylene glycol moiety,

In an embodiment, which may be in accordance with any embodiment described above, L₁′ is a branched PEG₂₋₅₀ polyethylene glycol moiety.

In an embodiment, which may be in accordance with any embodiment described above, L₁′ is a substituted branched PEG₂₋₅₀ polyethylene glycol moiety.

In an embodiment, which may be in accordance with any embodiment described above, Sₚ is either absent or any one of the groups a-n:
a. dipeptide,
b. tripeptide,
c. tetrapeptide,
d. valine-citrulline,
e. phenylalanine-lysine,
f. valine-alanine,
g. valine-serine,
h. asparagine,
i. alanine-asparagine,
j. alanine-alanine-asparagine,
k. a hydrazone,
l. an ester,
m. a disulfide, or
n. a glycoside.

In an embodiment, which may be in accordance with any embodiment described above, Sₚ is absent.

In an embodiment, which may be in accordance with any embodiment described above, Sₚ is a dipeptide.

In an embodiment, which may be in accordance with any embodiment described above, Sₚ is a tripeptide.

In an embodiment, which may be in accordance with any embodiment described above, Sₚ is a tetrapeptide.

In an embodiment, which may be in accordance with any embodiment described above, Sₚ is valine-citrulline.

In an embodiment, which may be in accordance with any embodiment described above, Sₚ is phenylalanine-lysine.

In an embodiment, which may be in accordance with any embodiment described above, Sₚ is valine-alanine.

In an embodiment, which may be in accordance with any embodiment described above, Sₚ is valine-serine.

In an embodiment, which may be in accordance with any embodiment described above, Sₚ is asparagine.

In an embodiment, which may be in accordance with any embodiment described above, Sₚ is alanine-asparagine.

In an embodiment, which may be in accordance with any embodiment described above, Sₚ is alanine-alanine-asparagine.

In an embodiment, which may be in accordance with any embodiment described above, Sₚ is a hydrazine.

In an embodiment, which may be in accordance with any embodiment described above, Sₚ is an ester.

In an embodiment, which may be in accordance with any embodiment described above, Sₚ is a disulfide.

In an embodiment, which may be in accordance with any embodiment described above, Sₚ is a glycoside.

In an embodiment, which may be in accordance with any embodiment described above, L₂′ is either absent or any one of the groups a-j:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety,
h. a substituted branched PEG₂₋₅₀ polyethylene glycol moiety,
i. a moiety represented by the formula XXVI, or
j. a moiety represented by the formula XXVII.

In an embodiment, which may be in accordance with any embodiment described above, L₂′ is absent.

In an embodiment, which may be in accordance with any embodiment described above, L₂′ is a C₁₋₁₂ alkylene.

In an embodiment, which may be in accordance with any embodiment described above, L₂′ is a substituted C₁₋₁₂ alkylene.

In an embodiment, which may be in accordance with any embodiment described above, L₂′ is a C₅₋₂₀ arylene.

In an embodiment, which may be in accordance with any embodiment described above, L₂′ is a substituted C₅₋₂₀ arylene.

In an embodiment, which may be in accordance with any embodiment described above, L₂′ is a PEG₁₋₅₀ polyethylene glycol moiety.

In an embodiment, which may be in accordance with any embodiment described above, L₂′ is a substituted PEG₁₋₅₀ polyethylene glycol moiety.

In an embodiment, which may be in accordance with any embodiment described above, L₂′ is a branched PEG₂₋₅₀ polyethylene glycol moiety.

In an embodiment, which may be in accordance with any embodiment described above, L₂′ is a substituted branched PEG₂₋₅₀ polyethylene glycol moiety.

In an embodiment, which may be in accordance with any embodiment described above, L₂′ is a moiety represented by the formula XXVI.

In an embodiment, which may be in accordance with any embodiment described above, L₂′ is a moiety represented by the formula XXVII.

Formulas XXVI and XXVII are described in detail below.

In an embodiment, which may be in accordance with any embodiment described above, R₈ is either absent or any one of the groups a-k:
a. —C(═O)NH—,
b. —C(═O)O—,
c. —NHC(═O)—,
d. —OC(═O)—,
e. —OC(═O)O—,
f. —NHC(═O)O—,
g. —OC(═O)NH—,
h. —NHC(═O)NH,
i. —NH—,
j. —O—, or
k. —S—.

In an embodiment, which may be in accordance with any embodiment described above, R₈ is absent.

In an embodiment, which may be in accordance with any embodiment described above, R₈ is —C(═O)NH—.

In an embodiment, which may be in accordance with any embodiment described above, R₈ is —C(═O)O—.

In an embodiment, which may be in accordance with any embodiment described above, R₈ is —NHC(═O)—.

In an embodiment, which may be in accordance with any embodiment described above, R₈ is —OC(═O)—.

In an embodiment, which may be in accordance with any embodiment described above, R₈ is —OC(═O)O—.

In an embodiment, which may be in accordance with any embodiment described above, R₈ is —NHC(═O)O—.

In an embodiment, which may be in accordance with any embodiment described above, R₈ is —OC(═O)NH—.

In an embodiment, which may be in accordance with any embodiment described above, R₈ is —NHC(═O)NH.

In an embodiment, which may be in accordance with any embodiment described above, R₈ is —NH—.

In an embodiment, which may be in accordance with any embodiment described above, R₈ is —O—.

In an embodiment, which may be in accordance with any embodiment described above, R₈ is —S—.

In an embodiment, which may be in accordance with any embodiment described above, each Sₜ is independently absent, a moiety represented by formula LI, wherein St₃ and St₄ are optionally absent, or a moiety represented by formula LII, LIII, LIV, LV, LVI, LVII, LVIII, LIX, LX, LXI, LXII, LXIII, LXIV, LXV, LXVI or LXVIII:

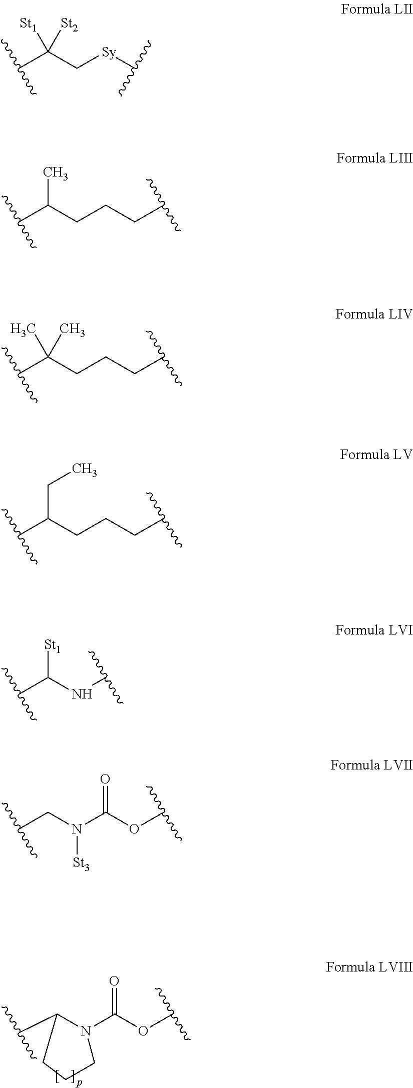

wherein p is from 1 to 2;

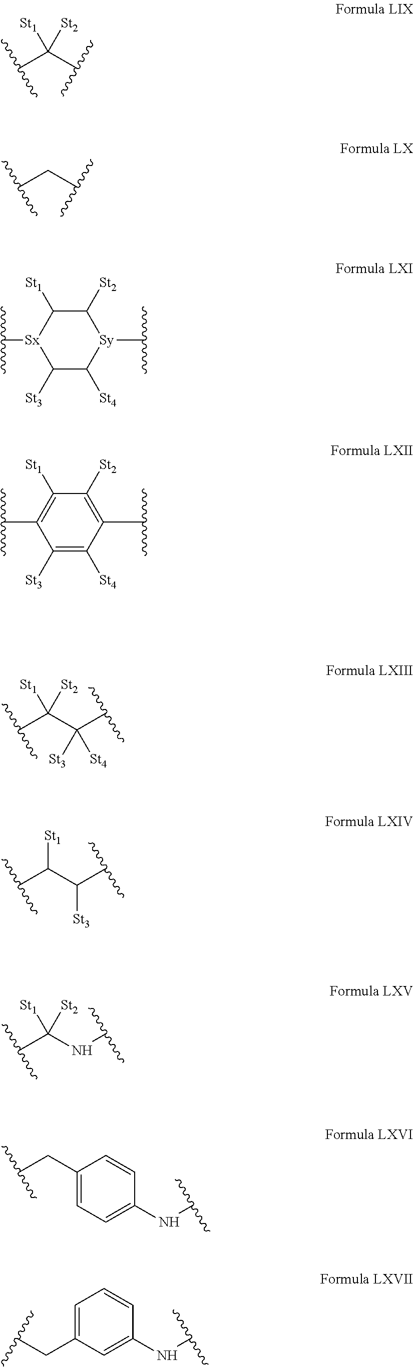

wherein the wavy lines in Formulas LII-LXVII show the bonds to the rest of the structure;

St₁, St₂, St₃, St₄, Sx, and Sy are as defined above or according to any embodiment described in this specification; and the stereochemical centers in any one of the Formulas LII-LXVII are in either the R or S configuration or a racemic mixture.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LI, wherein St₃ and St₄ are optionally absent, or a moiety represented by formula LII, LIII, LIV, LV, LVI, LVII, LVIII, LIX, LX, LXI, LXII, LXIII, LXIV, LXV, LXVI or LXVII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LII, LIII, LIV, LV, LVI, LVII, LVIII, LIX, LX, LXI, LXII, LXIII, LXIV, LXV, LXVI or LXVII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LIII.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LIV.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LV.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LVI according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LVII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LVIII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LIX according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LX.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXI according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXIII according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXIV according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXV according to one or more embodiments described in this specification.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXVI.

In an embodiment, which may be in accordance with any embodiment described above, L₂ is a stretcher unit comprising a moiety represented by the formula —Sₜ-L₂′-, wherein L₂′ is absent or a stretcher moiety; and Sₜ is a moiety represented by formula LXVII according to one or more embodiments described in this specification.

A pharmaceutical composition comprising the conjugate according to one or more embodiments described in this specification is also disclosed.

The conjugate according to one or more embodiments described in this specification or a pharmaceutical composition comprising the conjugate according to one or more embodiments described in this specification for use as a medicament, for use in the modulation or prophylaxis of the growth of tumour cells, or for use in the treatment of cancer, is also disclosed.

The conjugate or the pharmaceutical composition for use according to one or more embodiments described in this specification is also disclosed, wherein the cancer is selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer.

A method for preparing the conjugate according to one or more embodiments described in this specification is also disclosed, the method comprising conjugating the payload molecule to the targeting unit.

The conjugate may comprise a targeting unit for delivery to a tumour.

The conjugate may comprise a targeting unit for delivery to a tumour, and a glycosylation inhibitor for inhibiting glycosylation in the tumour, thereby decreasing the immunosuppressive activity of the tumour.

The conjugate may be a conjugate for decreasing the immunosuppressive activity of a target cell, which is a tumour cell, and/or of a second tumour cell.

The conjugate may thus comprise a targeting unit for delivery to the tumour, and a glycosylation inhibitor for inhibiting glycosylation in the tumour, for example in the target cell or in the second tumour cell, thereby decreasing the immunosuppressive activity of the tumour, for example the immunosuppressive activity of the target cell and/or of the second tumour cell.

Many tumours are known to be formed of not only malignant or cancer cells, but also of non-malignant or non-cancer cells of the subject having the tumour. Such non-malignant or non-cancer cells may be migrated to the tumour, so that they are located within the tumour or the tumour microenvironment or otherwise be intimately associated with the tumour. For example, such nonmalignant or non-cancer cells may be located between the malignant or cancer cells, or they may be in direct physical contact with the malignant or cancer cells.

In the context of this specification, the term “tumour cell” may refer to any cell of any cell type that forms a part of or is associated with a tumour. The term may encompass malignant or cancer cells and, additionally or alternatively, non-cancer or non-malignant cells that form a part of or are associated with the tumour. The term may also encompass any non-cancer or non-malignant cell present in the tumour microenvironment. The tumour cells may include, for example, cells of the immune system. Examples of such tumour cells may include tumour infiltrating immune cells, such as tumour infiltrating lymphocytes, cells of the immune system, cells of the tumour vasculature and lymphatics, as well as fibroblasts, pericytes and adipocytes. Specific examples of such non-cancer tumour cells may include T cells (T lymphocytes); CD8+ cells including cytotoxic CD8+ T cells; CD4+ cells including T helper 1 (TH1) cells, TH2 cells, TH17 cells, Tregs; γδ T lymphocytes; B lymphocytes including B cells and Bregs (B10 cells); NK cells; NKT cells; tumour-associated macrophages (TAMs); myeloid-derived suppressor cells (MDSCs); dendritic cells (DCs); tumour-associated neutrophils (TANs); CD11b+ bone-marrow-derived myeloid cells; fibroblasts including myofibroblasts and cancer-associated fibroblasts; endothelial cells; smooth muscle cells; myoepithelial cells; stem cells including multipotent stem cells, lineagespecific stem cells, progenitor cells, pluripotent stem cells, cancer stem cells (cancer-initiating cells), mesenchymal stem cells and hematopoietic stem cells; adipocytes; vascular endothelial cells; stromal cells; perivascular stromal cells (pericytes); and lymphatic cells including lymphatic endothelial cells (Balkwill et al. 2012. J. Cell Sci. 125:5591-6), provided they form a part of or are associated with the tumour.

In other words, the tumour cells, which thus may form a tumour, may comprise at least malignant or cancer cells and noncancer or non-malignant cells that form a part of or are associated with the tumour. The target cell may be at least one of the malignant or cancer cells or the non-cancer or non-malignant cells (for example, cells of the immune system). Likewise, the second tumour cell may be at least one of the malignant or cancer cells or the non-cancer or non-malignant cells (for example, cells of the immune system).

The targeting unit may be suitable for delivery to the tumour in various ways, for example for binding the tumour, e.g. the target cell or a molecule within the tumour.

In an embodiment, the targeting unit may bind or be capable of binding to a tumour molecule, thereby facilitating the delivery of the conjugate to the tumour or to any cells of the tumour.

In the context of this specification, the term “tumour molecule” may refer to any molecule of any molecule type that forms a part of or is associated (for example, intimately associated) with a tumour. The term may encompass molecules produced by the malignant or cancer cells and, additionally or alternatively, molecules produced by the non-cancer or non-malignant cells that form a part of or are associated with the tumour and, additionally or alternatively, molecules that are produced by non-tumour cells and that form a part of or are associated with the tumour. The term may also encompass any molecule present in the tumour microenvironment. The tumour molecules may include, for example, proteins, lipids, glycans, nucleic acids, or combinations thereof. The tumour molecule may, in some embodiments, be specific to the tumour or enriched in the tumour.

Upon or after binding to a tumour molecule, the conjugate may release the payload molecule, such that the payload molecule may, for example, enter or otherwise interact with the target cell or, in some embodiments, the second tumour cell.

By inhibiting glycosylation or galectins in the tumour, for example in the target cell, the conjugate may be capable of decreasing the immunosuppressive activity of the tumour, for example of the target cell. However, additionally or alternatively, by inhibiting glycosylation or galectins in the target cell, the conjugate may be capable of decreasing the immunosuppressive activity of the second tumour cell. For example, the inhibition may cause the target cell to have altered glycosylation structures, e.g. as a part of membrane-bound or secreted tumour proteins. These altered glycosylation structures may then interact with the second tumour cell within the tumour microenvironment, thereby decreasing the immunosuppressive activity of the second tumour cell. Alternatively or additionally, the conjugate may be capable of (or suitable for) decreasing the immunosuppressive activity of the first and/or the second tumour cell by inhibiting galectins in the tumour.

In an embodiment, the conjugate is a conjugate for decreasing the immunosuppressive activity of the target cell.

In an embodiment, the conjugate is a conjugate for decreasing the immunosuppressive activity of the second tumour cell.

In an embodiment, the conjugate is a conjugate for decreasing the immunosuppressive activity of the target cell and of the second tumour cell.

The tumour cells may have immunosuppressing receptors. The conjugate may thus be suitable for decreasing, or configured to decrease, the immunosuppressive activity of the tumour, e.g. of the target cell and/or of the second tumour cell, for example by reducing the activity of one or more of the immunosuppressing receptors of the target cell and/or of the second tumour cell. In an embodiment, the conjugate may be suitable for reducing, or configured to reduce, glycosylation-cellular receptor interactions, for example glycosylation-lectin interactions. The conjugate may thereby reduce immunosuppression by reducing the activity of one or more of the immunosuppressing receptors of the the target cell and/or of the second tumour cell.

In an embodiment, the conjugate is suitable for decreasing, or configured to decrease, interactions between immunosuppressive receptors and glycan ligands of the target cell and/or of the second tumour cell.

In an embodiment, the conjugate is suitable for decreasing, or configured to decrease, galectin-galectin ligand interactions and/or Siglec-Siglec ligand interactions. The term “Siglec” may be understood as referring to any sialic acid-recognizing receptor within the Siglec subgroup of mammalian I-type lectins. There are at least 17 Siglecs discovered in mammals, of which at least Siglec-1, -2, -3, -4, -5, -6, -7, -8, -9, -10, -11, -12, -14, -15, -16 and -17 have been identified in humans (Varki et al., eds., Essentials of Glycobiology, 2017, 3rd edition, Cold Spring Harbor Laboratory Press, New York; Chapter 35). The term “galectin” may be understood as referring to any S-type lectin, which is a galactoside-recognizing receptor. There are at least 15 galectins discovered in mammals, encoded by the LGALS genes, of which at least galectin-1, -2, -3, -4, -7, -8, -9, -10, -12 and -13 have been identified in humans (Essentials of Glycobiology 2017; Chapter 36).

The conjugate may thus be suitable for increasing, or configured to increase, the activity of the target cell, which may be a cell of the immune system, against the second tumour cell, such as a malignant or cancer cell.

The conjugate may thus be suitable for increasing, or configured to increase, the activity of the second tumour cell, which may be a cell of the immune system, against the target cell, such as a malignant or cancer cell.

As the payload molecule and the targeting unit are conjugated at least partially covalently, it may assist in delivering the payload molecule to the target cell and/or to the second tumour cell. The conjugate may also exhibit improved pharmacodynamics and/or pharmacokinetics. Preparing of the conjugate may also be relatively feasible and cost-effective.

In the context of this specification, the term “tumour” may refer to a solid tumour, a diffuse tumour, a metastasis, a tumour microenvironment, a group of tumour cells, a single tumour cell and/or a circulating tumour cell.

In the context of this specification, the term “target cell” may refer to one or more embodiments of the tumour cells, including malignant or cancer cells and/or non-malignant or non-cancer cells, for example cells of the immune system. The target cell may refer to one or more of the tumour cell types. In an embodiment, the target cell may be at least one of a malignant or cancer cell or a non-malignant or non-cancer cell. In an embodiment, the target cell may be a malignant or cancer cell. In an embodiment, the target cell may be a tumour cell that is nonmalignant or non-cancer cell, such as a tumour-infiltrating immune cell. The conjugate or a part thereof, for example the payload molecule, such as a glycosylation inhibitor or a galectin inhibitor, may subsequently be transported or otherwise move to other tumour cells. Additionally or alternatively, the target cell may be a non-malignant or non-cancer cell, such as a tumour-infiltrating immune cell, and the glycosylation inhibitor may inhibit glycosylation in the target cell itself, thereby reducing the activity of at least a part of the immunosuppressing receptors of the target cell.

In the context of this specification, the term “second tumour cell” may refer to one or more embodiments of the tumour cells, including malignant or cancer cells and/or non-malignant or non-cancer cells, for example cells of the immune system. The second tumour cell may refer to or comprise one or more of the tumour cell types. In an embodiment, the second tumour cell may be at least one of a malignant or cancer cell or a non-malignant or non-cancer cell. In an embodiment, the second tumour cell may be a malignant or cancer cell. In an embodiment, the second tumour cell may be a tumour cell that is non-malignant or non-cancer cell, such as a tumour-infiltrating immune cell.

In the context of this specification, the term “target molecule” may refer to one or more embodiments of the tumour molecules.

In the context of this specification, the term “targeting unit” may refer to a group, moiety or molecule capable of recognizing and binding to the target cell or the target molecule.

The targeting unit may be capable of binding to the target cell specifically. The targeting unit may be capable of binding to the target molecule specifically.

In the context of this specification, the term “glycosylation inhibitor” may refer to any group, moiety or molecule which is capable of inhibiting glycosylation in the target cell or in the second tumour cell, to which the conjugate or a part thereof may be transported or otherwise moved after binding to the target cell or the target molecule. As glycosylation is a complex process involving various biosynthetic steps and mechanisms, the glycosylation inhibitor may in principle inhibit any step or aspect of the glycosylation, such that it decreases, interferes with or prevents the incorporation of glycan structures at the cell surface of one or more embodiments of the tumour cells, for example into glycoproteins and/or glycolipids.

In the context of this specification, the term “to conjugate” or “conjugated” may be understood as referring to linking groups, moieties or molecules, for example the payload molecule and the targeting unit, to each other least partially covalently; however such that the linking may, in some embodiments, be arranged at least partially non-covalently. For example, the targeting unit and the payload molecule may be conjugated via a linker unit, such that separate ends of the linker unit are conjugated covalently to the targeting unit and to the payload molecule, respectively. The targeting unit and the payload molecule may, in an embodiment, be conjugated covalently.

However, they may be conjugated such that at least a part of the linker unit may comprise units, groups, moieties or molecules that are linked non-covalently, for example via a non-covalent interaction. An example of such a non-covalent interaction may be biotin-avidin interaction or other non-covalent interaction with a sufficient affinity.

A sufficient affinity for the non-covalent linkage or non-covalent interaction may be e.g. one having a dissociation constant (Kd) in the order of nanomolar Kd, picomolar Kd, femtomolar Kd, attomolar Kd, or smaller. In an embodiment, the affinity is substantially the same as the affinity of biotin-avidin interaction. The affinity may be an affinity with a Kd of about 10⁻¹⁴ mol/l, or to a Kd between 10⁻¹⁵ mol/l and 10⁻¹² mol/l (femtomolar), or a Kd below 10⁻¹⁵ mol/l (attomolar). In an embodiment, the affinity is substantially the same as the affinity of an antibody-antigen interaction, such as an affinity having a Kd of about 10⁻⁹ mol/l, or a Kd of between 10⁻¹² mol/l and 10⁻⁹ mol/l (picomolar), or a Kd of between 10⁻⁹ mol/l and 10⁻⁷ mol/l (nanomolar). In an embodiment, the affinity may be an affinity with a Kd that is below 10⁻⁷ mol/l, below 10⁻⁸ mol/l, below 10⁻⁹ mol/l, below 10⁻¹⁰ mol/l, below 10-11 mol/l, below 10⁻¹² mol/l, below 10⁻¹ mol/l, below 10-14 mol/l, or below 10-15 mol/l.

The conjugate may comprise one or more chemical substituents as described by the variables of the chemical formulas of the present disclosure. A person skilled in the art is able to determine what structures are encompassed in the specific substituents based on their names. In the context of this specification, the term “to substitute” or “substituted” may be understood as referring to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known to a person skilled in the art.

In the context of the present specification, the substituents may further comprise certain chemical structures as described in the following embodiments.

In an embodiment, the term “alkyl” means a monovalent moiety obtained or obtainable by removing a hydrogen atom from a carbon atom of a hydrocarbon compound, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, and the like. In an embodiment, the term “C₁₋₁₂ alkyl” means an alkyl moiety having from 1 to 12 carbon atoms.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include isopropyl (C₃), iso-butyl (C₄), sec-butyl (C₄), tert-butyl (C₄), isopentyl (C₅), and neo-pentyl (C₅).

In an embodiment, the term “alkenyl” means an alkyl group having one or more carbon-carbon double bonds. In an embodiment, the term “C₂₋₁₂ alkenyl” means an alkenyl moiety having from 2 to 12 carbon atoms.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

In an embodiment, the term “alkynyl” means an alkyl group having one or more carbon-carbon triple bonds. In an embodiment, the term “C₂₋₁₂ alkynyl” means an alkynyl moiety having from 2 to 12 carbon atoms.

Examples of unsaturated alkynyl groups include, but are not limited to, ethynyl (ethinyl, —C≡CH) and 2-propynyl (propargyl, —CH₂—C≡CH).

In an embodiment, the term “cycloalkyl” means an alkyl group which is also a cyclyl group; that is, a monovalent moiety obtained by removing a hydrogen atom from an alicyclic ring atom of a cyclic hydrocarbon (carbocyclic) compound. In an embodiment, the term “C₃₋₂₀ cycloalkyl” means a cycloalkyl moiety having from 3 to 20 carbon atoms, including from 3 to 8 ring atoms.

Examples of cycloalkyl groups include, but are not limited to, those derived from:

saturated monocyclic hydrocarbon compounds: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

saturated polycyclic hydrocarbon compounds: norcarane (C₇), norpinane (C₇), norbornane (C₇).

In an embodiment, the term “heterocyclyl” means a monovalent moiety obtained by removing a hydrogen atom from a ring atom of a heterocyclic compound, which moiety has from 3 to 20 ring atoms, of which from 1 to 10 are ring heteroatoms. In an embodiment, each ring has from 3 to 8 ring atoms, of which from 1 to 4 are ring heteroatoms.

In this context, the prefixes (e.g. C₃₋₂₀, C₃₋₈, C₅₋₆, etc.) denote the number of ring atoms, or range of number of ring atoms, whether carbon atoms or heteroatoms. For example, the term “C₅₋₆ heterocyclyl”, means a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆); N₂O₁: oxadiazine (C₆);

O₁S₁: oxathiole (C₅) and oxathiane (thioxane) (C₆); and, N₁O₁S₁: oxathiazine (C₆).

Examples of substituted monocyclic heterocyclyl groups include those derived from saccharides, in cyclic form, for example, furanoses (C₅), such as arabinofuranose, ribofuranose, and xylofuranose, and pyranoses (C₆), such as fucopyranose, glucopyranose, mannopyranose, idopyranose, and galactopyranose.

In an embodiment, the term “aryl” means a monovalent moiety obtained by removing a hydrogen atom from an aromatic ring atom of an aromatic compound, which moiety has from 3 to 20 ring atoms. For example, each ring may have from 5 to 8 ring atoms.

In this context, the prefixes (e.g. C₃₋₂₀, C₅₋₈, etc.) denote the number of ring atoms, or range of number of ring atoms, whether carbon atoms or heteroatoms. For example, the term “C₅₋₆ aryl” as used herein, means an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryls which comprise fused rings, include, but are not limited to:

C₉ (with 2 fused rings) derived from benzofuran (O₁), isobenzofuran (O₁), indole (N₁), isoindole (_{N1}), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ (with 2 fused rings) derived from chromene (O₁), isochromene (O₁), chroman (O₁), isochroman (O₁), benzodioxan (O₂) quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ (with 2 fused rings) derived from benzodiazepine (N₂);

C₁₃ (with 3 fused rings) derived from carbazole (N₁), dibenzofuran (O₁), dibenzothiophene (S₁), carboline (N₂), perimidine (N₂), pyridoindole (N₂); and,

C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₂S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below. Further, the substituents listed below may themselves be substituents.

Halo: —F, —Cl, —Br, and —I.

Hydroxy: —OH.

Ether: —OR, wherein R is an ether substituent, for example, a C₁₋₁₀ alkyl group (also referred to as a C₁₋₁₀ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₁₀ alkyl group.

Alkoxy: —OR′, wherein R′ is an alkyl group, for example, a C₁₋₁₀ alkyl group. Examples of C₁₋₁₀ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR′₁) (OR′₂), wherein R′₁ and R′₂ are independently acetal substituents, for example, a C₁₋₁₀ alkyl group, a C₃₋₂₀ heterocyclyl group, or a C₅₋₂₀ aryl group, preferably a C₁₋₁₀ alkyl group, or, in the case of a “cyclic” acetal group, R′₁ and R′₂, taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe) (OEt).

Hemiacetal: —CH(OH) (OR′₁), wherein R′₁ is a hemiacetal substituent, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —CH(OH) (OMe) and —CH(OH) (OEt).

Ketal: —CR′ (OR′₁) (OR′₂), where R′₁ and R′₂ are as defined for acetals, and R′ is a ketal substituent other than hydrogen, for example, a C₁₋₁₀ alkyl group, a C₃₋₂₀ heterocyclyl group, or a C₅₋₂₀ aryl group, preferably a C₁₋₁₀ alkyl group. Examples ketal groups include, but are not limited to, —C(Me) (OMe)₂, —C(Me) (OEt)₂, —C(Me) (OMe) (OEt), —C(Et) (OMe)₂, —C(Et) (OEt)₂, and —C(Et) (OMe) (OEt).

Hemiketal: —CR′ (OH) (OR′₁), where R′₁ is as defined for hemiacetals, and R′ is a hemiketal substituent other than hydrogen, for example, a C₁₋₁₀ alkyl group, a C₃₋₂₀ heterocyclyl group, or a C₅₋₂₀ aryl group, preferably a C₁₋₁₀ alkyl group. Examples of hemiacetal groups include, but are not limited to, —C(Me) (OH) (OMe), —C(Et) (OH) (OMe), —C(Me) (OH) (OEt), and —C(Et) (OH) (OEt).

Oxo (keto, -one): ═O.

Thione (thioketone): ═S.

Imino (imine): ═NR′, wherein R′ is an imino substituent, for example, hydrogen, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, ═NH, ═NMe, =NEt, and ═NPh.

Formyl (carbaldehyde, carboxaldehyde): —C(═O)H.

Acyl (keto): —C(═O)R′, wherein R′ is an acyl substituent, for example, a C₁₋₁₀ alkyl group (also referred to as C₁₋₁₀ alkylacyl or C₁₋₁₀ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₁₀ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O) OR′, wherein R′ is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R′, wherein R′ is an acyloxy substituent, for example, a C₁₋₁₀ alkyl group, a C₃₋₂₀ heterocyclyl group, or a C₅₋₂₀ aryl group, preferably a C₁₋₁₀ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₁₀ alkyl group, a C₃₋₂₀ heterocyclyl group, or a C₅₋₂₀ aryl group, preferably a C₁₋₁₀ alkyl group. Examples of ester groups include, but are not limited to, —OC(═O)OCH₃, —OC(═O) OCH₂CH₃, —OC(═O) OC(CH₃)₃, and —OC(═O) OPh.

Amino: —NR′₁R′₂, wherein R′₁ and R′₂ are independently amino substituents, for example, hydrogen, a C₁₋₁₀ alkyl group (also referred to as C₁₋₁₀ alkylamino or di-C₁₋₁₀ alkylamino), a C₃₋₂₀ heterocyclyl group, or a C₅₋₂₀ aryl group, preferably H or a C₁₋₁₀ alkyl group, or, in the case of a “cyclic” amino group, R′₁ and R′₂, taken together with the nitrogen atom to which they are attached, form a heterocyclic ring having from 4 to 8 ring atoms. Amino groups may be primary (—NH₂), secondary (—NHR′₁), or tertiary (—NHR′₁R′₂), and in cationic form, may be quaternary (—NR′₁R′₂R′₃). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR′₁R′₂, wherein R′₁ and R′₂ are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′₁ and R′₂, together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S) NR′₁R′₂, wherein R′₁ and R′₂ are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR′₁C(═O)R′₂, wherein R′₁ is an amide substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group, and R′₂ is an acyl substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of acylamide groups include, but are not limited to, —NHC(═O)CH₃, —NHC(═O)CH₂CH₃, and —NHC(═O)Ph. R′₁ and R′₂ may together form a cyclic structure, as in, for example, succinimidyl, maleimidyl, and phthalimidyl:

Aminocarbonyloxy: —OC(═O)NR′₁R′₂, wherein R′₁ and R′₂ are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R′₁)C(═O)NR′₂R′₃ wherein R′₂ and R′₃ are independently amino substituents, as defined for amino groups, and R′₁ is a ureido substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of ureido groups include, but are not limited to, —NHCONH₂, —NHCONHMe, —NHCONHEt, —NHCONMe₂, —NHCONEt₂.

NMeCONH₂, —NMeCONHMe, —NMeCONHEt, —NMeCONMe₂, and —NMeCONEt₂.

Guanidino: —NH—C(═NH) NH₂.

Tetrazolyl: a five membered aromatic ring having four nitrogen atoms and one carbon atom.

Imino: ═NR′, wherein R′ is an imino substituent, for example, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of imino groups include, but are not limited to, ═NH, ═NMe, and =NEt.

Amidine (amidino): —C(═NR′₁) NR′₂, wherein each R′, is an amidine substituent, for example, hydrogen, a C₁₋₁₀ alkyl group, a C₃₋₂₀ heterocyclyl group, or a C₅₋₂₀ aryl group, preferably hydrogen or a C₁₋₁₀ alkyl group. Examples of amidine groups include, but are not limited to, —C(═NR′₁)NH₂, —C(═NH)NMe₂, and —C(═NMe)NMe₂.

Nitro: —NO2.

Nitroso: —NO.

Azido: —N3.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR′, wherein R′ is a thioether substituent, for example, a C₁₋₁₀ alkyl group (also referred to as a C₁₋₁₀ alkylthio group), a C₃₋₂₀ heterocyclyl group, or a C₅₋₂₀ aryl group, preferably a C₁₋₁₀ alkyl group. Examples of C₁₋₁₀ alkylthio groups include, but are not limited to, —SCH₃ and —SCH₂CH₃.

Disulfide: —SS—R′, wherein R′ is a disulfide substituent, for example, a C₁₋₁₀ alkyl group, a C₃₋₂₀ heterocyclyl group, or a C₅₋₂₀ aryl group, preferably a C₁₋₁₀ alkyl group (also referred to herein as C₁₋₁₀ alkyl disulfide). Examples of C₁₋₁₀ alkyl disulfide groups include, but are not limited to, —SSCH₃ and —SSCH₂CH₃.

Sulfine (sulfinyl, sulfoxide): —S(═O)R′, wherein R′ is a sulfine substituent, for example, a C₁₋₁₀ alkyl group, a C₃₋₂₀ heterocyclyl group, or a C₅₋₂₀ aryl group, preferably a C₁₋₁₀ alkyl group. Examples of sulfine groups include, but are not limited to, —S(═O)CH₃ and —S(═O)CH₂CH₃.

Sulfone (sulfonyl): —S(═O)₂R′, wherein R′ is a sulfone substituent, for example, a C₁₋₁₀ alkyl group, a C₃₋₂₀ heterocyclyl group, or a C₅₋₂₀ aryl group, preferably a C₁₋₁₀ alkyl group, including, for example, a fluorinated or perfluorinated C₁₋₁₀ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O) OH, —SO₂H.

Sulfonic acid (sulfo): —S(═O)₂OH, —SO₃H.

Sulfinate (sulfinic acid ester): —S(═O) OR′; wherein R′ is a sulfinate substituent, for example, a C₁₋₁₀ alkyl group, a C₃₋₂₀ heterocyclyl group, or a C₅₋₂₀ aryl group, preferably a C₁₋₁₀ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR′, wherein R′ is a sulfonate substituent, for example, a C₁₋₁₀ alkyl group, a C₃₋₂₀ heterocyclyl group, or a C₅₋₂₀ aryl group, preferably a C₁₋₁₀ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R′, wherein R is a sulfinyloxy substituent, for example, a C₁₋₁₀ alkyl group, a C₃₋₂₀ heterocyclyl group, or a C₅₋₂₀ aryl group, preferably a C₁₋₁₀ alkyl group. Examples of sulfinyloxy groups include, but are not limited to, —OS(═O)CH₃ and —OS(═O)CH₂CH₃.

Sulfonyloxy: —OS(═O)₂R′, wherein R′ is a sulfonyloxy substituent, for example, a C₁₋₁₀ alkyl group, a C₃₋₂₀ heterocyclyl group, or a C₅₋₂₀ aryl group, preferably a C₁₋₁₀ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR′; wherein R′ is a sulfate substituent, for example, a C₁₋₁₀ alkyl group, a C₃₋₂₀ heterocyclyl group, or a C₅₋₂₀ aryl group, preferably a C₁₋₁₀ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR′₁R′₂, wherein R′₁ and R′₂ are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR′₁R′₂, wherein R′₁ and R′₂ are independently amino substituents, as defined for amino groups. Examples of sulfonamido groups include, but are not limited to, —S(═O)₂NH₂. —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR′S(═O)₂OH, wherein R′ is an amino substituent, as defined for amino groups. Examples of sulfamino groups include, but are not limited to, —NHS(═O)₂OH and —N(CH₃) S(═O)₂OH.

Sulfonamino: —NR′₁S(═O)₂R′₂, wherein R′₁ is an amino substituent, as defined for amino groups, and R′₂ is a sulfonamino substituent, for example, a C₁₋₁₀ alkyl group, a C₃₋₂₀ heterocyclyl group, or a C₅₋₂₀ aryl group, preferably a C₁₋₁₀ alkyl group. Examples of sulfonamino groups include, but are not limited to, —NHS(═O)₂CH₃ and —N(CH₃) S(═O)₂C₆H₅.

Phosphino (phosphine): —P(R′)₂, wherein R′ is a phosphino substituent, for example, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O) (R′)₂, wherein R′ is a phosphinyl substituent, for example, a C₁₋₁₀ alkyl group, a C₃₋₂₀ heterocyclyl group, or a C₅₋₂₀ aryl group, preferably a C₁₋₁₀ alkyl group or a C₅₋₂₀ aryl group. Examples of phosphinyl groups include, but are not limited to, —P(═O)(CH₃)₂, —P(═O)(CH₂CH₃)₂, —P(═O)(tBu)₂, and —P(═O)(Ph)₂.

Phosphonic acid (phosphono): —P(═O)(OH)₂.

Phosphonate (phosphono ester): —P(═O)(OR′)₂, where R′ is a phosphonate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR′)₂, where R′ is a phosphate substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR′)₂, where R′ is a phosphite substituent, for example, hydrogen, a C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR′₁)—N(R′₂)₂, where R′₁ and R′₂ are phosphoramidite substituents, for example, hydrogen, a (optionally substituted) C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR′₁)—N(R′₂)₂, where R′₁ and R′₂ are phosphoramidate substituents, for example, hydrogen, a (optionally substituted) C₁₋₁₀ alkyl group, a C₃₋₂₀ heterocyclyl group, or a C₅₋₂₀ aryl group, preferably hydrogen, a C₁₋₁₀ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

In an embodiment, the term “alkylene” (or “alkanediyl”) means a bidentate or bivalent moiety obtainable by removing two hydrogen atoms, either both from the same carbon atom, or one from each of two different carbon atoms, of a hydrocarbon compound, which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

In an embodiment, the term “arylene” (or “arenediyl”) means a bivalent group or moiety that is derived from an aromatic hydrocarbon obtainable by removing a hydrogen atom from two carbon atoms. Examples of arylenes include e.g. phenylene.

In an embodiment, the term “glycoside” means a carbohydrate or glycan moiety that is joined by a glycosidic bond.

The glycosidic bond may be an O-, N-, C- or S-glycosidic bond, meaning that the bond is formed to the anomeric carbon of the glycan moiety by an oxygen, nitrogen, carbon or sulphur atom, respectively. The glycosidic bond may be an acetal bond. The glycan may be any monosaccharide, disaccharide, oligosaccharide or polysaccharide, and it may be further substituted by any of the substituents listed above.

Examples of glycoside groups include, but are not limited to, β-D-O-galactoside, N-acetyl-β-D-O-galactosaminide, N-acetyl-α-D-O-galactosaminide, N-acetyl-β-D-O-glucosaminide, N-acetyl-β-D-N-glucosaminide, β-D-O-glucuronide, α-L-O-iduronide, α-D-O— galactoside, α-D-O-glucoside, α-D-C-glucoside, β-D-O-glucoside, α-D-O-mannoside, β-D-O-mannoside, β-D-C-mannoside, α-L-O— fucoside, β-D-O-xyloside, N-acetyl-α-D-O-neuraminide, lactoside, maltoside, dextran, and any analogue or modification thereof.

In an embodiment, an anomeric bond of a glycan moiety may be represented by a wavy line, which indicates that the stereochemistry of the anomeric carbon is not defined and it may exist in either the R or S configuration, in other words beta or alpha configuration, meaning that when the glycan is drawn as a ring the bond may be directed either above or below the ring. In a further embodiment, if the anomeric carbon is drawn with a wavy bond to a hydroxyl group (thus forming a hemiacetal) the wavy bond indicates that the glycan can also exist in the open-ring form (aldehyde or ketone).

In an embodiment, the term “polyethylene glycol” means a polymer comprising repeating “PEG” units of the formula —[CH₂CH₂O]ₙ—. In an embodiment, the term “PEG₁₅₀” means polyethylene glycol moiety having from 1 to 50 PEG units. In an embodiment, the term “substituted polyethylene glycol” means a polyethylene glycol substituted with one or more of the substituents listed above. In an embodiment, the term “branched polyethylene glycol” means a polyethylene glycol moiety substituted with one or more of polyethylene glycol substituents forming a branched structure.

In formula I, when n is greater than 1, each D may, in principle, be selected independently. Each L may likewise be selected independently. In other words, T may be linked to one or more linker-payload units. In such embodiments, the linker-payload units (for example, those indicated as D-O-L- in formula I) may be the same or different as any linker-payload units described in this specification.

In formula I, n may be an integer, for example an integer of at least 1.

In formula I, n may be in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 2000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, or 2000, or greater than 2000.

II) Payload Molecules

The payload molecule (in particular, its sugar moiety) may have a hydroxyl group, wherein the hydroxyl group has been incorporated in the conjugate such that the oxygen atom of the hydroxyl group is the oxygen atom of Formula I. In other words, in embodiments in which the payload molecule comprises a sugar moiety, wherein the sugar moiety has a hydroxyl group in unconjugated form, the payload molecule may be conjugated such that the hydroxyl group is incorporated in the conjugate such that the oxygen atom of the hydroxyl group is the oxygen atom shown as —O— in the conjugate represented by Formula I. The moiety —O-L- of Formula I may thus be considered to replace the hydroxyl group of the sugar moiety.

The hydroxyl group of the sugar moiety may be a primary or a secondary hydroxyl group. For example, in embodiments in which the sugar moiety is a D-glucopyranose or any other aldohexose unit, the primary hydroxyl group is the hydroxyl group at C-6 (carbon 6), and in an aldopyranose unit such as a D-glucopyranose, any hydroxyl groups at C-1, C-2, C-3 and/or C-4 are secondary hydroxyl groups.

Primary hydroxyl groups may be less sterically hindered and therefore easier to incorporate in the conjugate. A primary hydroxyl group may be a terminal hydroxyl group.

Secondary hydroxyl groups may be more hindered than primary hydroxyl groups and therefore more challenging to incorporate in the conjugate. However, it may be that due to hindrance, when incorporated in the conjugate, they may be less susceptible to cleavage and provide a more stable conjugate. A secondary hydroxyl group may be a non-terminal hydroxyl group.

However, the reactivities of primary and/or secondary hydroxyl groups may vary at least to some extent.

For example, the oxygen atom may be bonded to any one of the carbon atoms at positions 1-6 of the sugar moiety in the pyranose ring configuration. Such a pyranose ring configuration is shown in the schematic below:

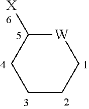

As a further example, the oxygen atom may be bonded to any one of the carbon atoms at positions 1-6 of the sugar moiety in the furanose ring configuration. Such a furanose ring configuration is shown in the schematic below:

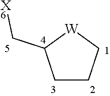

In the above schematics, the positions of the carbons in the ring are indicated. W indicates the position of oxygen (O) or other possible heteroatom, such as nitrogen or sulphur. X indicates a carbon atom that may not be present in situations in which the ring has 5 members (instead of 6). A skilled person will understand that the above schematics are not to be understood as chemical formulas of the sugar moiety; for example, each one of the carbon atoms at positions 1-6 of the sugar moiety can be independently substituted with any substituent disclosed in the present specification, but in the schematics above, any substituents have been omitted for simplicity.

For example, 2-deoxy-2-fluoro-D-glucopyranose contains four hydroxyl groups at C-1, C-3, C-4 and C-6, of which the first three are secondary hydroxyl groups, and the last one is a primary hydroxyl group, as shown below:

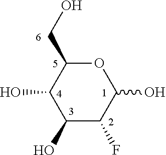 [SMILES: OC[C@H]1OC(O)[C@H](F)[C@@H](O)[C@@H]1O]

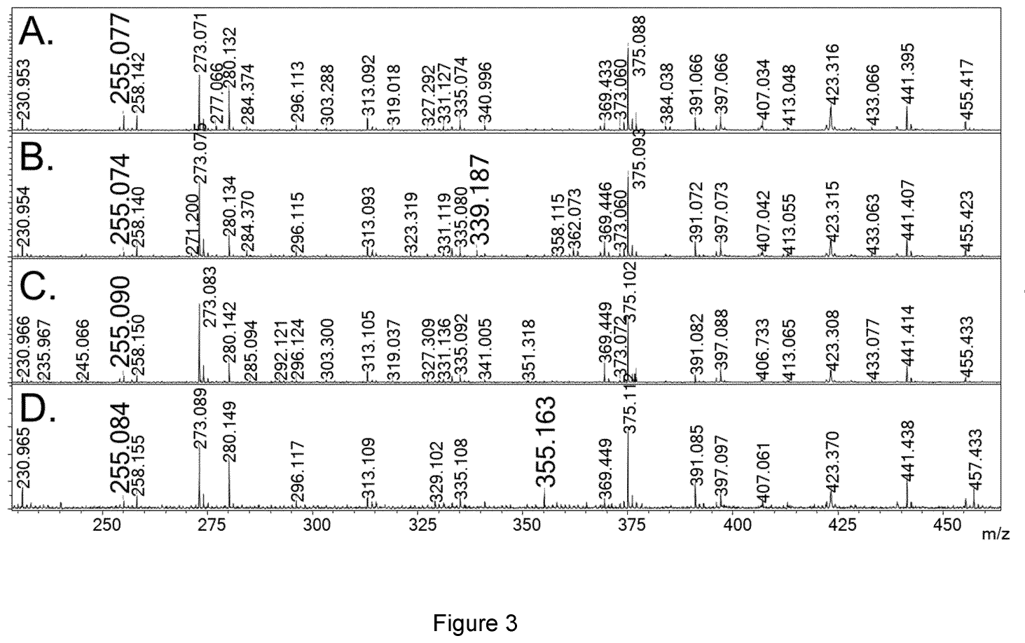

However, the numbering of carbon atoms may be variable in sugar moieties of other molecules than simple monosaccharides. For example, in kifunensine, the primary hydroxyl group may be considered to be attached to carbon 9 (C-9) of the kifunensine molecule as numbered according to the standard numbering, or to carbon 6 (C-6) when numbered differently (e.g. as shown in Vallee et al. 2000, J. Biol. Chem. 275:41287-98, FIG. 3).

In an embodiment, the payload molecule is a glycosylation inhibitor described in any one of the following publications: Esko et al. 2017, in Essentials of Glycobiology, 3^{rd} edition, Chapter 55; Chapman et al. 2004, Angew Chem Int Ed Engl 43:3526-48; Dorfmueller et al. 2006, J Am Chem Soc 128:16484-5; Brown et al. 2007, Crit Rev Biochem Mol Biol 42:481-515; Chaudhary et al. 2013, Mini Rev Med Chem 13:222-36; Tu et al. 2013. Chem Soc Rev 42:4459-75; Galley et al. 2014, Bioorg Chem 55:16-26; Gouin 2014, Chemistry 20:11616-28; Kallemeijn et al. 2014, Adv Carbohydr Chem Biochem 71:297-338; Kim et al. 2014, Crit Rev Biochem Mol Biol 49:327-42. Shayman & Larsen 2014, J Lipid Res 55:1215-25.

In an embodiment, the glycosylation inhibitor is a hydrophilic glycosylation inhibitor, such as a nonacetylated saccharide analog. The hydrophilicity may have the benefit that the hydrophilic glycosylation inhibitor may have a poor ability to enter non-target cells if it is prematurely released from the conjugate before reaching the target tissue such as the tumour or the target cell. For example, UDP-GlcNAc levels do not necessarily change significantly in response to unacetylated 4-fluoro-GlcNAc treatment, from the outside of the cell, of either human leukemia cell line KG1a or T cells, whereas treatment with peracetylated 4-fluoro-GlcNAc may significantly decrease UDP-GlcNAc levels in these cells and thereby may be capable of effectively inhibiting glycosylation in any cell, without discriminating between different cell types (Barthel et al. 2011, J. Biol. Chem. 286:21717-31). Hydrophilic glycosylation inhibitors may also be substantially non-toxic.

In an embodiment, the glycosylation inhibitor is a hydrophobic glycosylation inhibitor, such as a peracetylated saccharide analog. The hydrophobicity may have the benefit that the hydrophobic glycosylation inhibitor may have a good ability to enter target cells if prematurely released from the conjugate after reaching the target tissue such as tumour, but before reaching the target cell. Moreover, the hydrophobic glycosylation inhibitor may have a good ability to enter another target cell or the second tumour cell after inhibiting glycosylation in the (first) target cell.

In an embodiment, the glycosylation inhibitor is selected from the groups of:
1) Metabolic inhibitors, which are capable of interfering with steps involved in formation of common intermediates of a glycosylation pathway, such as nucleotide sugars;
2) Cellular trafficking inhibitors, which are capable of impeding the structure of or transit between the endoplasmic reticulum (ER), Golgi, and/or trans-Golgi network;
3) Tunicamycin, which is capable of inhibiting N-linked glycosylation through inhibition of dolichol-PP-GlcNAc formation and peptidoglycan biosynthesis through inhibition of undecaprenyl-PP-GlcNAc assembly;
4) Plant alkaloids, which are capable of inhibiting N-linked glycosylation through inhibition of processing glycosidases;
5) Substrate analogs, which are capable of inhibiting specific glycosyltransferases or glycosidases;
6) Glycoside primers, which are capable of inhibiting glycosylation pathways by diverting the assembly of glycans from endogenous acceptors to exogenous primers; and
7) Specific inhibitors of glycosylation, which may include, for example, interfering RNA to specific glycosyltransferases, and the like.

In an embodiment, the glycosylation inhibitor is selected from the groups 1)-7) above and any analogs or modifications thereof.

In an embodiment, the glycosylation inhibitor comprises or is a metabolic inhibitor (group 1).

In an embodiment, the glycosylation inhibitor comprises or is a cellular trafficking inhibitor (group 2).

In an embodiment, the glycosylation inhibitor comprises or is a tunicamycin (group 3).

In an embodiment, the glycosylation inhibitor comprises or is a plant alkaloid (group 4).

In an embodiment, the glycosylation inhibitor comprises or is a substrate analog (group 5). Such substrate analog may be capable of inhibiting a specific glycosyltransferase and/or glycosidase.

In an embodiment, the glycosylation inhibitor comprises or is a glycoside primer (group 6).

In an embodiment, the glycosylation inhibitor comprises or is a specific inhibitor (group 7).

In an embodiment, the glycosylation inhibitor comprises or is a metabolic inhibitor (group 1); a cellular trafficking inhibitor (group 2); a tunicamycin (group 3); a plant alkaloid (group 4); a substrate analog (group 5); a glycoside primer (group 6); and/or a specific inhibitor (group 7).

The glycosylation inhibitor may be selected from the group of a metabolic inhibitor, a cellular trafficking inhibitor, tunicamycin, a plant alkaloid, a substrate analog, a glycoside primer, a specific inhibitor of glycosylation, an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a glycosaminoglycan biosynthesis inhibitor, a glycosphingolipid biosynthesis inhibitor, a sulphation inhibitor, 2-deoxyglucose, a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluorosialic acid, 3-deoxy-3ax-fluorosialic acid, 3-deoxy-3eq-fluorosialic acid, 3-deoxy-3-fluoro-Neu5Ac, 3-deoxy-3ax-fluoro-Neu5Ac, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluorosialic acid, castanospermine, australine, deoxynojirimycin, N-butyldeoxynojirimycin, deoxymannojirimycin, kifunensin, swainsonine, mannostatin A, streptozotocin, 2-acetamido-2,5-dideoxy-5-thioglucosamine, 2-acetamido-2,4-dideoxy-4-thioglucosamine, PUGNAc (O-[2-acetamido-2-deoxy-D-glucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, a glycoside primer, a R-xyloside, a β-N-acetylgalactosaminide, a β-glucoside, a R-galactoside, β-N-acetylglucosaminide, a β-N-acetyllactosaminide, a disaccharide glycoside and a trisaccharides glycosideglucosylceramide epoxide, 2-amino-2-deoxymannose, a 2-acyl-2-deoxy-glucosyl-phosphatidylinositol, Neu5Ac-2-ene (DANA), 4-amino-DANA, 4-guanidino-DANA, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, a concanamycin, concanamycin A, concanamycin B, concanamycin C, a bafilomycin, bafilomycin A1, epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, an N-acyldeoxynojirimycin, N-acetyldeoxynojirimycin, an N-acyldeoxymannojirimycin, N-acetyldeoxymannojirimycin, 3-deoxy-3-fluoroNeu5N, 3-deoxy-3ax-fluoro-Neu5N, 3-deoxy-3eq-fluoroNeu5N, 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, 3′-fluoro-2′,3′-dideoxycytidine, and any analogs, modifications, acylated analogs, acetylated analogs, methylated analogs, or combinations thereof.

In an embodiment, the metabolic inhibitor (group 1) is selected from the group of a sulphation inhibitor, 2-deoxyglucose, 2-amino-2-deoxymannose, a 2-acyl-2-deoxy-glucosyl-phosphatidylinositol, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, a glycosaminoglycan biosynthesis inhibitor, a glycosphingolipid biosynthesis inhibitor, and any analogs, modifications, acylated analogs, acetylated analogs, methylated analogs, or combinations thereof.

In an embodiment, the cellular trafficking inhibitor (group 2) is selected from the group of a concanamycin, concanamycin A, concanamycin B, concanamycin C, a bafilomycin, bafilomycin A1, and any analogs, modifications, acylated analogs, acetylated analogs, methylated analogs, or combinations thereof.

In an embodiment, the tunicamycin (group 3) is selected from the group of tunicamycin and any analogs, modifications, acylated analogs, acetylated analogs, methylated analogs, or combinations thereof.

In an embodiment, the plant alkaloid (group 4) is selected from the group of an N-acyldeoxynojirimycin, N-acetyldeoxynojirimycin, an N-acyldeoxymannojirimycin, N-acetyldeoxymannojirimycin, epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, castanospermine, australine, deoxynojirimycin, N-butyldeoxynojirimycin, deoxymannojirimycin, kifunensin, swainsonine, mannostatin A, and any analogs, modifications, acylated analogs, acetylated analogs, methylated analogs, or combinations thereof.

In an embodiment, the substrate analog (group 5) is selected from the group of a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluorosialic acid, 3-deoxy-3ax-fluorosialic acid, 3-deoxy-3eq-fluorosialic acid, 3-deoxy-3-fluoro-Neu5Ac, 3-deoxy-3ax-fluoroNeu5Ac, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluorosialic acid, alloxan, streptozotocin, 2-acetamido-2,5-dideoxy-5-thioglucosamine, 2-acetamido-2,4-dideoxy-4-thioglucosamine, PUGNAc (O-[2-acetamido-2-deoxy-Dglucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, Neu5Ac-2-ene (DANA), 4-amino-DANA, 4-guanidino-DANA, (3R, 4R, 5S)-4-acetamido-5-amino-3-(1-ethylpropoxyl)-1-cyclohexane-1-carboxylic acid, (3R, 4R, 5S)-4-acetamido-5-amino-3-(1-ethylpropoxyl)-1-cyclohexane-1-carboxylic acid ethyl ester, N-acetyl-D-glucosamine-oxazoline, 6-methylphosphonate-N-acetyl-D-glucosamine-oxazoline, 6-methylphosphonate-N-acetyl-D-glucosamine-thiazoline, 3-deoxy-3-fluoroNeu-5N, 3-deoxy-3ax-fluoro-Neu5N, 3-deoxy-3eq-fluoro-Neu5N, and any analogs, modifications, acylated analogs, acetylated analogs, methylated analogs, or combinations thereof.

In an embodiment, the glycoside primer (group 6) is selected from the group of a glycoside primer, a β-xyloside, a β-N-acetylgalactosaminide, a β-glucoside, a β-galactoside, β-N-acetylglucosaminide, a β-N-acetyllactosaminide, a disaccharide glycoside and a trisaccharides glycoside, glucosylceramide epoxide, and any analogs, modifications, acylated analogs, acetylated analogs, methylated analogs, or combinations thereof.

In an embodiment, the specific inhibitor of glycosylation (group 7) is selected from the group of an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, and any analogs, modifications, acylated analogs, acetylated analogs, methylated analogs, or combinations thereof.

In an embodiment, the N-glycosylation inhibitor is selected from the group of a tunicamycin, a tunicamycin analog, a UDP-N-acetylglucosamine: dolichyl-phosphate N-acetylglucosaminephosphotransferase (GlcNAc-1-P-transferase) inhibitor, an oligosaccharyltransferase inhibitor, an N-glycan precursor synthesis inhibitor and an N-glycan processing inhibitor.

In an embodiment, the N-glycan processing inhibitor is selected from the group of a glucosidase inhibitor, a glucosidase I inhibitor, a glucosidase II inhibitor, a mannosidase inhibitor, a mannosidase I inhibitor, a mannosidase II inhibitor and an N-acetyl-glucosaminyltransferase inhibitor.

In an embodiment, the N-acetylglucosaminylation inhibitor is selected from the group of 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, a UDP-GlcNAc analog, a hexosamine pathway inhibitor, and any analogs or modifications thereof.

In an embodiment, the sialylation inhibitor is selected from the group of 3-deoxy-3-fluorosialic acid, 3-deoxy-3ax-fluorosialic acid, 3-deoxy-3eq-fluorosialic acid, 3-deoxy-3-fluoroNeu5Ac, 3-deoxy-3ax-fluoro-Neu5Ac, 3-deoxy-3eq-fluoro-Neu5Ac, 3-fluorosialic acid, a CMP-Neu5Ac analog, a β-N-acetyllactosaminide, Neu5Ac-2-ene (DANA), 4-amino-DANA, 4-guanidino-DANA, (3R, 4R, 5S)-4-acetamido-5-amino-3-(1-ethylpropoxyl)-1-cyclohexane-1-carboxylic acid, (3R, 4R, 5S)-4-acetamido-5-amino-3-(1-ethylpropoxyl)-1-cyclohexane-1-carboxylic acid ethyl ester, a sialyltransferase inhibitor, a CMP-sialic acid synthase inhibitor, 3-deoxy-3-fluoro-Neu5N, 3-deoxy-3ax-fluoro-Neu5N, 3-deoxy-3eq-fluoro-Neu5N, a hexosamine pathway inhibitor, and any analogs or modifications thereof.

In an embodiment, the galactosylation inhibitor is selected from the group of a galactosyltransferase inhibitor, a UDP-Gal analog, galactosyltransferase inhibitor, and any analogs or modifications thereof.

In an embodiment, the hexosamine pathway inhibitor is selected from the group of a glutamine-fructose-6-phosphate aminotransferase (GFPT1) inhibitor, a phosphoacetylglucosamine mutase (PGM3) inhibitor, a UDP-GlcNAc synthase inhibitor, N-acetyl-Dglucosamine-oxazoline, 6-methyl-phosphonate-N-acetyl-D-glucosamine-oxazoline, 6-methyl-phosphonate-N-acetyl-D-glucosamine-thiazoline, and any analogs, homologs or modifications thereof.

In an embodiment, the tunicamycin is selected from the group of tunicamycin I, tunicamycin II, tunicamycin III, tunicamycin IV, tunicamycin V, tunicamycin VI, tunicamycin VII, tunicamycin VIII, tunicamycin IX and tunicamycin X, and tunicamycins A, A0, A1, A2, A3, A4, B, B1, B2, B3, B4, B5, B6, C, C1, C2, C3, D, D1, D2, Tun 16:0A, Tun 16:0B, Tun 17:2, Tun 17:0A, Tun 17:0B, Tun 17:0C, Tun 18:1A and Tun 18:1B, and as described in Ito et al. 1980 (Agric. Biol. Chem. 44:695-8) and references therein and in Tsvetanova & Price 2001 (Anal. Biochem. 289:147-56) and references therein, and any analogs, homologs or modifications thereof.

In an embodiment, the glucosidase inhibitor is selected from the group of a glucosidase I inhibitor, a glucosidase II inhibitor, and a combination thereof.

In an embodiment, the glucosidase inhibitor is selected from the group of australine, epi-kifunensine, 1-deoxynojirimycin, an N-acyldeoxynojirimycin, N-acetyldeoxynojirimycin, and any analogs, combinations or modifications thereof.

In an embodiment, the mannosidase inhibitor is selected from the group of a mannosidase I inhibitor, a mannosidase II inhibitor, a lysosomal mannosidase inhibitor and a combination thereof.

In an embodiment, the mannosidase inhibitor is a combination of a mannosidase I inhibitor and a mannosidase II inhibitor. In an embodiment, the mannosidase inhibitor is a combination of kifunensine and swainsonine.

In an embodiment, the mannosidase I inhibitor is selected from the group of kifunensine, 1-deoxymannojirimycin, N-acyl-1-deoxymannojirimycin, N-acetyl-1-deoxymannojirimycin, N-alkyl-1-deoxymannojirimycin, N-butyl-1-deoxymannojirimycin, tamoxifen, raloxifene, sulindac, and any analogs or modifications thereof.

In an embodiment, the mannosidase II inhibitor is selected from the group of swainsonine, mannostatin A, and any analogs or modifications thereof.

The glycosylation inhibitor may be represented by formula II:

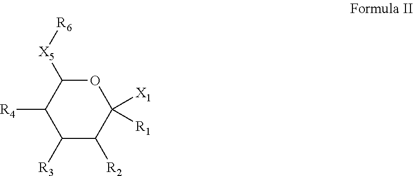

wherein X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to the oxygen atom of the sugar moiety;

each Z is independently selected from COCH₃, a C₁-C₁₂ acyl and a substituted C₁-C₁₂ acyl; and

Y is selected from F, Cl, Br, I, H and CH₃;

with the proviso that not more than one of R₁, R₂, R₃, R₄ and R₆ is Y, and that D contains not more than one L′.

The phrase “R₁ (or R₂, R₃, R₄, X₅, R₆, or any other substituent or radical described in this specification) is absent” may, in an embodiment, be understood as R₁ (or R₂, R₃, R₄, X₅, R₆, or any other substituent or radical described in this specification) being H. In other words, when a substituent or radical is “absent”, it may in some embodiments be understood as being H.

The phrase “L′ is a bond to the oxygen atom of the sugar moiety” may be understood throughout this specification such that L′ is a bond to the oxygen atom —O— shown in Formula I. The oxygen atom —O— shown in Formula I may be understood as forming a part of the payload molecule and/or its sugar moiety. L′ may be either a hydroxyl group of the glycosylation inhibitor or the oxygen atom incorporated in the bond to L (as shown in Formula I).

It may also be understood that not all atoms are drawn in the formulas described in this specification. Only substituents and groups that may vary have been drawn; H atoms may have been omitted for the sake of clarity.

The glycosylation inhibitor may, alternatively or additionally, be represented by formula II, wherein

X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, C₁-C₁₂ alkyl, or substituted C₁-C₁₂ alkyl;

R₆ is absent, Y, OH, OZ or L′;

L′ is a bond to the oxygen atom of the sugar moiety;

each Z is independently selected from COCH₃, C₁-C₁₂ acyl and substituted C₁-C₁₂ acyl;

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;

with the proviso that not more than one of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H.

In this context, the phrase “not more than one of R₁, R₂, R₃, R₄ and R₆ are Y” may be understood so that not more than one of R₁, R₂, R₃, R₄ and R₆ is selected from F, Cl, Br, I, H and CH₃.

The glycosylation inhibitor may, alternatively or additionally, be represented by formula II, wherein

X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′ R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to the oxygen atom of the sugar moiety;

each Z is independently selected from COCH₃, a C₁-C₁₂ acyl and a substituted C₁-C₁₂ acyl; and

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;

with the proviso that two of R₁, R₂, R₃, R₄ and R₆ are Y, that the glycosylation inhibitor contains not more than one L′, and when one of R₄′ and R₄″ is either COR₄′″ or COOR₄′″, then one of R₄′ and R₄″ is H.

In this context, the phrase “two of R₁, R₂, R₃, R₄ and R₆ are Y” may be understood so that two of R₁, R₂, R₃, R₄ and R₆ are selected from F, Cl, Br, I, H and CH₃.

The glycosylation inhibitor may, alternatively or additionally, be represented by formula II, wherein X₁ is H, COOH, COOCH₃ or COOL′;

R₁ is absent, OH, OZ or L′;

R₂ is absent, Y, OH, OZ, NHCOCH₃ or L′;

R₃ is absent, Y, OH, OZ or L′;

R₄ is absent, Y, OH, OZ, NH₂, NR₄′ R₄″, NHCOCH₃ or L′;

X₅ is absent, CH₂, CH(OH)CH₂, CH(OZ)CH₂, CH(OH)CH(OH)CH₂, CH(OZ)CH(OZ)CH₂, a C₁-C₁₂ alkyl, or a substituted C₁-C₁₂ alkyl;

R₆ is absent, Y, OH, OZ or L′;

L′ is a bond to the oxygen atom of the sugar moiety;

each Z is independently selected from COCH₃, a C₁-C₁₂ acyl and a substituted C₁-C₁₂ acyl;

Y is selected from F, Cl, Br, I, H and CH₃; and

R₄′ and R₄″ are each independently selected from H, C₁-C₁₂ alkyl, substituted C₁-C₁₂ alkyl, C₆-C₁₂ aryl, substituted C₆-C₁₂ aryl, COR₄′″ and COOR₄′″, wherein R₄′″ is selected from C₁-C₁₂ alkyl, substituted C₁-C₁₂ alkyl, C₆-C₁₂ aryl and substituted C₆-C₁₂ aryl;

with the proviso that three of R₁, R₂, R₃, R₄ and R₆ are Y, that the glycosylation inhibitor contains not more than one L′, and when one of R₄′ and R₄″ is either COR₄′″ and COOR₄′″, then one of R₄′ and R₄″ is H.

In this context, the phrase “three of R₁, R₂, R₃, R₄ and R₆ are Y” may be understood so that three of R₁, R₂, R₃, R₄ and R₆ are selected from F, Cl, Br, I, H and CH₃.

The term “substituted” in the context of Formula II may refer to being substituted by any one of the substituents described above.

Y may, in an embodiment of Formula II, be selected from F, Cl, Br, and I, or from F and Cl.

Y may, in an embodiment of Formula II, be F. Such fluorinated sugar analogs may be relatively effective glycosylation inhibitors, because the presence of the fluorine atom may prohibit the incorporation of the fluorinated sugar analog into various glycan structures. The fluorine atom also does not cause significant steric hindrance.

The glycosylation inhibitor may, alternatively or additionally, be represented by formula IIIa, IIIb, IIIc, IIIe, IIIf or IIIg:

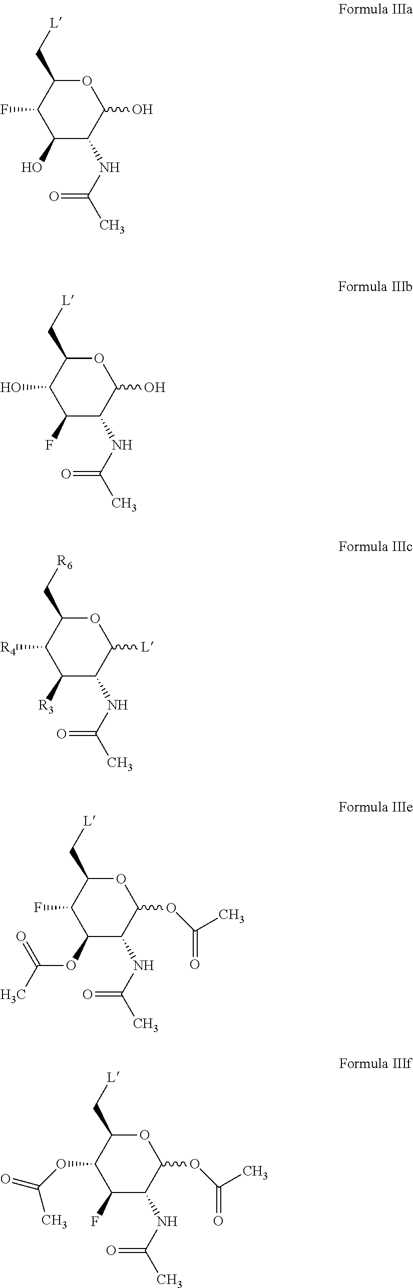

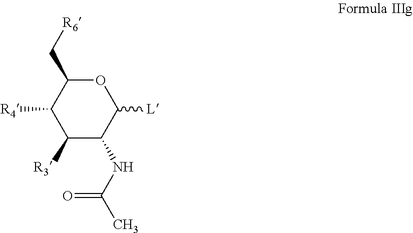

wherein L′ is a bond to the oxygen atom of the sugar moiety;

R₃, R₄ and R₆ are each independently either OH or F, with the proviso that only one of R₃, R₄ and R₆ is F; and

R₃′, R₄′ and R₆′ are each independently either OCOCH₃ or F, with the proviso that only one of R₃′, R₄′ and R₆′ is F.

The glycosylation inhibitor may, alternatively or additionally, be represented by any one of formulas IIIa, IIIb, IIIc, IIIe, IIIf or IIIg, wherein L′ is a bond to the oxygen atom of the sugar moiety;

R₃, R₄ and R₆ are each independently either OH or F, with the proviso that two of R₃, R₄ and R₆ are F; and

R₃′, R₄′ and R₆′ are each independently either OCOCH₃ or F, with the proviso that two of R₃′, R₄′ and R₆′ are F.

The glycosylation inhibitor may, alternatively or additionally, be represented by any one of formulas IIIa, IIIb, IIIc, IIIe, IIIf or IIIg, wherein L′ is a bond to the oxygen atom of the sugar moiety;

R₃, R₄ and R₆ are each F; and

R₃′, R₄′ and R₆′ are each F.

In an embodiment, the glycosylation inhibitor is a 3-deoxy-3-fluorosialic acid. In an embodiment, the 3-deoxy-3-fluorosialic acid is a 3-deoxy-3ax-fluorosialic acid or a 3-deoxy-3eq-fluorosialic acid.

The 3-deoxy-3-fluorosialic acid may, alternatively or additionally, be represented by any one of formulas IVa, IVb, IVc, IVd, IVe or IVf:

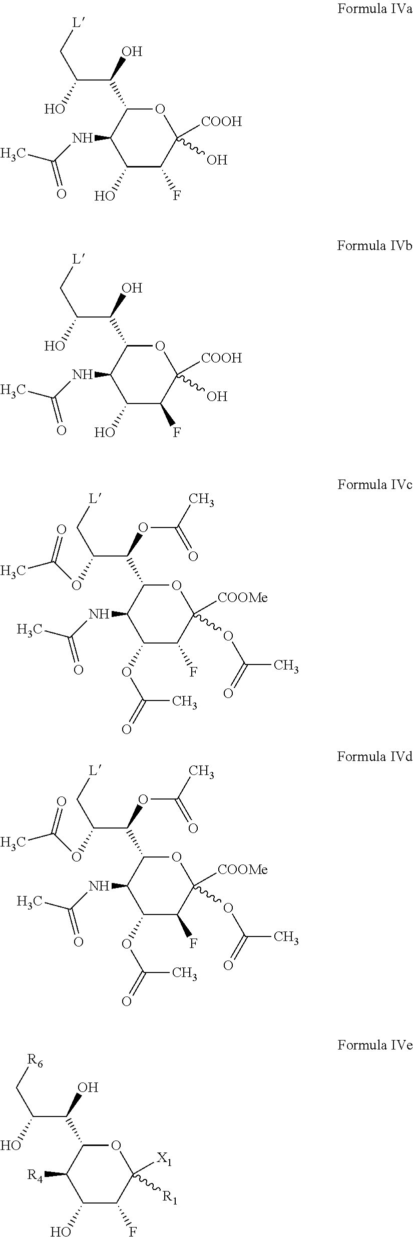

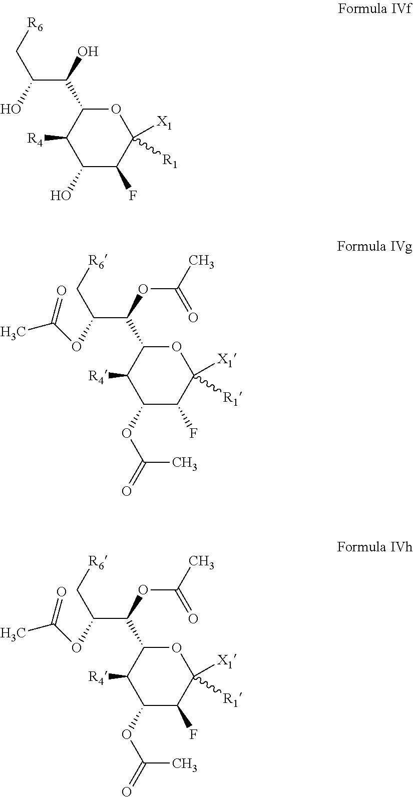

wherein

L′ is a bond to the oxygen atom of the sugar moiety;

R₁ and R₆ are each independently either OH or L′, R₄ is independently either NHCOCH₃ or L′, and X₁ is independently either COOH or L′, with the proviso that only one of R₁, R₄, R₆ and X₁ is L′; and

R₁′ and R₆′ are each independently either OCOCH₃ or L′;

R₄′ is independently either NHCOCH₃ or L′, and

X₁′ is independently either COOCH₃ or L′, with the proviso that only one of R₁′, R₄′, R₆′ and X₁′ is L′.

In the context of this specification, the phrase “3-deoxy-3-fluorosialic acid” may be understood so that one of the hydrogen atoms bonded to carbon-3 of the sialic acid is replaced by a fluorine atom. In this context, the phrase “3-deoxy-3ax-fluorosialic acid” may be understood so that the axial hydrogen atom bonded to carbon-3 of the sialic acid is replaced by a fluorine atom. In this context, the phrase “3-deoxy-3eq-fluorosialic acid” may be understood so that the equatorial hydrogen atom bonded to carbon-3 of the sialic acid is replaced by a fluorine atom.

The 3-deoxy-3-fluorosialic acid may, alternatively or additionally, be represented by any one of formulas IVe, IVf, IVg or IVh, wherein:

L′ is a bond to the oxygen atom of the sugar moiety;

R₁ and R₆ are each independently either OH, OZ or L′;

R₄ and R₄′ are independently either absent, OH, OZ, NH₂, NR₄″R₄′″, NHL′, NHCOCH₃ or L′;

X₁ is independently either COOH, COOMe, COOL′ or L′;

each Z is independently selected from COCH₃, a C₁-C₁₂ acyl and a substituted C₁-C₁₂ acyl;

R₁′ and R₆′ are each independently either OH, OZ, OCOCH₃ or L′;

R₄″ and R₄′″ are each independently selected from H, C₁-C₁₂ alkyl, substituted C₁-C₁₂ alkyl, C₆-C₁₂ aryl, substituted C₆-C₁₂ aryl, COR₄″″ and COOR₄″″, L′, L″-L′, Y, NH₂, OH, NHCOCH₃, NHCOCH₂OH, NHCOCF₃, NHCOCH₂Cl, NHCOCH₂OCOCH₃, NHCOCH₂N₃, NHCOCH₂CH₂CCH, NHCOOCH₂CCH, NHCOOCH₂CHCH₂, NHCOOCH₃, NHCOOCH₂CH₃, NHCOOCH₂CH(CH₃)₂, NHCOOC(CH₃)₃, NHCOO-benzyl, NHCOOCH₂-1-benzyl-1H-1,2,3-triazol-4-yl, NHCOO(CH₂)₃CH₃, NHCOO(CH₂)₂OCH₃, NHCOOCH₂CCl₃ and NHCOO(CH₂)₂F (wherein benzyl=CH₂C₆H₅);

wherein R₄″″ is selected from C₁-C₁₂ alkyl, substituted C₁-C₁₂ alkyl, C₆-C₁₂ aryl and substituted C₆-C₁₂ aryl;

L″ is selected from L′-substituted C₁-C₁₂ alkyl, L′ substituted C₆-C₁₂ aryl, COL′″, COOL′″, NH—, O—, NHCOCH₂—, NHCOCH₂O—NHCOCF₂—, NHCOCH₂OCOCH₂—, NHCOCH₂triazolyl-, NHCOOCH₂CHCH—, NHCOOCH₂CH₂CH₂S—, NHCOOCH₂—, NHCOOCH₂CH₂—, NHCOOCH₂CHCH₂CH₂—, NHCOO-benzyl-, NHCOO(CH₂)₃CH₂—, NHCOOCH₂-1-benzyl-1H-1,2,3-triazol-4-yland NHCOO(CH₂)₂OCH₂— (wherein benzyl is CH₂C₆H₅ and - is the bond to L′);

wherein L″ ′ is either L′-substituted C₁-C₁₂ alkyl or L′ substituted C₆-C₁₂ aryl,

with the proviso that the glycosylation inhibitor contains not more than one L′, and when R₄′ is either COR₄′″ or COOR₄′″ then R₄″ is H, and when R₄″ is either COR₄′″ or COOR₄′″ then R₄′ is H.

In the context of this specification, the term “L′ substituted” may be understood as referring to comprising L′, i.e. a bond to the oxygen atom of the sugar moiety. In other words, L′″ may be bonded to the oxygen atom of the sugar moiety.

The 3-deoxy-3-fluorosialic acid may, alternatively or additionally, be represented by any one of formulas IVj, IVk, IVl or IVm:

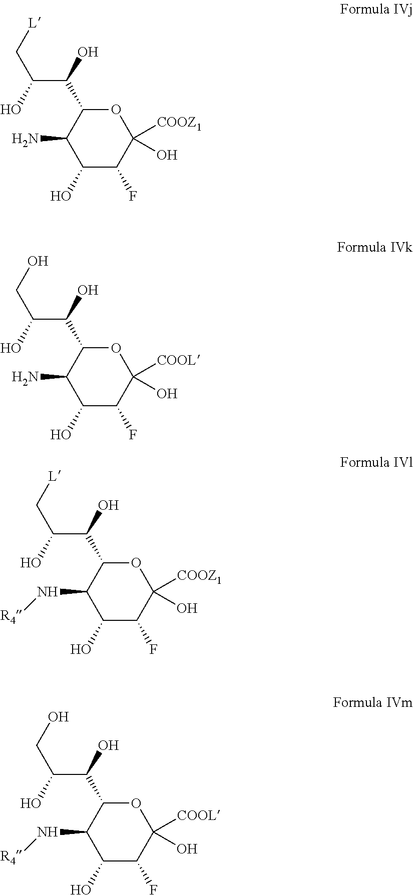

wherein

L′ is a bond to the oxygen atom of the sugar moiety;

Z₁ is selected from H, CH₃, C₁-C₁₂ alkyl, substituted C₁-C₁₂ alkyl, C₆-C₁₂ aryl and substituted C₆-C₁₂ aryl; and

R₄″ is selected from C₁-C₁₂ alkyl, substituted C₁-C₁₂ alkyl, C₆-C₁₂ aryl, substituted C₆-C₁₂ aryl, COR₄″″, COOR₄″″, COCH₃, COCH₂OH, COCF₃, COCH₂Cl, COCH₂OCOCH₃, COCH₂N₃, COCH₂CH₂CCH, COOCH₂CCH, COOCH₂CHCH₂, COOCH₃, COOCH₂CH₃, COOCH₂CH(CH₃)₂, COOC(CH₃)₃, COO-benzyl, COOCH₂-1-benzyl-1H-1, 2, 3-triazol-4-yl, COO(CH₂)₃CH₃, COO(CH₂)₂OCH₃, COOCH₂CCl₃ and COO(CH₂)₂F (wherein benzyl=CH₂C₆H₅);

wherein R₄″″ is selected from C₁-C₁₂ alkyl, substituted C₁-C₁₂ alkyl, C₆-C₁₂ aryl and substituted C₆-C₁₂ aryl.

The glycosylation inhibitor may, alternatively or additionally, be represented by formula A:

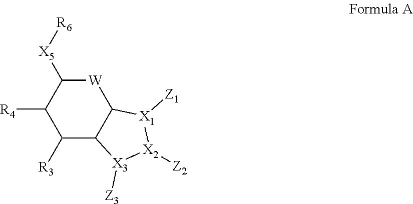

wherein

W is CH₂, NH, O or S;

X₁, X₂ and X₃ are each independently selected from S, O, C, CH and N;

with the proviso that when one or both of X₁ and X₃ are either O or S, then X₂ is either absent, a bond between X₁ and X₂, or CH;

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₃ and R₄ are each independently either absent or selected from H, OH, OZ or L′;

X₅ is absent, OH, OZ, O, CH₂, C₁-C₁₂ alkyl, or substituted C₁-C₁₂ alkyl;

R₆ is absent, H, OH, OZ, a phosphate, a phosphate ester, a phosphate analog, a boronophosphate, a boronophosphate ester, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;

L′ is a bond to the oxygen atom of the sugar moiety;

each Z is independently selected from COCH₃, C₁-C₁₂ acyl and substituted C₁-C₁₂ acyl; and

each of the bonds between the ring carbon and X₃, X₂ and X₃, X₁ and X₂, and the ring carbon and X₁, are independently either a single bond or a double bond or absent;

with the proviso than when both of the bonds between X₂ and X₃, and X₁ and X₂, are absent, then both X₂ and Z₂ are also absent;

with the proviso that the glycosylation inhibitor contains not more than one L′.

The glycosylation inhibitor may, alternatively or additionally, be represented by formula Aa, Ab, Ac or Ad:

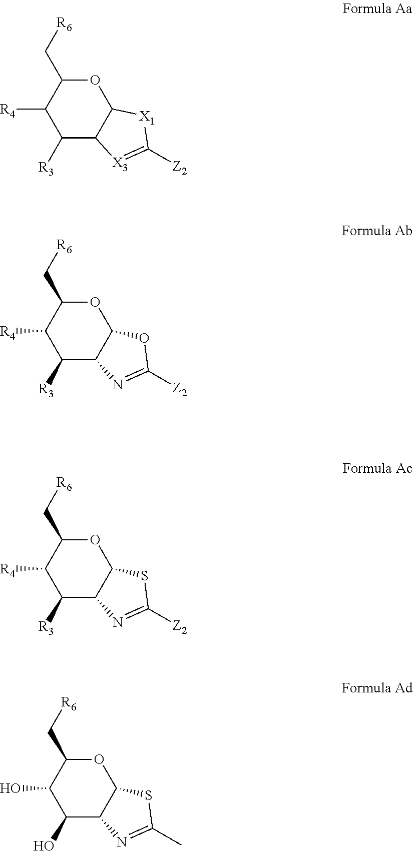

wherein

X₁ is selected from S, O, CH₂ and NH;

X₃ is selected from CH and N;

Z₂ is either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₃ and R₄ are each independently either absent or selected from H, OH, OZ or L′;

R₆ is absent, H, OH, OZ, a phosphate, a phosphate ester, a phosphate analog, a thiophosphate, a thiophosphate ester, a halophosphate, a halophosphate ester, a vanadate, a phosphonate, a phosphonate ester, a thiophosphonate, a thiophosphonate ester, a halophosphonate, a halophosphonate ester, methylphosphonate, methylphosphonate ester or L′;

L′ is a bond to the oxygen atom of the sugar moiety; and

each Z is independently selected from COCH₃, C₁-C₁₂ acyl and substituted C₁-C₁₂ acyl;

with the proviso that the glycosylation inhibitor contains not more than one L′.

The glycosylation inhibitor may, alternatively or additionally, be represented by formula B:

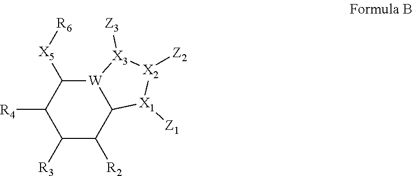

wherein

W is CH, N, O or S;

X₁, X₂ and X₃ are each independently selected from S, O, CH and N;

with the proviso that when one or both of X₁ and X₃ are either O or S, then X₂ is either absent, a bond between X₁ and X₃, C or CH;

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₂, R₃ and R₄ are each independently either absent or selected from H, OH, OZ or L′:

X₅ is absent, OH, OZ, O, CH₂, C₁-C₁₂ alkyl, or substituted C₁-C₁₂ alkyl;

R₆ is absent, H, OH, OZ or L′;

L′ is a bond to the oxygen atom of the sugar moiety;

each Z is independently selected from COCH₃, C₁-C₁₂ acyl and substituted C₁-C₁₂ acyl; and

each of the bonds between W and X₃, X₂ and X₃, X₁ and X₂, and the ring carbon and X₁, are independently either a single bond or a double bond or absent;

with the proviso than when both of the bonds between X₂ and X₃, and X₁ and X₂, are absent, then both X₂ and Z₂ are also absent;

with the proviso that the glycosylation inhibitor contains not more than one L′.

The glycosylation inhibitor may, alternatively or additionally, be represented by formula Ba, Bb, Bc, Bd, Be, Bf, Bg or Bh:

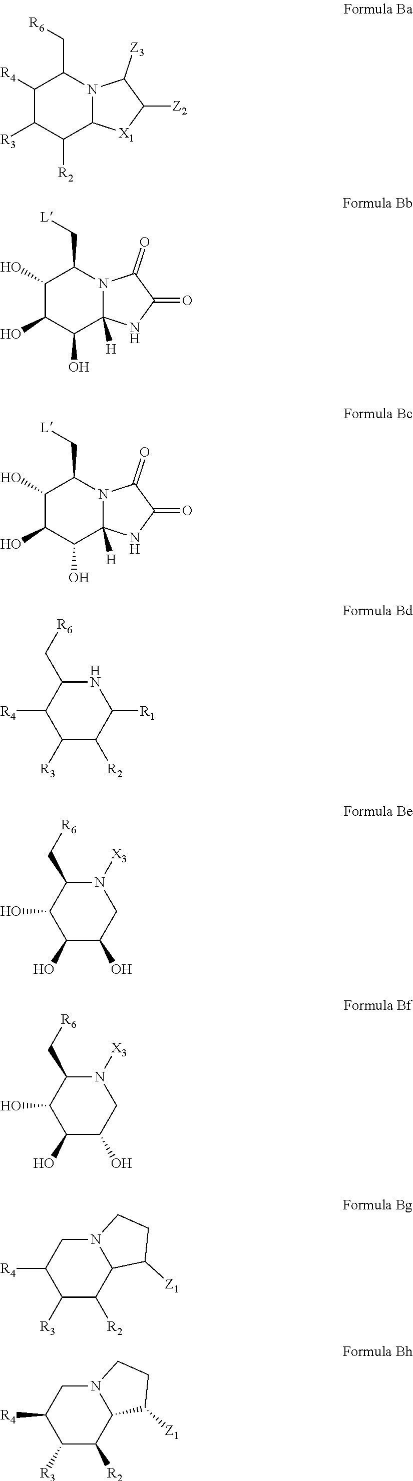

wherein

X₁ is selected from S, O, CH₂ and NH;

X₃ is selected from H, C₁-C₁₂ alkyl, substituted C₁-C₁₂ alkyl, C₁-C₁₂ acyl, substituted C₁-C₁₂ acyl, C₆-C₁₂ aryl or substituted C₆-C₁₂ aryl;

Z₁, Z₂ and Z₃ are each independently either absent or selected from H, OH, OZ, ═O, (═O)₂, C₁-C₁₂ alkyl, substituted C₁-C₁₂ alkyl, C₆-C₁₂ aryl, substituted C₆-C₁₂ aryl or L′;

R₁, R₂, R₃ and R₄ are each independently either absent or selected from H, OH, OZ or L′;

R₆ is absent, H, OH, OZ or L′;

L′ is a bond to the oxygen atom of the sugar moiety; and

each Z is independently selected from COCH₃, C₁-C₁₂ acyl and substituted C₁-C₁₂ acyl;

with the proviso that the glycosylation inhibitor contains not more than one L′.

The glycosylation inhibitor may, alternatively or additionally, be represented by formula Ca, Cb or Cc:

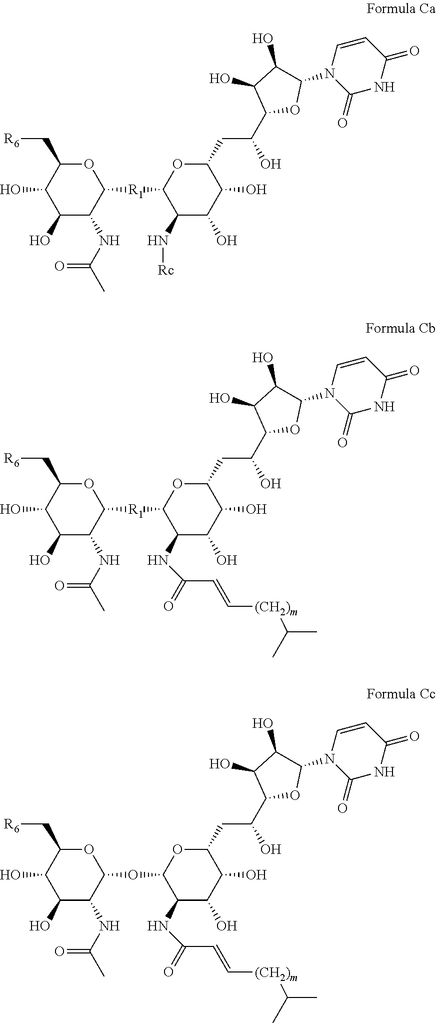

wherein

R₁ is O, NH, NRb, S, SO, SO₂ or CH₂;

Rb is C₁-C₁₀ alkyl, substituted C₁-C₁₀ alkyl, C₁-C₁₀ acyl or substituted C₁-C₁₀ acyl;

R₆ is OH or L′;

Rc is C₂-C₂₀ acyl, substituted C₂-C₂₀ acyl, C₆-C₂₀ aryl or substituted C₆-C₂₀ aryl;

m is 6, 7, 8, 9, 10, 11, 12, 13 or 14; and

L′ is a bond to the oxygen atom of the sugar moiety.

The glycosylation inhibitor may, alternatively or additionally, be represented by formula Dc:

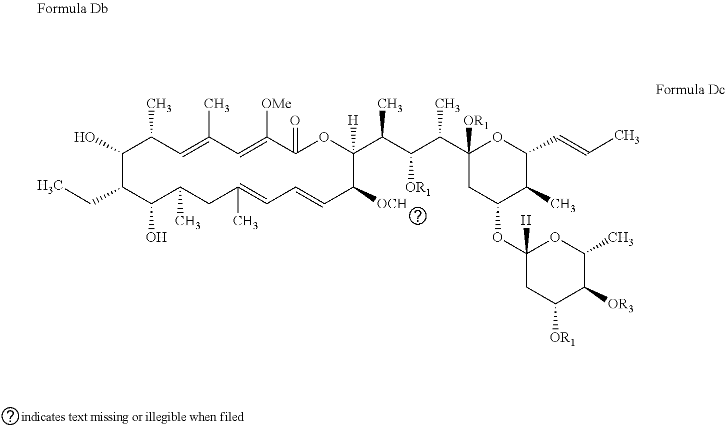

wherein

each R₁ is independently either H or L′;

R₃ is H, OH, CONH₂, CONHL′ or L′; and

L′ is a bond to the oxygen atom of the sugar moiety;

with the proviso that each of the Formulas Da, db and Dc contain only one L′.

The glycosylation inhibitor according to one or more embodiments described in this specification may be conjugated to the targeting unit in various ways.

In an embodiment, the payload molecule is a galectin inhibitor.

Galectins are a class of proteins that are capable of binding specifically to β-galactoside sugars. The structures of the β-galactose binding sites of galectin-1, 2 and 3 have been described (Lobsanov and Rini, Trends Glycosci Glycotech 1997, 45, 145-154; Seetharaman et al., J Biol Chem 1998, 273, 13047-13052; Saraboji et al., Biochemistry 2012, 51, 296-306). The term “Galectin” or “galectin” may be understood as referring to any S-type lectin, which is a galactoside-recognizing receptor. There are at least 15 galectins discovered in mammals, encoded by the LGALS genes, of which at least galectin-1, -2, -3, -4, -7, -8, 9, -10, -12 and -13 have been identified in humans (Essentials of Glycobiology 2017; Chapter 36). Several galectins have been found or at least implicated to play a role in diseases such as cancer, HIV, autoimmune disease, chronic inflammation, graft vs host disease and allergic reactions. For example, tumours may evade immune responses through galectin interactions. The roles galectin interactions may play in e.g. cancer may be quite complex and depend on the specific galectin.

In an embodiment, the conjugate is a conjugate for inhibition of inflammation, inhibition of fibrosis, inhibition of angiogenesis, inhibition of infection, inhibition of HIV-1 infection, or inhibition of autoimmune disease or autoimmune reactions in the target tissue.

In an embodiment, the conjugate is a conjugate for inhibition of any galectin-mediated condition in the target tissue.

In the context of this specification, the term “galectin inhibitor” may refer to a molecule capable of specifically binding one or more galectins. The galectin inhibitor may thereby be capable of inhibiting the function of the galectin to which it binds or interactions of the galectin, to which it binds, with one or more other molecules. The galectin inhibitor may directly bind to and/or interact with a galectin, for example by attaching, i.e. directly binding, to a galectin. The galectin inhibitor may directly bind to and/or interact with the galectin by non-covalent interactions, such as hydrogen bonds, hydrophobic interactions and/or ionic bonds. In an embodiment, the galectin inhibitor may be capable of specifically binding to a β-galactose binding site or a galectin. The galectin inhibitor may, in an embodiment, be capable of reversibly binding to and thereby inhibiting the galectin. The galectin inhibitor may, in an embodiment, be capable of non-covalently binding to and thereby inhibiting a galectin. Alternatively or additionally, the galectin inhibitor may be capable of binding irreversibly and/or covalently to a galectin, thereby inhibiting the galectin.

In an embodiment, the galectin inhibitor is not capable of inhibiting glycosylation (at least not to a significant extent).

Examples of galectins are galectin-1, galectin-2, galectin-3, galectin-4, galectin-5, galectin-6, galectin-7, galectin-8, galectin-9, galectin-10, galectin-11, galectin-12, galectin-13, galectin-14, and galectin-15. The galectin inhibitor may be capable of specifically binding to and inhibiting one or more of these galectins.

In an embodiment, the galectin inhibitor is a galectin-3 inhibitor. Galectin-3 may be expressed at high levels in various cancers and may thus be considered to be a tumour marker. Galectin-3 may be associated with immunosuppression and thus its inhibition may decrease immunosuppression.

In an embodiment, the galectin inhibitor is a galectin-1 inhibitor. Galectin-1 may be expressed at high levels in various cancers and may thus be considered to be a tumour marker. Galectin-1 is associated with immunosuppression and thus its inhibition may decrease immunosuppression.

In an embodiment, the galectin inhibitor is a galectin-9 inhibitor. Galectin-9 may be expressed at high levels in various cancers and may thus be considered to be a tumour marker. Galectin-9 is associated with immunosuppression and thus its inhibition may decrease immunosuppression.

In an embodiment, the galectin inhibitor has an ability to inhibit a plurality of galectins. In an embodiment, the galectin inhibitor inhibits a plurality of galectins including at least galectin-1 and galectin-3. In an embodiment, the galectin inhibitor inhibits a plurality of galectins including at least galectin-1 and galectin-9. In an embodiment, the galectin inhibitor inhibits a plurality of galectins including at least galectin-3 and galectin-9. In an embodiment, the galectin inhibitor inhibits a plurality of galectins including at least galectin-3 and galectin-9. In an embodiment, the galectin inhibitor inhibits a plurality of galectins including at least galectin-1, galectin-3 and galectin-9.

In this context, the term “a plurality of galectins” may refer to at least two, i.e. two or more, galectins; or in some embodiments, at least three galectins.

In an embodiment, the galectin inhibitor has an ability to specifically inhibit a galectin or a group of galectins; in other words it has substantially higher affinity to the galectin or the group of galectins than to other galectins.

In an embodiment, the galectin inhibitor is a specific inhibitor of galectin-1. In an embodiment, the galectin inhibitor is a specific inhibitor of galectin-3. In an embodiment, the galectin inhibitor is a specific inhibitor of galectin-9. In an embodiment, the galectin inhibitor is a specific inhibitor of galectin-1 and galectin-3. In an embodiment, the galectin inhibitor is a specific inhibitor of galectin-1 and galectin-9. In an embodiment, the galectin inhibitor is a specific inhibitor of galectin-3 and galectin-9. In an embodiment, the galectin inhibitor is a specific inhibitor of galectin-1, galectin-3 and galectin-9. In the context of this specification, the term “substantially higher affinity” means that there is large difference in the dissociation constantS (Kd) between the two affinities in question. In an embodiment, the difference between the Kd values is at least 5-fold. In an embodiment, the difference between the Kd values is at least 10-fold, at least 100-fold, at least 1000-fold, at least 10000-fold, at least 100000-fold, or at least 1000000-fold.

Various galectin inhibitors may be known, examples and embodiments of which are described below. However, other galectin inhibitors may also be contemplated.

In an embodiment, the galectin inhibitor is selected from the group of galactose, a 3-substituted galactose, a β-D-galactoside, a galactoside, a 3-substituted galactoside, a β-D-galactoside, a 3-substituted β-D-galactoside, lactose, a 3′-substituted lactose, a lactoside, a 3′-substituted lactoside, N-acetyllactosamine, a 3′-substituted N-acetyllactosamine, an N-acetyllactosaminide, a 3′-substituted N-acetyllactosaminide, N,N′-di-N-acetyllactosediamine, a 3′-substituted N,N′-di-N-acetyllactosediamine, an N,N′-di-N-acetyllactosediaminide, a 3′-substituted N,N′-di-N-acetyllactosediaminide, a taloside, a 3′-substituted taloside, a β-D-taloside, a 3′-substituted β-D-taloside, a mannoside, a 3′-substituted mannoside, a β-D-mannoside, a 3′-substituted β-D-mannoside, thiodigalactose (TDG), a 3-substituted thiodigalactose, a 3,3′-disubstituted thiodigalactose, 3,3′-dideoxy-3,3′-bis-[4-(3-fluorophenyl)-1H1,2,3-triazol-1-yl]-1,1′-sulfanediyl-di-β-D-galactopyranoside (33DFTG or TD139), 6-acyl-33DFTG, 6-succinyl-33DFTG, di-6-acyl-33DFTG, di-6-succinyl-33DFTG, a 6-substituted 33DFTG, a 6,6′-disubstituted 33DFTG, (E)-methyl-2-phenyl-4-(β-D-galactopyranosyl)-but-2-enoate, Galβ1-4Fuc, a 3′-substituted Galβ1-4Fuc, GM-CT-01, GR-MD-02, a pectin, reduced pectin, modified citrus pectin, GCS-100, a poly-N-acetyllactosaminide, lactulose, a lactuloside, a 3′-substituted lactulose, a 3′-substituted lactuloside, lactulosyl-L-leucine, a 3′-substituted lactulosyl-L-leucine, a galectin-binding molecule that inhibits galectin-galectin ligand interaction, an RNAi inhibiting galectin expression, GB1107, and any analog, modification, combination or multivalent combination thereof.

In an embodiment, the multivalent combination is a dimer of a galectin inhibitor. In an embodiment, the dimer of a galectin inhibitor is dimer of 33DFTG, dimer of 6-succinyl-33DFTG or dimer of 6-acetyl-33DFTG. In an embodiment, the dimer is conjugated (i.e. the two galectin inhibitor moieties are conjugated) with a spacer. In an embodiment, the spacer is a polyethylene glycol (PEG) chain.

In an embodiment, the multivalent combination is a trimer of a galectin inhibitor. In an embodiment, the trimer of a galectin inhibitor is trimer of 33DFTG, trimer of 6-succinyl-33DFTG or trimer of 6-acetyl-33DFTG. In an embodiment, the trimer is conjugated with a spacer. In an embodiment, the spacer is a polyethylene glycol (PEG) chain.

In an embodiment, the multivalent combination is a tetramer of a galectin inhibitor. In an embodiment, the tetramer of a galectin inhibitor is tetramer of 33DFTG, tetramer of 6-succinyl-33DFTG or tetramer of 6-acetyl-33DFTG. In an embodiment, the tetramer is conjugated with a spacer. In an embodiment, the spacer is a polyethylene glycol (PEG) chain.

In the context of this specification, the term “3-substituted” or “6-substituted” may mean that the structure has a substituent joined to the atom in the 3-position or 6-position, respectively, of either the central ring of a monosaccharide inhibitor or a monosaccharide analog inhibitor, or the reducing terminal ring (drawn on the right-hand side in molecular structures) of a disaccharide inhibitor or a disaccharide analog inhibitor. In the context of this specification, the term “3′-substituted” or “6′-substituted” may mean that the structure has a substituent joined to the atom in the 3-position or 6-position, respectively, of the non-reducing terminal ring (drawn on the lefthand side in molecular structures) of a disaccharide inhibitor or a disaccharide analog inhibitor. In the context of this specification, the term “3,3′-disubstituted” or “6,6′-disubstituted” means that the structure has a substituent joined to the atom in the 3-position or 6-position, respectively, of the both rings of the disaccharide inhibitor or the disaccharide analog inhibitor.

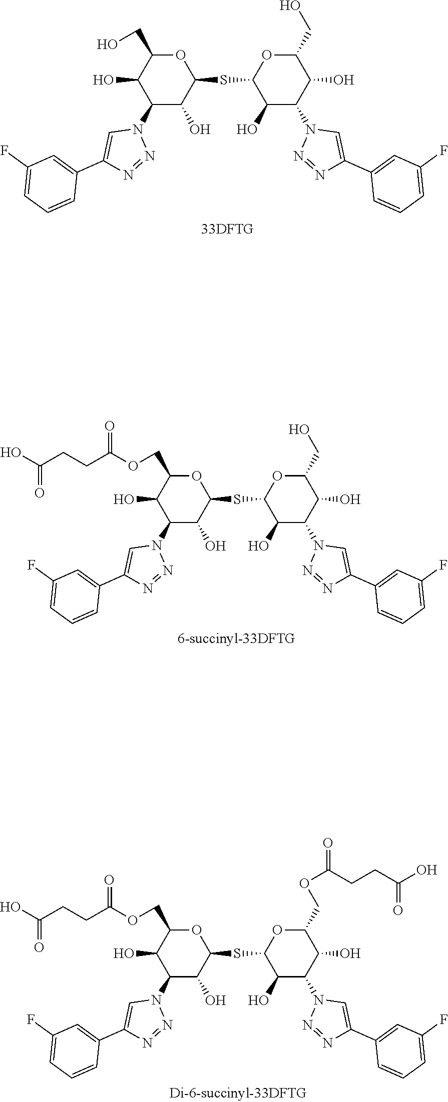 [SMILES: OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O ; O=C(O)CCC(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O ; O=C(O)CCC(=O)OC[C@H]1O[C@@H](S[C@@H]2O[C@H](COC(=O)CCC(=O)O)[C@H](O)[C@H](n3cc(-c4cccc(F)c4)nn3)[C@H]2O)[C@H](O)[C@@H](n2cc(-c3cccc(F)c3)nn2)[C@H]1O]

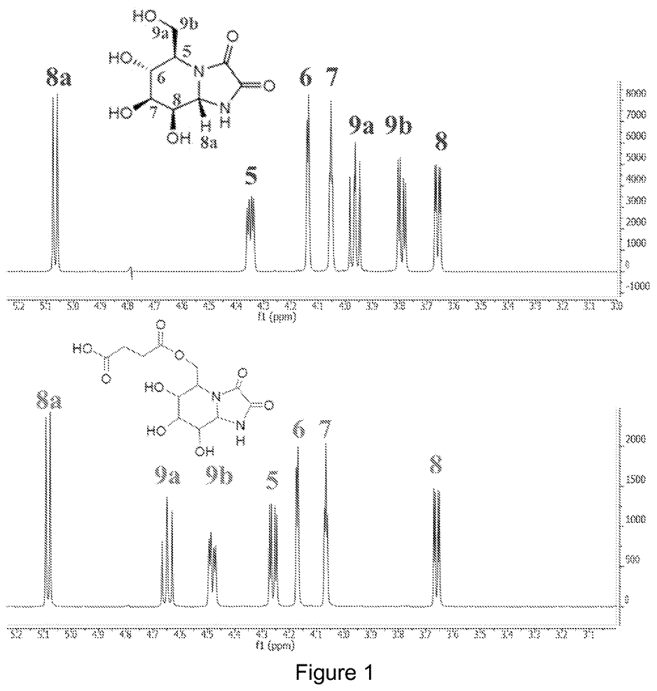

In an embodiment, the galectin inhibitor is selected from the group of molecules described in Blanchard et al. 2016 (Expert Opinion on Therapeutic Patents 26, issue 5; text, FIG. 1 and Table 1).

In an embodiment, the galectin inhibitor is selected from the group of molecules described in any of the patent documents US20030109464, U.S. Pat. Nos. 9,050,352, 6,849,607B2, 7,700,763, US20140336146, WO2014067986, U.S. Pat. Nos. 7,012,068, 7,893,252, 8,722,645, 8,658,787, 8,962,824, US20140086932, US20140235571, US20150147338, U.S. Pat. No. 8,877,263, US20150133399, US20030004132, US20040121981, US20060014719, US20060074050, US2007010438, WO2006128027, U.S. Pat. Nos. 7,339,023, 8,716,343, WO2012131079, WO2014070214, EP2858681, WO2012061395, U.S. Pat. No. 9,034,325, WO2015013388, U.S. Pat. Nos. 8,968,740, 7,662,385, 7,964,575, EP2771367, US20070185014, US20100004163, WO2002089831, U.S. Pat. Nos. 5,948,628, 6,225,071, 8,598,323, 6,890,531, 8,513,208, US20040023855, TW201410702A, and WO2018011093.

In an embodiment, the galectin inhibitor is represented by formula EII:

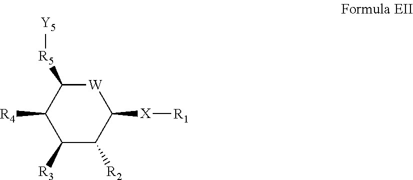

wherein W is O, S, NH, NY₁, CH₂, CY₁H or C(Y₁)₂;

X is O, S, S(═O), S(═O)₂, NH, NY₁, CH₂, CY₁H, C(Y₁)₂ or a bond;

R₁ is H, a saccharide, a saccharide substituted with L′, Z, M, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl or L′;

R₂ is H, OH, OZ, OM, NHCOCH₃, NHZ, NHM or L′;

R₃ is H, OH, OZ, OM, NHZ, NHM, L′ or Y₃;

R₄ is H, OH, OZ, OM or L′;

R₅ is H, CH₂, a saccharide, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl or a bond;

Y₅ is either absent or H, OH, OZ, OM or L′;

L′ is a bond to the oxygen atom of the sugar moiety;

M is a removable masking substituent, independently selected from the group of an acetal, hemiacetal, ketal, hemiketal, imino, formyl, acyl, carboxy, thiocarboxy, thiolocarboxy, thionocarboxy, imidic acid, hydroxamic acid, ester, acyloxy, oxycarboyloxy, amino, amido, thioamido, acylamido, aminocarbonyloxy, ureido, guanidino, tetrazolyl, imino, amidine, nitro, nitroso, azide, cyano, isocyano, cyanato, isocyanato, thiocyano, isothiocyano, sulfhydryl, thioether, disulfide, sulfine, sulfone, sulfinic acid, sulfonic acid, sulfinate, sulfonate, sulfinyloxy, sulfonyloxy, sulfate, sulfamyl, sulfonamido, sulfamino, sulfonamino, phospho, phosphinic acid, phosphonate, phosphoric acid, phosphate, phosphorous acid, phosphite, phosphoramidite, or phosphoramidate substituent, or a glycoside or peptide substituent; each Z is independently selected from the group of a C₁-C₁₀ acyl or a substituted C₁-C₁₀ acyl;

each Y₁ is independently selected from a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl;

with the proviso that not more than one of R₁, R₂, R₃, R₄ and Y₅ is L′, and that the galectin inhibitor D contains not more than one L′; and wherein

Y₃ is a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E, and FY3-F:

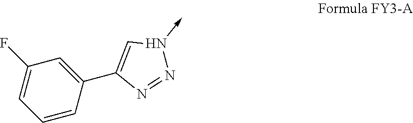

wherein the arrow shows the bond to rest of the structure (i.e. the galectin inhibitor);

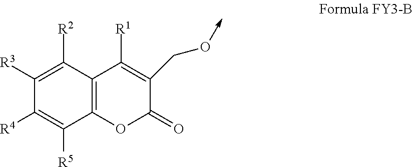

wherein the arrow shows the bond to rest of the structure; and

wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group;

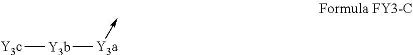

wherein the arrow shows the bond to rest of the structure; and

wherein Y₃a is either O or NH,

Y₃b is selected from the group of CO, SO₂, SO, PO₂, PO, and CH₂, or is a bond, and

Y₃c is selected from the group of:

a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group;

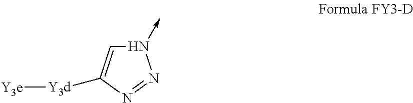

wherein the arrow shows the bond to rest of the structure; and

Y_{3d} is selected from the group of CH₂, CO, SO₂, and phenyl or is a bond; R₁ₐ is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y₃ₑ is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group;

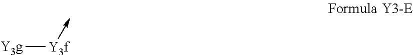

wherein the arrow shows the bond to rest of the structure; and

Y_{3f} is either CONH or a 1H-1,2,3-triazole ring; and

Y_{3g} is selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group;

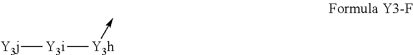

wherein the arrow shows the bond to rest of the structure; and

Y₃ₕ is NH, CH₂, NRₓ or a bond; Y₃ᵢ is CO, SO, SO₂, PO or PO₂H; Y₃ is selected from the group of an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; R_{1b} is H, a saccharide, an alkyl group, an alkenyl group, or an aryl group and wherein Rx is H, an alkyl group, an alkenyl group, an aryl group, a heteroaryl group or a heterocycle.

In an embodiment, the galectin inhibitor is represented by formula EII;

Y₃ is a structure described by formula FY3-G:

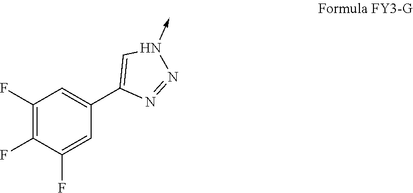

wherein the arrow shows the bond to rest of the structure (i.e. the galectin inhibitor);

R₁ is selected from the group of H, a saccharide, a saccharide substituted with L′, Z, M, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl, L′, 4-methylphenylthio, ethylthio, 3-chlorophenylthio, 4-chlorophenylthio, phenylthio, 3-bromophenylthio, 3-iodophenylthio, 3,4-dichlorophenylthio, 3-chloro-4-cyanophenylthio, 2,3-dichlorophenylthio and 3,4-dichlorophenoxy; and X is a bond to R₁ in either α or β configuration.

In an embodiment, the galectin inhibitor is represented by formula EIII:

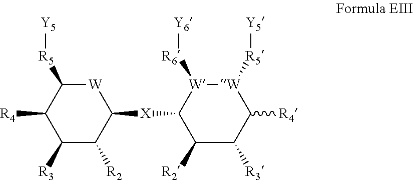

wherein W′ and W″ are each independently selected from the group of O, S, N, NH, NY₁, CH, CH₂, CY₁H and C(Y₁)₂;

R₂′ is H, OH, OZ, OM, NHCOCH₃, NHZ, NHM or L′;

R₃′ is H, OH, OZ, OM, NHCOCH₃, NHZ, NHM, L′ or Y₃′;

R₄′ is either absent or H, OH, OZ, OM and L′;

R₅′ and R₆′ are each independently either absent or selected from the group of H, CH₂, a saccharide, a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl, a substituted C₆-C₂₀ aryl and a bond;

Y₅′ and Y₆′ are each independently either absent or selected from the group of H, OH, OZ, OM and L′;

Y₃′ is a C₁-C₁₀ alkyl, a substituted C₁-C₁₀ alkyl, a C₂-C₁₀ alkenyl, a substituted C₂-C₁₀ alkenyl, a C₂-C₁₀ alkynyl, a substituted C₂-C₁₀ alkynyl, a C₆-C₂₀ aryl and a substituted C₆-C₂₀ aryl, an azide, or a structure described by any one of formulas FY3-A, FY3-B, FY3-C, FY3-D, FY3-E or FY3-F as described above in the context of Formula EII;

and wherein the other substituents are as described above in the context of Formula EII;

with the proviso that not more than one of R₁, R₂, R₃, R₄, Y₅, R₁′, R₂′, R₃′, R₄′, Y₅′, and Y₆′ is L′, and that the galectin inhibitor contains not more than one L′.

The wavy bond between C-4 of the second ring and its substituent R′ may point to either above or below the ring. In other words, C-4 may be either in the R or S configuration.

In an embodiment, the galectin inhibitor is represented by any one of formulas EIV to EIX:

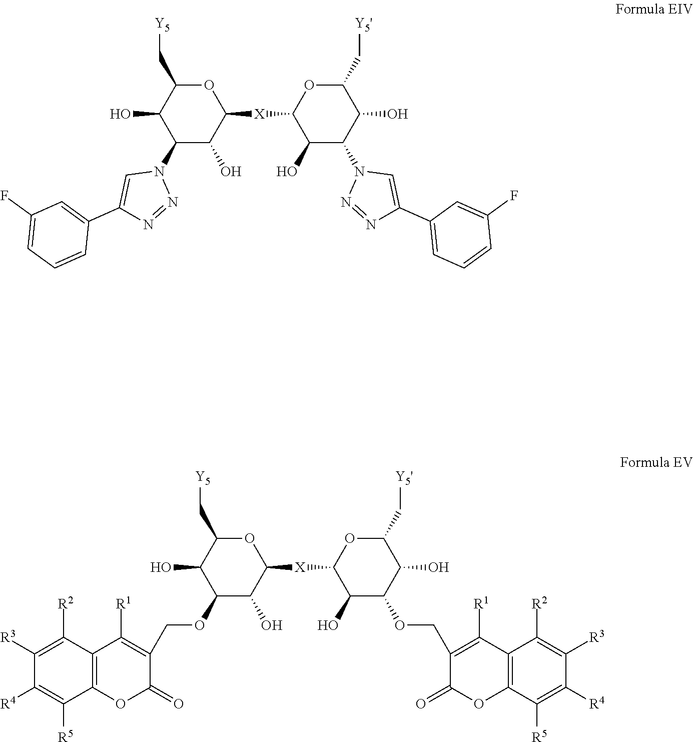

wherein R¹, R², R³, R⁴ and R⁵ are independently selected from the group of H, optionally substituted alkyl groups, halogens, optionally substituted alkoxy groups, OH, substituted carbonyl groups, optionally substituted acyloxy groups, and optionally substituted amino groups; wherein two, three, four or five of R¹, R², R³, R⁴ and R⁵ in adjacent positions may be linked to form one or more rings, and the remaining of R¹, R², R³, R⁴ and R⁵ is/are independently selected from the above group;

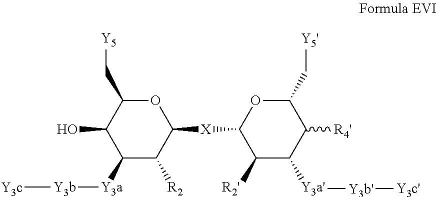

wherein Y₃a and Y₃a′ are independently either O or NH,

Y₃b and Y₃b′ are independently selected from the group of CO, SO₂, SO, PO₂, PO, and CH₂, or is a bond, and

Y₃c and Y₃c′ are independently selected from the group of:

a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamino group, a phenyl group substituted with at least one hydroxy group, a phenyl group substituted with at least one carbonyl group and a phenyl group substituted with at least one substituted carbonyl group, or

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, or

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group;

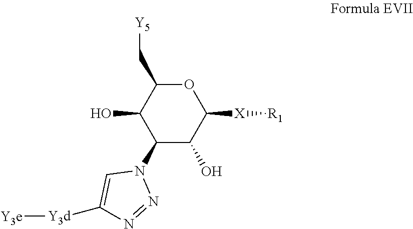

wherein Y_{3d} is selected from the group of CH₂, CO, SO₂, and phenyl or is a bond; R₁ₐ is selected from the group of D-galactose, C3-substituted D-galactose, C3-1,2,3-triazol-1-yl-substituted D-galactose, H, a C₁-C₁₀ alkyl, a C₁-C₁₀ alkenyl, a C₆-C₂₀ aryl, an imino group and a substituted imino group; Y₃ₑ is selected from the group of an amino group, a substituted amino group, an alkyl group, a substituted alkyl group, an alkoxy group, a substituted alkoxy group, an alkylamino group, a substituted alkylamino group, a substituted naphthyl group, a thienyl group, and a substituted thienyl group: wherein said substituent is one or more selected from the group consisting of halogen, alkoxy, alkyl, nitro, sulfo, amino, hydroxy or carbonyl group;

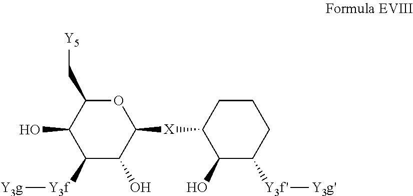

Y_{3f} and Y_{3f}′ are each independently either CONH or a 1H1,2,3-triazole ring; Y_{3g} and Y_{3g}′ are each independently selected from the group of an alkyl group of at least 4 carbons, an alkenyl group of at least 4 carbons, an alkynyl group of at least 4 carbons, a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, a carbamoyl group substituted with an substituted aryl group, a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group, a phenyl group substituted with at least one trifluoromethoxy group, a phenyl group substituted with at least one sulfo group, a phenyl group substituted with at least one hydroxy group, a phenyl group substituted with at least one carbonyl group, a phenyl group substituted with at least one substituted carbonyl group, a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, a naphthyl group substituted with at least one substituted carbonyl group, a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, a heteroaryl group substituted with at least one substituted carbonyl group, a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group;

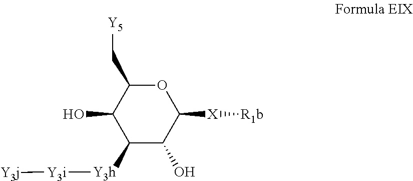

wherein Y₃ₕ is NH, CH₂, NRₓ or a bond; Y₃ᵢ is CO, SO, SO₂, PO or PO₂H; Y₃ⱼ is selected from the group of an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, an alkyl group of at least 4 carbon atoms Substituted with a halogen, a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxyl group, a phenyl group substituted with a carbonyl group, a phenyl group substituted with a substituted carbonyl group and a phenyl amino group; R_{1b} is H, a saccharide, an alkyl group, an alkenyl group, or an aryl group and wherein RX is H, an alkyl group, an alkenyl group, an aryl group, a heteroaryl group or a heterocycle;

wherein Y₅, X and Y₅′ are as described above.

The wavy bond between C-4 of the second ring and its substituent, or a wavy bond elsewhere in the present specification, means that the stereochemistry is either R or S; in other words the bond may be directed to either above or below the ring.

Examples of galectin inhibitors represented by the above Formulas are described e.g. in U.S. Pat. No. 9,353,141 (Formula EV of the present disclosure); WO 2005/113568 (Formula EVI of the present disclosure); WO 2005/113569 (Formula EVII of the present disclosure); WO 2010/126435 (Formula EVIII of the present disclosure); U.S. Pat. No. 7,230,096 (Formula EIX of the present disclosure), which are herein incorporated in their entirety.

The effectiveness or activity and/or selectivity of individual galectin inhibitors may vary. For example, embodiments in which R₃ and/or R₃′, or corresponding substituents, comprise cyclic and/or hydrophobic groups or moieties, may have a relatively high affinity to one or more galectins.

In an embodiment, the the galectin inhibitor is masked with a removable masking substituent (i.e. a removable group), such that the galectin inhibitor is capable of binding to a galectin only after removal of the removable masking substituent.

In an embodiment, the galectin inhibitor comprises a removable masking substituent M.

Suitable removable masking substituents or groups may include, for example, an ester group, a carbamate group, a glycoside, a hydrazone group, a peptide, a glycoside, or an acetal group.

In an embodiment, when the masking moiety M is comprised in at least one of R₂, R₂′, R₄, R₄′, Y₅ and Y₅′ in any of the Formulas described in this specification, the Kd of the binding of the galectin inhibitor to a galectin is sufficiently large so that the galectin inhibitor is not capable of binding to galectin, unless M is first removed.

In an embodiment, the galectin inhibitor is represented by any one of galectin inhibitors represented by

Formula EII, wherein R₁ is M, or at least one of R₂, R₃, R₄, R₅ or Y₅ is OM or NHM;

Formula EIII, wherein at least one of R₂′, R₃′, R₄′, Y₅, Y₅′ or Y₆′ is OM or NHM;

Formula EIV, Formula EV, or Formula EVI, wherein at least one of Y₅ or Y₅′ is OM;

Formula EVII or Formula EVIII, or Formula EIX, wherein Y₅ is OM.

In an embodiment, the galectin inhibitor is represented by any one of Formulas EII-EIX, wherein Y₅ or Y₅′ (where present) is OM.

In an embodiment, the galectin inhibitor is represented by any one of Formulas EII-EIX, wherein Y₅ is OM.

In an embodiment, at least one of R₂, R₂′, R₄, R₄′, Y₅ and Y₅′ is OM. In an embodiment, one of R₂, R₂′, R₄, R₄′, Y₅ and Y₅′ is OM. In an embodiment, at least one of R₂ and R₂′ is OM. In an embodiment, one of R₂ and R₂′ is OM. In an embodiment, at least one of R₄ and R₄′ is OM. In an embodiment, one of R₄ and R₄′ is OM. In an embodiment, at least one of Y₅ and Y₅′ is OM. In an embodiment, one of Y₅ and Y₅′ is OM.

In the context of the present specification, the term “capable of binding to galectin” may mean that the Kd of the binding interaction of the galectin inhibitor with the galectin is sufficiently low. A sufficient affinity for being capable of binding to galectin may be e.g. one having a dissociation constant (Kd) in the order of micromolar Kd, nanomolar Kd, picomolar Kd, or smaller. In an embodiment, the Kd is below 10⁻³ mol/l (about millimolar or smaller). In an embodiment, the Kd is below 10⁻⁴ mol/l, below 10⁻⁵ mol/l, below 10⁻⁶ mol/l, below 10-? mol/l, below 10⁻⁸ mol/l, or below 10⁻⁹ mol/l.

Conversely, in an embodiment, when the galectin inhibitor comprises the removable masking substituent, the Kd may be in the order of milliomolar Kd or larger. In an embodiment, when the galectin inhibitor comprises the removable group, the Kd is above 10⁻³ mol/l (about millimolar or larger). In an embodiment, the Kd is above 10⁻² mol/l, above 0.1 mol/l, or above 1 mol/l. Embodiments in which the galectin inhibitor is masked with a removable group, such that the galectin inhibitor is capable of binding to galectin only after removal of the removable group, may reduce or avoid binding of the galectin inhibitor within tissues in which galectin inhibition is not necessarily desired. The removable group may prevent or reduce interaction of the galectin inhibitor at off-tumour locations. For example, in a target tissue such as tumour or cancer tissue, the removable group may be cleaved off, after which the galectin inhibitor may bind to a galectin within the tumour or cancer tissue. Such embodiments may thus function in a prodrug-like manner.

The removable group may be removable within a cell, for example a cell of the target tissue.

The removable group may be removable by low pH, by reducing conditions, by a protease or a peptidase, or by a glycosidase; for example in a target cell, in a target cell lysosome, in a target cell cytosol, or in a target tissue.

III) Linker Units

Various types of linker units may be suitable, and many are known in the art. The linker unit may comprise one or more linker groups or moieties. It may also comprise one or more groups formed by a reaction between two functional groups. A skilled person will realize that various different chemistries may be utilized when preparing the conjugate, and thus a variety of different functional groups may be reacted to form groups comprised by the linker unit L. In an embodiment, the functional groups are selected from the group consisting of sulfhydryl, amino, alkenyl, alkynyl, azidyl, aldehyde, carboxyl, maleimidyl, succinimidyl and hydroxylamino. A skilled person is capable of selecting the functional groups so that they may react in certain conditions.

The terms “linker unit” and “linker” may be used interchangeably in this specification.

The linker unit may be configured to release the payload molecule after the conjugate is bound to the target cell. The linker unit may, for example, be cleavable. The cleavable linker unit may be cleavable under intracellular conditions, such that the cleavage of the linker unit may release the payload molecule in the intracellular environment. The cleavable linker unit may be cleavable under conditions of the tumour microenvironment, such that the cleavage of the linker unit may release the payload molecule in the tumour.

The linker unit may be configured to release the payload molecule after the conjugate is delivered to the tumour and/or bound to the target molecule or to the target cell.

The linker unit may be non-cleavable.

The linker unit may be cleavable by a cleaving agent that is present in the intracellular environment (e.g., within a lysosome or endosome) or in the tumour microenvironment. The linker unit can be e.g. a peptidyl linker unit that is cleaved by an intracellular peptidase or protease enzyme, for example a lysosomal or endosomal protease, or a peptidase or a protease of the tumour microenvironment. In some embodiments, the peptidyl linker unit is at least two amino acids long or at least three amino acids long. Cleaving agents can include e.g. cathepsins B and D, plasmin, and a matrix metalloproteinase. The peptidyl linker unit cleavable by an intracellular protease or a tumour microenvironment protease may be a Val-Cit linker or a Phe-Lys linker or an Asn, Ala-Asn or Ala-Ala-Asn linker.

The linker unit may be cleavable by a lysosomal hydrolase or a hydrolase of the tumour microenvironment. In an embodiment, the linker unit can comprise a glycosidic bond that is cleavable by an intracellular glycosidase enzyme, for example a lysosomal or endosomal glycosidase, or a glycosidase of the tumour microenvironment. In some embodiments, the glycosidic linker unit comprises a monosaccharide residue or a larger saccharide. Cleaving agents can include e.g. β-glucuronidase, β-galactosidase and β-glucosidase. The glycosidic linker unit cleavable by an intracellular glycosidase or a tumour microenvironment glycosidase may be a β-D-glucuronide linker unit, a β-galactoside linker unit or a β-glucoside linker unit.

The cleavable linker unit may be pH-sensitive, i.e. sensitive to hydrolysis at certain pH values, for example under acidic conditions. For example, an acid-labile linker unit that is hydrolyzable in the lysosome or the tumour microenvironment {e.g., a hydrazone, semicarbazone, thiosemicarbazone, cis-aconitic amide, orthoester, acetal, ketal, or the like) can be used. Such linker units are relatively stable under neutral pH conditions, such as those in the blood, but are unstable at below pH 5.5 or 5.0, or at at below pH 4.5 or 4.0, the approximate pH of the lysosome. In an embodiment, the hydrolyzable linker unit is a thioether linker unit.

The linker unit may be cleavable under reducing conditions, e.g. a disulfide linker unit, examples of which may include disulfide linker units that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonylalpha-methyl-alpha-(2- pyridyl-dithio)toluene), SPDB and SMPT.

The linker unit may comprise or be a malonate linker, a maleimidobenzoyl linker, or a 3′-N-amide analog.

In Formula I, L may be represented by Formula C:

—R₇-L₁-Sₚ-L₂-R₈— Formula C

wherein

R₇ is absent or a group covalently bonded to said oxygen atom (i.e. the oxygen atom of said sugar moiety);

L₁ is a spacer unit of the formula —Sₜ-L₁′-, wherein L₁′ is absent or a spacer moiety;

Sₚ is absent or a specificity unit;

L₂ is absent or a stretcher unit, wherein the stretcher unit optionally comprises a moiety represented by the formula —Sₜ-L₂′-, wherein L₂′ is absent or a stretcher moiety;

R_{B} is absent or a group covalently bonded to the targeting unit;

each Sₜ is independently absent or a moiety represented by any one of the formulas LI to LXVII set forth below;

wherein L comprises at least one Sₜ.

The moiety Sₜ, where present, may be referred to as a stability unit in the present disclosure.

R₇ may be a cleavable group.

R₇ may be absent or any one of the groups a-i:
a. —C(═O)—,
b. —C(═O)NH—,
c. —C(═O)O—,
d. —NHC(═O)—,
e. —NHC(═O)O—,
f. —C(═O)NH—,
g. —NHC(═O)NH—,
h. —P(═O)(OH)—, or
i. —S(═O)₂—.

In an embodiment, R₇ may be absent or any one of the groups a, b, c, f, h, or i.

In an embodiment, R₇ may be absent or —C(═O)—.

In an embodiment, R₇ may be —C(═O)—.

In such an embodiment, L may be considered to form an ester bond with D, such that the oxygen atom of the sugar moiety of the payload molecule is incorporated in the ester group (—O—C(═O)—) linking the payload molecule to L₁. The ester bond may be cleavable. For example, intercellular esterases may be capable of hydrolyzing the ester bond and thereby releasing the payload molecule where desired.

R₈ may, for example, be selected from:

—C(═O)NH—,

—C(═O)O—,

—NHC(═O)—,

—OC(═O)—,

—OC(═O)O—,

—NHC(═O)O—,

—OC(═O)NH—,

—NHC(═O)NH,

—NH—,

—S— and

—O—.

The above groups or moieties may be present in either orientation as R₈. In other words, in the example in which R₈ is —C(═O)NH—, it may be linked to L₁, Sₚ, or L₂ (whichever present) and to T either in the orientation —C(═O)NH-T or T-C(═O)NH—.

In an embodiment, the group —O— may in the context of R₈ be understood as an oxygen atom forming a glycosidic bond between the targeting unit and L₁, L₂ or Sₚ (whichever present).

IV) Stability Units

In an embodiment, each Sₜ is independently absent or a moiety represented by Formula LI

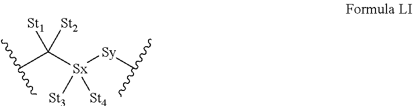

wherein St₁, St₂, St₃, and St₄ are each independently selected from H, CH₃, CH₂CH₃, unsubstituted or substituted C₁-C₆ alkyl, unsubstituted or substituted C₁-C₆ cycloalkyl, unsubstituted or substituted aryl, OH, OCH₃, OR_{O}, wherein R_{O} is either a C₁-C₆ alkyl or a C₁-C₆ substituted alkyl, and an amino acid side chain;

or wherein St₁ together with the carbon to which it is attached, with Sx and optionally with St₃ form an unsubstituted or substituted carbocyclyl or heterocyclyl group;

Sx is either C or N, wherein St₄ is absent if Sx is N;

Sy is either absent, —C(═O)O— or —(CH₂)ₘ—, wherein m is 1 to 4;

the wavy lines in Formula LI show the bonds to the rest of the structure;

the stereochemical center(s) in Formula LI are in either the R or S configuration or a racemic mixture;
and

wherein L comprises at least one Sₜ.

In the context of Formula LI (or any other Formula described in this specification), any “substituted” group or moiety may be substituted by any substituent described e.g. in section I) Definitions.

In embodiments in which St₁, St₂, St₃, and/or St₄ is an unsubstituted or substituted C₁-C₆ alkyl, it/they may be e.g. a C₁-C₄ alkyl or a C₁-C₄ substituted alkyl; a C₁-C₃ alkyl or a C₁-C₃ substituted alkyl; or a C₁-C₂ alkyl or a C₁-C₂ substituted alkyl.

In embodiments in which R_{O} is a C₁-C₆ alkyl or a C₁-C₆ substituted alkyl, it may be e.g. a C₁-C₄ alkyl or a C₁-C₄ substituted alkyl; a C₁-C₃ alkyl or a C₁-C₃ substituted alkyl; or a C₁-C₂ alkyl or a C₁-C₂ substituted alkyl.

In an embodiment, in formula LI, St₁ and St₂ are not both H. In other words, at least one of Sₜ, or St₂ is other than H.

In embodiments wherein St₁ together with the carbon to which it is attached, with Sx and optionally with St₃ form an unsubstituted or substituted carbocyclyl or heterocyclyl group, said carbocyclyl and/or heterocyclyl group may be any carbocyclyl or heterocyclyl group described in this specification, e.g. in section I) Definitions.

In an embodiment, the amino acid side chain may be any amino acid side chain shown below (side chains indicated in the formulas of the amino acids with the shading):

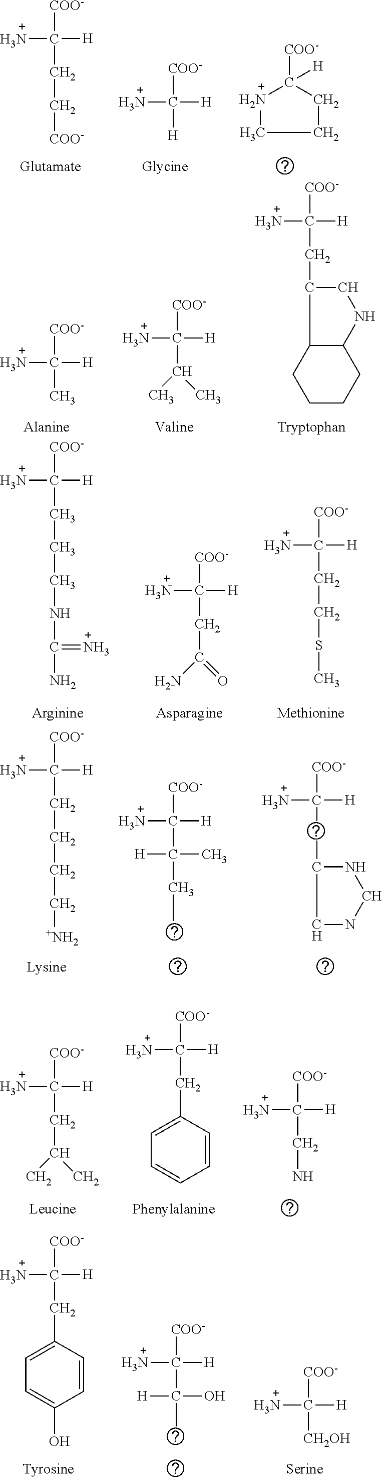

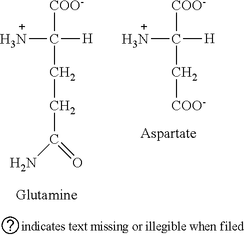

Each Sₜ may, at least in some embodiments, comprise at least 4, or at least 5, or at least 6 carbon atoms. In an embodiment, Sₜ is present in L₁ and comprises at least 4 carbon atoms, or e.g. at least 5 carbon atoms.

In an embodiment, each Sₜ is independently absent, a moiety represented by formula LI, wherein St₃ and St₄ are optionally absent (or both H), or a moiety represented by any one of formulas LII to LXVII

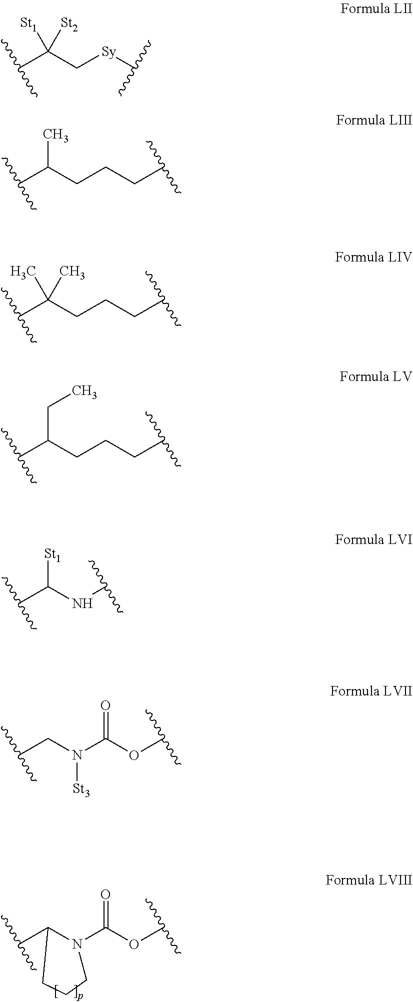

wherein p is from 1 to 2;

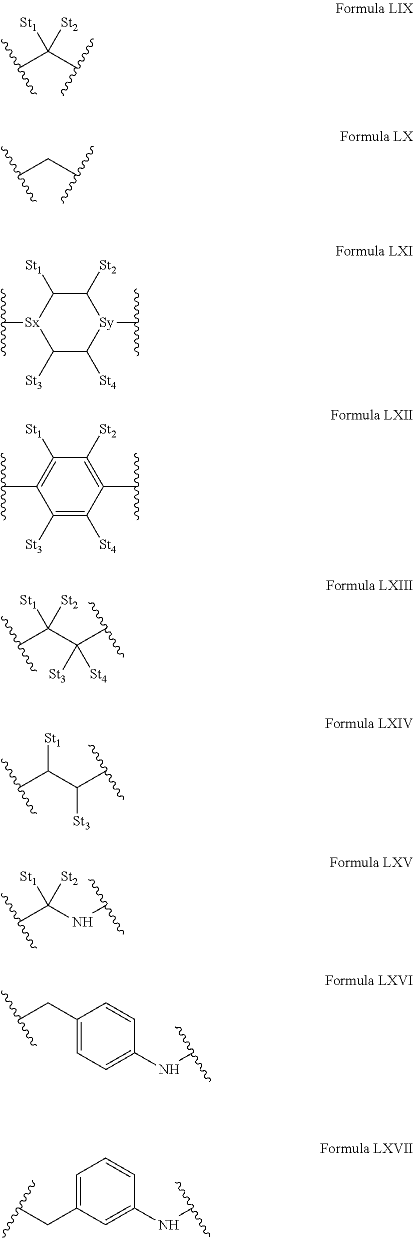

wherein St₁, St₂, St₃, St₄, Sx, and Sy in any one of the formulas LII to LXVII are as defined above and/or according to one or more embodiments described in this specification;

the wavy lines in Formulas LII-LXVII show the bonds to the rest of the structure; and

the stereochemical centers in any one of the Formulas LII-LXVII are in either the R or S configuration or a racemic mixture.

In an embodiment, Sₜ is present in L₁ and a moiety represented by Formula LI as defined above and/or according to one or more embodiments described in this specification.

In an embodiment, Sₜ is present in L₁ and a moiety represented by Formula LII as defined above and/or according to one or more embodiments described in this specification.

In an embodiment, Sₜ is present in L₁ and a moiety represented by Formula LIII as defined above and/or according to one or more embodiments described in this specification.

In an embodiment, Sₜ is present in L₁ and a moiety represented by Formula LIV as defined above and/or according to one or more embodiments described in this specification.

In an embodiment, Sₜ is present in L₁ and a moiety represented by Formula LV as defined above and/or according to one or more embodiments described in this specification.

In an embodiment, Sₜ is present in L₁ and a moiety represented by Formula LVI as defined above and/or according to one or more embodiments described in this specification.

In an embodiment, Sₜ is present in L₁ and a moiety represented by Formula LVII as defined above and/or according to one or more embodiments described in this specification.

In an embodiment, Sₜ is present in L₁ and a moiety represented by Formula LVIII as defined above and/or according to one or more embodiments described in this specification.

In an embodiment, Sₜ is present in L₁ and a moiety represented by Formula LIX as defined above and/or according to one or more embodiments described in this specification.

In an embodiment, Sₜ is present in L₁ and a moiety represented by Formula LX as defined above and/or according to one or more embodiments described in this specification.

In an embodiment, Sₜ is present in L₁ and a moiety represented by Formula LXI as defined above and/or according to one or more embodiments described in this specification.

In an embodiment, Sₜ is present in L₁ and a moiety represented by Formula LXII as defined above and/or according to one or more embodiments described in this specification.

In an embodiment, Sₜ is present in L₁ and a moiety represented by Formula LXIII as defined above and/or according to one or more embodiments described in this specification.

In an embodiment, Sₜ is present in L₁ and a moiety represented by Formula LXIV as defined above and/or according to one or more embodiments described in this specification.

In an embodiment, Sₜ is present in L₁ and a moiety represented by Formula LXV as defined above and/or according to one or more embodiments described in this specification.

In an embodiment, Sₜ is present in L₁ and a moiety represented by Formula LXVI as defined above and/or according to one or more embodiments described in this specification.

In an embodiment, Sₜ is present in L₁ and a moiety represented by Formula LXVII as defined above and/or according to one or more embodiments described in this specification.

In an embodiment, Sₜ is present in L₂ and a moiety represented by Formula LI as defined above and/or according to one or more embodiments described in this specification.

In an embodiment, Sₜ is present in L₂ and a moiety represented by Formula LII as defined above and/or according to one or more embodiments described in this specification.

In an embodiment, Sₜ is present in L₂ and a moiety represented by Formula LIII as defined above and/or according to one or more embodiments described in this specification.

In an embodiment, Sₜ is present in L₂ and a moiety represented by Formula LIV as defined above and/or according to one or more embodiments described in this specification.

In an embodiment, Sₜ is present in L₂ and a moiety represented by Formula LV as defined above and/or according to one or more embodiments described in this specification.

In an embodiment, Sₜ is present in L₂ and a moiety represented by Formula LVI as defined above and/or according to one or more embodiments described in this specification.

In an embodiment, Sₜ is present in L₂ and a moiety represented by Formula LVII as defined above and/or according to one or more embodiments described in this specification.

In an embodiment, Sₜ is present in L₂ and a moiety represented by Formula LVIII as defined above and/or according to one or more embodiments described in this specification.

In an embodiment, Sₜ is present in L₂ and a moiety represented by Formula LIX as defined above and/or according to one or more embodiments described in this specification.

In an embodiment, Sₜ is present in L₂ and a moiety represented by Formula LX as defined above and/or according to one or more embodiments described in this specification.

In an embodiment, Sₜ is present in L₂ and a moiety represented by Formula LXI as defined above and/or according to one or more embodiments described in this specification.

In an embodiment, Sₜ is present in L₂ and a moiety represented by Formula LXII as defined above and/or according to one or more embodiments described in this specification.

In an embodiment, Sₜ is present in L₂ and a moiety represented by Formula LXIII as defined above and/or according to one or more embodiments described in this specification.

In an embodiment, Sₜ is present in L₂ and a moiety represented by Formula LXIV as defined above and/or according to one or more embodiments described in this specification.

In an embodiment, Sₜ is present in L₂ and a moiety represented by Formula LXV as defined above and/or according to one or more embodiments described in this specification.

In an embodiment, Sₜ is present in L₂ and a moiety represented by Formula LXVI as defined above and/or according to one or more embodiments described in this specification.

In an embodiment, Sₜ is present in L₂ and a moiety represented by Formula LXVII as defined above and/or according to one or more embodiments described in this specification.

V) Targeting Units

In an embodiment, the targeting unit is a targeting unit that is capable of binding an immune checkpoint molecule. In an embodiment, the immune checkpoint molecule is any molecule involved in immune checkpoint function. In an embodiment, the immune checkpoint molecule is a checkpoint protein as defined by the NCI Dictionary of Cancer Terms available at https://www.cancer.gov/publications/dictionaries/cancer-terms/def/immune-checkpoint-inhibitor. In an embodiment, the immune checkpoint molecule is a target molecule of an immune checkpoint inhibitor as defined by the NCI Dictionary of Cancer Terms available at https://www.cancer.gov/publications/dictionaries/cancerterms/def/immune-checkpoint-inhibitor. In an embodiment, the immune checkpoint molecule is any molecule described in Marin-Acevedo et al. 2018, J Hematol Oncol 11:39.

In an embodiment, the immune checkpoint molecule is selected from the group of PD-1, PD-L1, CTLA-4, lymphocyte activation gene-3 (LAG-3, CD223), T cell immunoglobulin-3 (TIM3), poly-N-acetyllactosamine, T (Thomsen-Friedenreich) antigen, Globo H, Lewis c (type 1 N-acetyllactosamine), galectin-1, galectin-2, galectin-3, galectin-4, galectin-5, galectin-6, galectin-7, galectin-8, galectin-9, galectin-10, galectin-11, galectin-12, galectin-13, galectin-14, galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and an arginase.

The targeting unit may comprise or be an antibody. For example, the targeting unit may be a tumour cell-targeting antibody, a cancer-targeting antibody and/or an immune cell-targeting antibody. The conjugate may therefore, in some embodiments, be an antibody-payload molecule conjugate, for example a glycosylation inhibitor conjugate or a galectin inhibitor conjugate.

In the context of this specification, the term “antibody” may be understood broadly. For example, an antibody may be e.g. an IgG antibody, an scFv, a single domain antibody, an Fv, a VHH antibody, a diabody, a tandem diabody, a Fab, a Fab′, a F(ab′)₂, a db, a dAb-Fc, a taFv, a scDb, a dAb₂, a DVD-Ig, a Bs(scFv)₄-IgG, a taFv-Fc, a scFv-Fc-scFv, a db-Fc, a scDb-Fc, a scDb-C_{H}3, or a dAb-Fc-dAb. Furthermore, an antibody may be present in monovalent monospecific, multivalent monospecific, bivalent monospecific, or multivalent multispecific forms.

The antibody may be e.g. a human antibody, a humanized antibody, or a chimeric antibody.

In an embodiment, the targeting unit is a bispecific targeting molecule capable of binding to two different target molecules at the same time. In an embodiment, the bispecific targeting unit is a bispecific antibody.

The targeting unit may, alternatively or additionally, comprise or be a peptide, an aptamer, or a glycan.

The targeting unit may, alternatively or additionally, comprise or be a cancer-targeting molecule, such as a ligand of a cancer-associated receptor. Examples of such cancer-targeting molecules include but are not limited to folate.

The targeting unit may further comprise one or more modifications, such as one or more glycosylations or glycans. For example, antibodies typically have one or more glycans. These glycans may be naturally occurring or modified. The payload molecule may, in some embodiments, be conjugated to a glycan of the targeting unit, such as an antibody. In some embodiments, the targeting unit may comprise one or more further groups or moieties, for example a functional group or moiety (e.g. a fluorescent or otherwise detectable label).

The targeting unit may comprise or be, for example, a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan.

The targeting unit may, in an embodiment, comprise or be an antibody selected from the group of an anti-EGFR1 antibody, an epidermal growth factor receptor 2 (HER2/neu) antibody, an antiCD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody and an anti-AXL antibody.

The target molecule may, in an embodiment, comprise or be a molecule selected from the group of EGFR1, epidermal growth factor receptor 2 (HER2/neu), CD22, CD30, CD33, Lewis y, CD20, CD3, PSMA, trophoblast cell-surface antigen 2 (TROP2) and tyrosine-protein kinase receptor UFO (AXL).

The targeting unit may, in an embodiment, comprise or be an immune checkpoint molecule-targeting antibody selected from the group of nivolumab, pembrolizumab, ipilimumab, atezolizumab, avelumab, durvalumab, BMS-986016, LAG525, MBG453, OMP-31M32, JNJ61610588, enoblituzumab (MGA271), MGD009, 8H9, MEDI9447, M7824, metelimumab, fresolimumab, IMC-TR1 (LY3022859), lerdelimumab (CAT152), LY2382770, lirilumab, IPH4102, 9B12, MOXR 0916, PF-04518600 (PF-8600), MEDI6383, MEDI0562, MEDI6469, INCAGN01949, GSK3174998, TRX-518, BMS-986156, AMG 228, MEDI1873, MK-4166, INCAGN01876, GWN323, JTX-2011, GSK3359609, MEDI-570, utomilumab (PF-05082566), urelumab, ARGX-110, BMS-936561 (MDX-1203), varlilumab, CP-870893, APX005M, ADC-1013, lucatumumab, Chi Lob 7/4, dacetuzumab, SEACD40, RO7009789, and MEDI9197.

The targeting unit may comprise or be a molecule selected from the group of an immune checkpoint inhibitor, an anti-immune checkpoint molecule, anti-PD-1, anti-PD-L1 antibody, anti-CTLA-4 antibody, or an antibody targeting an immune checkpoint molecule selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), galectin-1, galectin-2, galectin-3, galectin-4, galectin-5, galectin-6, galectin-7, galectin-8, galectin-9, galectin-10, galectin-11, galectin-12, galectin-13, galectin-14, galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

The target molecule may comprise or be a molecule selected from the group of an immune checkpoint molecule, PD-1, PD-L1, CTLA-4, lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), galectin-1, galectin-2, galectin-3, galectin-4, galectin-5, galectin-6, galectin-7, galectin-8, galectin-9, galectin-10, galectin-11, galectin-12, galectin-13, galectin-14, galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3Kγ), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

VI) Stretcher Units

The term “stretcher unit” may refer to any group, moiety or linker portion capable of linking R₇, L₁, or Sₚ (whichever present) to R₈ (if present) or to the targeting unit. Various types of stretcher units may be suitable, and many are known in the art.

The stretcher unit may be a group, moiety or linker portion capable of covalently linking R₇, L₁, or Sₚ (whichever present) to R₈ (if present) or to the targeting unit.

In embodiments in which L₂ is present and a stretcher unit, it may comprise a moiety represented by the formula —Sₜ-L₂′-, wherein L₂′ is absent or a stretcher moiety.

Again, L₂′ or stretcher moiety may be any group or moiety capable of linking Sₜ to R₈ (if present) or to the targeting unit.

In an embodiment, L₂′ is either absent or a any one of the groups a-j:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety,
h. a substituted branched PEG₂₋₅₀ polyethylene glycol moiety,
i. a moiety represented by the formula XXVI as set out below, or
j. a moiety represented by the formula XXVII as set out below.

In an embodiment, L₂′ is a C₁₋₁₂ alkylene, for example a C₁₋₆ alkylene, a C₁₋₄ alkylene or a C₁₋₂ alkylene, or a substituted C₁₋₁₂ alkylene, for example a substituted C₁₋₆ alkylene, a substituted C₁₋₄ alkylene, or a substituted C₁₋₂ alkylene.

The stretcher unit L₂ may have a functional group that can form a bond with a functional group of the targeting unit. The stretcher unit may also have a functional group that can form a bond with a functional group of either R₇, L₁, or Sₚ. Useful functional groups that can be present on the targeting unit, either naturally or via chemical manipulation, include, but are not limited to, sulfhydryl (—SH), amino, hydroxyl, carboxy, the anomeric hydroxyl group of a carbohydrate, and carboxyl. The functional groups of the targeting unit may, in an embodiment, be sulfhydryl and amino. The stretcher unit can comprise for example, a maleimide group, an aldehyde, a ketone, a carbonyl, or a haloacetamide for attachment to the targeting unit.

In one example, sulfhydryl groups can be generated by reduction of the intramolecular disulfide bonds of a targeting unit, such as an antibody. In another embodiment, sulfhydryl groups can be generated by reaction of an amino group of a lysine moiety of an antibody or other targeting unit with 2-iminothiolane (Traut's reagent) or other sulfhydryl generating reagents. In certain embodiments, the targeting unit is a recombinant antibody and is engineered to carry one or more lysines. In certain other embodiments, the recombinant antibody is engineered to carry additional sulfhydryl groups, e.g. additional cysteines.

In an embodiment, the stretcher unit has an electrophilic group that is reactive to a nucleophilic group present on the targeting unit (e.g. an antibody). Useful nucleophilic groups on the targeting unit include but are not limited to, sulfhydryl, hydroxyl and amino groups. The heteroatom of the nucleophilic group of the targeting unit is reactive to an electrophilic group on a stretcher unit and forms a covalent bond to the stretcher unit. Useful electrophilic groups include, but are not limited to, maleimide and haloacetamide groups. For an antibody as the targeting unit, the electrophilic group may provide a convenient site for antibody attachment for those antibodies having an accessible nucleophilic group.

In another embodiment, the stretcher unit has a reactive site which has a nucleophilic group that is reactive to an electrophilic group present on a targeting unit (e.g. an antibody). Useful electrophilic groups on a targeting unit include, but are not limited to, aldehyde and ketone and carbonyl groups. The heteroatom of a nucleophilic group of the stretcher unit can react with an electrophilic group on the targeting unit and form a covalent bond to the targeting unit, e.g. an antibody. Useful nucleophilic groups on the stretcher unit include, but are not limited to, hydrazide, hydroxylamine, amino, hydrazine, thiosemicarbazone, hydrazine carboxylate, and arylhydrazide. For an antibody as the targeting unit, the electrophilic group on the antibody may provide a convenient site for attachment to a nucleophilic stretcher unit.

In an embodiment, the stretcher unit has a reactive site which has an electrophilic group that is reactive with a nucleophilic group present on a targeting unit, such as an antibody. The electrophilic group provides a convenient site for the targeting unit (e.g., antibody) attachment. Useful nucleophilic groups on an antibody include but are not limited to, sulfhydryl, hydroxyl and amino groups. The heteroatom of the nucleophilic group of an antibody is reactive to an electrophilic group on the stretcher unit and forms a covalent bond to the stretcher unit. Useful electrophilic groups include, but are not limited to, maleimide and haloacetamide groups and NHS esters.

In another embodiment, a stretcher unit has a reactive site which has a nucleophilic group that is reactive with an electrophilic group present on the targeting unit. The electrophilic group on the targeting unit (e.g. antibody) provides a convenient site for attachment to the stretcher unit. Useful electrophilic groups on an antibody include, but are not limited to, aldehyde and ketone carbonyl groups. The heteroatom of a nucleophilic group of the stretcher unit can react with an electrophilic group on an antibody and form a covalent bond to the antibody. Useful nucleophilic groups on the stretcher unit include, but are not limited to, hydrazide, oxime, amino, hydrazine, thiosemicarbazone, hydrazine carboxylate, and arylhydrazide.

In some embodiments, the stretcher unit forms a bond with a sulfur atom of the targeting unit via a maleimide group of the stretcher unit. The sulfur atom can be derived from a sulfhydryl group of the targeting unit. Representative stretcher units of this embodiment include those within the square brackets of Formulas Xa and Xb, wherein the wavy line indicates attachment within the conjugate and R¹⁷ is —C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O— (C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkyl)-NH, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylenearylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—. Any of the R¹⁷ substituents can be substituted or nonsubstituted. In an embodiment, the R¹⁷ substituents are unsubstituted. In another embodiment, the R¹⁷ substituents are optionally substituted. In some embodiments, the R¹⁷ groups are optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

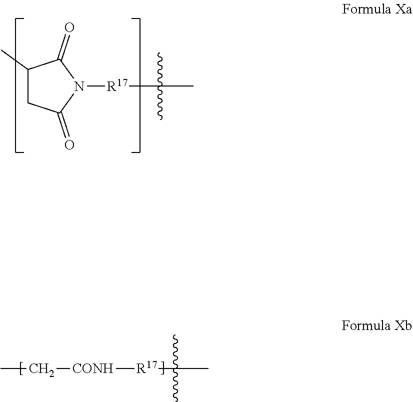

In the context of the embodiments of the stretcher unit, the wavy line may (although not necessarily) indicate attachment within the conjugate to either R₇, L₁, or Sₚ, whichever present. The free bond without the wavy line, typically at the opposite end of the stretcher unit, may indicate the bond to the targeting unit.

An illustrative stretcher unit is that of Formula Xa wherein R¹⁷ is —C₂-C₅ alkylene-C(═O)— wherein the alkylene is optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group. Exemplary embodiments are as follows:

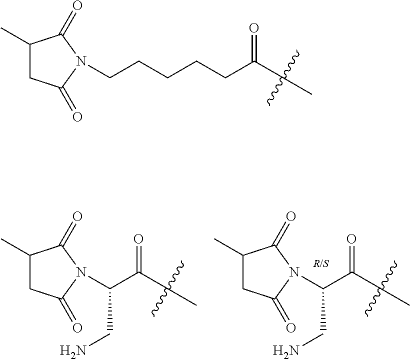 [SMILES: *C(=O)CCCCCN1C(=O)CC(C)C1=O ; *C(=O)[C@H](CN)N1C(=O)CC(C)C1=O ; *C(=O)[C@H](CN)N1C(=O)CC(C)C1=O]

It will be understood that the substituted succinimide may exist in a hydrolyzed form as shown below:

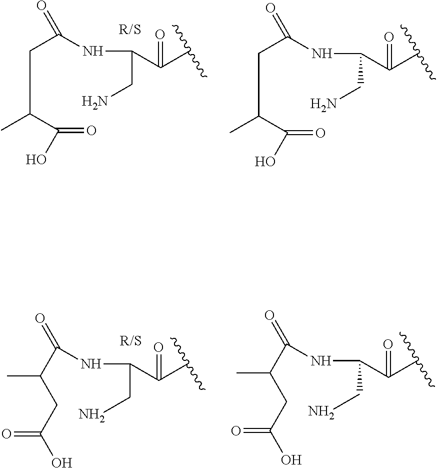 [SMILES: *C(=O)C(CN)NC(=O)CC(C)C(=O)O ; *C(=O)[C@@H](CN)NC(=O)CC(C)C(=O)O ; *C(=O)C(CN)NC(=O)C(C)CC(=O)O ; *C(=O)[C@@H](CN)NC(=O)C(C)CC(=O)O]

Illustrative stretcher units prior to conjugation to the targeting unit include the following:

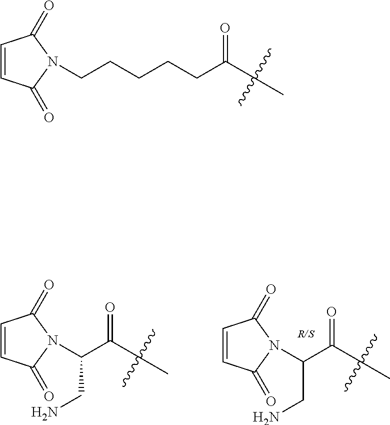 [SMILES: *C(=O)CCCCCN1C(=O)C=CC1=O ; *C(=O)[C@H](CN)N1C(=O)C=CC1=O ; *C(=O)C(CN)N1C(=O)C=CC1=O]

It will be understood that the amino group of the stretcher unit may be suitably protected by a amino protecting group during synthesis, e.g., an acid labile protecting group (e.g, BOC).

Yet another illustrative stretcher unit is that of Formula Xb wherein R¹⁷ is —(CH₂)₅—:

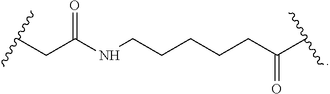 [SMILES: *CC(=O)NCCCCCC(*)=O]

In another embodiment, the stretcher unit is linked to the targeting unit via a disulfide bond between a sulfur atom of the targeting unit and a sulfur atom of the stretcher unit. A representative stretcher unit of this embodiment is depicted within the square brackets of Formula XI, wherein the wavy line indicates attachment within the conjugate and R¹⁷ is as described above for Formula Xa and Xb.

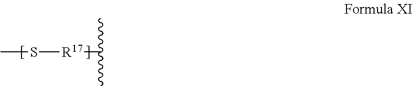

In yet another embodiment, the reactive group of the stretcher unit contains a reactive site that can form a bond with a primary or secondary amino group of the targeting unit. Example of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XIIa, XIIb, and XIIc wherein the wavy line indicates attachment within the within the conjugate and R¹⁷ is as described above for Formula Xa and Xb.

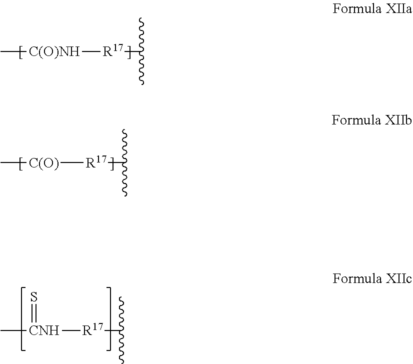

In yet another embodiment, the reactive group of the stretcher unit contains a reactive site that is reactive to a modified carbohydrate's (—CHO) group that can be present on the targeting unit. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XIIIa, XIIIb, and XIIIc, wherein the wavy line indicates attachment within the conjugate and R¹⁷ is as described above for Formula Xa and Xb.

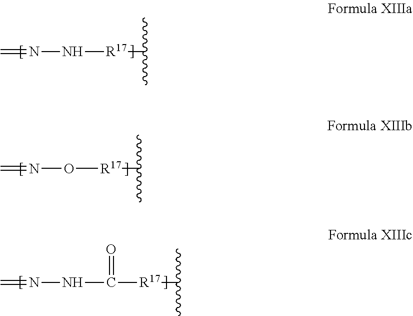

In some embodiments, it may be desirable to extend the length of the stretcher unit. Accordingly, a stretcher unit can comprise additional components. For example, a stretcher unit can include those within the square brackets of formula XIVa1:

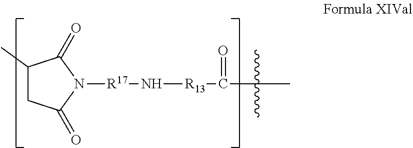

wherein the wavy line indicates attachment to the remainder of the conjugate and the free bond to the targeting unit;

and R¹⁷ is as described above. For example, R¹⁷ may be —C₂-C₅ alkylene-C(═O)— wherein the alkylene is optionally substituted by a basic unit, e.g —(CH₂)ₓNH₂, —(CH₂)ₓNHR^{a}, and —(CH₂)ₓNR^{a}₂, wherein x is an integer in the range of 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and

R¹³ is —C₁-C₆ alkylene-, —C₃-C₈ carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene(C₃-C₈ heterocyclo)-, or —(C₃-C₈ heterocyclo)-C₁-C₁₀ alkylene-. In an embodiment, R¹³ is —C₁-C₆ alkylene-.

The stretcher unit may, in some embodiments, have a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

In an embodiment, the stretcher unit forms a bond with a sulfur atom of the targeting unit, for example an antibody. The sulfur atom can be derived from a sulfhydryl group of the antibody. Representative stretcher units of this embodiment are depicted within the square brackets of Formulas XVa and XVb, wherein R¹⁷ is selected from —C₁-C₁₀ alkylene-, —C₁-C₁₀ alkenylene-, —C₁-C₁₀ alkynylene-, carbocyclo-, —O—(C₁-C₈ alkylene)-, O—(C₁-C₈ alkenylene)-, —O—(C₁-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, -heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₁-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynylene, aryl, carbocycle, carbocyclo, heterocyclo, and arylene radicals, whether alone or as part of another group, are optionally substituted. In some embodiments, said alkyl, alkenyl, alkynyl, alkylene, alkenylene, alkynylene, aryl, carbocycle, carbocyclo, heterocyclo, and arylene radicals, whether alone or as part of another group, are unsubstituted. In some embodiments, R¹⁷ is selected from —C₁-C₁₀ alkylene-, -carbocyclo-, —O—(C₁-C₈ alkylene)-arylene-, —C₁-C₁₀ alkylene-arylene-, -arylene-C₁-C₁₀ alkylene-, —C₁-C₁₀ alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O)ᵣ—CH₂—; and r is an integer ranging from 1-10, wherein said alkylene groups are unsubstituted and the remainder of the groups are optionally substituted.

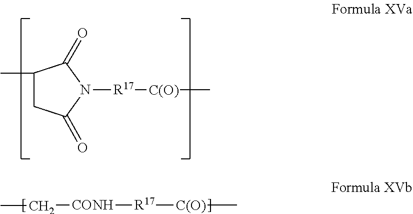

It is to be understood from all the exemplary embodiments that even where not denoted expressly, one or more payload molecule moieties can be linked to a targeting unit, i.e. n may be 1 or more.

An illustrative stretcher unit is that of Formula XVa wherein R¹⁷ is —(CH₂CH₂O)ᵣ—CH₂—: and r is 2:

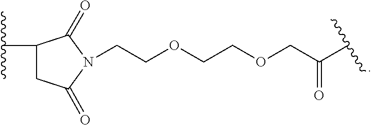 [SMILES: *C(=O)COCCOCCN1C(=O)CC(*)C1=O]

An illustrative stretcher unit is that of Formula XVa wherein R¹⁷ is arylene- or arylene-C₁-C₁₀ alkylene-. In some embodiments, the aryl group is an unsubstituted phenyl group.

In certain embodiments, the stretcher unit is linked to the targeting unit via a disulfide bond between a sulfur atom of the targeting unit and a sulfur atom of the stretcher unit. A representative stretcher unit of this embodiment is depicted in Formula XVI, wherein R¹⁷ is as defined above.

—S [symbol] S—R¹⁷—C(O) [symbol] Formula XVI

The S moiety in the formula XVI above may refer to a sulfur atom of the targeting unit, unless otherwise indicated by context.

In yet other embodiments, the stretcher unit contains a reactive site that can form a bond with a primary or secondary amino group of the targeting unit, such as an antibody. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative stretcher units of this embodiment are depicted within the square brackets of Formulas XVIIa and XVIIb, wherein —R¹⁷ is as defined above:

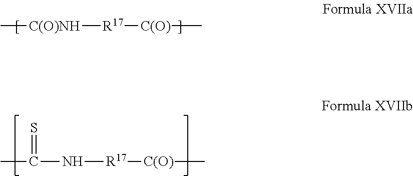

In some embodiments, the stretcher unit contains a reactive site that is reactive to a modified carbohydrate's (—CHO) group that can be present on the targeting unit, for example an antibody. For example, a carbohydrate can be mildly oxidized using a reagent such as sodium periodate and the resulting (—CHO) unit of the oxidized carbohydrate can be condensed with a stretcher unit that contains a functionality such as a hydrazide, an oxime, a primary or secondary amine, a hydrazine, a thiosemicarbazone, a hydrazine carboxylate, and an arylhydrazide. Representative stretcher units of this embodiment are depicted within the square brackets of Formulas XVIIIa, XVIIIb, and XVIIIc, wherein —R¹⁷— is as defined as above.

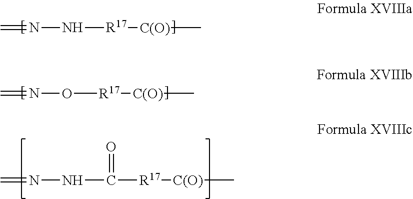

In embodiments in which the targeting unit is a glycoprotein, for example an antibody, the glycoprotein, i.e. the targeting unit, may be contacted with a suitable substrate, such as UDP-GalNAz, in the presence of a GalT or a GalT domain catalyst, for example a mutant GalT or GalT domain. Thus the targeting unit may have a GalNAz residue incorporated therein. The payload molecule may then be conjugated via a reaction with the GalNAz thus incorporated in the targeting unit.

WO/2007/095506, WO/2008/029281 and WO/2008/101024 disclose methods of forming a glycoprotein conjugate wherein the glycoprotein is contacted with UDP-GalNAz in the presence of a GalT mutant, leading to the incorporation of GalNAz at a terminal non-reducing GlcNAc of an antibody carbohydrate. Subsequent copper-catalyzed or copper-free (metal-free) click chemistry with a terminal alkyne or Staudinger ligation may then be used to conjugate a molecule of interest, in this case the payload molecule, optionally via a suitable linker unit or stretcher unit, to the attached azide moiety.

If no terminal GlcNAc sugars are present on the targeting unit, such as an antibody, endoenzymes Endo H, Endo A, Endo F, Endo D, Endo T, Endo S and/or Endo M and/or a combination thereof, the selection of which depends on the nature of the glycan, may be used to generate a truncated chain which terminates with one N-acetylglucosamine residue attached in an antibody Fc region.

In an embodiment, the endoglycosidase is Endo S, Endo S49, Endo F or a combination thereof.

In an embodiment, the endoglycosidase is Endo S, Endo F or a combination thereof.

Endo S, Endo A, Endo F, Endo M, Endo D and Endo H are known to the person skilled in the art. Endo S49 is described in WO/2013/037824 (Genovis AB). Endo S49 is isolated from Streptococcus pyogenes NZ131 and is a homologue of Endo S. Endo S49 has a specific endoglycosidase activity on native IgG and cleaves a larger variety of Fc glycans than Endo S.

Galactosidases and/or sialidases can be used to trim galactosyl and sialic acid moieties, respectively, before attaching e.g. GalNAz moieties to terminal GlcNAcs. These and other deglycosylation steps, such as defucosylation, may be applied to G2F, G1F, G0F, G2, G1, and G0, and other glycoforms.

Mutant GalTs include but are not limited to bovine beta-1,4-galactosyltransferase I (GalT1) mutants Y289L, Y289N, and Y289I disclosed in Ramakrishnan and Qasba, J. Biol. Chem., 2002, vol. 277, 20833) and GalT1 mutants disclosed in WO/2004/063344 and WO/2005/056783 and their corresponding human mutations.

Mutant GalTs (or their GalT domains) that catalyze the formation of i) a glucose-((1,4)-N-acetylglucosamine bond, ii) an N-acetylgalactosamine-β(1,4)-N-acetylglucosamine bond, iii) a N-acetylglucosamine-β(1,4)-N-acetylglucosamine bond, iv) a mannose-β(1,4)-N-acetylglucosamine bond are disclosed in WO 2004/063344. The disclosed mutant GalT (domains) may be included within full-length GalT enzymes, or in recombinant molecules containing the catalytic domains, as disclosed in WO2004/063344.

In an embodiment, GalT or GalT domain is for example Y284L, disclosed by Bojarovn et al., Glycobiology 2009, 19, 509.

In an embodiment, GalT or GalT domain is for example R228K, disclosed by Qasba et al., Glycobiology 2002, 12, 691.

In an embodiment, the mutant GalT1 is a bovine β(1,4)-galactosyltransferase 1.

In an embodiment, the bovine GalT1 mutant is selected from the group consisting of Y289L, Y289N, Y289I, Y284L and R228K.

In an embodiment, the mutant bovine GalT1 or GalT domain is Y289L.

In an embodiment, the GalT comprises a mutant GalT catalytic domain from a bovine β(1,4)-galactosyltransferase, selected from the group consisting of GalT Y289F, GalT Y289M, GalT Y289V, GalT Y289G, GalT Y289I and GalT Y289A. These mutants may be provided via site-directed mutagenesis processes, in a similar manner as disclosed in WO 2004/063344, in Qasba et al., Prot. Expr. Pur. 2003, 30, 219 and in Qasba et al., J. Biol. Chem. 2002, 277, 20833 for Y289L, Y289N and Y289I.

Another type of a suitable GalT is α(1,3)-N-galactosyltransferase (α3Gal-T).

In an embodiment, a (1,3)-N-acetylgalactosaminyltransferase is α3GalNAc-T as disclosed in WO2009/025646. Mutation of α3Gal-T can broaden donor specificity of the enzyme, and make it an α3GalNAc-T. Mutation of α3GalNAc-T can broaden donor specificity of the enzyme. Polypeptide fragments and catalytic domains of α(1,3)-N-acetylgalactosaminyltransferases are disclosed in WO/2009/025646.

In an embodiment, the GalT is a wild-type galactosyltransferase.

In an embodiment, the GalT is a wild-type β(1,4)-galactosyltransferase or a wild-type β(1,3)-N-galactosyltransferase.

In an embodiment, GalT is β(1,4)-galactosyltransferase I.

In an embodiment, the β(1,4)-galactosyltransferase is selected from the group consisting of a bovine β(1,4)-Gal-T1, a human β(1,4)-Gal-T1, a human β(1,4)-Gal-T2, a human β(1,4)-GalT3, a human β(1,4)-Gal-T4 and B (1,3)-Gal-T5.

In an embodiment, acetylgalactosaminyltransferase is selected from the mutants disclosed in WO 2016/170186.

The linker unit or the stretcher unit may comprise an alkyne group, for example a cyclic alkyne group, capable of reacting with the azide group of the GalNAz incorporated in the targeting unit, thereby forming a triazole group. Examples of suitable cyclic alkyne groups may include DBCO, OCT, MOFO, DIFO, DIFO2, DIFO3, DIMAC, DIBO, ADIBO, BARAC, BCN, Sondheimer diyne, TMDIBO, S-DIBO, COMBO, PYRROC, or any modifications or analogs thereof.

BCN and its derivatives are disclosed in WO/2011/136645. DIFO, DIFO2 and DIFO 3 are disclosed in US 2009/0068738. DIBO is disclosed in WO 2009/067663. DIBO may optionally be sulfated (S-DIBO) as disclosed in J. Am. Chem. Soc. 2012, 134, 5381. BARAC is disclosed in J. Am. Chem. Soc. 2010, 132, 3688-3690 and US 2011/0207147. ADIBO derivatives are disclosed in WO/2014/189370.

The stretcher unit may thus comprise an optionally substituted triazole group formed by a reaction between a (cyclic) alkyne group and an azide group of a GalNAz group incorporated at a terminal non-reducing GlcNAc of the targeting unit.

VII) Specificity Units

The term “specificity unit” or Sₚ may refer to any group, moiety or linker portion capable of linking R₇ or L₁ (if present) to L₂ (if present), to R₈ (if present) or to the targeting unit.

The specificity unit may be any group, moiety or linker portion capable of covalently linking R₇ or L₁ (if present) to L₂ (if present), to R₈ (if present) or to the targeting unit.

The specificity unit may, in some embodiments, be cleavable. Thereby it can confer cleavability to the linker unit.

The specificity unit may comprise a labile bond configured to be cleavable in suitable conditions. It may thus confer specificity to the cleavability of the conjugate. For example, the stretcher unit may be cleavable only after the cleavage of the specificity unit.

In an embodiment, Sₚ is either absent or any one of the groups a-n:
a. dipeptide,
b. tripeptide,
c. tetrapeptide,
d. valine-citrulline,
e. phenylalanine-lysine,
f. valine-alanine,
g. valine-serine,
h. asparagine,
i. alanine-asparagine,
j. alanine-alanine-asparagine,
k. a hydrazone,
l. an ester,
m. a disulfide, or
n. a glycoside.

Sₚ may be a disulfide.

In an embodiment, Sₚ is present, for example a disulfide, and Sₜ is present in L₂. The Sₜ present in L₂ may be any Sₜ according to any one of the embodiments described in this specification. In such embodiments, a second Sₜ according to one or more embodiments described in this specification may be present in L₁.

The specificity unit can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each Sₚ unit independently may have the formula XIXa or XIXb denoted below in the square brackets:

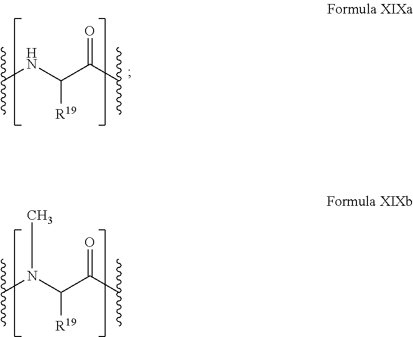

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH) NH₂, —(CH₂)₃NH₂, —(CH₂)₃NHCOCH₃, —(CH₂)₃NHCHO, —(CH₂)₄NHC(═NH) NH₂, —(CH₂)₄NH₂, (CH₂)₄NHCOCH₃, —(CH₂)₄NHCHO, —(CH₂)₃NHCONH₂, —(CH₂)₄NHCONH₂, —CH₂CH₂CH(OH) CH₂NH₂, 2-pyridylmethyl-, 3-pyridylmethyl-, 4-pyridylmethyl-, phenyl, cyclohexyl,

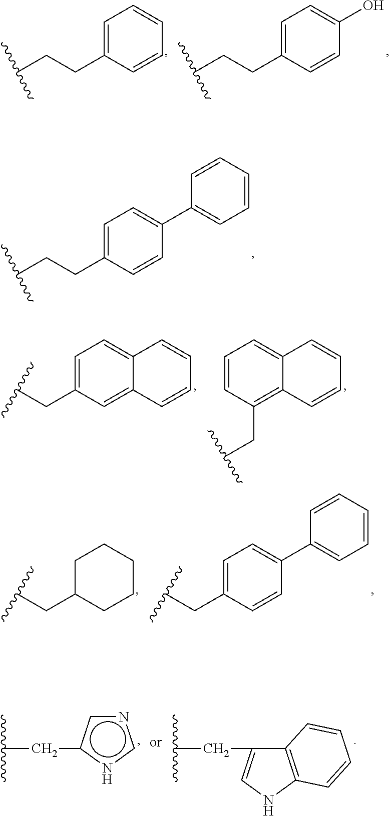

In some embodiments, the specificity unit can be enzymatically cleavable by one or more enzymes, including a cancer or tumor-associated protease, to liberate the payload molecule.

In certain embodiments, the specificity unit can comprise natural amino acids. In other embodiments, the specificity unit can comprise non-natural amino acids. Illustrative specificity units are represented by formulas (XX)-(XXII):

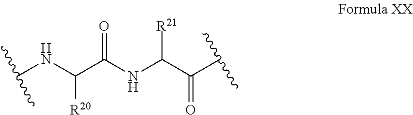

wherein R²⁰ and R²¹ are as follows:

| R²⁰ | R²¹ |
|---|---|
| Benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 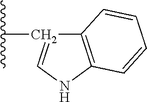 [SMILES: *c1c[nH]c2ccccc12] | (CH₂)₃NHCONH₂; |
| benzyl | methyl; |
| benzyl | (CH₂)₃NHC (═NH)NH₂; |

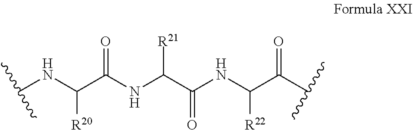

wherein R²⁰, R²¹ and R²² are as follows:

| R²⁰ | R²¹ | R²² |
|---|---|---|
| benzyl | benzyl | (CH²)⁴NH²; |
| isopropyl | benzyl | (CH²)⁴NH²; and |
| H | benzyl | (CH²)⁴NH² |

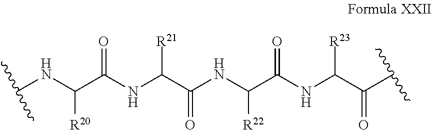

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl |

Exemplary specificity units include, but are not limited to, units of formula XX wherein R²⁰ is benzyl and R²¹ is —(CH₂)₄NH₂; R²⁰ is isopropyl and R₂₁ is —(CH₂)₄NH₂; or R²⁰ is isopropyl and R₂₁ is —(CH₂)₃NHCONH₂. Another exemplary specificity unit is a specificity unit of formula XXI wherein R²⁰ is benzyl, R²¹ is benzyl, and R²² is —(CH₂)₄NH₂.

Useful specificity units can be designed and optimized in their selectivity for enzymatic cleavage by a particular enzyme, for example, a tumour-associated protease. In one embodiment, the specificity unit is cleavable by cathepsin B, C and D, or a plasmin protease.

In an embodiment, the specificity unit is a dipeptide, tripeptide, tetrapeptide or pentapeptide. When R¹⁹, R²⁰, R²¹, R²² or R²³ is other than hydrogen, the carbon atom to which R¹⁹, R²⁰, R₂₁, R₂₂ or R²³ is attached is chiral. Each carbon atom to which R¹⁹, R²⁰, R²¹, R²² or R²³ is attached may be independently in the (S) or (R) configuration.

In an embodiment, the specificity unit comprises or is valine-citrulline (vc or val-cit). In another embodiment, the the specificity unit comprises or is phenylalanine-lysine (i.e. fk). In yet another embodiment, the specificity unit comprises or is N-methylvaline-citrulline. In yet another embodiment, the specificity unit comprises or is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and/or isonepecotic acid. In a further embodiment, the specificity unit comprises or is an asparagine (Asn), alanine-asparagine (Ala-Asn) and/or alaninealanine-asparagine (Ala-Ala-Asn).

VIII) Spacer Units

The term “spacer unit” may refer to any group, moiety or linker portion capable of linking R₇ to Sₚ (if present), L₂ (if present) or the targeting unit. Various types of spacer units may be suitable, and many are known in the art. The spacer unit may be capable of covalently linking R₇ to Sₚ (if present), L₂ (if present) or the targeting unit.

In an embodiment, L₁ or the spacer unit is represented by or comprises formula —Sₜ-L₁′-, wherein L₁′ is absent or a spacer moiety.

Sₜ in the spacer unit may be absent or any Sₜ described in this specification.

L₁ may be a spacer unit of the formula —Sₜ-L₁′- covalently connecting R₇ to Sₚ (when R₇ is present).

Again, L₁′ or spacer moiety may be any group or moiety capable of linking R₇ to Sₚ, L₂ or R₈ (whichever present).

L₁′ may be either absent or any one of the groups a-h:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety, or
h. a substituted branched PEG₂₋₅₀ polyethylene glycol moiety.

L₁′ may be a substituted or unsubstituted C₁₋₁₂ alkylene, for example a substituted or unsubstituted C₁₋₆ alkylene, a substituted or unsubstituted C₁₋₄ alkylene, or a substituted or unsubstituted C₁₋₂ alkylene.

Spacer units may be of two general types: non self-immolative or self-immolative. A non self-immolative spacer unit is one in which part or all of the spacer unit remains bound to the payload molecule moiety after cleavage, for example enzymatic cleavage, of a specificity unit from the conjugate. Examples of a non self-immolative spacer unit include, but are not limited to, a (glycine-glycine) spacer unit and a glycine spacer unit. When a conjugate containing a glycine-glycine spacer unit or a glycine spacer unit undergoes enzymatic cleavage via an enzyme (e.g., a tumour-cell associated-protease, a cancer-cell-associated protease or a lymphocyte-associated protease), a glycine-glycine-R₇-payload molecule moiety or a glycine-R₇-payload molecule moiety is cleaved from —Sₚ-L₂-R₈-T (whichever, if any, of Sₚ-L₂-R₈ is present). In one embodiment, an independent hydrolysis reaction takes place within the target cell, cleaving the glycine-R₇-payload molecule moiety bond and liberating the payload molecule (and R₇).

In some embodiments, the non self-immolative spacer unit (-L₁-) is -Gly-. In some embodiments, the non self-immolative spacer unit (-L₁-) is -Gly-Gly-.

However, the spacer unit may also be absent.

Alternatively, a conjugate containing a self-immolative spacer unit can release -D, i.e. the payload molecule, or D-R₇—. In the context of this specification, the term “self-immolative spacer unit” may refer to a bifunctional chemical moiety that is capable of covalently linking together two spaced chemical moieties into a stable tripartite molecule. It may spontaneously separate from the second chemical moiety if its bond to the first moiety is cleaved.

In some embodiments, the spacer unit is a p-aminobenzyl alcohol (PAB) unit (see Schemes 1 and 2 below) the phenylene portion of which is substituted with Qₘ wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

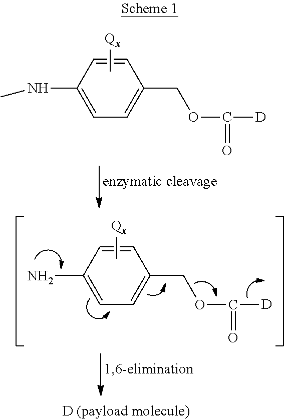

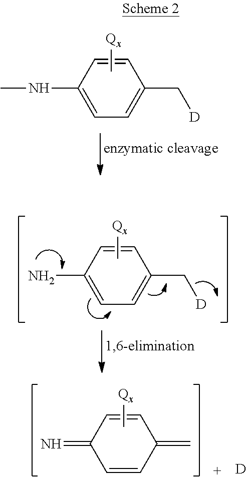

In some embodiments, the spacer unit is a PAB group that is linked to —Sₚ—, -L₂-, —R₈— or -T via the amino nitrogen atom of the PAB group, and connected directly to —R₇— or to -D via a carbonate, carbamate or ether group. Without being bound by any particular theory or mechanism, Scheme 1 depicts a possible mechanism of release of a PAB group which is attached directly to -D or R⁷ via a carbamate or carbonate group.

In Scheme 1, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0 4. The alkyl, alkenyl and alkynyl groups, whether alone or as part of another group, can be optionally substituted.

Without being bound by any particular theory or mechanism, Scheme 2 depicts a possible mechanism of payload molecule release of a PAB group which is attached directly to -D or to —R₇-D via an ether or amine linkage, wherein D may include the oxygen or nitrogen group that is part of the payload molecule.

In Scheme 2, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted.

Other examples of self-immolative spacer units include, but are not limited to, aromatic compounds that are electronically similar to the PAB group such as 2-aminoimidazol-5-methanol derivatives and ortho or para-aminobenzylacetals. Other possible spacer units may be those that undergo cyclization upon amide bond hydrolysis, such as substituted and unsubstituted 4-aminobutyric acid amides, appropriately substituted bicyclo[2.2.1] and bicyclo[2.2.2] ring systems and 2-aminophenylpropionic acid amides. Elimination of amine-containing payload molecules (e.g. glycosylation inhibitors) that are substituted at the α-position of glycine are also examples of self-immolative spacers.

In an embodiment, the spacer unit is a branched bis(hydroxymethyl)-styrene (BHMS) unit as depicted in Scheme 3, which can be used to incorporate and release multiple payload molecules.

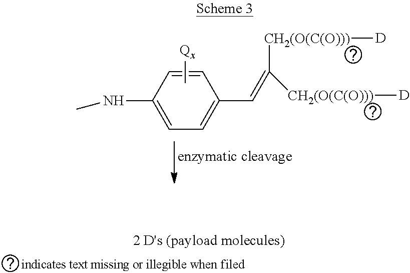

In Scheme 3, Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; m is an integer ranging from 0-4; and n is 0 or 1. The alkyl, alkenyl and alkynyl groups, whether alone or as part of another group, can be optionally substituted.

In some embodiments, the -D moieties are the same. In yet another embodiment, the -D moieties are different.

In an embodiment, the spacer unit is represented by any one of Formulas (XXIII)-(XXV):

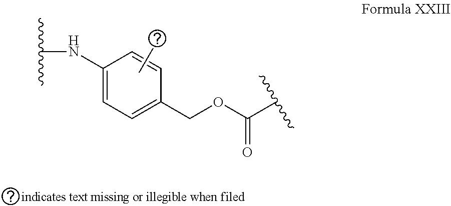

wherein Q is —C₁-C₈ alkyl, —C₁-C₈ alkenyl, —C₁-C₈ alkynyl, —O—(C₁-C₈ alkyl), —O—(C₁-C₈ alkenyl), —O—(C₁-C₈ alkynyl), -halogen, -nitro or -cyano; and m is an integer ranging from 0-4. The alkyl, alkenyl and alkynyl groups, whether alone or as part of another group, can be optionally substituted;

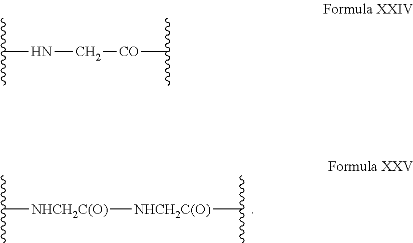

IX) Further Linker Units

The linker unit may, in some embodiments, comprise a polymer moiety. Such polymer moieties are described e.g. in WO 2015/189478.

In an embodiment, the linker unit L comprises a moiety represented by the formula XXVI, or L2′ is represented by the formula XXVI:

—Y—(CH₂)ₒ—O]_{q}—P— Formula XXVI

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

O is in the range of 1 to 10;

q is at least 1; and

each Y is independently selected from the group consisting of S, NH and 1,2,3-triazolyl, wherein 1,2,3-triazolyl is optionally substituted.

In the above formula, P may be linked to T and Y to D, i.e. the payload molecule. Y may be linked to D directly, or further groups, moieties or units may be present between Y and D.

Dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan each comprise at least one hydroxyl group. The presence of the at least one hydroxyl group allows the linking of one or more substituents to the polymer as described herein. Many of these polymers also comprise saccharide units that may be further modified, e.g. oxidatively cleaved, to introduce functional groups to the polymer. P may thus also be a polymer derivative.

In this specification, the term “saccharide unit” should be understood as referring to a single monosaccharide moiety.

In this specification, the term “saccharide” should be understood as referring to a monosaccharide, disaccharide or an oligosaccharide.

The value of q may depend e.g. on the polymer, on the payload molecule, the linker unit, and the method of preparing the conjugate. Typically, a large value of q may lead to higher efficiency of the conjugate; on the other hand, a large value of q may in some cases affect other properties of the conjugate, such as pharmacokinetic properties or solubility, adversely. In an embodiment, q is in the range of 1 to about 300, or in the range of about 10 to about 200, or in the range of about 20 to about 100, or in the range of about 20 to about 150. In an embodiment, q is in the range of 1 to about 20, or in the range of 1 to about 15 or in the range of 1 to about 10. In an embodiment, q is 1, 2, 3, 4, 5, 6, 7, 8, 9, 10, 11, 12, 13, 14, 15, 16, 17, 18, 19, or 20. In an embodiment, q is 2-16. In an embodiment, q is in the range of 2 to 10. In other embodiments, q is in the range of 2 to 6; 2 to 5; 2 to 4; 2 or 3; or 3 or 4.

In an embodiment, about 25-45% of carbons of the polymer bearing a hydroxyl group are substituted by a substituent of the formula D-Y—(CH₂)ₙ—O—.

In embodiments in which the polymer comprises a plurality of saccharide units, the ratio of q to the number of saccharide units of the polymer may be e.g. 1:20 to 1:3 or 1:4 to 1:2.

In an embodiment, O is 1, 2, 3, 4, 5, 6, 7, 8, 9 or 10. In an embodiment, O is in the range of 2 to 9, or in the range of 3 to 8, or in the range of 4 to 7, or in the range of 1 to 6, or in the range of 2 to 5, or in the range of 1 to 4.

Each o may, in principle, be independently selected. Each o in a single conjugate may also be the same.

In an embodiment, Y is S.

In an embodiment, Y is NH.

In an embodiment, Y is 1,2,3-triazolyl. In this specification, the term “1,2,3-triazolyl” should be understood as referring to 1,2,3-triazolyl, or to 1,2,3-triazolyl which is substituted. In an embodiment, the 1,2,3-triazolyl is a group formed by click conjugation comprising a triazole moiety. Click conjugation should be understood as referring to a reaction between an azide and an alkyne yielding a covalent product-1,5-disubstituted 1,2,3-triazole—such as copper(I)-catalysed azide-alkyne cycloaddition reaction (CuAAC). Click conjugation may also refer to copper-free click chemistry, such as a reaction between an azide and a cyclic alkyne group such as dibenzocyclooctyl (DBCO). “1,2,3-triazolyl” may thus also refer to a group formed by a reaction between an azide and a cyclic alkyne group, such as DBCO, wherein the group comprises a 1,2,3-triazole moiety.

In an embodiment, the linker unit L comprises a moiety represented by the formula XXVII, or L₂′ is represented by the formula XXVII

—Y′—(CH₂)ₚ—S—(CH₂)ₒ—O_{q}—P— Formula XXVII

wherein

P is a polymer selected from the group consisting of dextran, mannan, pullulan, hyaluronic acid, hydroxyethyl starch, chondroitin sulphate, heparin, heparin sulphate, polyalkylene glycol, Ficoll, polyvinyl alcohol, amylose, amylopectin, chitosan, cyclodextrin, pectin and carrageenan, or a derivative thereof;

q is at least 1;

O is in the range of 1 to 10;

p is in the range of 1 to 10; and

each Y′ is independently selected from the group consisting of NH and 1,2,3-triazolyl, wherein 1,2,3-triazolyl is optionally substituted.

In the context of Formula XXVII, each of P, o and q may be as defined for Formula XXVI.

In an embodiment, p is 3, 4, 5, 6, 7, 8, 9 or 10. In an embodiment, p is in the range of 3 to 4, or in the range of 3 to 5, or in the range of 3 to 6, or in the range of 3 to 7, or in the range of 3 to 8, or in the range of 3 to 9. Each p may, in principle, be independently selected. Each p in a single conjugate may also be the same.

In an embodiment, Y′ is selected from the group consisting of NH and 1,2,3-triazolyl.

In an embodiment, P is a polymer derivative comprising at least one saccharide unit.

In an embodiment, P is a polymer derivative comprising at least one saccharide unit, and the polymer derivative is bound to the targeting unit (for example, an antibody) via a bond formed by a reaction between at least one aldehyde group formed by oxidative cleavage of a saccharide unit of the polymer derivative and an amino group of the targeting unit.

In an embodiment, the saccharide unit is a D-glucosyl, Dmannosyl, D-galactosyl, L-fucosyl, D-N-acetylglucosaminyl, D-N-acetylgalactosaminyl, D-glucuronidyl, or D-galacturonidyl unit, or a sulphated derivative thereof.

In an embodiment, the D-glucosyl is D-glucopyranosyl.

In an embodiment, the polymer is selected from the group consisting of dextran, mannan, pullulan, hyaluronic acid, hydroxyethyl starch, chondroitin sulphate, heparin, heparin sulphate, amylose, amylopectin, chitosan, cyclodextrin, pectin and carrageenan. These polymers have the added utility that they may be oxidatively cleaved so that aldehyde groups are formed.

In an embodiment, the polymer is dextran.

In this specification, “dextran” should be understood as referring to a branched glucan composed of chains of varying lengths, wherein the straight chain consists of a α-1,6 glycosidic linkages between D-glucosyl (D-glucopyranosyl) units. Branches are bound via α-1,3 glycosidic linkages and, to a lesser extent, via α-1,2 and/or α-1,4 glycosidic linkages. A portion of a straight chain of a dextran molecule is depicted in the schematic representation below.

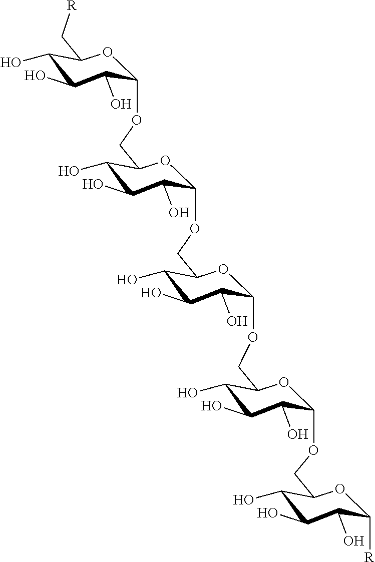

“D-glucosyl unit” should be understood as referring to a single D-glucosyl molecule. Dextran thus comprises a plurality of D-glucosyl units. In dextran, each D-glucosyl unit is bound to at least one other D-glucosyl unit via a α-1,6 glycosidic linkage, via a α-1,3 glycosidic linkage or via both.

Each D-glucosyl unit of dextran comprises 6 carbon atoms, which are numbered 1 to 6 in the schematic representation below. The schematic representation shows a single D-glucosyl unit bound to two other D-glucosyl units (not shown) via α-1,6 glycosidic linkages.

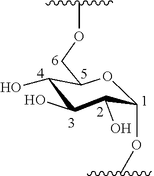

Carbons 2, 3 and 4 may be substituted by free hydroxyl groups. In D-glucosyl units bound to a second D-glucosyl unit via a α-1,3 glycosidic linkage, wherein carbon 3 of the D-glucosyl unit is bound via an ether bond to carbon 1 of the second D-glucosyl unit, carbons 2 and 4 may be substituted by free hydroxyl groups. In D-glucosyl units bound to a second D-glucosyl unit via a α-1,2 or α-1,4 glycosidic linkage, wherein carbon 2 or 4 of the D-glucosyl unit is bound via an ether bond to carbon 1 of the second D-glucosyl unit, carbons 3 and 4 or 2 and 3, respectively, may be substituted by free hydroxyl groups.

A skilled person will understand that other polymers described in this specification also contain free hydroxyl groups bound to one or more carbon atoms and have also other similar chemical properties.

Carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293).

In this specification, the term “Ficoll” refers to an uncharged, highly branched polymer formed by the co-polymerisation of sucrose and epichlorohydrin.

In an embodiment, the polymer is a dextran derivative comprising at least one D-glucosyl unit;

O is in the range of 3 to 10;

Y is S;

the dextran derivative comprises at least one aldehyde group formed by oxidative cleavage of a D-glucosyl unit; and

the dextran derivative is bound to the targeting unit (for example, an antibody) via a bond formed by a reaction between at least one aldehyde group of the dextran and an amino group of the targeting unit.

Saccharide units of the polymer, for instance the D-glucosyl units of dextran, may be cleaved by oxidative cleavage of a bond between two adjacent carbons substituted by a hydroxyl group.

The oxidative cleavage cleaves vicinal diols, such as D-glucosyl and other saccharide units in which two (free) hydroxyl groups occupy vicinal positions. Saccharide units in which carbons 2, 3 and 4 are substituted by free hydroxyl groups may thus be oxidatively cleaved between carbons 2 and 3 or carbons 3 and 4. Thus a bond selected from the bond between carbons 2 and 3 and the bond between carbons 3 and 4 may be oxidatively cleaved. D-glucosyl units and other saccharide units of dextran and other polymers may be cleaved by oxidative cleavage using an oxidizing agent such as sodium periodate, periodic acid and lead(IV) acetate, or any other oxidizing agent capable of oxidatively cleaving vicinal diols.

Oxidative cleavage of a saccharide unit forms two aldehyde groups, one aldehyde group at each end of the chain formed by the oxidative cleavage. In the conjugate, the aldehyde groups may in principle be free aldehyde groups. However, the presence of free aldehyde groups in the conjugate is typically undesirable. Therefore the free aldehyde groups may be capped or reacted with an amino group of the targeting unit, or e.g. with a tracking molecule.

In an embodiment, the polymer derivative is bound to the targeting unit via a bond formed by a reaction between at least one aldehyde group formed by oxidative cleavage of a saccharide unit of the polymer derivative and an amino group of the targeting unit.

In an embodiment, the polymer derivative may also be bound to the targeting unit via a group formed by a reaction between at least one aldehyde group formed by oxidative cleavage of a saccharide unit of the polymer derivative and an amino group of the targeting unit.

The aldehyde group formed by oxidative cleavage readily reacts with an amino group in solution, such as an aqueous solution. The resulting group or bond formed may, however, vary and is not always easily predicted and/or characterised. The reaction between at least one aldehyde group formed by oxidative cleavage of a saccharide unit of the polymer derivative and an amino group of the targeting unit may result e.g. in the formation of a Schiff base. Thus the group via which the polymer derivative is bound to the targeting unit may be e.g. a Schiff base (imine) or a reduced Schiff base (secondary amine).

X) Conjugates

The conjugate may be any conjugate described in this specification; a skilled person may derive various conjugates by combining any one of the above units and payload molecules described in this specification.

In an embodiment,

R₇ is —C(═O)—;

L₁′ is a C₁-C₁₂ alkyl, optionally a C₁-C₆ alkyl;

Sₜ in L₁ is optionally absent;

Sₚ is disulfide;

Sₜ is present in L₂ and is according to one or more embodiments described in this specification. For example, Sₜ present in L₂ may be e.g. a moiety represented by any one of formulas LI to LXVII set out in this specification.

In an embodiment,

R₇ is —C(═O)—;

L₁′ is a substituted or unsubstituted C₁-C₁₂ alkylene, optionally a substituted or unsubstituted C₁-C₆ alkylene;

Sₜ in L₁ is optionally absent;

Sₚ is disulfide;

Sₜ is present in L₂ and is according to one or more embodiments described in this specification. For example, Sₜ present in L₂ may be e.g. a moiety represented by any one of formulas LI to LXVII set out in this specification.

In an embodiment, R₇ is —C(═O)—;

L₁′ is a substituted or unsubstituted C₁-C₁₂ alkylene, optionally a substituted or unsubstituted C₁-C₆ alkylene;

Sₜ in L₁ is NH;

Sₚ is peptide forming a peptide bond with Sₜ, optionally comprising asparagine;

Sₜ is present in L₂ and is according to one or more embodiments described in this specification. For example, Sₜ present in L₂ may be e.g. a moiety represented by any one of formulas LI to LXVII set out in this specification.

In an embodiment,

R₇ is —C(═O)O—;

L₁′ is a C₁-C₁₂ alkyl, optionally a C₁-C₆ alkyl;

Sₜ in L₁ is optionally absent;

Sₚ is disulfide;

L₂ and R₈ are absent and Sₚ is directly bonded to a thiol group in a targeting unit so that the other S atom in the disulfide comes from the thiol group in the targeting unit.

In an embodiment, the conjugate is represented by Formula CD:

[D-O—R₇—Sₜ-Sₚ]ₙ-T Formula CD

wherein D is a payload molecule comprising a sugar moiety;

O is an oxygen atom of said sugar moiety;

T is a targeting unit capable of binding a target molecule, cell and/or tissue;

n is at least 1; and

R₇ is —C(═O)O—;

Sₚ is a sulfur atom forming a disulfide with a sulfur atom of T;

each Sₜ is independently absent or a moiety represented by formula LI:

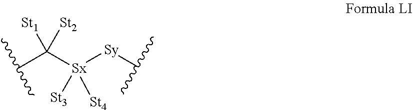

wherein St₁, St₂, St₃, and St₄ are each independently selected from H, CH₃, CH₂CH₃, unsubstituted or substituted C₁-C₆ alkyl, unsubstituted or substituted C₁-C₆ cycloalkyl, unsubstituted or substituted aryl, OH, OCH₃, OR_{O}, wherein R_{O} is either a C₁-C₆ alkyl or a C₁-C₆ substituted alkyl, and an amino acid side chain;

or wherein St₁ together with the carbon to which it is attached, with Sx and optionally with St₃ form an unsubstituted or substituted carbocyclyl or heterocyclyl group;

Sx is either C or N, wherein St₄ is absent if Sx is N; and

Sy is either absent, —C(═O)O— or —(CH₂)ₘ—, wherein m is 1 to 4.

In an embodiment, the conjugate is represented by Formula CD wherein other variables are as described above and Sₜ is according to formula LXIII:

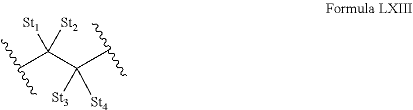

wherein St₂ and St₄ are H, and St₁ and St₃ are CH₃.

In an embodiment, the conjugate is represented by Formula CD wherein other variables are as described above; Sₜ is according to formula LXIII wherein St₂ and St₄ are H, St₁ and St₃ are CH₃; and D is kifunensine.

In an embodiment, the conjugate is represented by Formula CD wherein other variables are as described above; and Sₜ is according to formula LXIII wherein St₄ is H, St₁ and St₂ and St₃ are (each) CH₃.

In an embodiment, the conjugate is represented by Formula CD wherein other variables are as described above; Sₜ is according to formula LXIII wherein St₄ is H, St₁ and St₂ and St₃ are (ech) CH₃; and D is kifunensine.

In an embodiment, the conjugate is represented by Formula CD wherein other variables are as described above; and Sₜ is according to formula LXIII wherein St₁ and St₂ and St₃ and St₄ are (each) CH₃.

In an embodiment, the conjugate is represented by Formula CD wherein other variables are as described above; Sₜ is according to formula LXIII wherein St₁ and St₂ and St₃ and St₄ are CH₃; and D is kifunensine.

In an embodiment, the conjugate is represented by Formula ED:

[D-O—Sₜ—Sₚ-L₂-R₈]ₙ-T Formula ED

wherein D is a payload molecule comprising a sugar moiety;

O is an oxygen atom of said sugar moiety;

T is a targeting unit capable of binding a target molecule, cell and/or tissue;

n is at least 1;

Sₚ is a specificity unit;

L₂ is absent or a stretcher unit;

R₈ is absent or a group covalently bonded to the targeting unit; and

Sₜ is a moiety represented by formula LXVI:

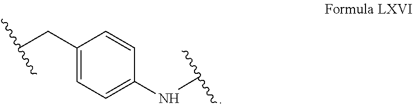 [SMILES: *Cc1ccc(N*)cc1]

In an embodiment, the conjugate is represented by Formula ED wherein other variables are as described above and Sₚ is a peptide selected from the group of Asn, Ala-Asn, Ala-Ala-Asn, Val-Cit, Ala-Ala, Val-Ala, and Phe-Lys.

In an embodiment, the conjugate is represented by Formula ED wherein other variables are as described above and Sₚ is a peptide selected from the group of Asn, Ala-Asn, Ala-Ala-Asn, Val-Cit, Ala-Ala, Val-Ala, and Phe-Lys; and D is kifunensine.

In an embodiment, the conjugate is represented by Formula ED wherein other variables are as described above; Sₚ is a peptide selected from the group of Asn, Ala-Asn, Ala-Ala-Asn, Val-Cit, Ala-Ala, Val-Ala, and Phe-Lys; and L₂-R₈ is a β-alanine-maleimidoacetyl group covalently bonded to a sulfur atom of the targeting unit.

In an embodiment, the conjugate is represented by Formula ED wherein other variables are as described above; Sₚ is a peptide selected from the group of Asn, Ala-Asn, Ala-Ala-Asn, Val-Cit, Ala-Ala, Val-Ala, and Phe-Lys; and L₂-R₈ is β-alanine-maleimidoacetyl group covalently bonded to a sulfur atom of the targeting unit; and D is kifunensine.

The conjugate may be any conjugate represented by any one of formulas CI onwards below, or a conjugate selected from the group consisting of conjugates represented by any one of formulas CI onwards below:

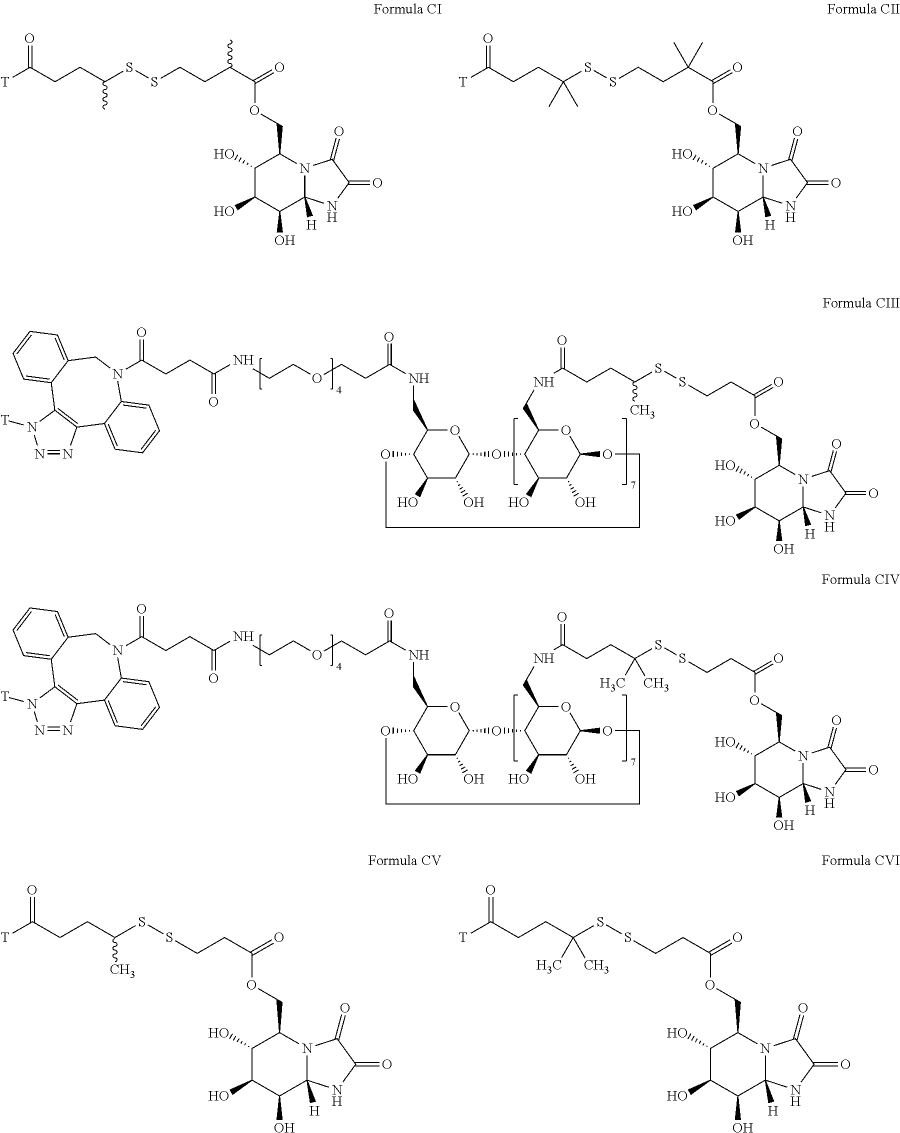

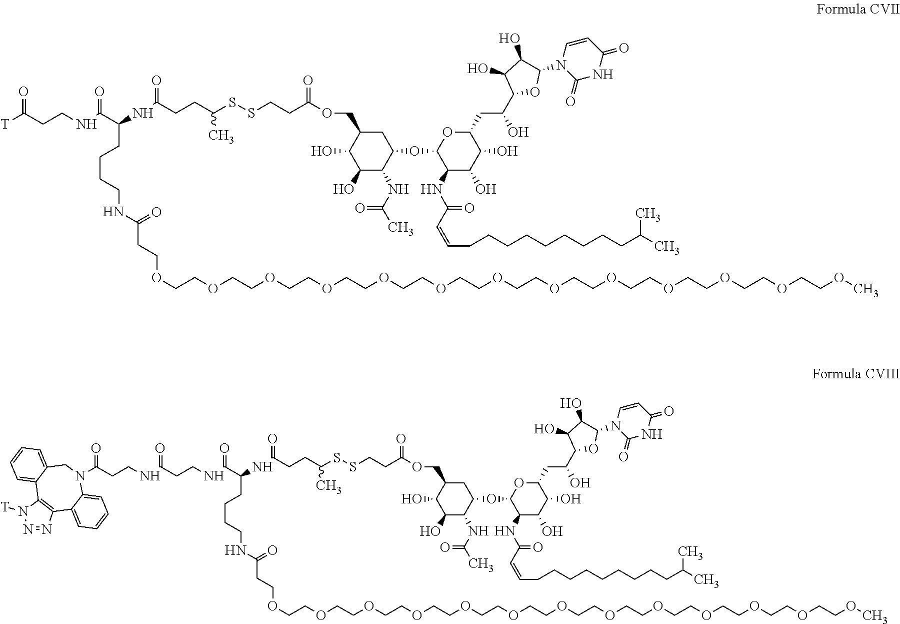

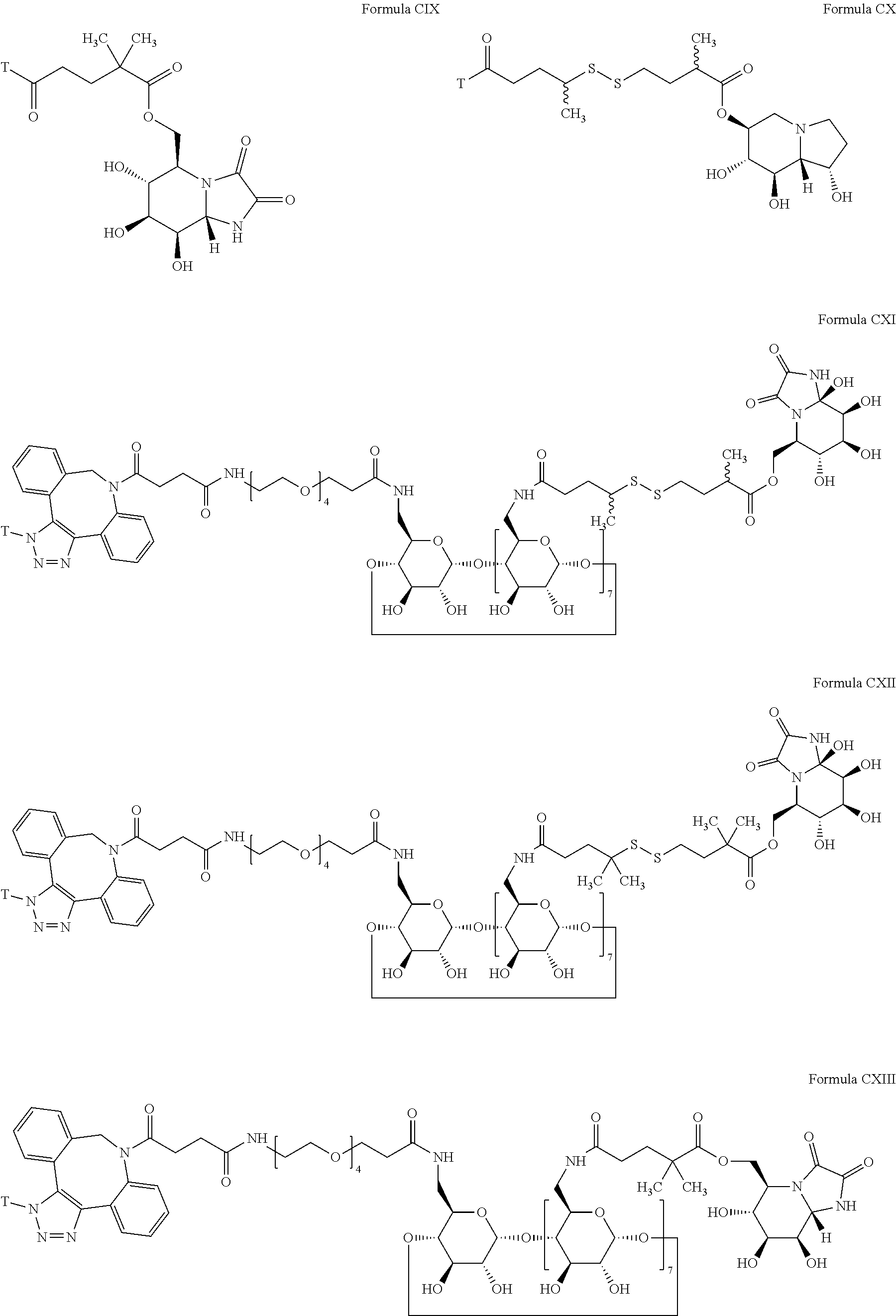

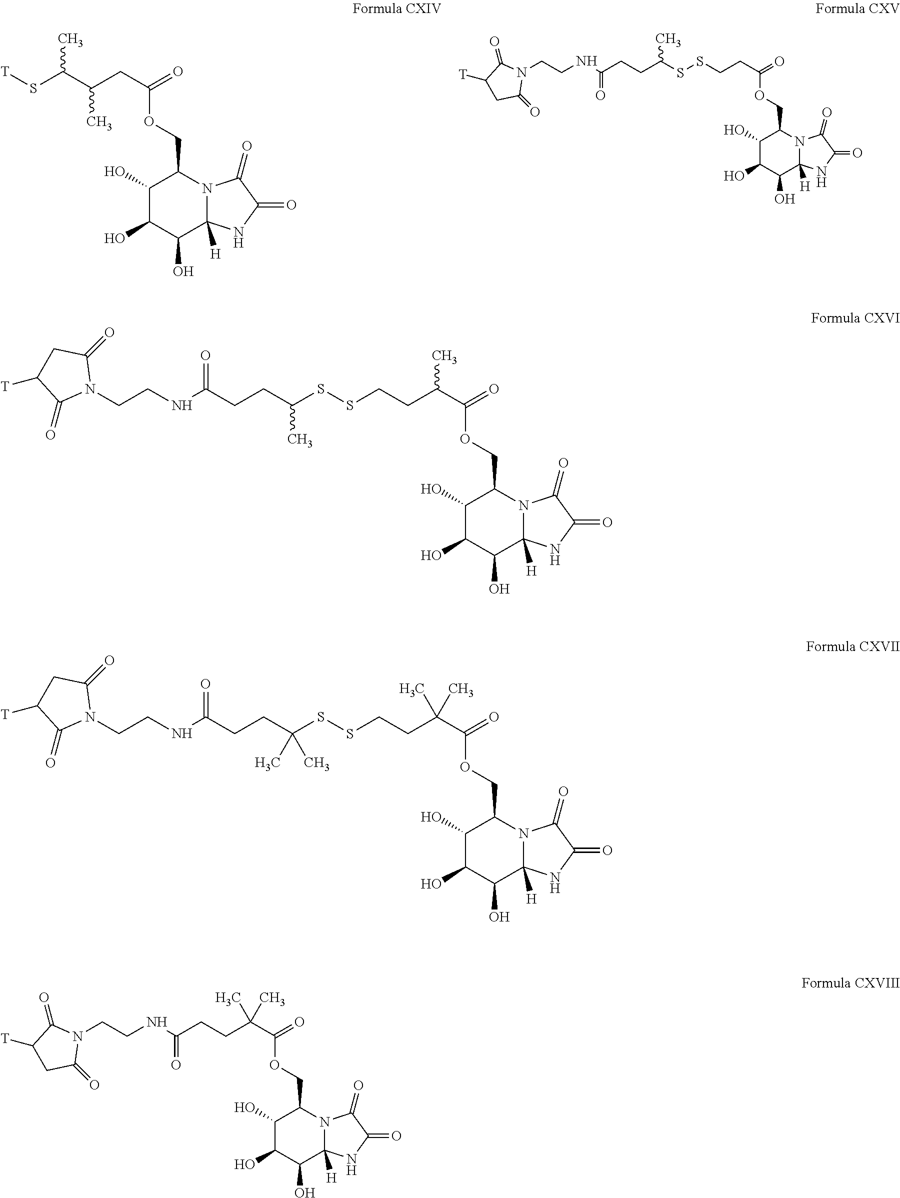

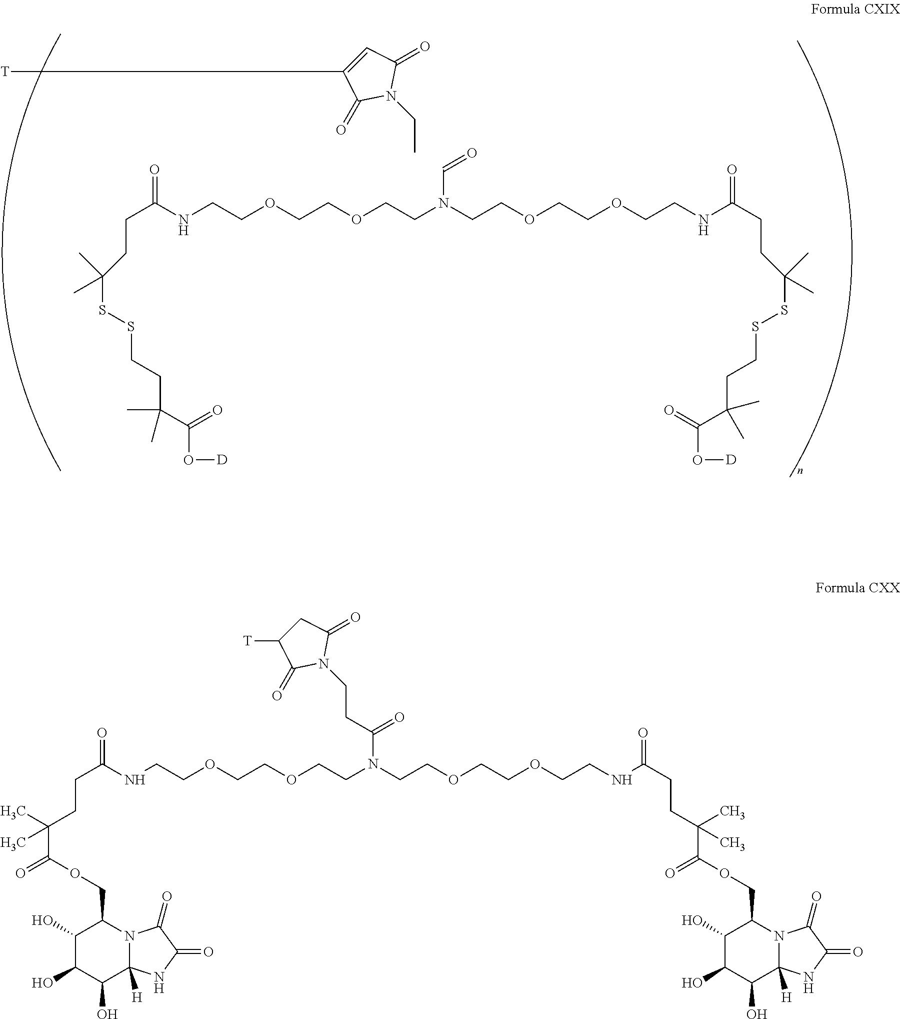

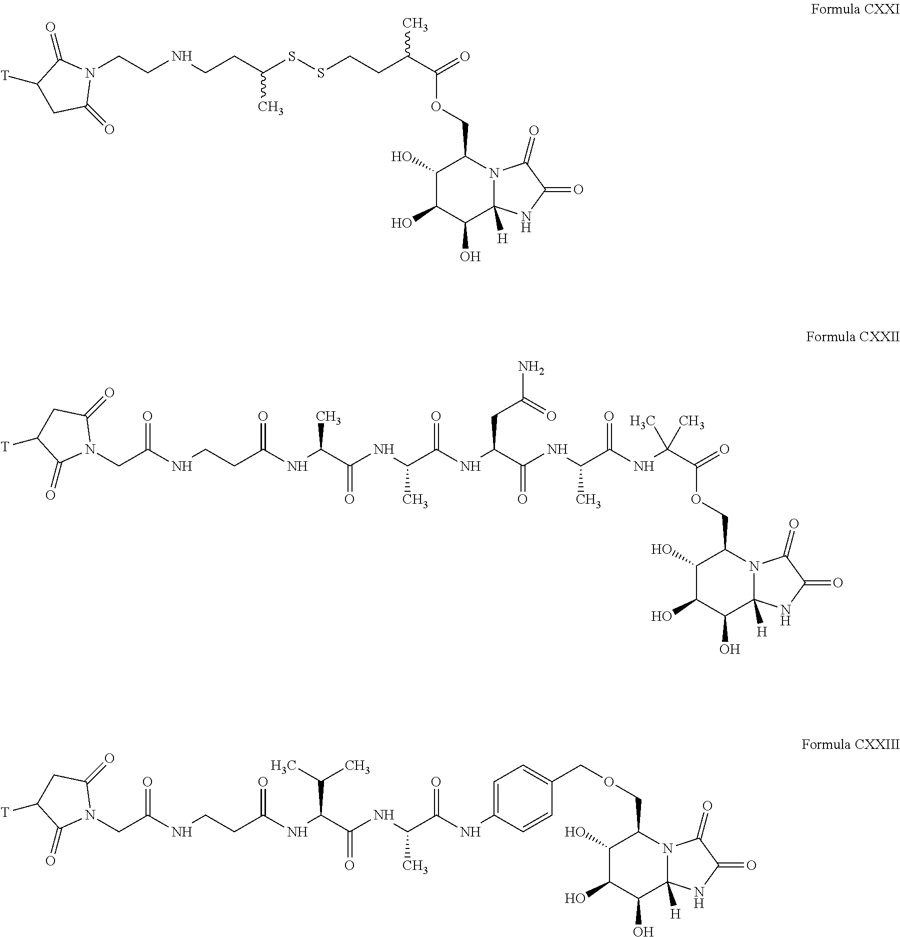

wherein T represents the targeting unit, and each T is optionally linked to one or more linker-payload units.

It should be understood that the maleimide groups of the above formulas represent both the intact rings as shown and the stabilized hydrolyzed open rings. The maleimide groups described herein may have an inherent property to self-hydrolyze at or above neutral pH after conjugation to the targeting unit as shown below in Scheme M-1. This has the added utility that the self-hydrolyzed maleimide conjugate may be more stable than the original maleimide thioether.

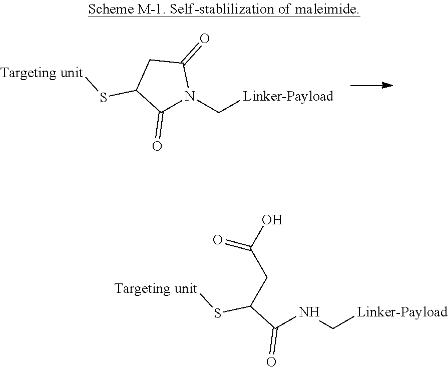

Thus, the conjugate may be further selected from the group consisting of conjugates represented by any one of formulas CXVm onwards below:

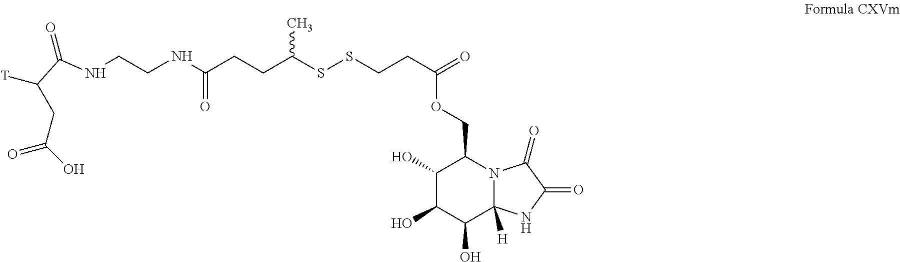

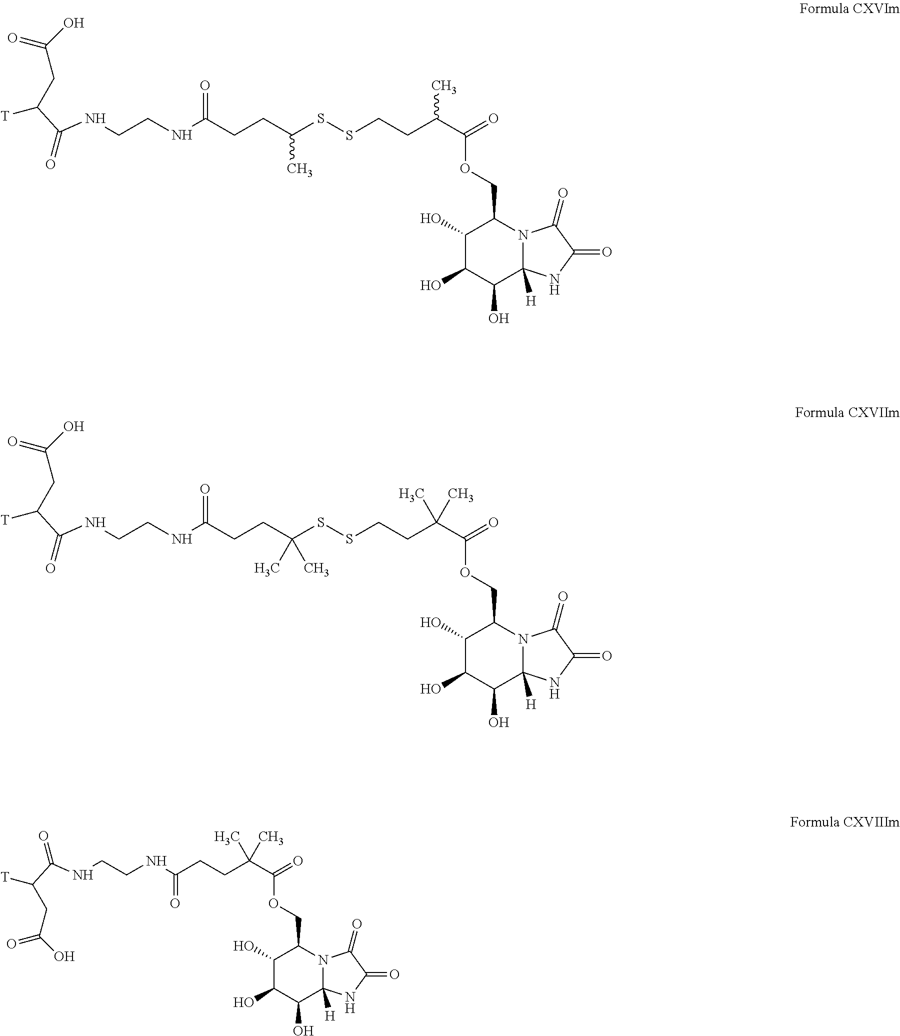

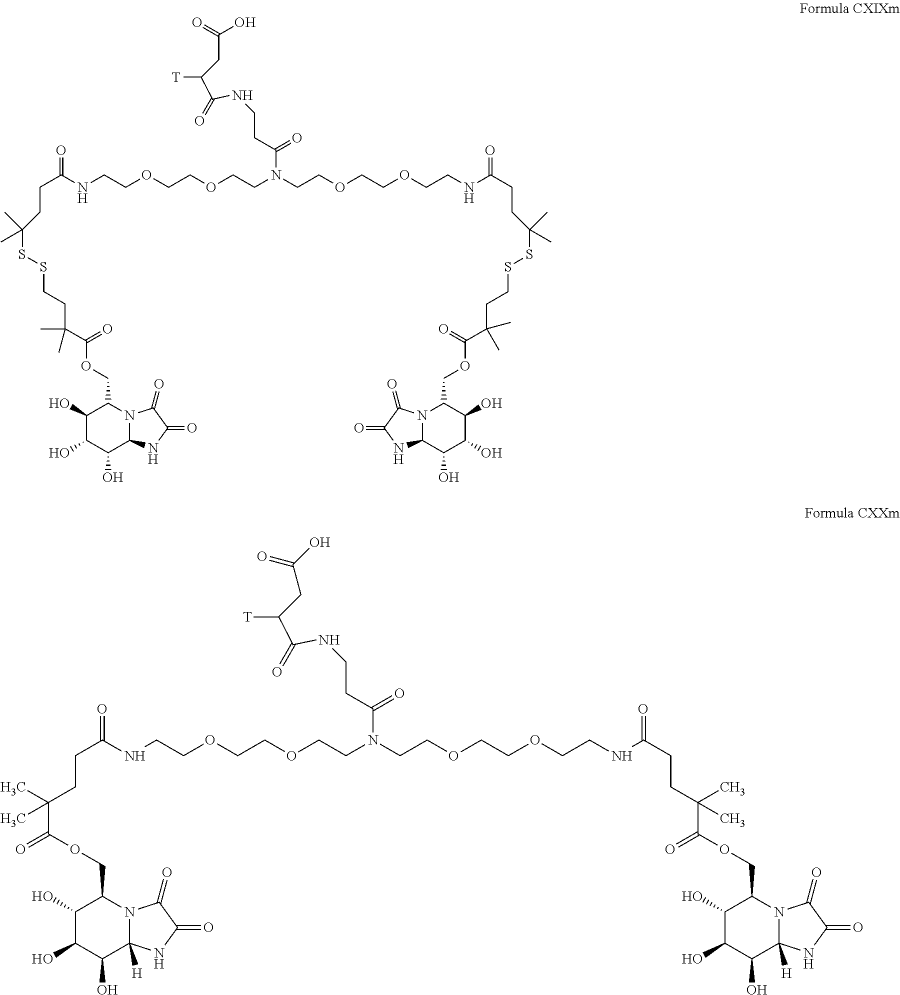

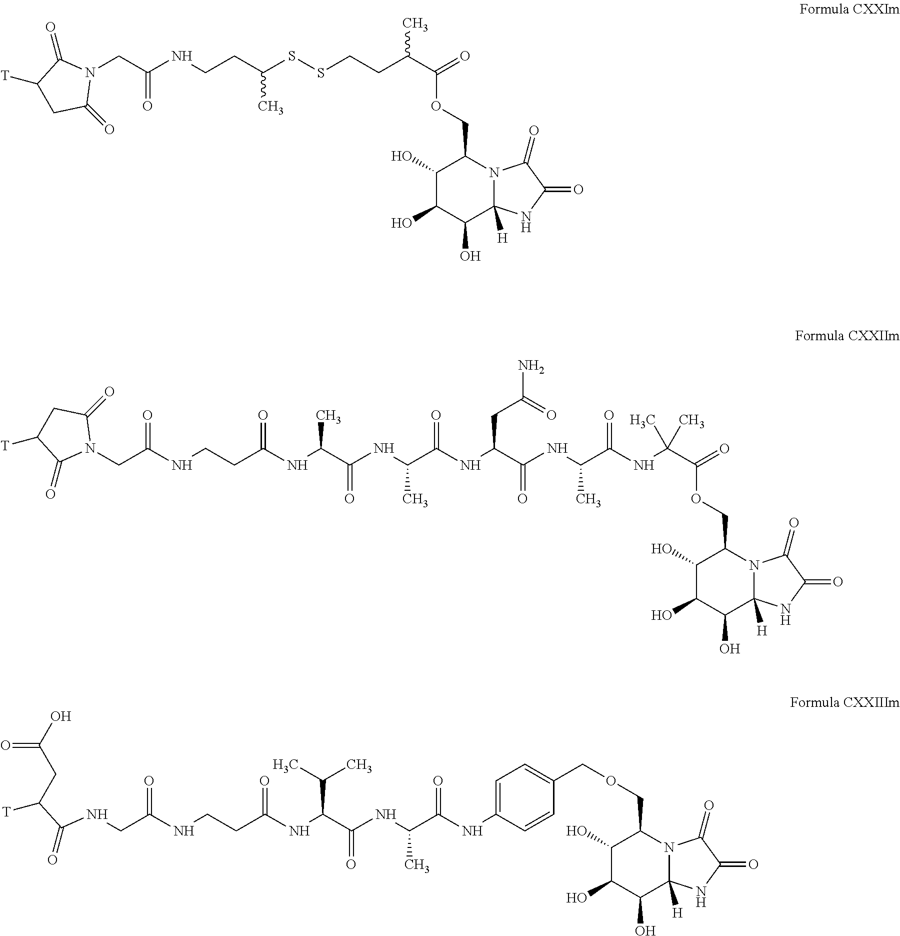

wherein T represents the targeting unit, and each T is optionally linked to one or more additional linker-payload units (e.g. one or more linker-payload units that are the same or other than the linker-payload unit set forth in the formulas). In an embodiment, each T is optionally linked to one or more additional linker-payload units according to Formula I, wherein n is the number of the linker-payload units D-O-L linked to T.

It should also be understood that the payload molecules described in the above formulas may be replaced by any one of the payload molecules described in this specification. In other words, e.g. the kifunensine moiety in the formulas may be replaced by D according to one or more embodiments described in this specification.

The conjugate may be a conjugate represented by any one of formulas DI onwards below, or a conjugate selected from the group consisting of conjugates represented by any one of formulas DI onwards below:

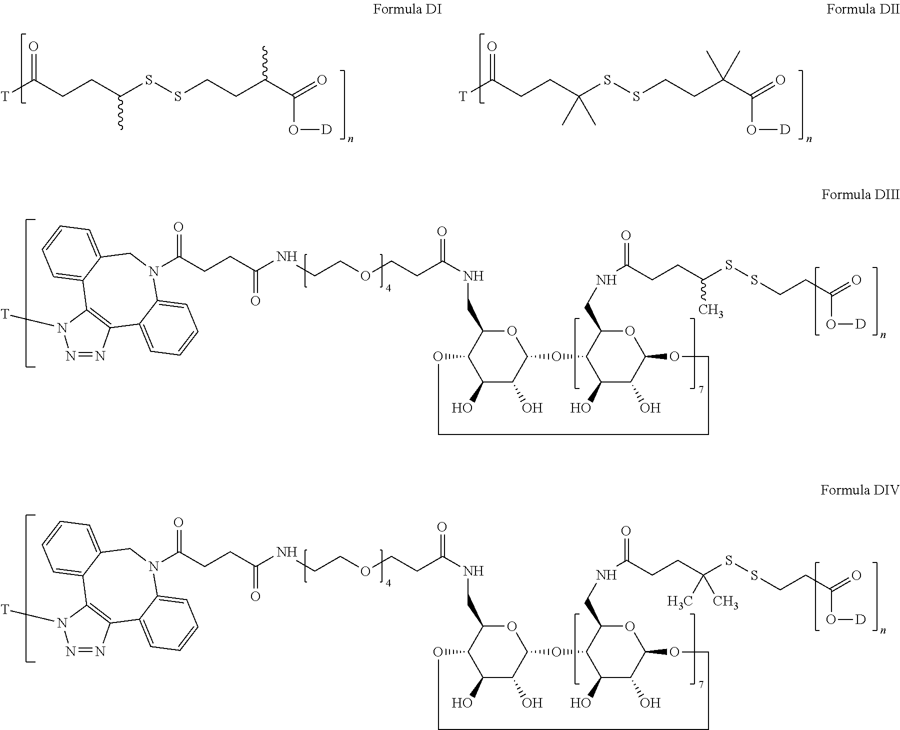

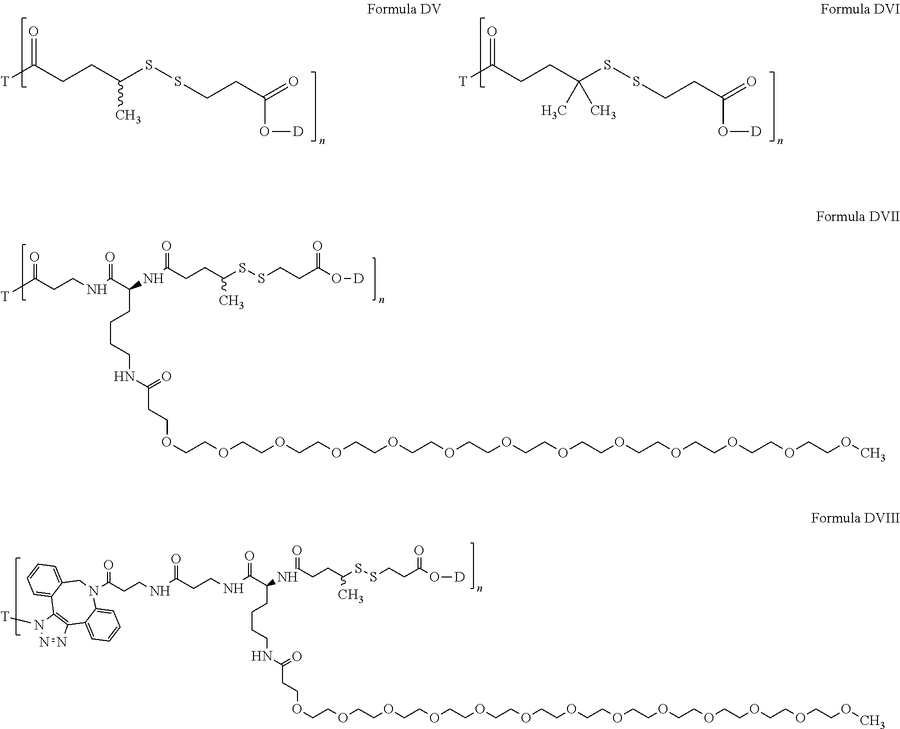

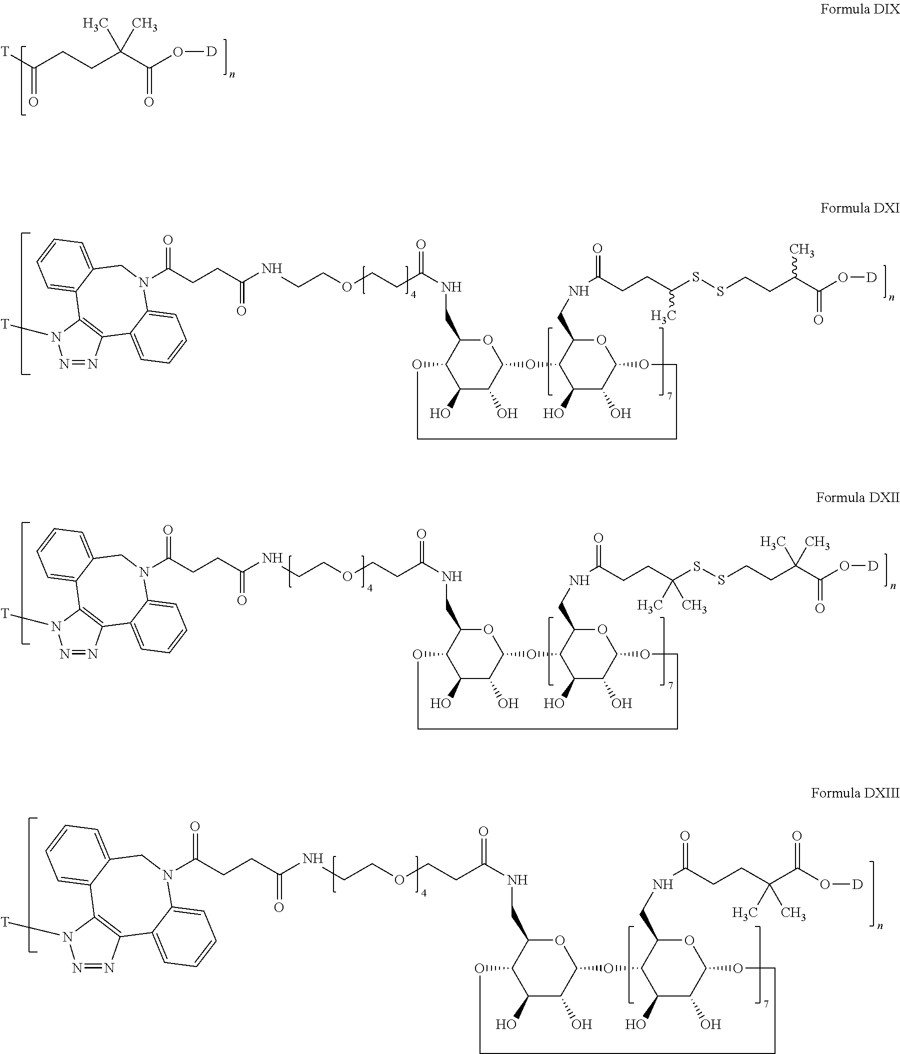

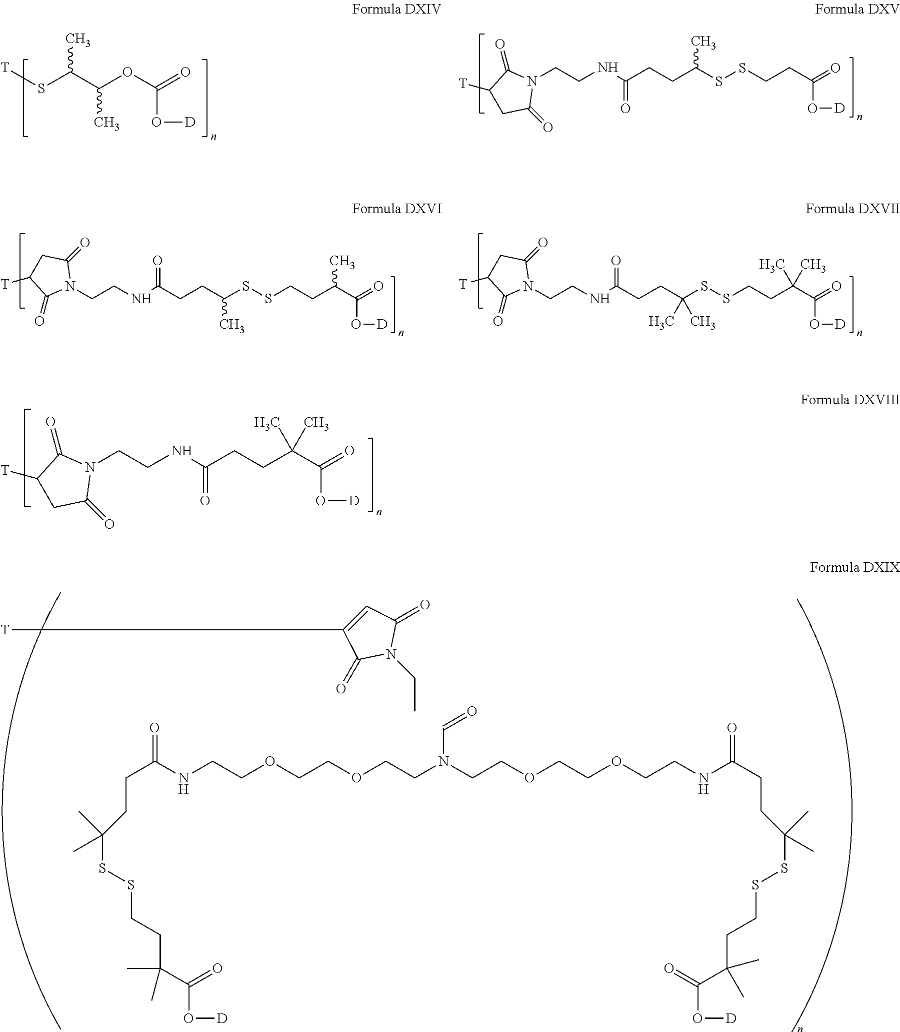

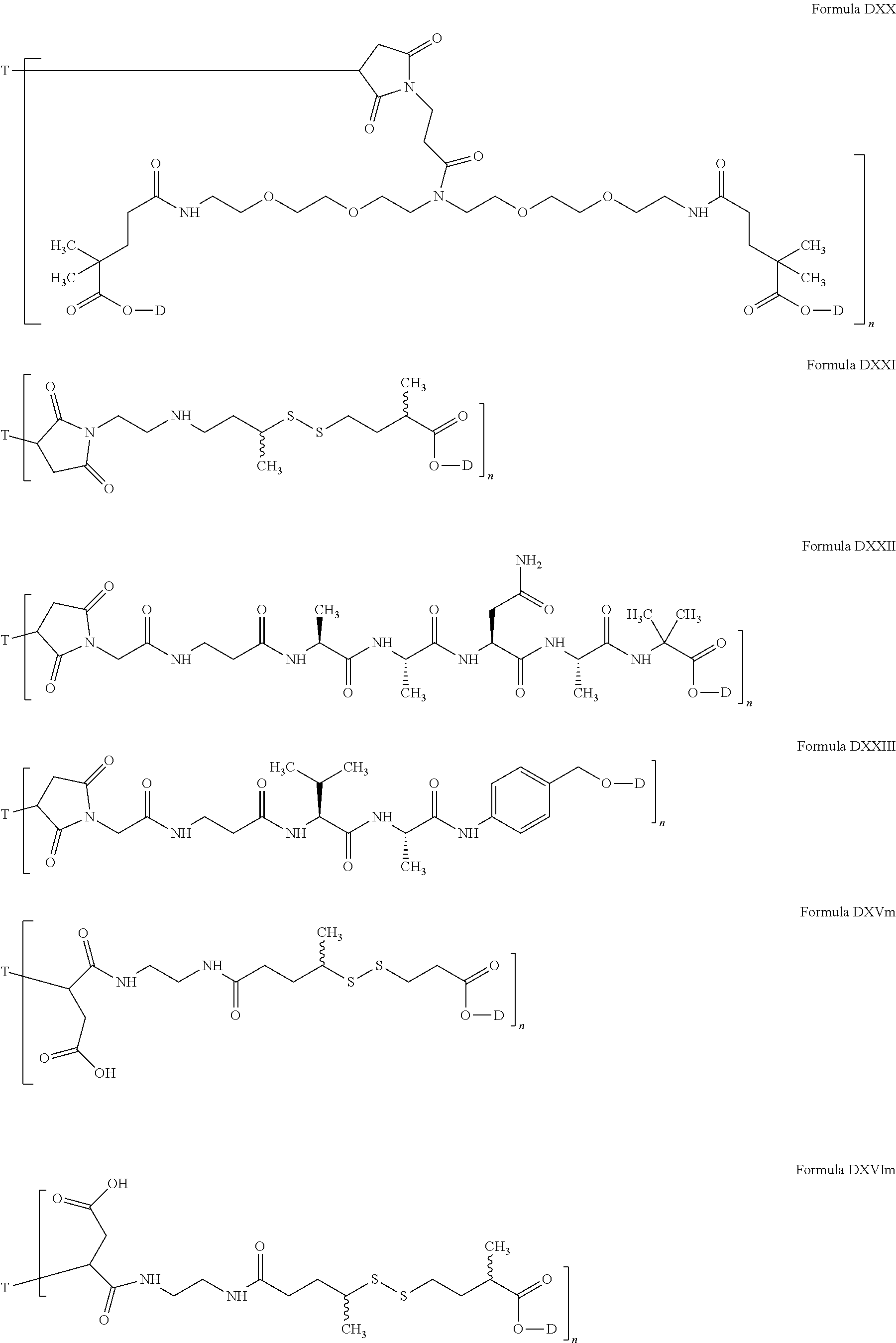

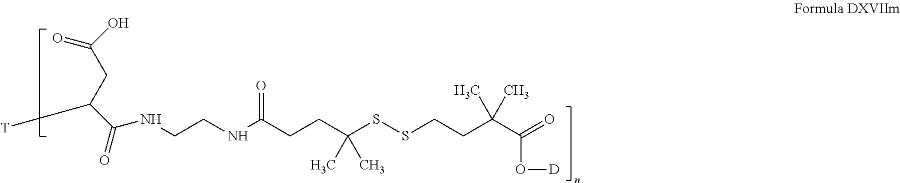

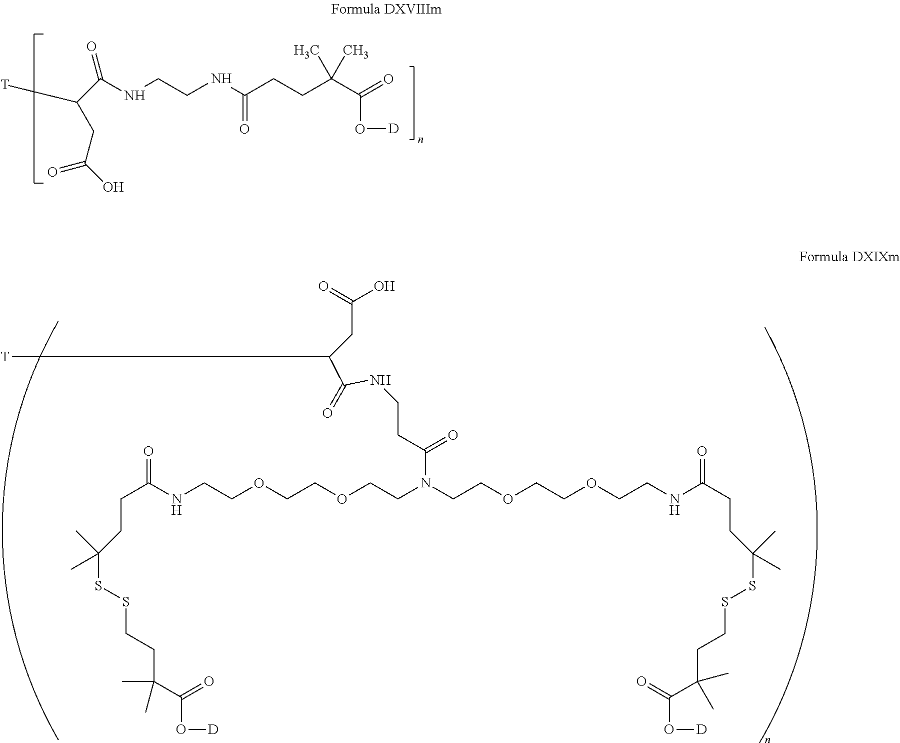

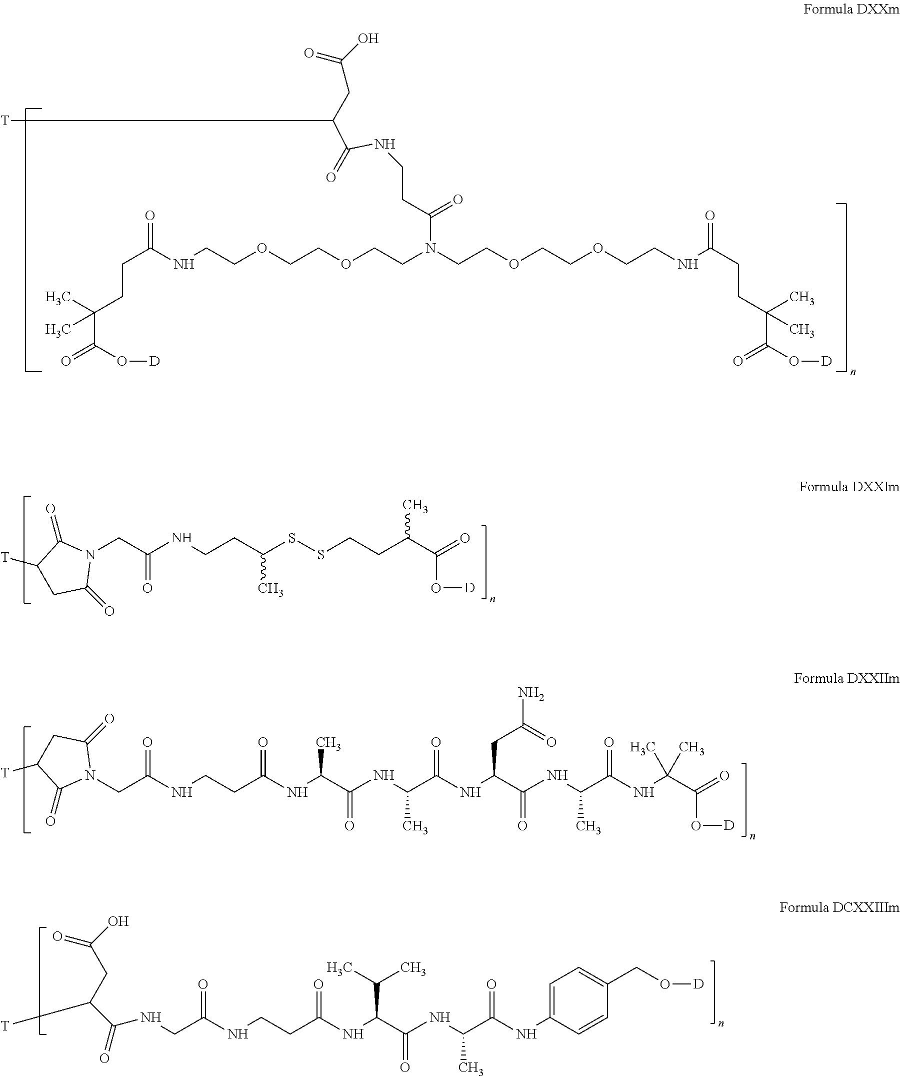

wherein O-D represents the payload molecule, wherein O is an oxygen atom of said payload molecule;

T is a targeting unit capable of binding a target molecule, cell and/or tissue; and

n is at least 1.

XI) Compositions and Methods

A pharmaceutical composition comprising the conjugate according to one or more embodiments described in this specification is disclosed.

The pharmaceutical composition may further comprise one or more further components, for example a pharmaceutically acceptable carrier. Examples of suitable pharmaceutically acceptable carriers are well known in the art and may include e.g. phosphate buffered saline solutions, water, oil/water emulsions, wetting agents, and liposomes. Compositions comprising such carriers may be formulated by methods well known in the art. The pharmaceutical composition may further comprise other components such as vehicles, additives, preservatives, other pharmaceutical compositions administrated concurrently, and the like.

In an embodiment, the pharmaceutical composition comprises an effective amount of the conjugate according to one or more embodiments described in this specification.

In an embodiment, the pharmaceutical composition comprises a therapeutically effective amount of the conjugate according to one or more embodiments described in this specification.

The term “therapeutically effective amount” or “effective amount” of the conjugate may be understood as referring to the dosage regimen for achieving a therapeutic effect, for example modulating the growth of cancer cells and/or treating a patient's disease. The therapeutically effective amount may be selected in accordance with a variety of factors, including the age, weight, sex, diet and medical condition of the patient, the severity of the disease, and pharmacological considerations, such as the activity, efficacy, pharmacokinetic and toxicology profiles of the particular conjugate used. The therapeutically effective amount can also be determined by reference to standard medical texts, such as the Physicians Desk Reference 2004. The patient may be male or female, and may be an infant, child or adult.

The term “treatment” or “treat” is used in the conventional sense and means attending to, caring for and nursing a patient with the aim of combating, reducing, attenuating or alleviating an illness or health abnormality and improving the living conditions impaired by this illness, such as, for example, with a cancer disease.

In an embodiment, the pharmaceutical composition comprises a composition for e.g. oral, parenteral, transdermal, intraluminal, intraarterial, intrathecal, intra-tumoral (i.t.), and/or intranasal administration or for direct injection into tissue. Administration of the pharmaceutical composition may be effected in different ways, e.g. by intravenous, intraperitoneal, subcutaneous, intramuscular, intra-tumoral, topical or intradermal administration.

A conjugate according to one or more embodiments described in this specification or a pharmaceutical composition comprising the conjugate according to one or more embodiments described in this specification for use as a medicament is disclosed.

A conjugate according to one or more embodiments described in this specification or a pharmaceutical composition comprising the conjugate according to one or more embodiments described in this specification for use in decreasing immunosuppressive activity in a tumour is disclosed.

A conjugate according to one or more embodiments described in this specification or a pharmaceutical composition comprising the conjugate according to one or more embodiments described in this specification for use in the treatment, modulation and/or prophylaxis of the growth of tumour cells in a human or animal is also disclosed.

A conjugate according to one or more embodiments described in this specification or a pharmaceutical composition comprising the conjugate according to one or more embodiments described in this specification for use in the treatment of cancer is disclosed.

The cancer may be selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer. However, other cancers and cancer types may also be contemplated.

A method of treating, modulating and/or prophylaxis of the growth of tumour cells in a human or animal is also disclosed. The method may comprise administering the conjugate according to one or more embodiments described in this specification or the pharmaceutical composition according to one or more embodiments described in this specification to a human or animal in an effective amount.

The tumour cells may be selected from the group of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, multidrug resistant cancer cells, and testicular cancer cells.

A method for preparing the conjugate according to one or more embodiments described in this specification is disclosed. The method may comprise conjugating the payload molecule to the targeting unit. The method may comprise conjugating the payload molecule via the oxygen atom of the sugar moiety to the targeting unit. The method may comprise conjugating the payload molecule via the oxygen atom of the sugar moiety with an ester bond to the targeting unit.

Anything disclosed above in the context of the conjugate may also be understood as being disclosed in the context of the method(s).

The activity of the conjugates may be measured depending on the individual payload molecule(s) e.g. by their inhibition of cellular glycosylation by numerous methods known in the art. Glycan profiling can be done by mass spectrometry, MALDI-TOF mass spectrometry, lectin binding, lectin microarray assays, or the like, to directly measure inhibition of specific glycosylation routes by assaying decrease in the relative abundance of specific glycans compared to other glycan types, for example. Examples of suitable glycan profiling methods are described in the Examples section and further methods are well known for a person skilled in the art.

Inhibition of lectin ligand synthesis may be measured by for example using recombinant galectins, Siglecs, or other lectins involved in immune checkpoints, and a suitable detection label. Examples of suitable lectin binding assay methods are described in the Examples section and further methods are well known for a person skilled in the art.

Inhibition of immune suppression may be measured by for example in vitro assays using target cells and immune cells, and measuring cell kill activity, cellular activation, cytokine production, or the like. Examples of suitable immune cell assay methods are well known for a person skilled in the art.

### EXAMPLES

### Example 1. Preparation of Payload-Ester Conjugates

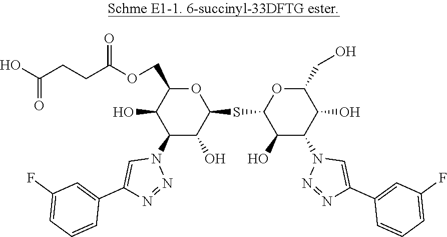 [SMILES: O=C(O)CCC(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O]

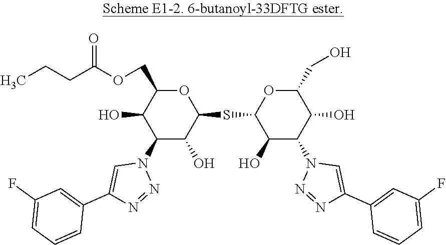 [SMILES: CCCC(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O]

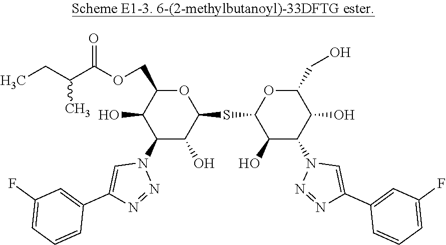 [SMILES: CCC(C)C(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O]

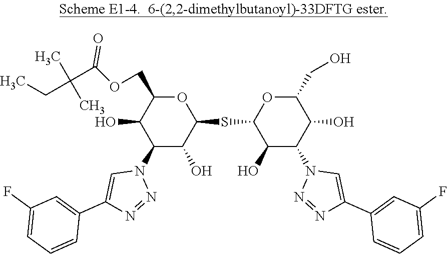 [SMILES: CCC(C)(C)C(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O]

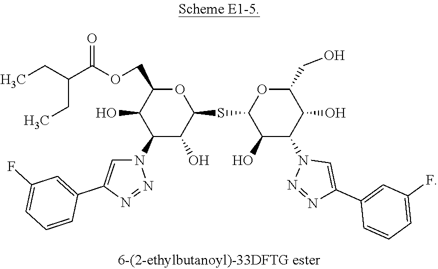 [SMILES: CCC(CC)C(=O)OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O]

Scheme E1-1: 5.3 mg (8 μmol) of 3,3′-dideoxy-3,3′-bis-[4-(3-fluorophenyl)-1H-1,2,3-triazol-1-yl]-1,1′-sulfanediyl-di-β-Dgalactopyranoside (33DFTG, or TD139; MedChemExpress Europe, Sollentuna, Sweden), 2.8 molar excess of succinic anhydride in pyridine (21 μl) and 79 μl pyridine were stirred at room temperature (RT) for 3.5 hours. The crude reaction mixture was analysed by MALDI-TOF mass spectrometry (MALDI-TOF MS) with Bruker Ultraflex III TOF/TOF instrument (Bruker Daltonics, Bremen, Germany) using 2,5-dihydroxybenzoic acid (DHB) matrix, showing expected masses for 6-succinyl-33DFTG (m/z 771 [M+Na]⁺) and di-6-succinyl-DFTG (m/z 871 [M+Na]⁺). The reaction was quenched by adding 0.5 ml ethanol.

The products were purified by Äkta purifier (GE Healthcare) HPLC instrument with Sdex peptide SE column (10×300 mm, 13 μm (GE Healthcare)) in aqueous ammonium acetate buffer. 6-succinyl-DFTG was recovered in one of the collected fractions, detected by MALDI-TOF MS similarly as above, and dried under vacuum.

Schemes E1-2 to E1-5: 33DFTG was dissolved in dry pyridine and combined with a molar excess of butyryl chloride, 2-methylbutyryl chloride, 2,2-dimethylbutyryl chloride, or 2-ethylbutyryl chloride, respectively. The reactions proceeded at room temperature (RT) and they were monitored by MALDI-TOF MS. The expected masses were detected for 6-butanoyl-33DFTG (m/z 741.21 [M+Na]⁺), 6-(2-methylbutanoyl)-33DFTG (m/z 755.22 [M+Na]⁺), 6-(2,2-dimethylbutanoyl)-33DFTG (m/z 747.23 [M+H]⁺), and 6-(2-ethylbutanoyl)-33DFTG (m/z 747.21 [M+H]⁺), respectively. The products were purified by HPLC and identified by MALDI-TOF MS similarly as above.

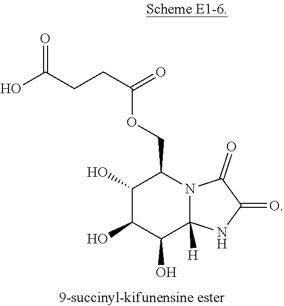 [SMILES: O=C(O)CCC(=O)OC[C@@H]1[C@@H](O)[C@H](O)[C@H](O)[C@H]2NC(=O)C(=O)N12]

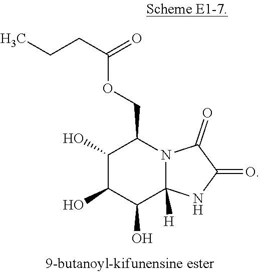 [SMILES: CCCC(=O)OC[C@@H]1[C@@H](O)[C@H](O)[C@H](O)[C@H]2NC(=O)C(=O)N12]

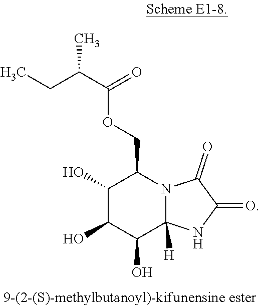 [SMILES: CC[C@H](C)C(=O)OC[C@@H]1[C@@H](O)[C@H](O)[C@H](O)[C@H]2NC(=O)C(=O)N12]

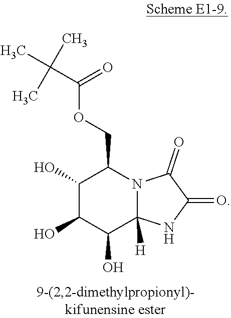 [SMILES: CC(C)(C)C(=O)OC[C@@H]1[C@@H](O)[C@H](O)[C@H](O)[C@H]2NC(=O)C(=O)N12]

Scheme E1-6: Kifunensine (GlycoSyn, New Zealand) was combined with an excess of succinic anhydride in pyridine and allowed to react at room temperature. The reaction mixture was analysed by MALDI-TOF MS as described above, showing the expected mass for 9-succinyl-kifunensine (m/z 355.18 [M+Na]⁺). The monosuccinyl product was purified by HPLC, detected by MALDI-TOF MS similarly as above and dried under vacuum. A sample of the product was analyzed by ¹H-NMR in D₂O (FIG. 1). The purity and the expected structure were confirmed by the analysis: as expected, the chemical shift of the protons next to the ester linkage had shifted the most (9a and 9b), followed by the proton of the adjacent ring carbon (5), whereas protons farther away from the ester linkage had shifted only marginally. According to the NMR analysis, the compound appeared homogeneous and no other structures were detected. Thus, it was shown that anhydride esterification to kifunensine was directed to the 9-hydroxyl group.

Schemes E1-7 to E1-9: Kifunensine was combined in pyridine, as described above, with an excess of either 9-butanoyl anhydride, 2-(S)-methylbutanoyl anhydride or trimethylacetic anhydride (all from Merck/Sigma). The reaction mixtures were analysed by MALDI-TOF MS, showing the expected masses for 9-butanoyl-kifunensine ester (m/z 325.18 [M+Na]⁺), 9-(2-methylbutanoyl)-kifunensine ester (m/z 339.22 [M+Na]⁺) and 9-(2,2-dimethylpropionyl)-kifunensine ester (m/z 339.29 [M+Na]⁺), respectively. The products were purified by HPLC, detected by MALDI-TOF MS similarly as above and dried under vacuum.

### Example 2. Stability Studies in Buffer and Serum

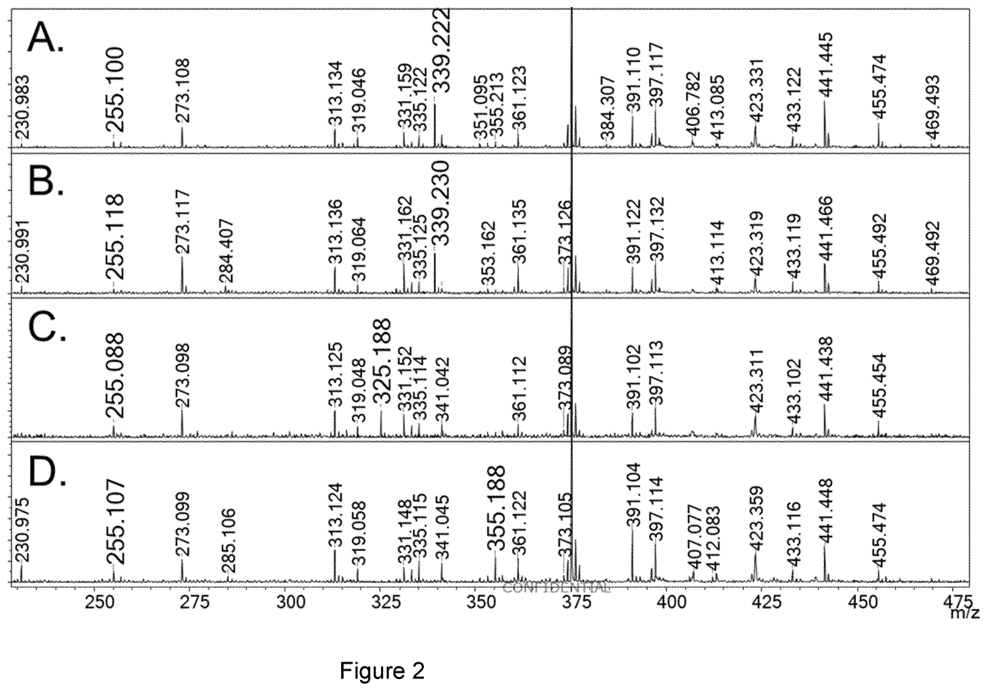

Serum was obtained from laboratory mice according to ethical permission, or from healthy human volunteers, and prepared with standard procedures. Buffer stability test were performed either in phosphate-buffered saline (PBS, Gibco) or standard cell culture medium recommended for SK-BR-3 cells by the ATCC (about neutral pH). Payload-linker molecules were incubated in serum, buffer or medium, after which the incubation mixture was analyzed by MALDI-TOF MS as described above. FIG. 2 shows MALDI-TOF mass spectra of kifunensine ester compounds after 5 days' incubation in buffered cell culture medium at +37° C., demonstrating that all studied compounds (6-(2-methyl)butanoyl-kifunensine, 6-(2,2-dimethyl)propanoyl-kifunensine, 6-butanoyl-kifunensine, 6-succinyl-kifunensine) were reasonably stable against chemical hydrolysis in neutral pH, since all compounds had higher signal intensity than the hydrolysis product kifunensine. However, as shown in FIG. 3, large differences were observed against ester hydrolysis in mouse serum after 4 days' incubation at +37° C.: both 6-(2-methyl)butanoyl-kifunensine and 6-butanoyl-kifunensine were not detected on day 4, whereas both 6-(2,2-dimethyl)propanoyl-kifunensine and 6-succinyl-kifunensine were still present, demonstrating that the latter compounds were more stable against enzymatic esterase hydrolysis than the former. Comparable results were obtained in human serum and PBS incubation experiments, indicating that steric groups in the carboxylic acid esterified to kifunensine protected the ester from hydrolysis by serum esterases: here, methyl groups, especially dimethyl modification, in 2-position of the carboxylic acid, or the acid function in 4-position. Similar studies were also conducted with the 33DFTG-ester compounds, further verifying the observation of stabilization by carboxylic acid modifications of the ester against esterase hydrolysis. However, all the kifunensine ester compounds, when applied in 10 μM concentration to SK-BR-3 cell culture for three days, were active in mannosidase I inhibition (as shown by MALDI-TOF MS analysis of neutral N-glycans of the SK-BR-3 cells after the incubation as described below), showing that regardless of the relative protection from serum esterase hydrolysis, the esters could still be hydrolysed in cells to liberate the active payload.

### Example 3. Generation of ADCs

Different payload-linker antibody conjugates were generated with trastuzumab (Herceptin, Roche) and omalizumab (Xolair, Novartis) as the antibodies. First, azide-modified antibodies were generated as follows: N-azidoacetylgalactosamine (GalNAz) residues were transferred to N-glycan antenna N-acetylglucosamine residues with mutant galactosyltransferase reaction after either cleaving the galactose residues with recombinant S. pneumoniae β-galactosidase (Merck) or cleaving the N-glycan after the core GlcNAc residue with endoglycosidase S2 (Glycinator, Genovis) according to manufacturers instructions. The cleaved antibodies were then incubated with recombinant Y289L mutant bovine B1,4-galactosyltransferase and UDP-GalNAz (both from Thermo) in the presence of Mn²⁺ containing buffer at +37° C. overnight essentially according to manufacturer's instructions. Azide-to-antibody ratios were determined by Fabricator enzyme digestion according to manufacturers instructions (Genovis) and MALDI-TOF MS essentially as described (Satomaa et al. 2018. Antibodies 7(2), 15). The MS analyses demonstrated that the azide-to-antibody ratios were 4:1 when the galactosidase was used and 2:1 when the endoglycosidase was used.

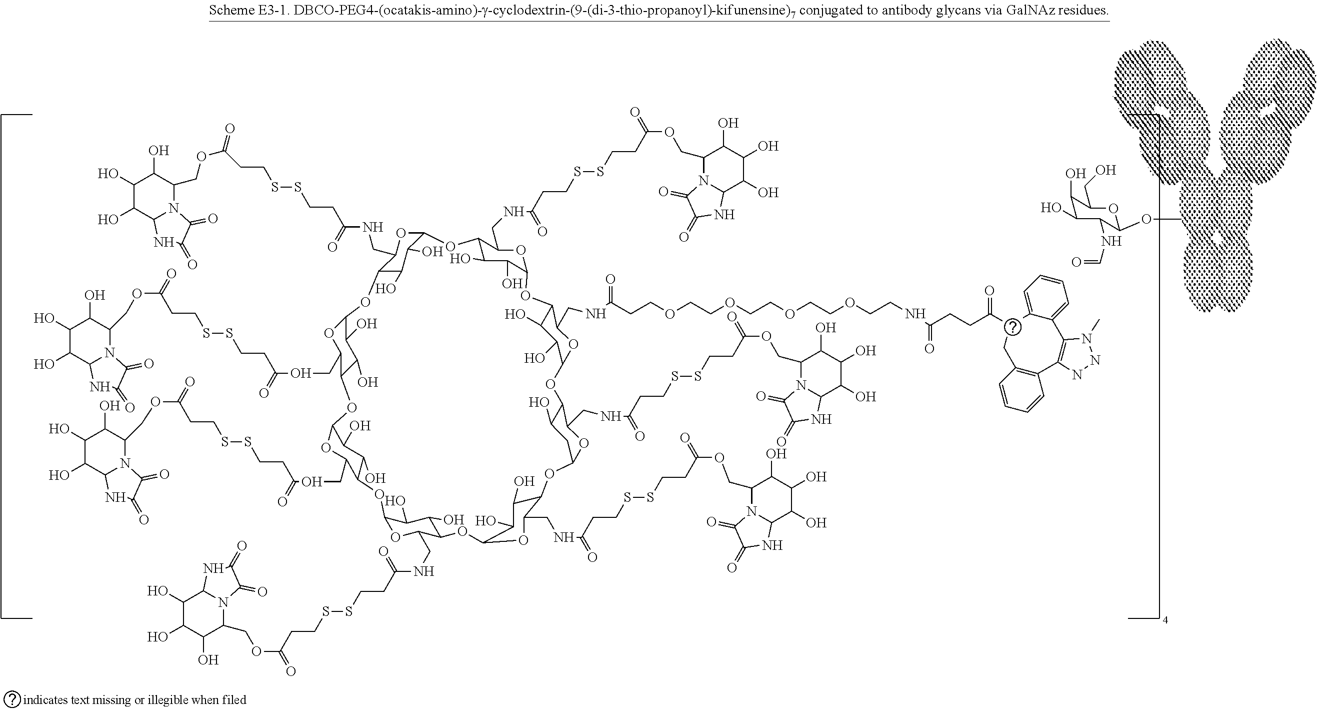

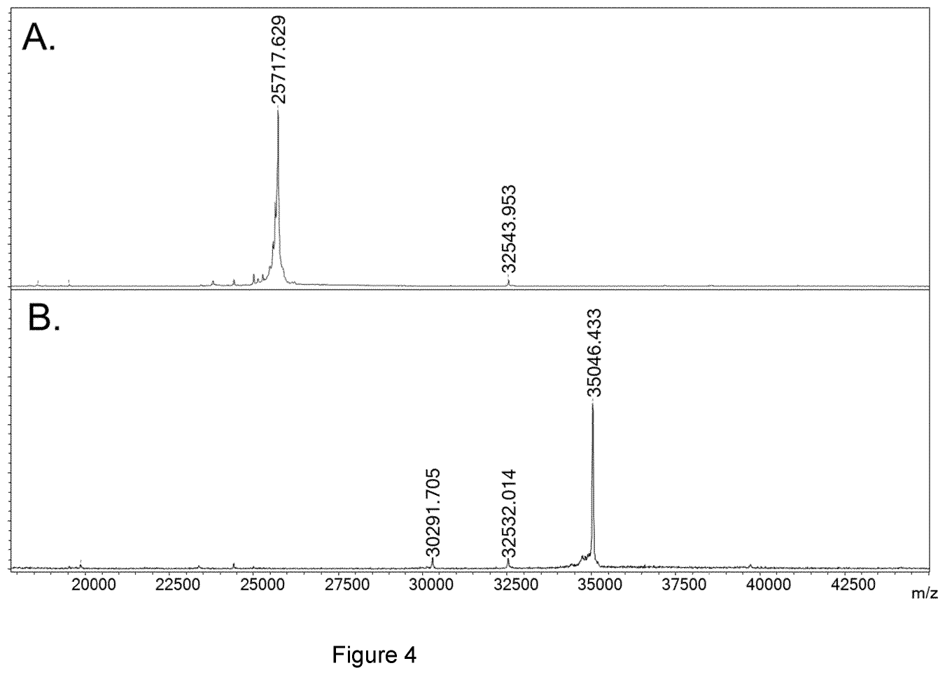

Scheme E3-1. For conjugation to azide-modified antibodies, DBCO-modified payloads were used. FIG. 4B shows the successful generation of kifunensine-conjugated trastuzumab, homogeneous DAR=28, from azide-modified trastuzumab, 4 azides/antibody. The MALDI-TOF mass spectrum of the heavy chain Fc domain showed the expected m/z value of homogeneous ADC with exactly four DBCO-PEG4-(octakis-amino)-γ-cyclodextrin-(9-(di-3-thio-propanoyl)-kifunensine)₇, so that the DAR for the complete ADC was 28. Similar ADCs with DAR=28 were generated also using DBCO-PEG4-(octakis-amino)-γ-cyclodextrin-(9-(4-methyl-4-thiobutanoyl-3-thiopropanoyl)-kifunensine)₇ (Scheme E3-2) and DBCO-PEG₄-(octakis-amino)-γ-cyclodextrin-(9-(4,4-dimethyl-4-thiobutanoyl-3-thiopropanoyl)-kifunensine)₇ (Scheme E3-3).

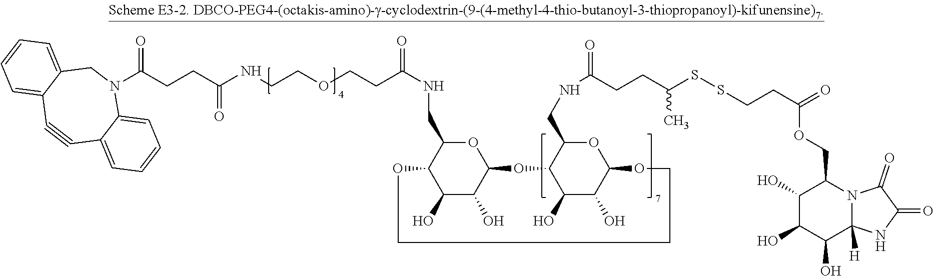

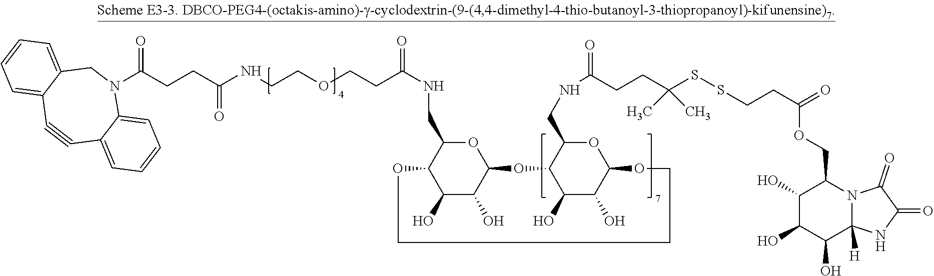

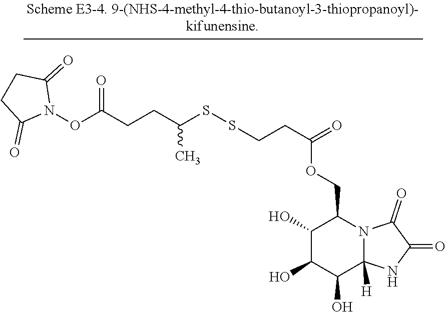 [SMILES: CC(CCC(=O)ON1C(=O)CCC1=O)SSCCC(=O)OC[C@@H]1[C@@H](O)[C@H](O)[C@H](O)[C@H]2NC(=O)C(=O)N12]

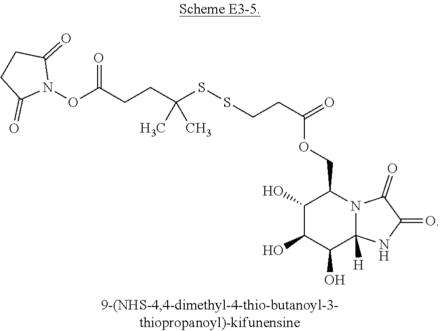 [SMILES: CC(C)(CCC(=O)ON1C(=O)CCC1=O)SSCCC(=O)OC[C@@H]1[C@@H](O)[C@H](O)[C@H](O)[C@H]2NC(=O)C(=O)N12]

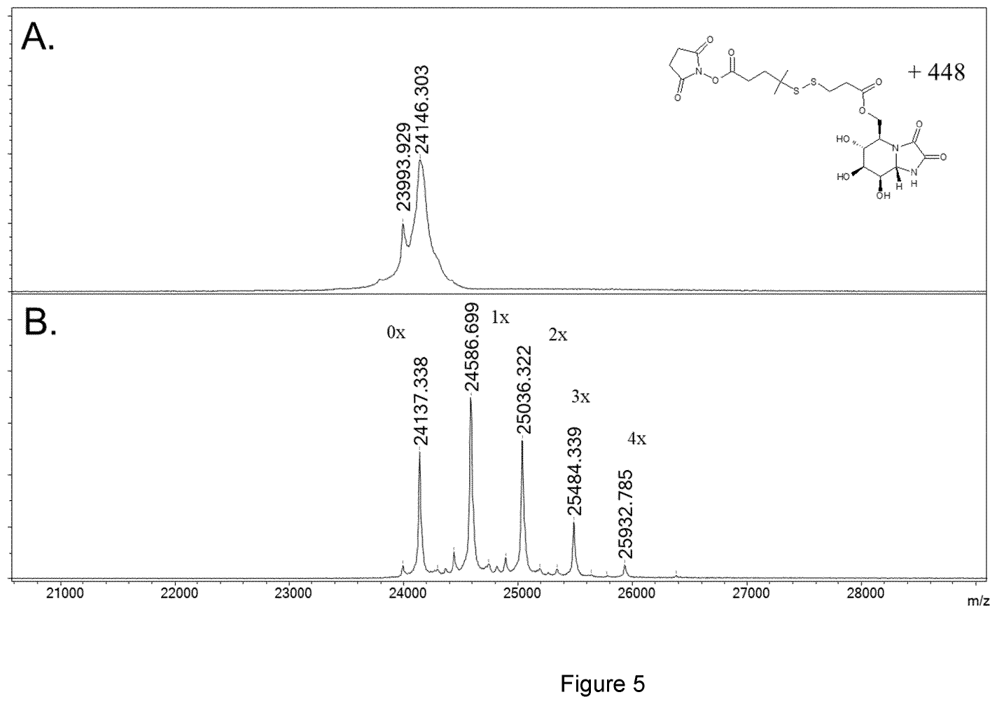

Scheme E3-4. FIG. 5 shows the successful generation of kifunensine-conjugated trastuzumab, average DAR=9, with 9-(NHS-4,4-dimethyl-4-thio-butanoyl-3-thiopropanoyl)-kifunensine, where the NHS-activated ester forms amide bonds with lysine side chain amino groups of the antibody. The MALDI-TOF mass spectrum of the heavy chain Fc domain was recorded and based on the Fc domain data the DAR of the complete ADC was calculated to be on average about 9 (Satomaa et al. 2018). Similar ADCs were generated also with 9-(NHS-4-methyl-4-thio-butanoyl-3-thiopropanoyl)-kifunensine (Scheme E3-5).

### Example 4. ADC Treatment of Cells

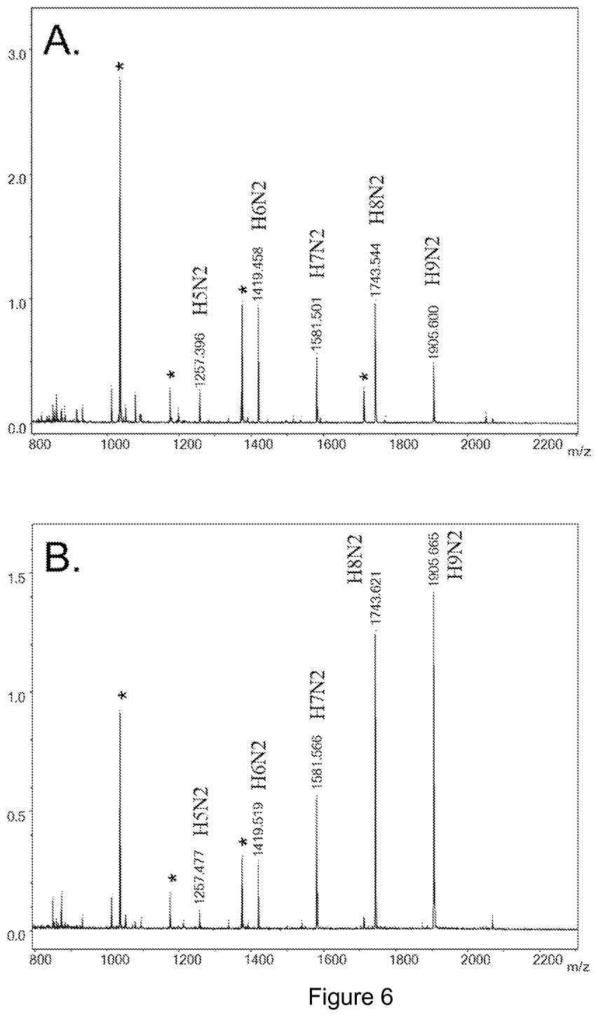

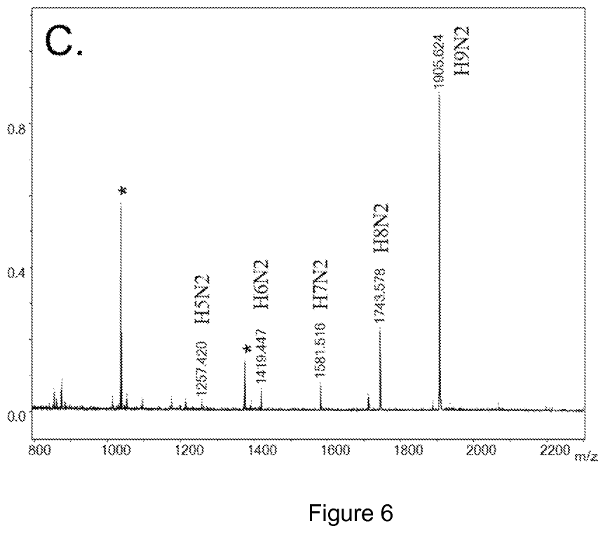

SK-BR-3 breast cancer cells (ATCC) were cultured in recommended conditions and incubated with glycosylation inhibitors and ADCs for three days. The cells were then subjected to N-glycan profiling with MALDI-TOF MS essentially as described in Leijon et al. 2017, J Clin Endocrinol Metab 102(11):3990-4000, although without the deparaffinization step. The N-glycan profiles comprising the cellular neutral N-glycans showed increased number of hexose residues in the high-mannose type N-glycan signals with assigned monosaccharide compositions Man₅₋₉GlcNAc₂ (m/z 1257, m/z 1419, m/z 1581, m/z 1743 and m/z 1905 for [M+Na]⁺ adduct ions, respectively; which could be relatively quantitated based on relative signal intensity as described in Leijon et al. 2017; data not shown) when the cells were subjected to either kifunensine or kifunensine-ADC treatment. In control cells (no treatment) as well as in cells treated with 1 pM trastuzumab for 3 days, the N-glycan profile was as shown in FIG. 6A, whereas in cells treated in parallel with 10 μM kifunensine, the average number of mannose residues (Man) in the Man₅₋₉GlcNAc₂ glycan signal series was increased to over 8.5. This demonstrated effective inhibition of mannosidase I activity in inhibitor treated cells.

FIG. 6B shows effective inhibition of N-glycan processing in SK-BR-3 cells by 100 pM trastuzumab-kifunensine conjugate (lysine-conjugated with 9-(NHS-4,4-dimethyl-4-thio-3-thiopropanoyl)-kifunensine, average DAR=21) treatment, whereas FIG. 6C shows the profile after treatment with 1 nM ADC. Based on the data, IC50 for inhibition of mannosidase I activity with the ADC was calculated to be about 100 pM by non-linear regression analysis.

To study the resistance to serum esterase hydrolysis, trastuzumab ADC was generated using payload linker as described in Schemes E3-1, and incubated in both human and mouse sera before addition to SK-BR-3 cell culture in 10 nM concentration for three days. Activity was retained even after 7 days' incubation in both human and mouse sera, however it was reduced over 50%.

To demonstrate that linker-stabilized ADCs were even more resistant to serum esterases, trastuzumab ADCs were generated using payload linkers as described in Schemes E3-2, E3-4 and E3-5, and incubated in both human and mouse sera before addition to SK-BR-3 cell culture in 10 nM concentration for three days. Subsequent analysis of the cells' glycosylation for increase in high-mannose N-glycan relative amounts demonstrated even more efficient inhibition of mannosidase I activity with these ADCs, with less than 50% reduction in activity.

### Example 5. In Vivo Xenograft Experiment

HCC-1954 cancer cells were obtained from the ATCC (USA) and cultured according to the manufacturer's instructions to study accumulation of antibody-drug conjugates in trastuzumab-resistant Her2-positive xenograft tumors in immunodeficient nude mice (Balb/cAnNRj-Foxn1nu-nu). The study was performed at the TCDM/Central Animal Laboratory, University of Turku, Finland, according to the appropriate ethical committee approval. Cells for inoculation to mice were prepared in vigorous exponential growth phase. 5 million cells in 50% Matrigel were inoculated s.c. to the flank of each mouse. Clinical signs and general behavior of the animals was observed regularly. No potential signs of toxicity were recorded. At the end of the study, the mice were examined for potential macroscopic changes in major organs, but none were detected. Tumor growth was followed by palpation. After caliper measurement, tumor volume was calculated according to 0.5×length×width². The first dosings were administered when average tumor volume reached >100 cm³. Mice were evenly divided into study groups so that each group received similar distribution of different-sized tumors and the average tumor volumes were similar in each group. The mice received two additional dosings at 3 days and 6 days after the first dosing. Control mice received no dosings. At the end of the study on day 8 after the first dosing, the mice were sacrificed and representative samples of the tumors, blood (serum), liver, spleen, skin and muscle were taken and frozen for analyses.

Test substances were prepared as described above. Three 10 mg/kg i.v. doses each of trastuzumab, trastuzumab-[DBCO-PEG4-(octakis-amino)-γ-cyclodextrin-(9-(di-3-thio-propanoyl)-kifunensine)₇]₄ (DAR=28; trastuzumab-cyclo-kifunensine), and trastuzumab-[GalNAz-DBCO-PEG4-(octakis-amino)-γ-cyclodextrin-(9-(4-methyl-4-thio-butanoyl-3-thiopropanoyl)-kifunensine)₇]4 (DAR=28; trastuzumab-cyclo-Me-kifunensine), were given to the tumor-bearing mice and tumors and tissues were collected on day 8. The samples were subjected to N-glycan profiling with MALDI-TOF MS essentially as described above. The tumor glycosylation had changed in the ADC-treated groups consistent with effective inhibition of mannosidase I activity, based on accumulation of large high-mannose type N-glycans and corresponding relative decrease in other glycan types including small high-mannose type N-glycans, complex-type N-glycans and sialylated glycans. When calculated similarly as above, the original cell line cultured in vitro had on average 7.13 mannose residues (Man) in the Man₅₋₉GlcNAc₂ glycan signal series, non-treated xenograft tumors had on average 7.14, and trastuzumab-treated tumors had on average 7.12 mannose residues (Man) in the Man₅₋₉GlcNAc₂ glycan signal series, whereas in the cyclo-Me-kifu treated tumors the average number of mannose residues had increased to 7.40 and in the cyclo-kifu treated tumors to 7.36. The proportion of high-mannose type N-glycans of the total neutral N-glycans in the tumors was studied essentially as described in Satomaa et al. 2009 (BMC Cell Biol. 10:42), showing that in both ADC-treated mice high-mannose type N-glycans had increased in proportion of total N-glycans compared to other N-glycan structure types, whereas naked antibody treatment had no such effect: In both non-treated and trastuzumab-treated mice, the proportion of high-mannose type N-glycans was 43-47% and the proportion of complex-type N-glycans was 33-38%; whereas in trastuzumab-cyclo-kifunensine-treated mice the proportion of high-mannose type N-glycans was 78% and the proportion of complex-type N-glycans was 11%; and in trastuzumab-cyclo-Me-kifunensine-treated mice the proportion of high-mannose type N-glycans was 80% and the proportion of complex-type N-glycans was 10%. Thus, the naked antibody trastuzumab had no effect on the glycosylation, but the kifunensine-conjugated ADCs had, and the trastuzumab-cyclo-Me-kifunensine-treated mice had more drastic glycosylation change than trastuzumab-cyclo-kifunensine-treated mice, consistent with the increased stability of the linker with the 4-thio-butanoyl group compared to the 3-thio-propanoyl group in the corresponding position.

In control tissue of the ADC treatment groups, for example liver, but not in the trastuzumab group, N-glycosylation also showed signs of similar changes but they were significantly less intense demonstrating tumor-targeted and tumor-specific glycosylation change in accordance with using the human HER2-specific antibody trastuzumab as the targeting unit in the ADCs. In all the mice no signs of toxicity nor body weight loss were observed.

### Example 6. Tunicamycin Conjugates

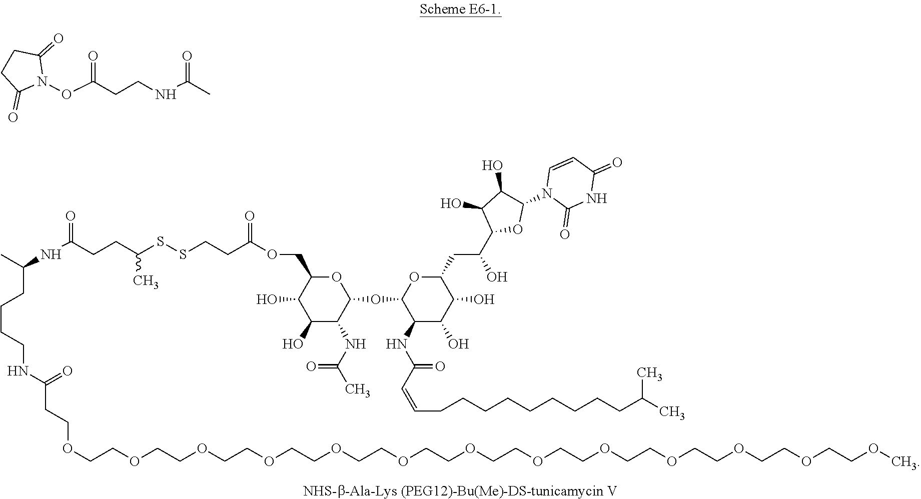 [SMILES: CC(=O)NCCC(=O)ON1C(=O)CCC1=O ; COCCOCCOCCOCCOCCOCCOCCOCCOCCOCCOCCOCCOCCC(=O)NCCCC[C@@H](C)NC(=O)CCC(C)SSCCC(=O)OC[C@H]1O[C@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C\CCCCCCCCCC(C)C)[C@H](NC(C)=O)[C@@H](O)[C@@H]1O]

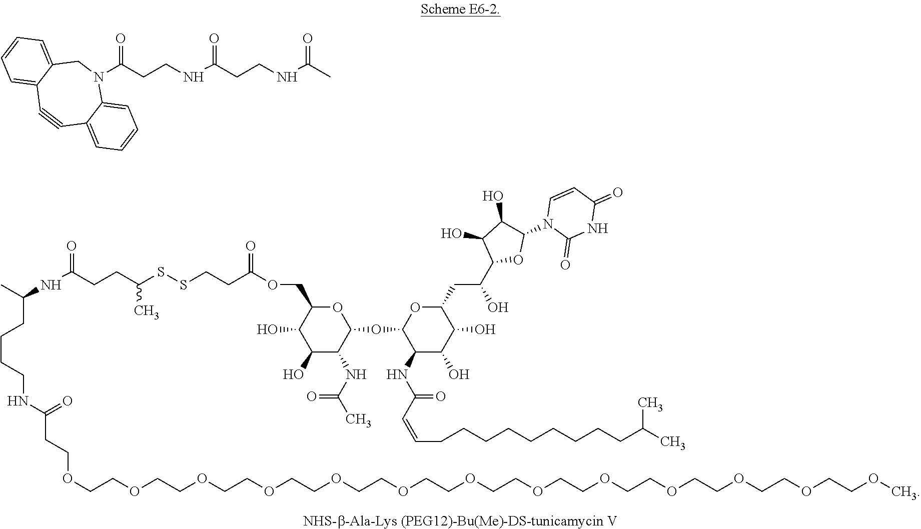 [SMILES: CC(=O)NCCC(=O)NCCC(=O)N1Cc2ccccc2C#Cc2ccccc21 ; COCCOCCOCCOCCOCCOCCOCCOCCOCCOCCOCCOCCOCCC(=O)NCCCC[C@@H](C)NC(=O)CCC(C)SSCCC(=O)OC[C@H]1O[C@H](O[C@@H]2O[C@H](C[C@@H](O)[C@H]3O[C@@H](n4ccc(=O)[nH]c4=O)[C@H](O)[C@@H]3O)[C@H](O)[C@H](O)[C@H]2NC(=O)/C=C\CCCCCCCCCC(C)C)[C@H](NC(C)=O)[C@@H](O)[C@@H]1O]

NHS-β-Ala-Lys(PEG12)-Bu(Me)-DS-tunicamycin V (Scheme E6-1, obtained by custom synthesis) was conjugated to amino-DBCO reagent (Jena Bioscience, CLK-A103) to form DBCO-β-Ala-Lys(PEG12)-Bu(Me)-DS-tunicamycin V (Scheme E6-2) by combining the reagents in aqueous buffered solution. MALDI-TOF MS was performed to verify that the correct product was formed. The DBCO-linker-payload was further conjugated to trastuzumab-GalNAz (DAR=2) as described above. In MALDI-TOF MS of the reaction products, Fabricator-digested Fc-GalNAz portion of trastuzumab-GalNAz was not anymore detectable at about m/z 24384, but instead the correct reaction product was detected at m/z 26494. The change in m/z, 2110 Da, corresponded well to the mass of the linker-payload (2123 Da). The complete ADC has DAR=2 and it can deliver the tunicamycin payload to HER2-positive target cells.

### Example 7. Peptide Conjugates

Multivalent kifunensine-peptide-DBCO linker-payload was constructed by conjugating the cell-penetrating peptide with amino acid sequence CGRKKRRQRRR (SEQ ID No: 1) (Bachem) with kifunensine. The products were analyzed by MALDI-TOF MS: the free peptide CGRKKRRQRRR (SEQ ID No: 1) was detected at m/z 1500.1 [M+H]⁺; after conjugation with DBCO-PEG4-maleimide the product was detected at m/z 2174.4 [M+H]⁺; and after further conjugation with two NHS-Bu(Me)-DS-kifunensine linker-payloads/peptide (corresponding to conjugation to side chain amino groups of the both lysine residues) the product was detected at m/z 3042.4 [M+H]⁺, while no original peptide signal was detected; demonstrating efficient conjugation. The kifunensine-peptide conjugate can be utilized to deliver kifunensine to cells. Further, the DBCO-peptide-kifunensine₂ linker-payload can be conjugated to any azide-labeled targeting unit such as antibody-GalNAz by simple incubation in aqueous solvent to obtain an ADC.

### Example 8. Kifunensine Conjugates

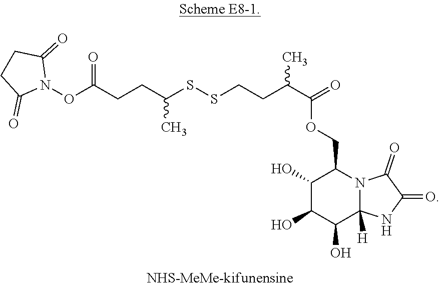

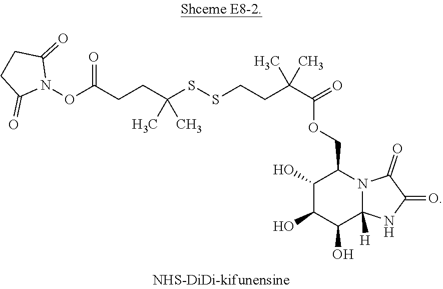 [SMILES: CC(C)(CCC(=O)ON1C(=O)CCC1=O)SSCCC(C)(C)C(=O)OC[C@@H]1[C@@H](O)[C@H](O)[C@H](O)[C@H]2NC(=O)C(=O)N12]

Two NHS-ester-activated disulfide-kifunensine linker-payloads were obtained as custom synthesis (Schemes E8-1 and E8-2), wherein both the disulfide group and the kifunensine-ester group were protected by methyl and dimethyl groups, respectively. Trastuzumab conjugates thereof were prepared by adding molar excesses of either linker-payload, dissolved in small amount of DMSO, to trastuzumab antibody diluted in PBS. After the reaction, the products were analyzed by MALDI-TOF MS as described above. Analysis of both Fabricator-digested Fc domain and the full antibody established average DAR of 18 and 20, when 130× and 180× excesses of NHS-MeMe-kifunensine were applied, respectively, and average DAR of 16 and 18, when 180× and 240× excesses of NHS-MeMe-kifunensine were applied, respectively. The resulting lysine side chain amide conjugates are useful for specifically delivering kifunensine to HER2-positive cells.

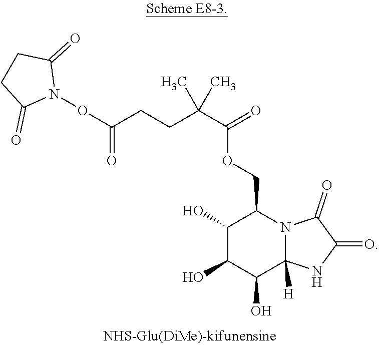 [SMILES: CC(C)(CCC(=O)ON1C(=O)CCC1=O)C(=O)OC[C@@H]1[C@@H](O)[C@H](O)[C@H](O)[C@H]2NC(=O)C(=O)N12]

NHS-ester-activated kifunensine linker-payload was obtained as custom synthesis (Scheme E8-3), wherein there is no disulfide group and the kifunensine-ester group is protected by dimethyl group. Trastuzumab conjugates thereof were prepared as described above in PBS. After the reaction, the products were analyzed by MALDI-TOF MS as described above. Analysis of both Fabricator-digested Fc domain and the full antibody established average DAR of over 10. The ADCs of this Example can effectively deliver the kifunensine payload to HER2-positive target cells.

### Example 9. Glucosidase Inhibitor Conjugates

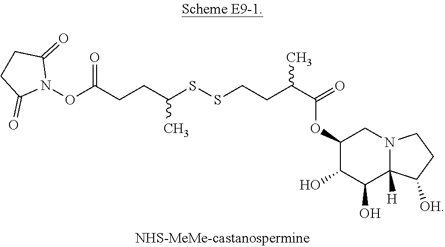

NHS-ester-activated castanospermine linker-payload was obtained as custom synthesis (Scheme E9-1), wherein both the disulfide group and the castanospermine-ester group were protected by methyl groups. Trastuzumab conjugates thereof are prepared by adding a molar excess of the linker-payload, dissolved in small amount of DMSO, to trastuzumab antibody diluted in PBS. After the reaction, the product is analyzed by MALDI-TOF MS as described above. The resulting lysine side chain amide conjugate is useful for specifically delivering castanospermine to HER2-positive cells, resulting in α-glucosidase inhibition, N-glycan profile change into glucosylated N-glycans, inhibition of N-glycan processing into hybrid-type and complex-type N-glycans; and inducing immunomodulatory activity thereof.

### Example 10. ADC Stability and Activity

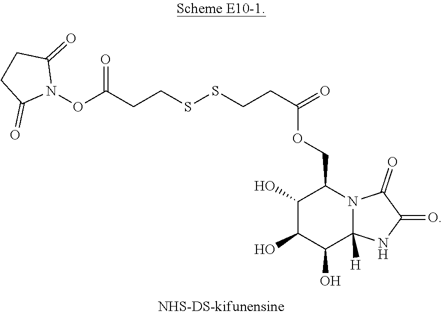 [SMILES: O=C(CCSSCCC(=O)ON1C(=O)CCC1=O)OC[C@@H]1[C@@H](O)[C@H](O)[C@H](O)[C@H]2NC(=O)C(=O)N12]

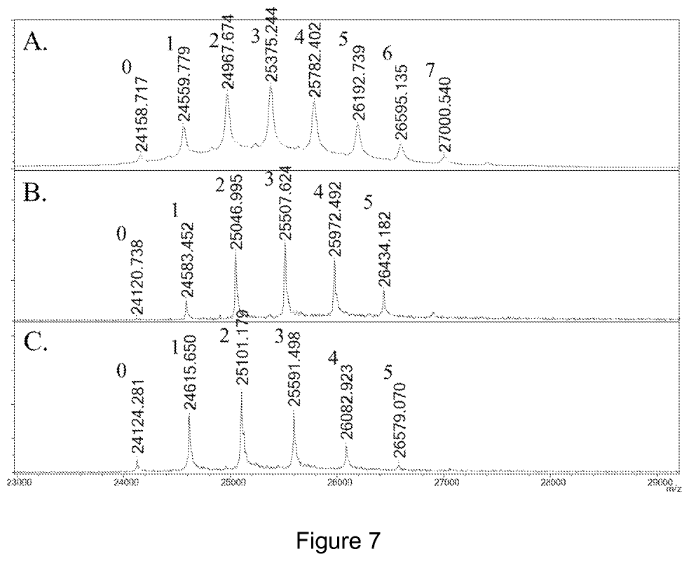

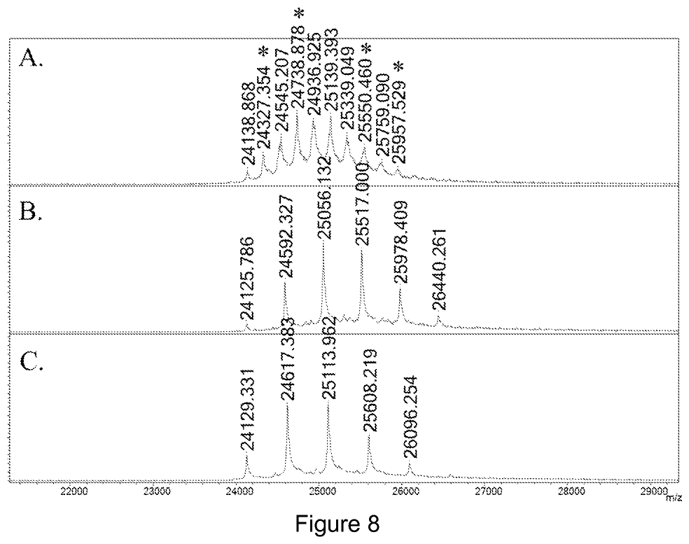

Trastuzumab conjugates were prepared through amide formation to lysine side chains with NHS-DS-kifunensine (Scheme E10-1, product: Tmab-Kifu), NHS-MeMe-kifunensine (Scheme E8-1, product: Tmab-MeMe-Kifu) and NHS-DiDi-kifunensine (Scheme E8-2, product: Tmab-DiDi-Kifu) as described above. Chemical stability of the resulting ADCs was studied by incubating the isolated ADCs in PBS at +37° C. for seven days. FIG. 7 shows MALDI-TOF mass spectra of Fabricator-digested Fc fragments of the ADCs before the incubation (t=0) and FIG. 8 shows them after the incubation (t=7d). Both trastuzumab-MeMe-kifunensine and trastuzumab-DiDi-kifunensine mass spectra had remained essentially similar, whereas trastuzumab-DS-kifunensine showed extensive loss of kifunensine residues (change of 214 Da) due to linker ester hydrolysis.

JIMT-1 cancer cells (DSMZ-German Collection of Microorganisms and Cell Cultures; variable/medium HER2 expression) and HCC-1954 cancer cells (ATCC; high HER2 expression) were cultured according to the manufacturers' instructions in standard cell culture conditions and incubated for 3-4 days with either 10 pM kifunensine or trastuzumab-kifunensine ADCs. Effect on N-glycan profiles of the cells was analyzed by isolating cellular N-glycans and MALDI-TOF MS as described above. In HCC-1954 cells, both kifunensine and either 10 nM or 100 nM trastuzumab-cyclo-Me-kifu (described below) were equally effective in inhibiting processing α-mannosidase and resulted in increase of the relative proportion of especially the Man9 high-mannose N-glycans in the N-glycome: before incubation, the Man9 glycan comprised below 20% of total oligomannose-type N-glycan signal intensity, whereas after the incubation, Man9 glycan comprised over 60% of total oligomannose-type N-glycan signal intensity. At the same time, relative proportion of complex-type N-glycans was drastically diminished. The same change, although less intensive, occurred also in JIMT-1 cells.

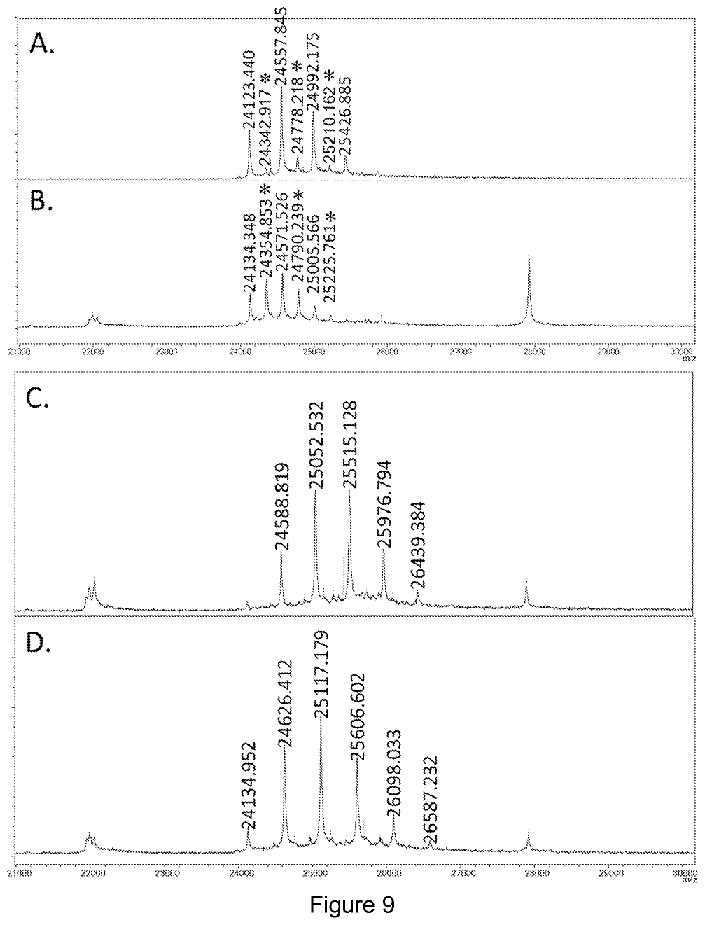

To study the biological stability of the same ADCs as above, aliquots were incubated in mouse serum for 2 days at +37° C. Similarly as in 7 days' incubation in PBS, Tmab-Kifu was not stable in mouse serum but had lost kifunensine groups at the ester linkage. FIG. 9 shows the difference between Tmab-Me-Kifu, Tmab-MeMe-Kifu and Tmab-DiDi-Kifu in the serum incubation. Tmab-Me-Kifu was not stable but instead was hydrolysed by esterase activity at the ester linkage to kifunensine, whereas Tmab-MeMe-Kifu and Tmab-DiDi-Kifu were stable against serum esterase activity and no loss of kifunensine was observed (FIG. 9).

### Example 11. Cyclodextrin Conjugates

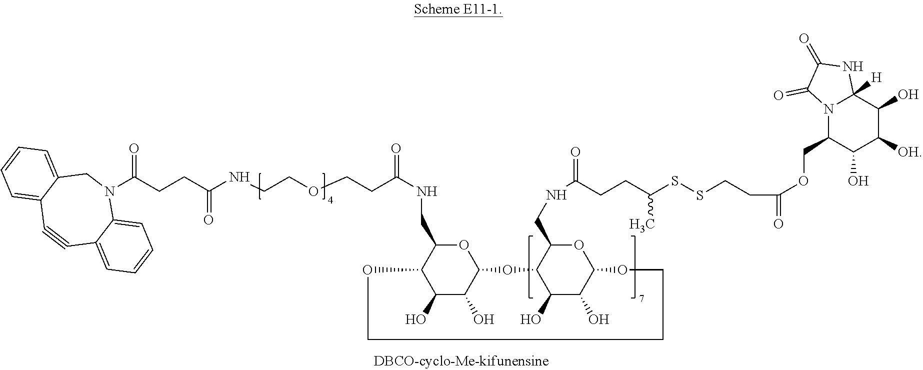

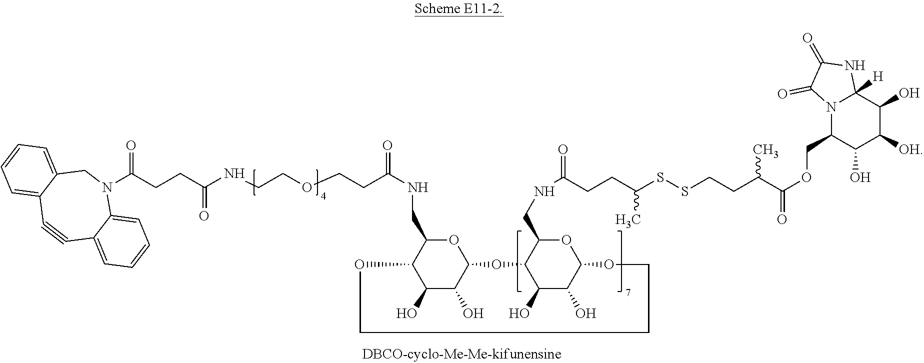

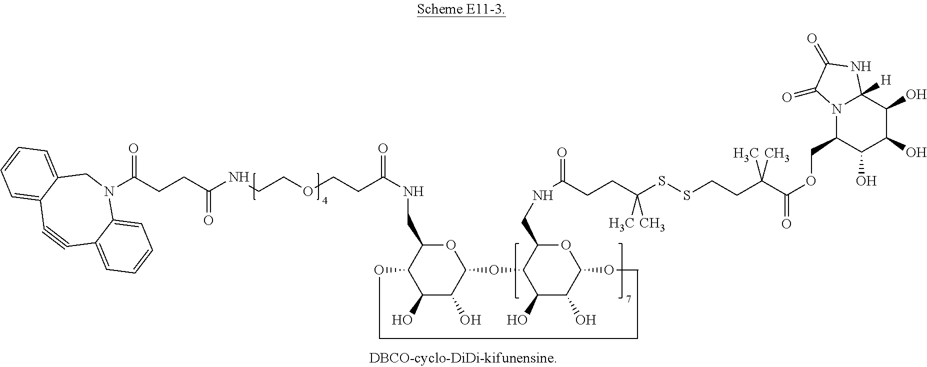

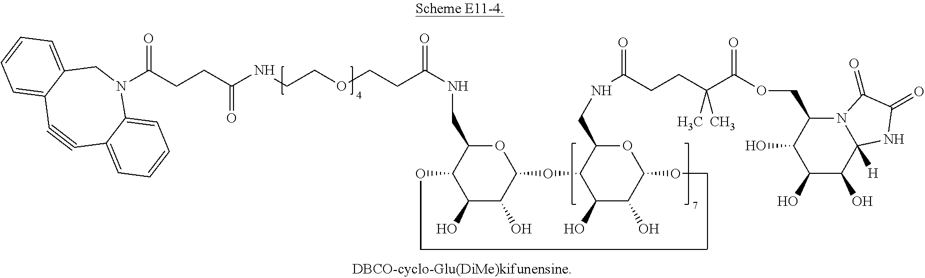

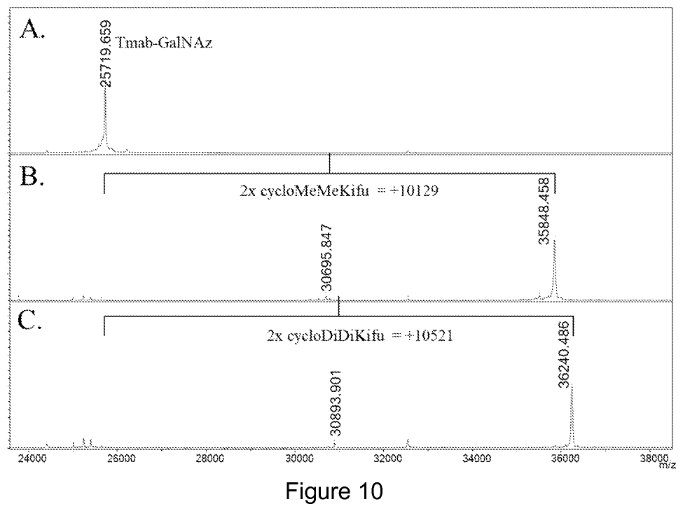

DBCO-linker-payloads with cyclodextrin scaffolds were prepared with kifunensine NHS-linker-payloads essentially as described above, to produce compounds according to Schemes E11-1 to E11-4. ADCs were produced with glycoconjugated trastuzumab-GalNAz as described above with 3-4 azide groups/antibody by dissolving the linker-payload in DMSO and combining a small molar excess of it with the trastuzumab-GalNAz in PBS, to yield ADCs with DAR=3-4. The end product was characterized by MALDI-TOF MS after Fabricator digestion (FIG. 10) and as full antibody to demonstrate that the click reaction had been essentially complete, so that the complete ADC essentially had DAR=28. Schemes E11-5 to E11-8 show the linkage structure of the ADCs.

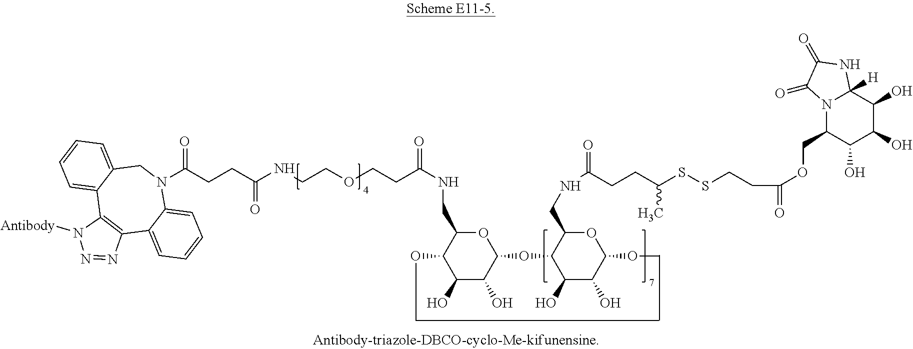

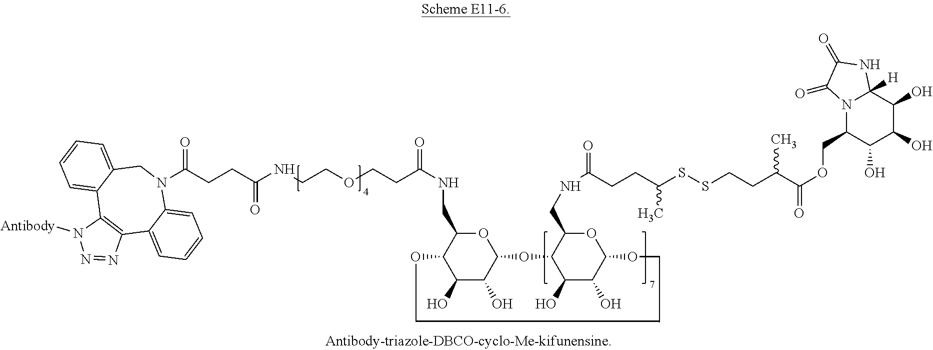

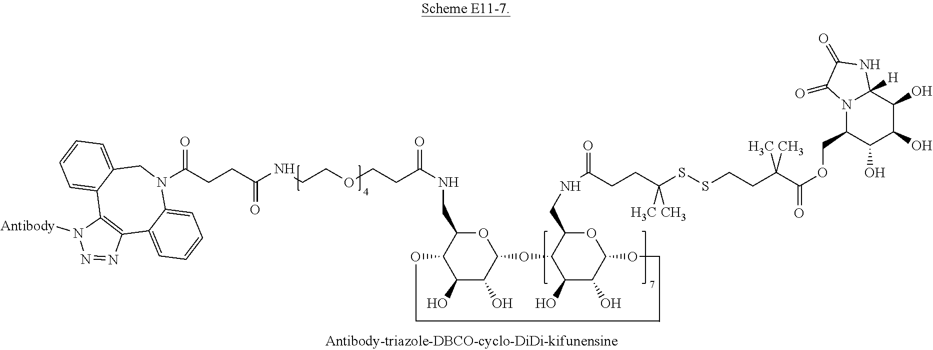

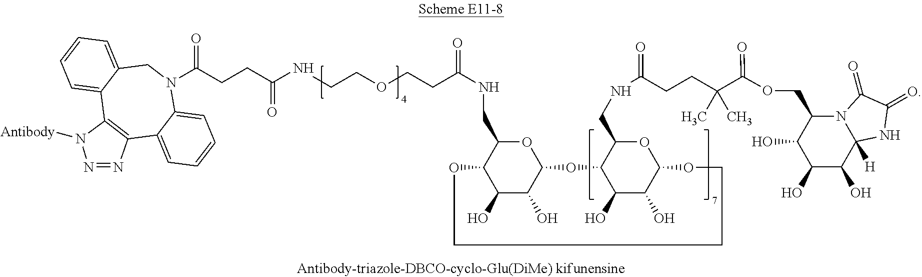

### Example 12. Carbonate Conjugates

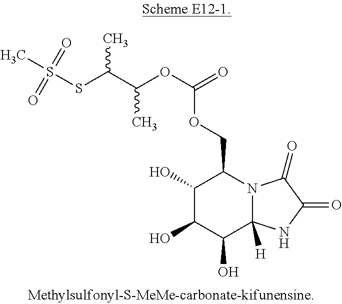

Hinge region S—S bonds of 10 mg antibody in PBS were reduced using 20× molar excess TCEP (Tris(2-carboxyethyl)phosphine) to IgG in presence of 1 mM DTPA (diethylene triamine pentaacetic acid) in 5 mL reaction column for 2 hours at +37° C. The reduced antibody was purified using Protein A HPLC to remove TCEP. 20× molar excess of the payload according to Scheme E12-1 was dissolved in DMSO, added to the reduced antibody solution and incubated for 1 hour at +37° C. The final product was purified using HPLC to obtain antibody-S—S-MeMe-carbonate-kifunensine ADC, DAR=8.

### Example 13. Maleimide Conjugates

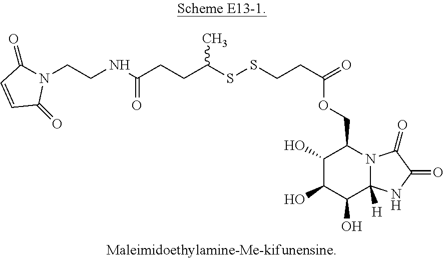 [SMILES: CC(CCC(=O)NCCN1C(=O)C=CC1=O)SSCCC(=O)OC[C@@H]1[C@@H](O)[C@H](O)[C@H](O)[C@H]2NC(=O)C(=O)N12]

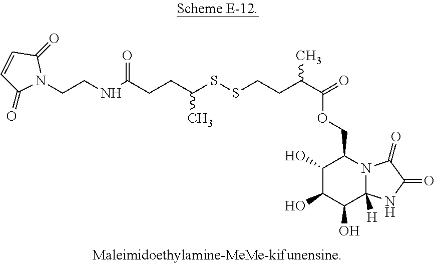

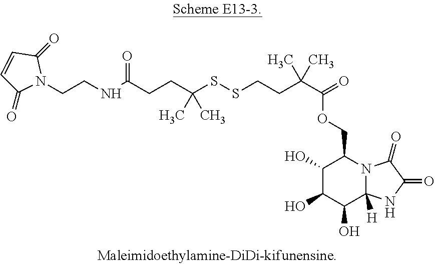 [SMILES: CC(C)(CCC(=O)NCCN1C(=O)C=CC1=O)SSCCC(C)(C)C(=O)OC[C@@H]1[C@@H](O)[C@H](O)[C@H](O)[C@H]2NC(=O)C(=O)N12]

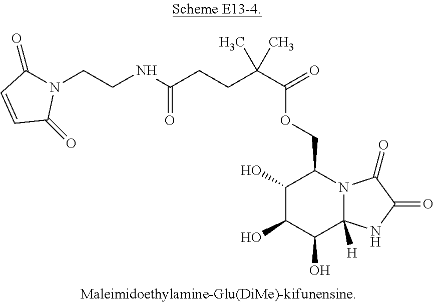 [SMILES: CC(C)(CCC(=O)NCCN1C(=O)C=CC1=O)C(=O)OC[C@@H]1[C@@H](O)[C@H](O)[C@H](O)[C@H]2NC(=O)C(=O)N12]

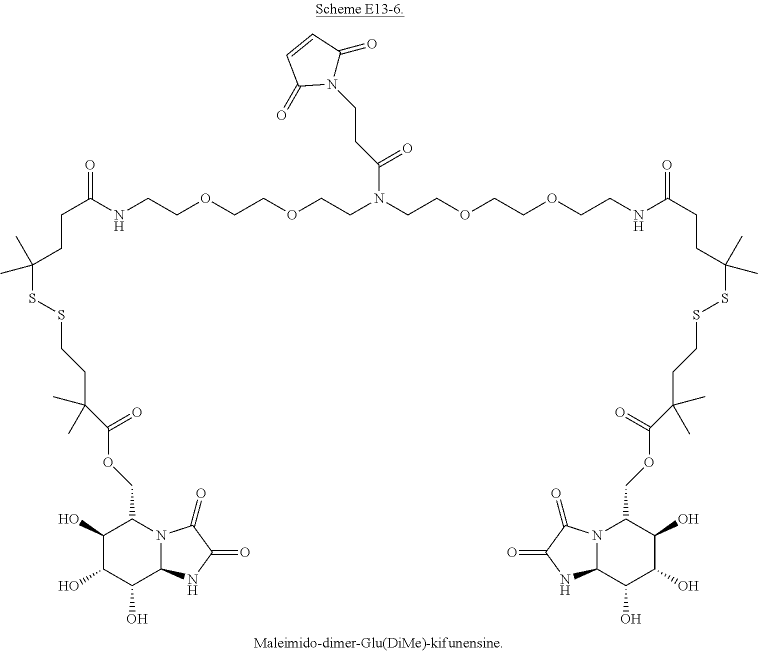

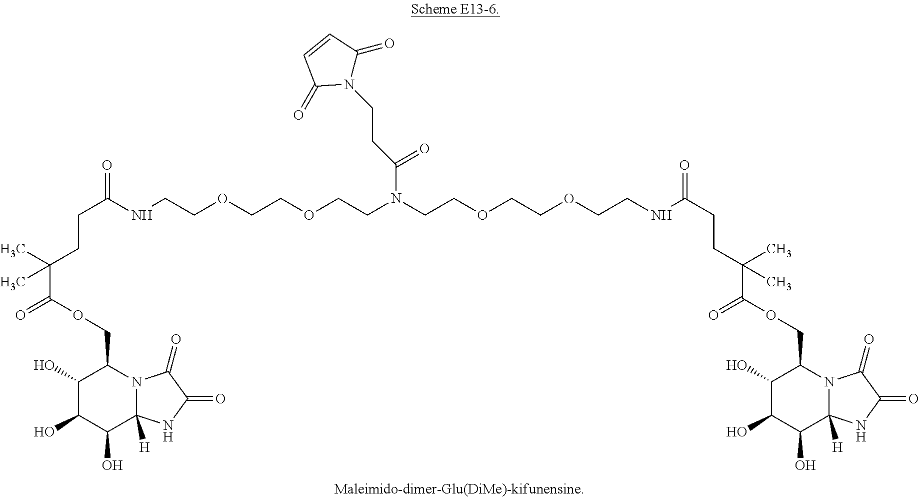 [SMILES: CC(C)(CCC(=O)NCCOCCOCCN(CCOCCOCCNC(=O)CCC(C)(C)C(=O)OC[C@@H]1[C@@H](O)[C@H](O)[C@H](O)[C@H]2NC(=O)C(=O)N12)C(=O)CCN1C(=O)C=CC1=O)C(=O)OC[C@@H]1[C@@H](O)[C@H](O)[C@H](O)[C@H]2NC(=O)C(=O)N12]

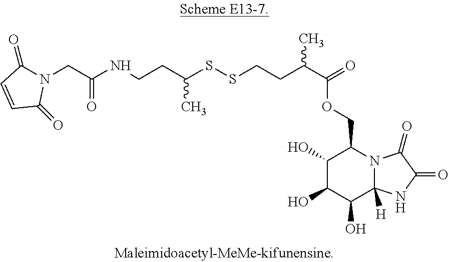

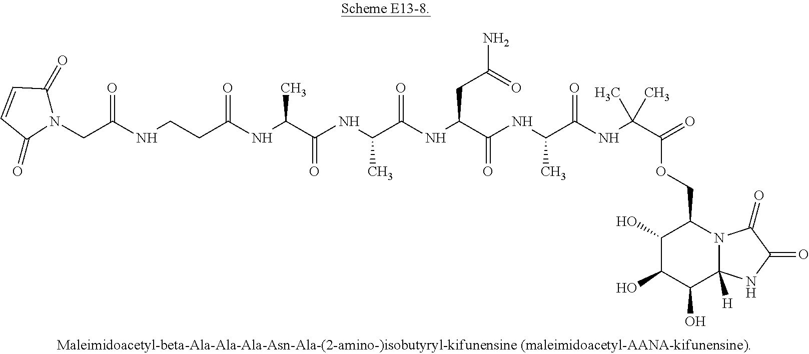 [SMILES: C[C@H](NC(=O)CCNC(=O)CN1C(=O)C=CC1=O)C(=O)N[C@@H](C)C(=O)N[C@@H](CC(N)=O)C(=O)N[C@@H](C)C(=O)NC(C)(C)C(=O)OC[C@@H]1[C@@H](O)[C@H](O)[C@H](O)[C@H]2NC(=O)C(=O)N12]

Kifunensine-linker payloads according to Schemes E13-1 to E13-8 were prepared. Trastuzumab conjugates thereof were prepared essentially as described above and characterized by MALDI-TOF MS. The maleimidoethylamine linker-payloads were prepared essentially as described below for Scheme E13-1: 5.5 mmol NHS-Me-kifunensine and 8.1 mmol N-(2-aminoethyl)maleimide were mixed in 100 mL DMSO. 23 mL 1:10 N,N-diisopropylethylamine(DIPEA):DMSO was added to the mixture. The reaction was performed at room temperature. Extent of reaction was followed using MALDI-TOF MS of the reaction mixture. The ready product was purified using RP-HPLC.

The ADCs were prepared essentially as described for E13-1: hinge region S—S bonds of 5.1 mg trastuzumab in PBS were reduced using 40× molar excess of TCEP (tris(2-carboxyethyl)phosphine) to IgG in presence of 1 mM DTPA (diethylene triamine pentaacetic acid). Reaction volume was 1 mL, reaction time 1 hour at 37° C. The resulting reduced trastuzumab was purified using RP-HPLC to remove TCEP. 40× molar excess of maleimidoethyl-Me-kifunensine linker-payload was dissolved in DMSO and added on the reduced trastuzumab solution. Reaction time was 1 hour at 37° C. The ADC was purified using RP-HPLC. MALDI-TOF MS showed essentially complete reaction with about DAR=8.

Dimeric linker-payloads were constructed essentially as follows for E13-5: 5 mmol NHS-DiDi-kifunensine and 720 nmol N-Mal-N-bis(Peg2-amine) which contains 1440 nmol free amines were mixed in 140 mL DMSO. 2 mL 1:2 N,N-diisopropylethylamine(DIPEA):DMSO was added to mixture. Reaction was performed at room temperature for 3 hours. Extent of reaction was followed using MALDI-TOF MS. Product was purified using RP-HPLC. The dimeric structure can be used to generate homogeneous ADCs with DAR=16.

ADCs with maleimide stabilization was prepared essentially as described for trastuzumab and E13-8: Hinge region S—S bonds of 20 mg trastuzumab in PBS were reduced using 20× molar excess of TCEP (Tris(2-carboxyethyl)phosphine) to IgG in presence of 1 mM DTPA (diethylene triamine pentaacetic acid). Reaction volume was 4 mL, reaction time 2 hours at 37° C. 40× molar excess to IgG maleimidoacetyl-AANA-kifunensine linker-payload was dissolved in 50 mL DMSO and added to the above reaction mixture containing reduced trastuzumab antibody. Reaction time was 3 hours at 37° C. 1.5 ml of 1M TRIS-HCl buffer pH 8.5 was added to the reaction mixture to stabilize i.e to open maleimide ring by selective hydrolysis. After overnight incubation the final reaction product was purified using protein A HPLC.

### Example 14. In Vivo Experiments with Syngeneic Tumors

Syngeneic mouse melanoma model B16-F10 mouse melanoma cancer cells were obtained from the ATCC (USA) and cultured according to the manufacturer's instructions to study accumulation of antibody-drug conjugates in vivo to subcutaneous syngeneic tumors in C57BL/6J mice. The study was performed at the TCDM/Central Animal Laboratory, University of Turku, Finland, according to the appropriate ethical committee approval. Cells for inoculation to mice were prepared in vigorous exponential growth phase. 0.5 million cells in 50% Matrigel were inoculated s.c. to the flank of each mouse. Clinical signs and general behavior of the animals was observed regularly. Tumor growth was followed by palpation. After caliper measurement, tumor volume was calculated according to 0.5×length×width². The first dosings were administered 2 days after inoculation and subsequent doses at 5 days' intervals. Mice were randomly divided into study groups. A set of preselected mice were sacrificed 2 days after the second dosing and representative samples of the tumors, blood and other tissues were taken and frozen for analyses.

Anti-PD-L1 ADCs

Anti-PD-L1 ADCs were prepared as described above for trastuzumab, using the recombinant fully human IgG1 antibody avelumab (Bavencio, Merck KGgA) that cross-reacts with mouse PDL1. The B16-F10 tumor-bearing mice (strain C57BL/6J) were administered as described above with three 10 mg/kg i.v. (intravenous infusion to the tail vein in PBS) doses each of avelumab, avelumab-cyclo-MeMe-kifunensine (DAR=21-28) and avelumab-AANA-kifunensine (DAR=8) as well as with three 10 mg/kg i.p. (intraperitoneal injection in PBS) doses of anti-mouse-PD-1 antibody (InvivoMAb, clone RMP1-14, rat IgG2a kappa). Control mice received no injections. All study groups included 7 tumor-bearing mice while the control group included 5 tumor-bearing mice.

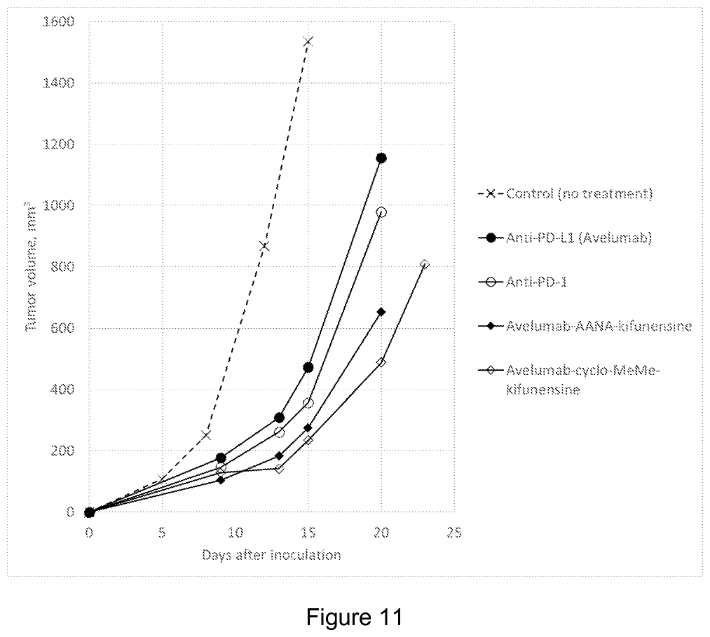

FIG. 11 shows average tumor volumes in the study groups during the follow-up period. Both avelumab-kifunensine ADC study groups had on average smaller tumors than the other study groups, demonstrating drastically increased anti-tumor efficacy of the ADC compared to the naked antibody and anti-PD-1/PD-L1 therapy. On day 15, the average tumor volumes in the groups were as follows: control group: 1534 mm³, avelumab/anti-PD-L1: 472 mm³, anti-PD-1: 357 mm³, avelumab-AANA-kifunensine: 275 mm³ (18% of control and 58% of avelumab), and avelumab-cyclo-MeMe-kifunensine: 234 mm³ (15% of control and 50% of avelumab).

Syngeneic Mouse Colon Cancer Model

MC38 mouse colon cancer cells were cultured in standard cell culture conditions to study accumulation of antibody-drug conjugates in vivo to subcutaneous syngeneic tumors in C57BL/6J mice. The study was performed at the TCDM/Central Animal Laboratory, University of Turku, Finland, according to the appropriate ethical committee approval. Cells for inoculation to mice were prepared in vigorous exponential growth phase. 0.5 million cells in 50% Matrigel were inoculated s.c. to the flank of each mouse. Clinical signs and general behavior of the animals was observed regularly. Anti-PD-L1 ADCs were prepared and dosed to the animals as described above: first dosings were administered 2 days after inoculation and the second dose after 5 day's interval. A set of preselected mice were sacrificed 2 days after the second dosing and representative samples of the tumors, blood and other tissues were taken and frozen for analyses. The tumor and tissue samples were subjected to N-glycan profiling with MALDI-TOF MS essentially as described above. The kifunensine ADCs specifically increase of the relative proportion of especially the Man9 high-mannose N-glycans in the N-glycome of the tumors, whereas other treatments/controls had no such effect, and at the same time, relative proportion of complex-type and sialylated N-glycans was diminished in the kifunensine ADC groups compared to the other treatments/controls. However, other tissues including kidney showed no detectable effect (avelumab-AANA-kifunensine) or much smaller effect as in the tumor (avelumab-cyclo-MeMe-kifunensine) compared to the controls including treatment with the naked antibody avelumab.

### Example 15. Dendritic Cell Activation Assay

Monocytes were isolated from human peripheral blood (buffy coat) from healthy adult donors (Finnish Red Cross Blood Service) by using magnetic-activated microbead based cell sorting for CD14 positive cells (Miltenyi Biotec). Monocytes were differentiated to immature dendritic cells (iDCs) by using Mo-DC Differentiation Medium (Miltenyi Biotec) according the manufacturer's instructions. On day 6, to analyze cell surface expression of maturation markers, the iDCs were labeled with specific monoclonal antibodies for CD209 (R&D Systems) and CD14 (eBioscience). Nonspecific antibody binding was assessed using appropriate isotype controls. The iDCs were used for co-culture experiment on day 7.

SK-BR-3 cells (ATCC) were cultured in DMEM (Gibco) containing GlutaMAX and supplemented with 10% heat-inactivated fetal bovine serum (FBS, Gibco). SK-BR-3 cells were plated in 96-well plate (5000 c/well) in the presence or absence of 10 pM kifunensine (Carbosynth Limited) and cultured for 5 days. Before start of the co-culture, SK-BR-3 cells were washed twice with RPMI 1640 medium (Gibco). Differentiated iDCs were collected and 0.11×10⁶ cells/well were added together with the test compounds to SK-BR-3 cells and cells were incubated for 24 h at +37° C. in 5% CO₂. The experiment medium was RPMI 1640 medium (Gibco) containing 10 mM Hepes and 2 mM glutamine and supplemented with 10% FBS (Gibco). All treatments were performed as duplicates. The test compounds were 5 nM trastuzumab (Herceptin®, Roche), 5 nM trastuzumab-maleimidoethylamine-MeMe-kifunensine (DAR=8), and 5 nM trastuzumab-cyclo-MeMe-kifunensine (DAR=28). Phorbol 12-myristate 13-acetate (PMA, Abcam) was used as positive control. After 24 h co-culture of iDCs and SKBR-3 cells, the cell supernatants were collected and centrifuged (3000 g, 5 min, +4° C.) to remove cells and debris. The supernatants were frozen and subjected to cytokine and chemokine analysis with Human Cytokine 48-Plex Discovery Assay (EVE Technologies, Canada).

Kifunensine pretreatment or kifunensine-ADC treatment of the cancer cells increased dendritic cell activation and secretion of cytokines, exemplified by fractalkine concentration analysis results below in Table F. Fractalkine concentration was higher if SK-BR-3 cells were pretreated with kifunensine, for example 121 pg/ml in SK-BR-3+iDCs treatment with kifunensine pretreatment compared to 34 pg/ml in SK-BR-3+IDCs treatment without kifunensine pretreatment. Fractalkine concentration was also higher if SK-BR-3 cells were treated with kifunensine-ADC, for example 173 pg/ml in SK-BR-3+iDCs+trastuzumab-maleimidoethylamine-MeMe-kifunensine treatment with kifunensine pretreatment and 161 pg/ml in SK-BR-3+iDCs+trastuzumab-cyclo-MeMe-kifunensine treatment with kifunensine pretreatment compared to 113 pg/ml in SK-BR-3+IDCs+trastuzumab treatment with kifunensine pretreatment. Thus, kifunensine-treated cancer cells were shown to induce myeloid cell activation and kifunensine-ADCs were shown to induce the same effect.

TABLE F
Fractalkine concentration.

| | Fractalkine |
|---|---|
| Treatment | pg/ml |
| SK-BR-3 | 22.40 |
| SK-BR-3 + IDCs | 34.43 |
| SK-BR-3 + iDCs + trastuzumab | 50.64 |
| SK-BR-3 + iDCs + trastuzumab- | 57.06 |
| maleimi-doethylamine- | |
| MeMe-kifunensine | |
| SK-BR-3 + iDCs + trastuzumab- | 58.59 |
| cyclo-MeMe-kifunensine | |
| SK-BR-3 + iDCs + PMA | 45.18 |
| SK-BR-3 + iDCs | 120.94 |
| SK-BR-3 + iDCs + trastuzumab | 113.23 |
| SK-BR-3 + iDCs + trastuzumab- | 172.59 |
| maleimi-doethylamine- | |
| MeMe-kifunensine | |
| SK-BR-3 + iDCs + tratuzumab- | 160.56 |
| cyclo-MeMe-kifunensine | |
| SK-BR-3 + iDCs + PMA | 158.90 |
| IDCs | 3.95 |
| iDCs + trastuzumab | 1.98 |
| iDCs + trastuzumab- | 0.00 |
| maleimidoethy- | |
| lamine-MeMe-kifunensine | |
| iDCs + tratuzumab-cyclo- | 0.43 |
| MeMe-ki-funensine | |
| iDCs + PMA | 8.27 |

### Example 16. ADCs and Stability Assays

The anti-EGFR recombinant therapeutic antibodies nimotuzumab (Biocon) and panitumumab (Amgen) were utilized for glycoconjugation reactions with UDP-GalNAz (Thermo Fisher Scientific) and (1,4-galactosyltransferase (Y289L; Life Technologies Europe, mutGalT) essentially as described above. To prepare nimotuzumab-GalNAz and panitumumab-GalNAz, 3 mg of each antibody was incubated overnight at ±3700 in 600 μl 50 mM MOPS pH 7.2, 5 mM MnCl₂ with UDP-GalNAz (1.6 mg/ml) and mutGalT (100 pig/ml). Excess UDP-GalNAz was removed from the reaction mixture and buffer was exchanged to PBS by filtration with Amicon ultra-0.5 ml 30K devices prior to click conjugation reactions. 1 mg of both nimotuzumab-GalNAz and panitumumab-GalNAz were conjugated with DBCO-cyclo-DS-kifunensine in a final antibody concentration of 4 mg/ml. Additionally, 1 mg of both was conjugated with DBCO-cyclo-DiDi-kifunensine in the same final antibody concentration and with 20% 1,2-propanediol. The conjugation reactions were incubated overnight at room temperature. The result of each reaction was evaluated by Fc analysis. 40 pg antibody was digested by FabRICATOR™ enzyme (Genovis) and purified by PorosR1. The resulting fragments were analyzed by MALDI-TOF MS. As expected, the DBCO-cyclo-DS-kifunensine ADC showed addition of 1-2 linker-payloads with mass of 4689, and the DBCO-cyclo-DiDi-kifunensine ADC showed addition of 1-2 linker-payloads with mass of 5253, showing essentially complete click cycloaddition reaction to the available GalNAz azide groups. The ADCs were subjected to similar stability assays by incubation in PBS and mouse serum at +37° C. According to MALDI-TOF MS analysis, the anti-EGFR ADCs with DBCO-cyclo-DiDi-kifunensine linker-payload were drastically more stable than the anti-EGFR ADCs with DBCO-cyclo-kifunensine linker-payload, since the former did not show any loss of kifunensine residues in the assays while the latter lost kifunensine residues during the incubations.

### Example 17. PAB Ether Conjugate

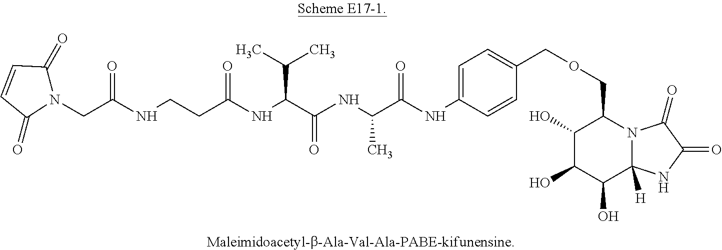 [SMILES: CC(C)[C@H](NC(=O)CCNC(=O)CN1C(=O)C=CC1=O)C(=O)N[C@@H](C)C(=O)Nc1ccc(COC[C@@H]2[C@@H](O)[C@H](O)[C@H](O)[C@H]3NC(=O)C(=O)N23)cc1]

Maleimidoacetyl-β-Ala-Val-Ala-PABE-kifunensine (Scheme E17-1) is prepared essentially as described in WO 03/026577 (Senter & Toki) by either the Mitsunobu reaction or the two-step imidate substitution reaction. Briefly, a protocol to obtain the final product starts with Fmoc- or Boc-protected Val-Ala-PAB-OH, which is reacted with kifunensine to form Fmoc- or Boc-protected Val-Ala-PAB-kifunensine. Site-specificity to kifunensine is achieved by protecting groups as desired. Amino-protecting group is removed and the product is reacted with maleimidoacetyl-β-alanine pentafluorophenyl ester and deprotected to obtain the final product. The product is purified by RP-HPLC and characterized by MALDI-TOF MS as described above. ADCs are prepared by conjugation to antibodies with reduced hinge cysteines as described in the previous Examples, including homogeneous DAR=8 ADC. Optionally the maleimides are specifically hydrolyzed by incubation at pH>8 buffer to obtain a stabilized ADC.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A product, a method, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1-20. (canceled)

21. A conjugate of Formula I:
[D-O-L]ₙ-T Formula I
wherein D is a payload molecule comprising a sugar moiety;
O is an oxygen atom of said sugar moiety;
T is a targeting unit capable of binding a target molecule, cell or tissue;
n is at least 1; and
L is of Formula C:
—R₇-L₁-Sₚ-L₂-R₈— Formula C
wherein
R₇ is absent or a group covalently bonded to said oxygen atom;
L₁ is a spacer unit of the formula —Sₜ-L₁′-, wherein L₁′ is absent or a spacer moiety;
Sₚ is absent or a specificity unit;
L₂ is absent or a stretcher unit, wherein the stretcher unit optionally comprises a moiety of the formula —Sₜ-L₂′-, wherein L₂′ is absent or a stretcher moiety;
R₈ is absent or a group covalently bonded to the targeting unit;
each Sₜ is independently absent or a moiety of Formula LI: 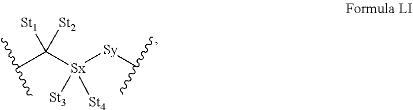
wherein St₁, St₂, St₃, and St₄ are each independently selected from H, CH₃, CH₂CH₃, unsubstituted or substituted C₁-C₆ alkyl, unsubstituted or substituted C₁-C₆ cycloalkyl, unsubstituted or substituted aryl, OH, OCH₃, OR_{O}, wherein R_{O} is either a C₁-C₆ alkyl or a C₁-C₆ substituted alkyl, and an amino acid side chain;
or,
wherein St₁ together with the carbon to which it is attached, with Sx and optionally with St₃ form an unsubstituted or substituted carbocyclyl or heterocyclyl group; Sx is either C or N, wherein St₄ is absent if Sx is N; Sy is either absent, —C(═O)O— or —(CH₂)ₘ—, wherein m is 1 to 4; and, L comprises at least one Sₜ.

22. The conjugate according to claim 21, wherein R₇ is absent or any one of the groups a-i:
a. —C(═O)—,
b. —C(═O)NH—,
c. —C(═O)O—,
d. —NHC(═O)—,
e. —NHC(═O)O—,
f. —C(═O)NH—,
g. —NHC(═O)NH—,
h. —P(═O)(OH)—, or
i. —S(═O)₂—.

23. The conjugate according to claim 21, wherein D is a glycosylation inhibitor or a galectin inhibitor, wherein the glycosylation inhibitor or the galectin inhibitor comprises a sugar moiety.

24. The conjugate according to claim 21, wherein R₇ is —C(═O)—.

25. The conjugate according to claim 21, wherein the payload molecule is a glycosylation inhibitor selected from the group of a metabolic inhibitor, a cellular trafficking inhibitor, tunicamycin, a plant alkaloid, a substrate analog, a glycoside primer, a specific inhibitor of glycosylation, an N-acetylglucosaminylation inhibitor, an N-acetylgalactosaminylation inhibitor, a sialylation inhibitor, a fucosylation inhibitor, a galactosylation inhibitor, a xylosylation inhibitor, a glucuronylation inhibitor, a mannosylation inhibitor, a mannosidase inhibitor, a glucosidase inhibitor, a glucosylation inhibitor, an N-glycosylation inhibitor, an O-glycosylation inhibitor, a glycosaminoglycan biosynthesis inhibitor, a glycosphingolipid biosynthesis inhibitor, a sulphation inhibitor, 2-deoxyglucose, a fluorinated sugar analog, 2-acetamido-2,4-dideoxy-4-fluoroglucosamine, 2-acetamido-2,3-dideoxy-3-fluoroglucosamine, 2-acetamido-2,6-dideoxy-6-fluoroglucosamine, 2-acetamido-2,5-dideoxy-5-fluoroglucosamine, 4-deoxy-4-fluoroglucosamine, 3-deoxy-3-fluoroglucosamine, 6-deoxy-6-fluoroglucosamine, 5-deoxy-5-fluoroglucosamine, 3-deoxy-3-fluorosialic acid, 3-deoxy-3ax-fluorosialic acid, 3-deoxy-3eq-fluorosialic acid, 3-deoxy-3-fluoro-NeuSAc, 3-deoxy-3ax-fluoro-NeuSAc, 3-deoxy-3eq-fluoro-Neu5Ac, 3-deoxy-3-fluorofucose, 2-deoxy-2-fluoroglucose, 2-deoxy-2-fluoromannose, 2-deoxy-2-fluorofucose, 3-fluorosialic acid, castanospermine, australine, deoxynojirimycin, N-butyldeoxynojirimycin, deoxymannojirimycin, kifunensin, swainsonine, mannostatin A, streptozotocin, 2-acetamido-2,5-dideoxy-5-thioglucosamine, 2-acetamido-2,4-dideoxy-4-thioglucosamine, PUGNAc (0-[2-acetamido-2-deoxy-Dglucopyranosylidene]amino-N-phenylcarbamate), Thiamet-G, N-acetylglucosamine-thiazoline (NAG-thiazoline), GlcNAcstatin, a nucleotide sugar analog, a UDP-GlcNAc analog, a UDP-GalNAc analog, a UDP-Glc analog, a UDP-Gal analog, a GDP-Man analog, a GDP-Fuc analog, a UDP-GlcA analog, a UDP-Xyl analog, a CMP-Neu5Ac analog, a nucleotide sugar bisubstrate, a glycoside primer, a β-xyloside, a β-N-acetylgalactosaminide, a β-glucoside, a β-galactoside, β-N-acetylglucosaminide, a β-N-acetyllactosaminide, a disaccharide glycoside and a trisaccharides glycosideglucosylceramide epoxide, 2-amino-2-deoxymannose, a 2-acyl-2-deoxy-glucosyl-phosphatidylinositol, Neu5Ac-2-ene (DANA), 4-amino-DANA, 4-guanidino-DANA, a mannosidase I inhibitor, a glucosidase I inhibitor, a glucosidase II inhibitor, an N-acetylglucosaminyltransferase inhibitor, an N-acetylgalactosaminyltransferase inhibitor, a galactosyltransferase inhibitor, a sialyltransferase inhibitor, a hexosamine pathway inhibitor, a glutamine-fructose-6-phosphate aminotransferase (GFPT1) inhibitor, a phosphoacetylglucosamine mutase (PGM3) inhibitor, a UDP-GlcNAc synthase inhibitor, a CMP-sialic acid synthase inhibitor, N-acetyl-D-glucosamine-oxazoline, 6-methyl-phosphonate-N-acetyl-D-glucosamine-oxazoline, 6-methyl-phosphonate-N-acetyl-D-glucosamine-thiazoline, a concanamycin, concanamycin A, concanamycin B, concanamycin C, a bafilomycin, bafilomycin A1, epi-kifunensine, deoxyfuconojirimycin, 1,4-dideoxy-1,4-imino-D-mannitol, 2,5-dideoxy-2,5-imino-D-mannitol, 1,4-dideoxy-1,4-imino-D-xylitol, an N-acyldeoxynojirimycin, N-acetyldeoxynojirimycin, an N-acyldeoxymannojirimycin, N-acetyldeoxymannojirimycin, 3-deoxy-3-fluoro-Neu5N, 3-deoxy-3ax-fluoro-Neu5N, 3-deoxy-3eq-fluoro-Neu5N, 3′-azido-3′-deoxythymidine, 3′-fluoro-3′-deoxythymidine, 3′-azido-3′-deoxycytidine, 3′-fluoro-3′-deoxycytidine, 3′-azido-2′,3′-dideoxycytidine, 3′-fluoro-2′,3′-dideoxycytidine, and any analogs, modifications, acylated analogs, acetylated analogs, methylated analogs, or combinations thereof.

26. The conjugate according to claim 21, wherein the payload molecule is a galectin inhibitor selected from the group of galactose, a 3-substituted galactose, a β-D-galactoside, a galactoside, a 3-substituted galactoside, a β-D-galactoside, a 3-substituted β-D-galactoside, lactose, a 3′-substituted lactose, a lactoside, a 3′-substituted lactoside, N-acetyllactosamine, a 3′-substituted N-acetyllactosamine, an N-acetyllactosaminide, a 3′-substituted N-acetyllactosaminide, N,N′-di-N-acetyllactosediamine, a 3′-substituted N,N′-di-N-acetyllactosediamine, an N,N′-di-N-acetyllactosediaminide, a 3′-substituted N,N′-diN-acetyllactosediaminide, a taloside, a 3′-substituted taloside, a β-D-taloside, a 3′-substituted β-D-taloside, a mannoside, a 3′-substituted mannoside, a β-D-mannoside, a 3′-substituted β-D-mannoside, thiodigalactose (TDG), a 3-substituted thiodigalactose, a 3,3′-disubstituted thiodigalactose, 3,3′-dideoxy-3,3′-bis-[4-(3-fluorophenyl)-1H-1,2,3-triazol-1-yl]-1,1′-sulfanediyl-di-β-D-galactopyranoside (33DFTG or TD139), 6-acyl-33DFTG, 6-succinyl-33DFTG, di-6-acyl-33DFTG, di-6-succinyl-33DFTG, a 6-substituted 33DFTG, a 6,6′-disubstituted 33DFTG, (E)-methyl-2-phenyl-4-(β-D-galactopyranosyl)-but-2-enoate, Galβ1-4Fuc, a 3′-substituted Galβ1-4Fuc, GM-CT-01, GR-MD-02, a pectin, reduced pectin, modified citrus pectin, GCS-100, a poly-N-acetyllactosaminide, lactulose, a lactuloside, a 3′-substituted lactulose, a 3′-substituted lactuloside, lactulosyl-L-leucine, a 3′-substituted lactulosyl-L-leucine, a galectin-binding molecule that inhibits galectin-galectin ligand interaction, an RNAi inhibiting galectin expression, GB1107, and any analog, modification, combination or multivalent combination thereof.

27. The conjugate according to claim 21, wherein L₁′ is either absent or any one of the groups a-h:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety, or
h. a substituted branched PEG₂₋₅₀ polyethylene glycol moiety.

28. The conjugate according to claim 21, wherein Sₚ is either absent or any one of the groups a-n:
a. dipeptide,
b. tripeptide,
c. tetrapeptide,
d. valine-citrulline,
e. phenylalanine-lysine,
f. valine-alanine,
g. valine-serine,
h. asparagine,
i. alanine-asparagine,
j. alanine-alanine-asparagine,
k. a hydrazone,
l. an ester,
m. a disulfide, or
n. a glycoside.

29. The conjugate according to claim 21, wherein L₂′ is either absent or any one of the groups a-j:
a. a C₁₋₁₂ alkylene,
b. a substituted C₁₋₁₂ alkylene,
c. a C₅₋₂₀ arylene,
d. a substituted C₅₋₂₀ arylene,
e. a PEG₁₋₅₀ polyethylene glycol moiety,
f. a substituted PEG₁₋₅₀ polyethylene glycol moiety,
g. a branched PEG₂₋₅₀ polyethylene glycol moiety,
h. a substituted branched PEG₂₋₅₀ polyethylene glycol moiety,
i. a moiety of the formula XXVI, or
j. a moiety of the formula XXVII.

30. The conjugate according to claim 21, wherein R₈ is either absent or any one of the groups a-k:
a. —C(═O)NH—,
b. —C(═O)O—,
c. —NHC(═O)—,
d. —OC(═O)—,
e. —OC(═O)O—,
f. —NHC(═O)O—,
g. —OC(═O)NH—,
h. —NHC(═O)NH—,
i. —NH—,
j. —O—, or
k. —S—.

31. The conjugate according to claim 1, wherein each Sₜ is independently absent, a moiety of formula LI, wherein St₃ and St₄ are optionally absent, or a moiety of formula LII, LIII, LIV, LV, LVI, LVII, LVIII, LIX, LX, LXI, LXII, LXIII, LXIV, LXV, LXVI or LXVII: 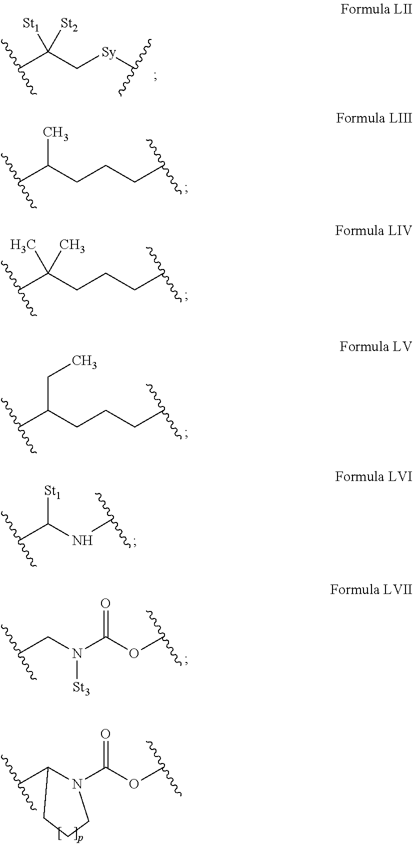
Formula LVIII wherein p is from 1 to 2; 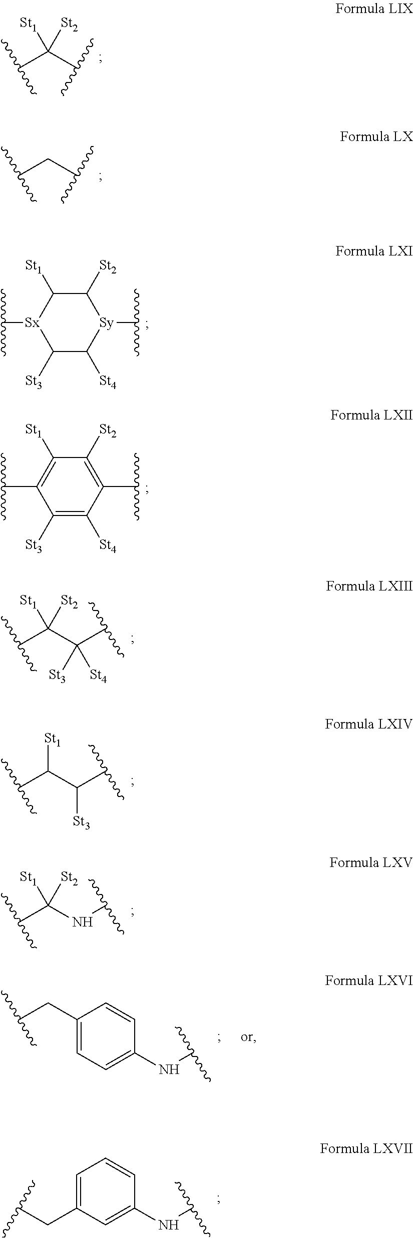
wherein the wavy lines in Formulas LII-LXVII show the bonds to the rest of the structure;
St₁, St₂, St₃, St₄, Sx, and Sy are as defined in any one of the preceding claims; and,
the stereochemical centers in any one of the Formulas LII-LXVII are in either the R or S configuration or a racemic mixture.

32. The conjugate according to claim 21, wherein
R₇ is —C(═O)—;
L₁′ is a substituted or unsubstituted C₁-C₁₂ alkylene, optionally a substituted or unsubstituted C₁-C₆ alkylene;
Sₜ in L₁ is optionally absent;
Sₚ is disulfide;
Sₜ is present in L₂ and is as defined in any one of claims 21-31; or
R₇ is —C(═O)—;
L₁′ is a substituted or unsubstituted C₁-C₁₂ alkylene, optionally a substituted or unsubstituted C₁-C₆ alkylene;
Sₜ in L₁ is NH;
Sₚ is a peptide forming a peptide bond with Sₜ, optionally comprising an asparagine residue;
Sₜ and L₂ are as defined in any one of claims 21-31; or
R₇ is —C(═O)O—;
L₁′ is a substituted or unsubstituted C₁-C₁₂ alkylene, optionally a substituted or unsubstituted C₁-C₆ alkylene;
Sₜ in L₁ is optionally absent;
Sₚ is disulfide;
L₂ and R₈ are absent and Sₚ is directly bonded to a thiol group in the targeting unit so that an S atom in the disulfide is derived from the thiol group in the targeting unit.

33. The conjugate according to claim 21, wherein the targeting unit T comprises an antibody, optionally wherein the antibody is a tumour cell-targeting antibody, a cancer-targeting antibody or, an immune cell-targeting antibody; a peptide; an aptamer; a ligand; or a glycan.

34. The conjugate according to claim 21, wherein the conjugate is selected from the group consisting of conjugates of any one of Formulas CI to CXXIII, CXVm to CXXIIIm, DI to DXXIII and DXVm to DXXIIIm: 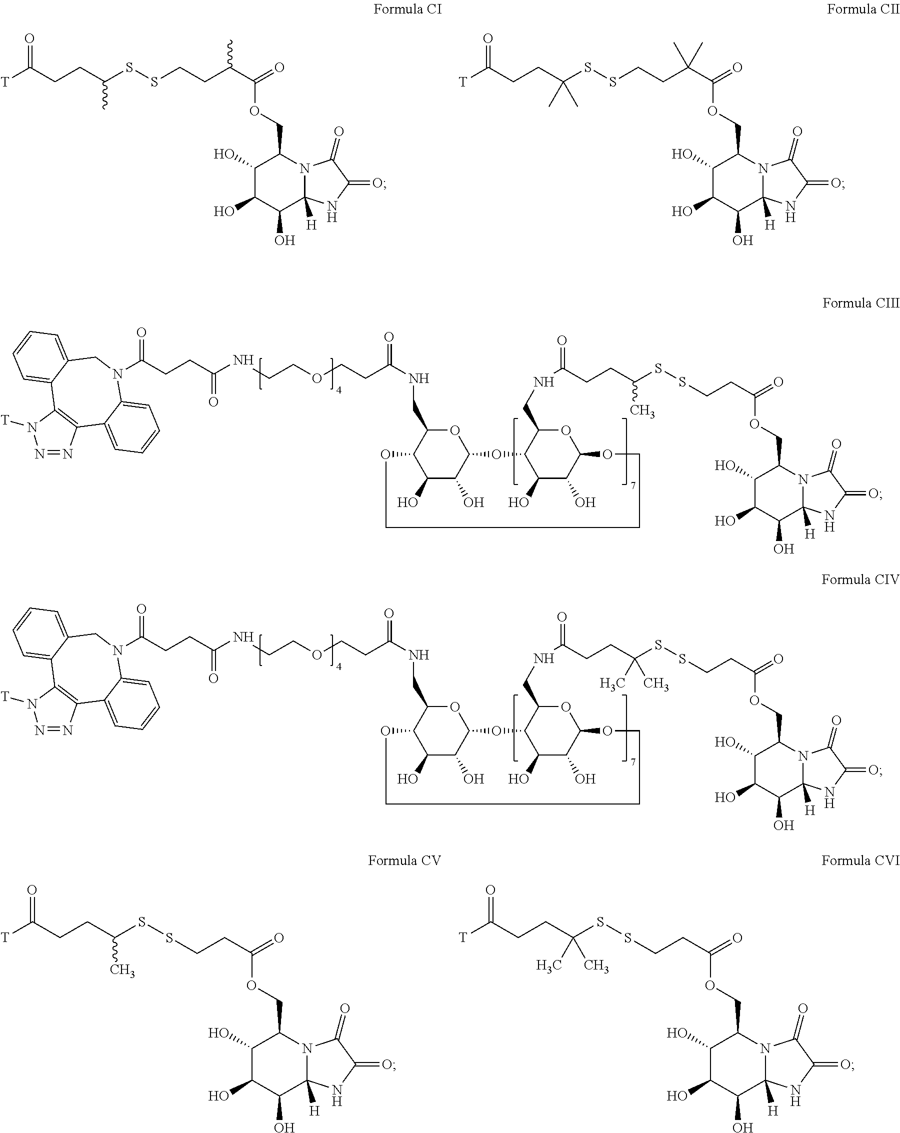 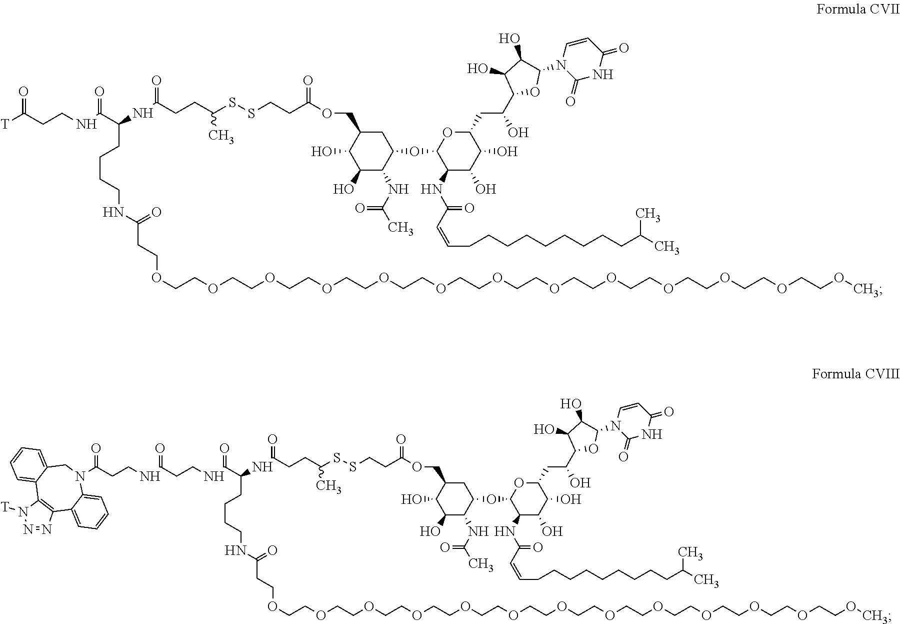 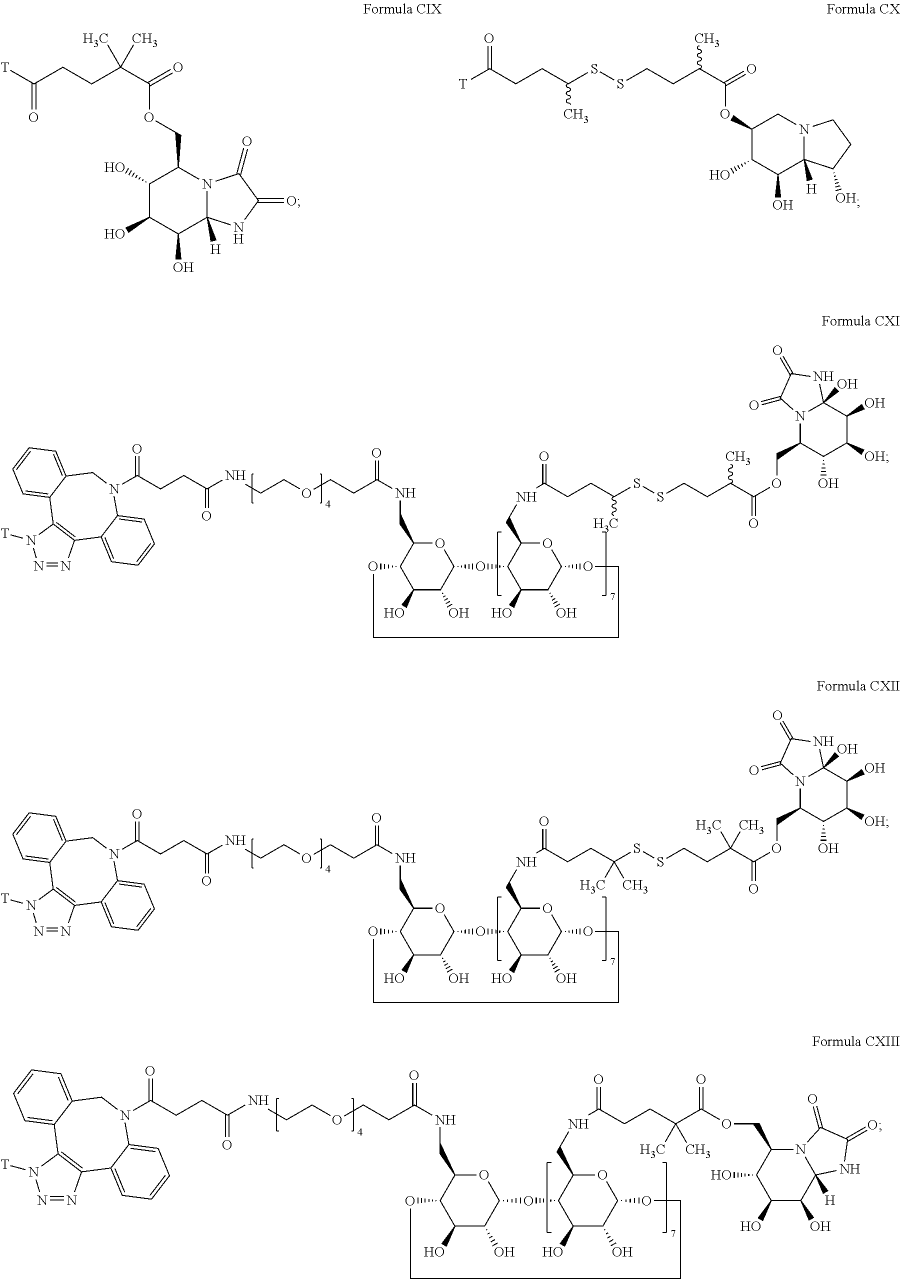 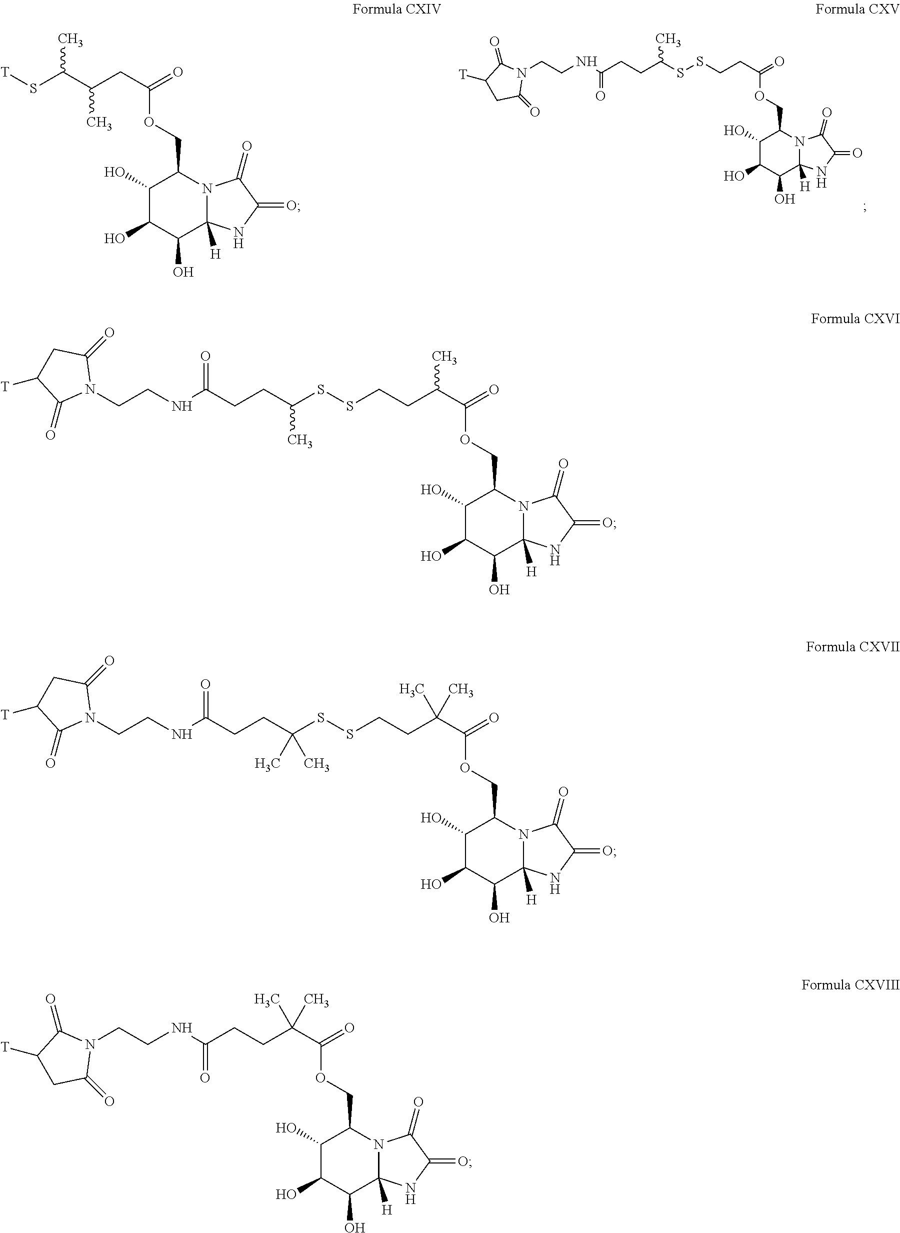 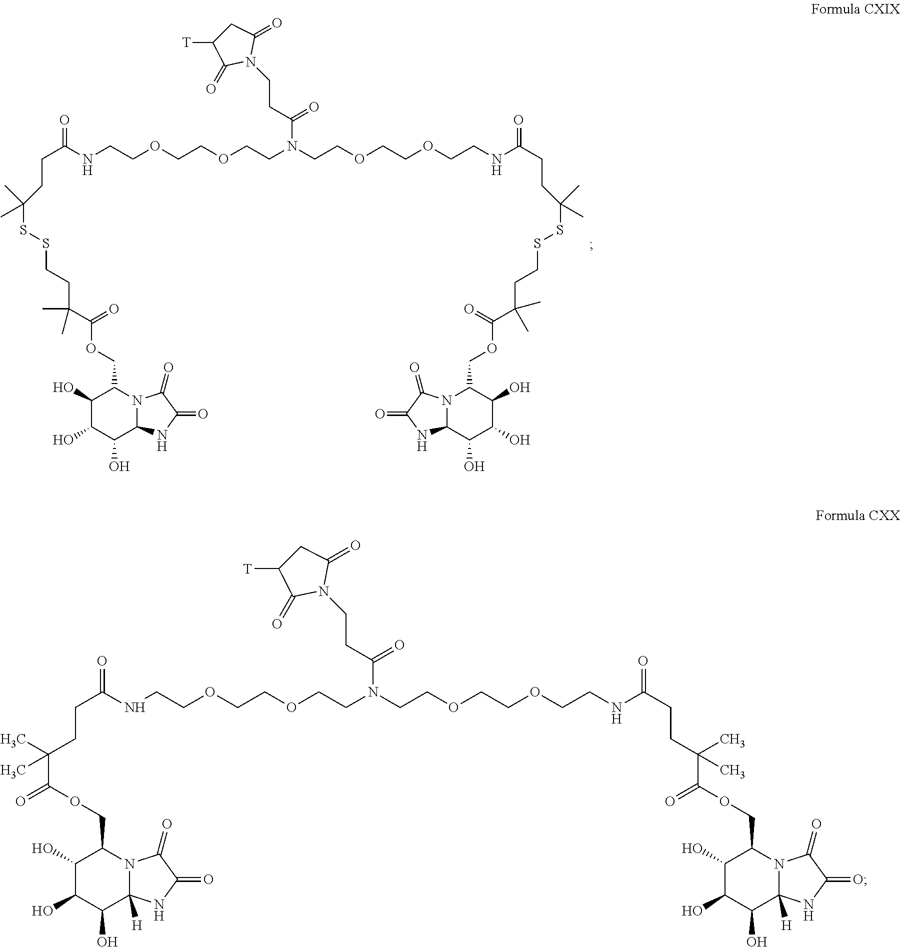 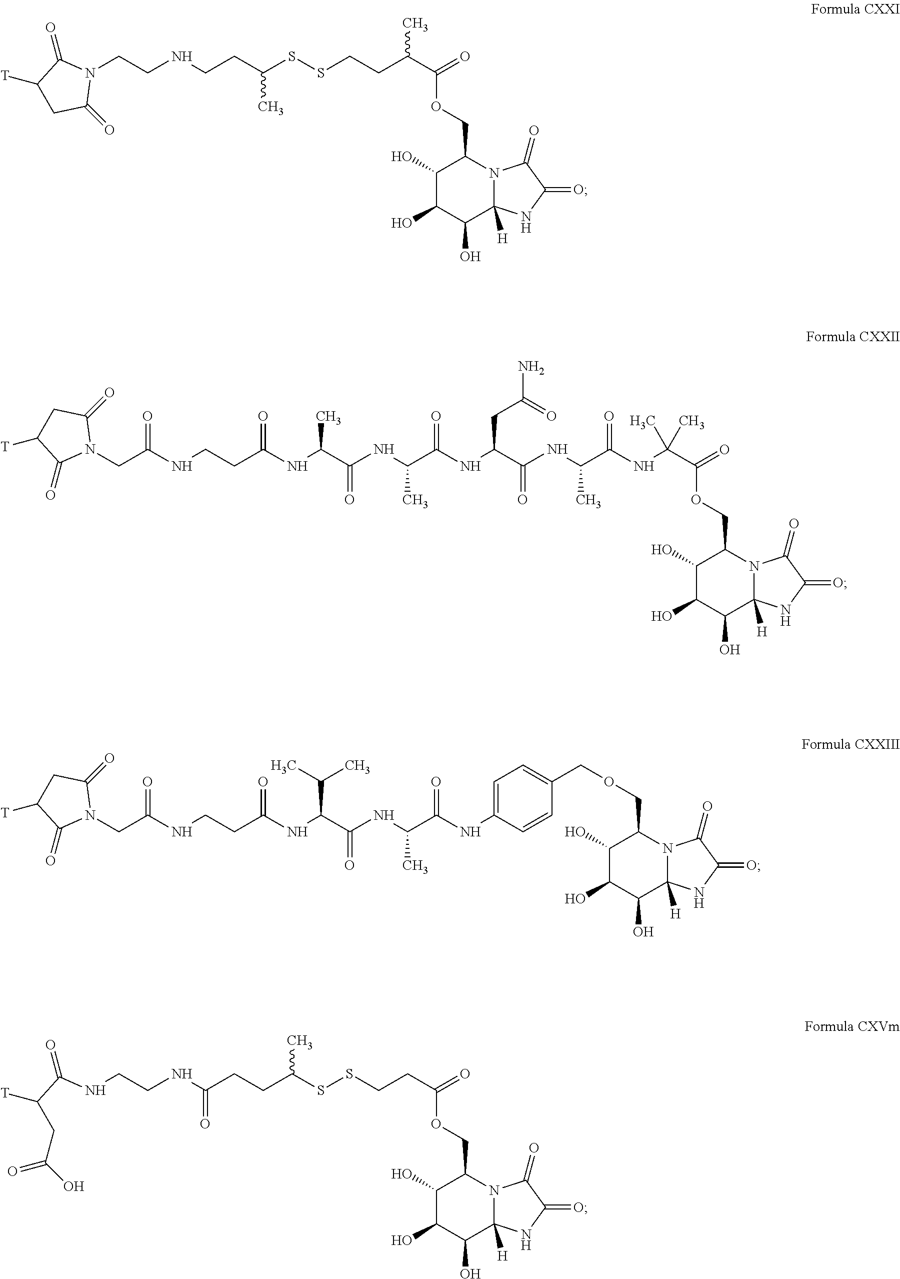 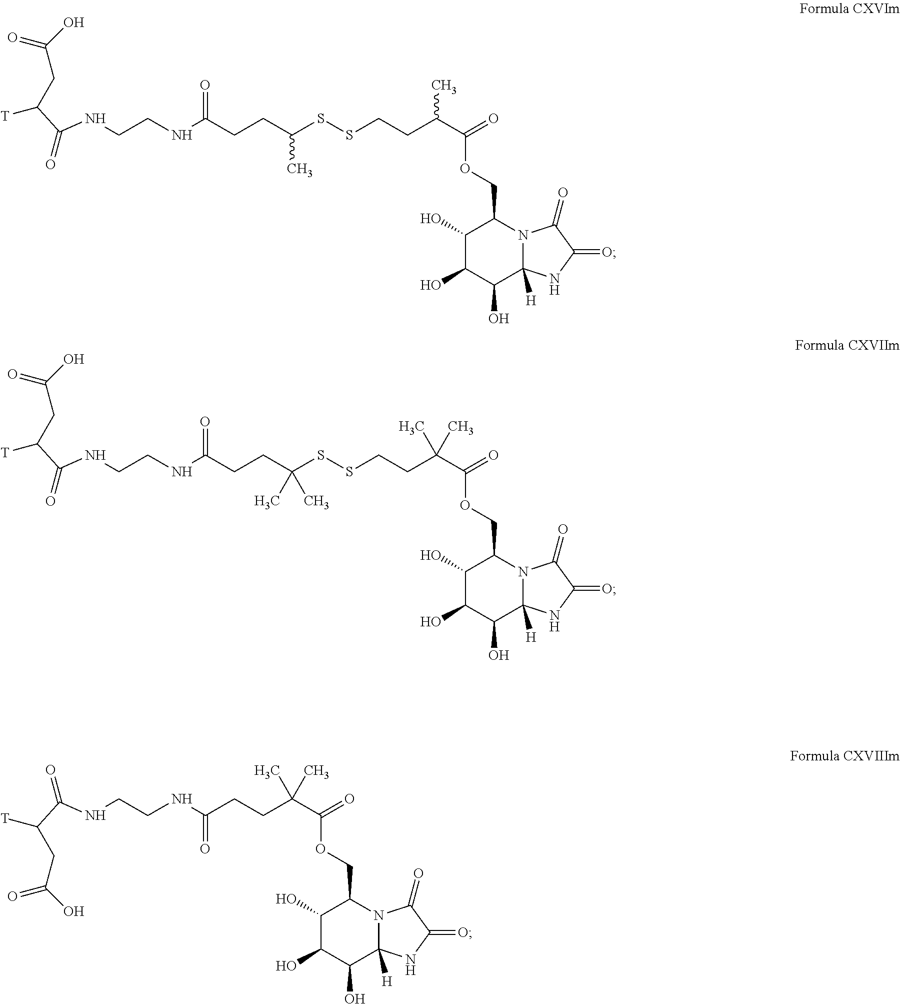 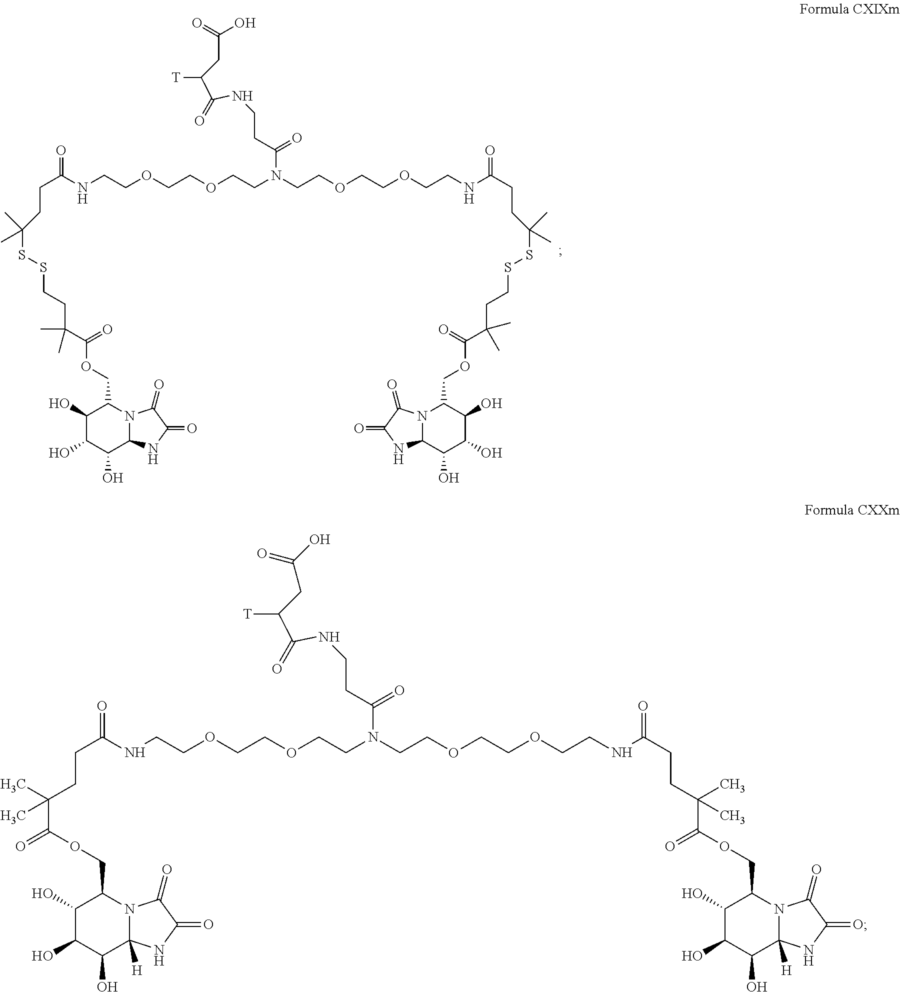 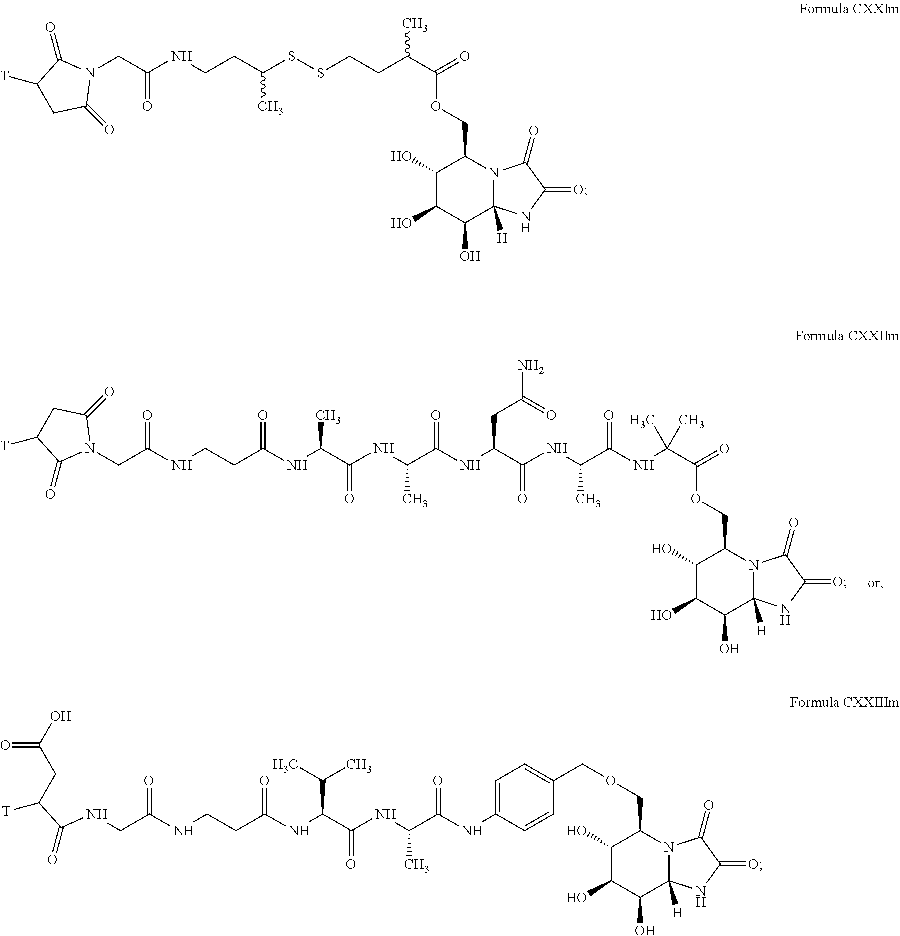
wherein T represents the targeting unit, and each T is optionally linked to one or more linker-payload units of: 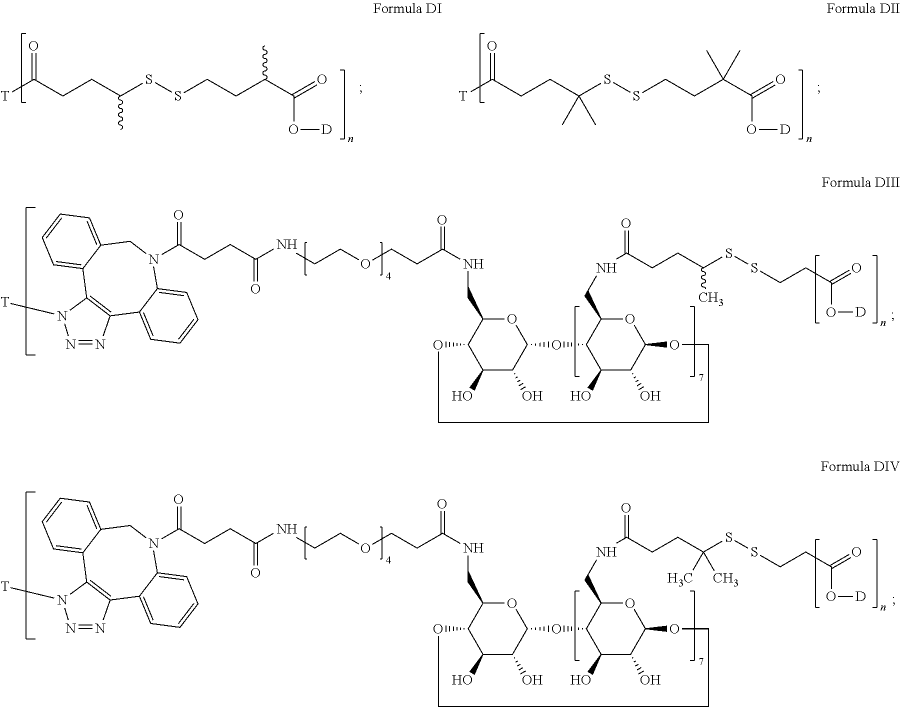 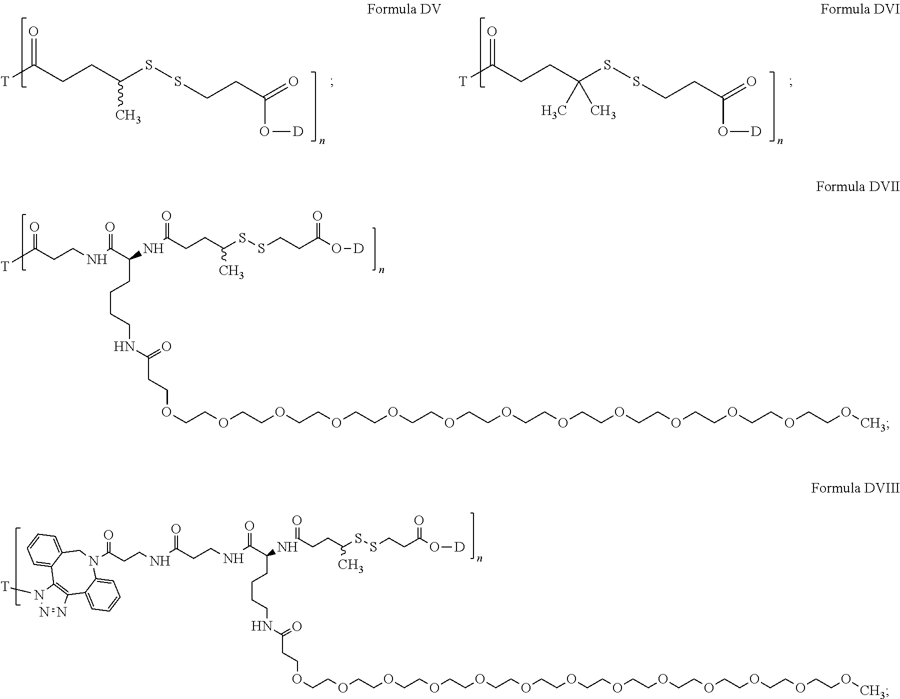 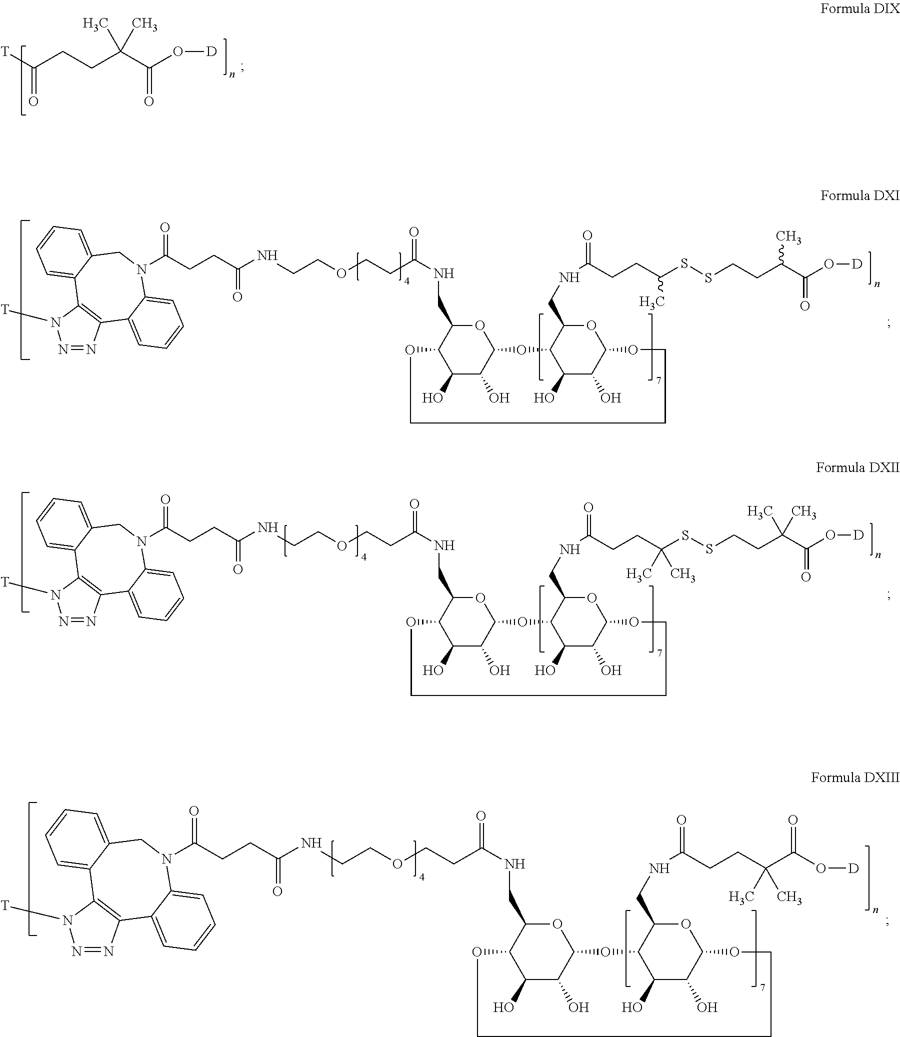 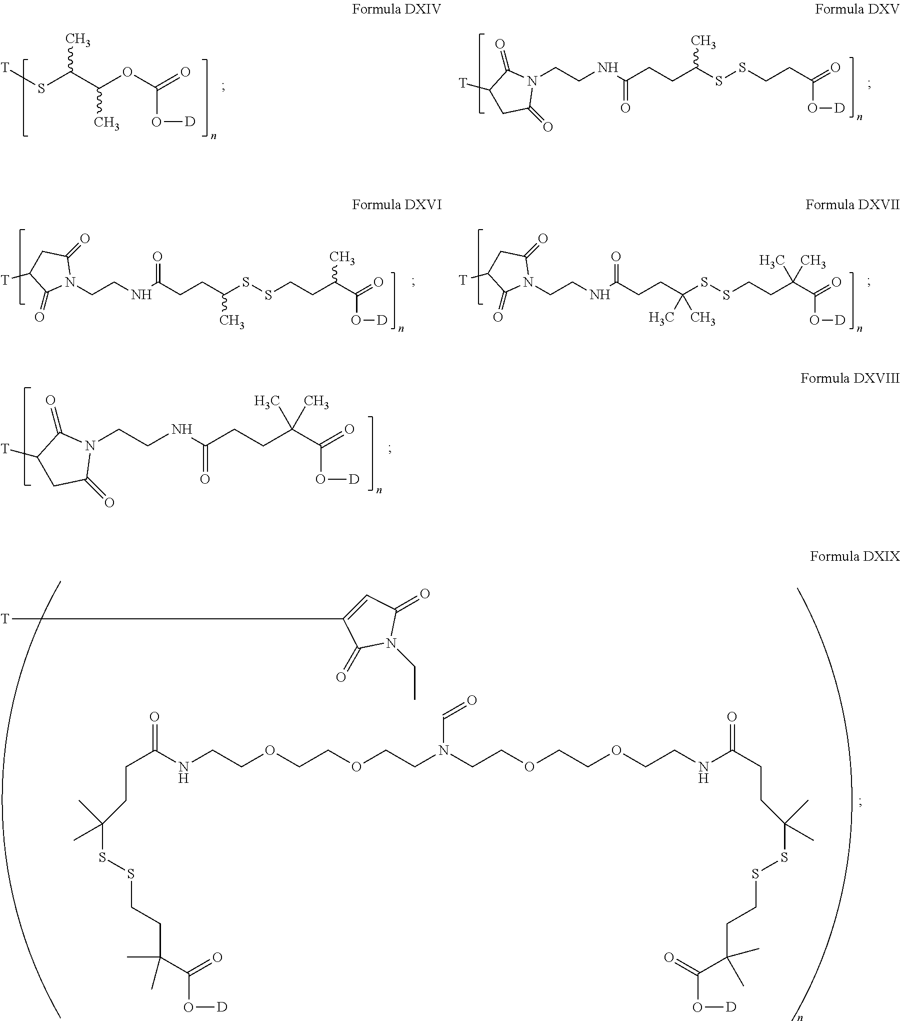 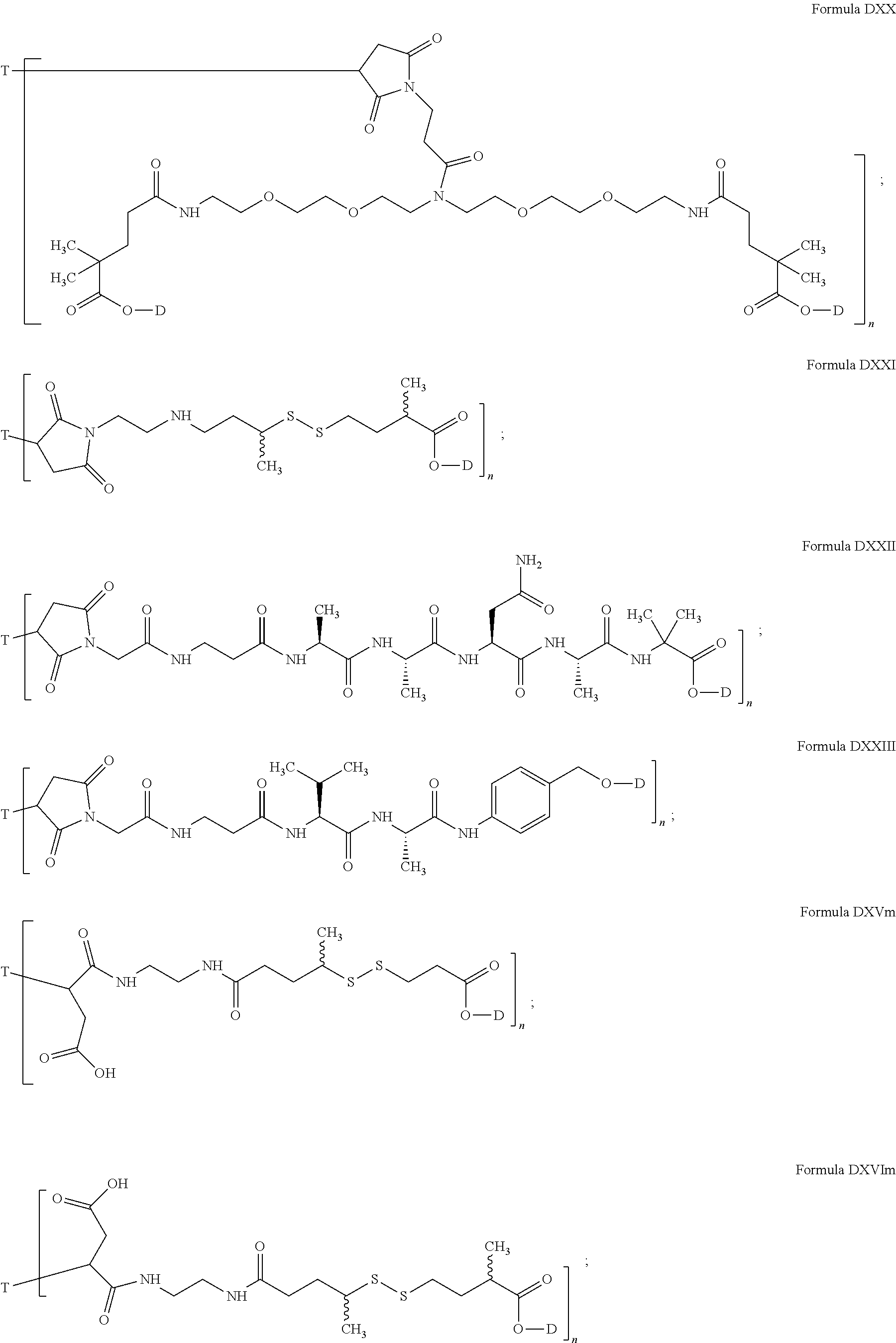 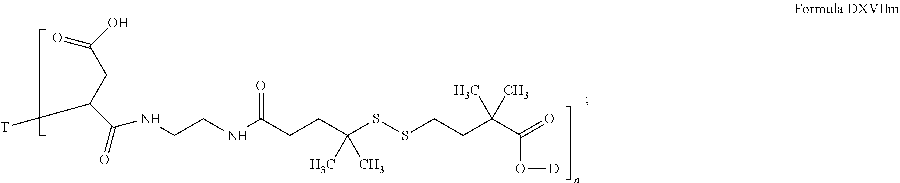 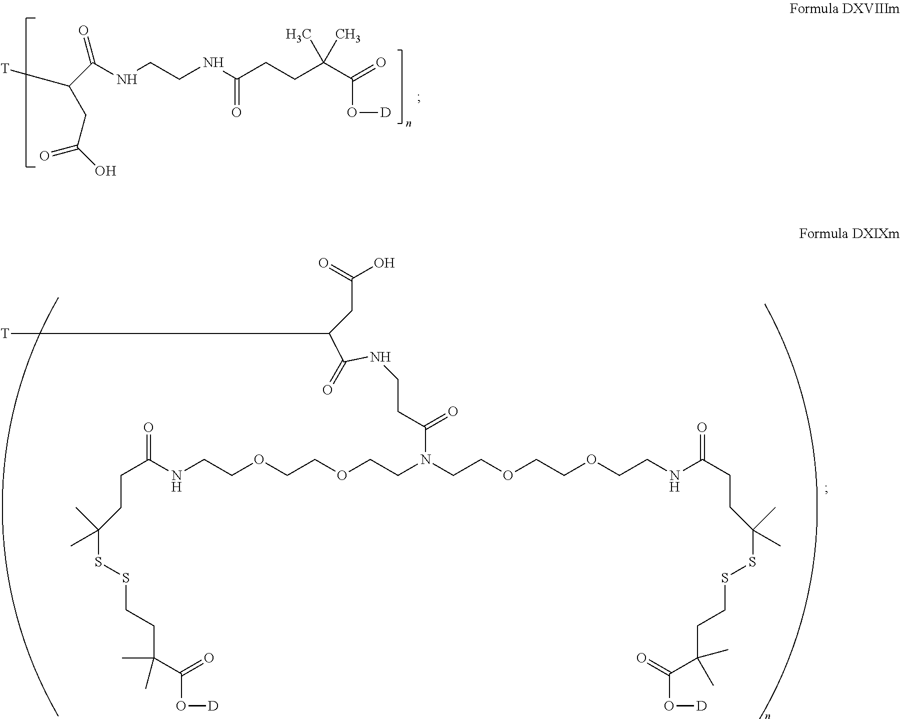 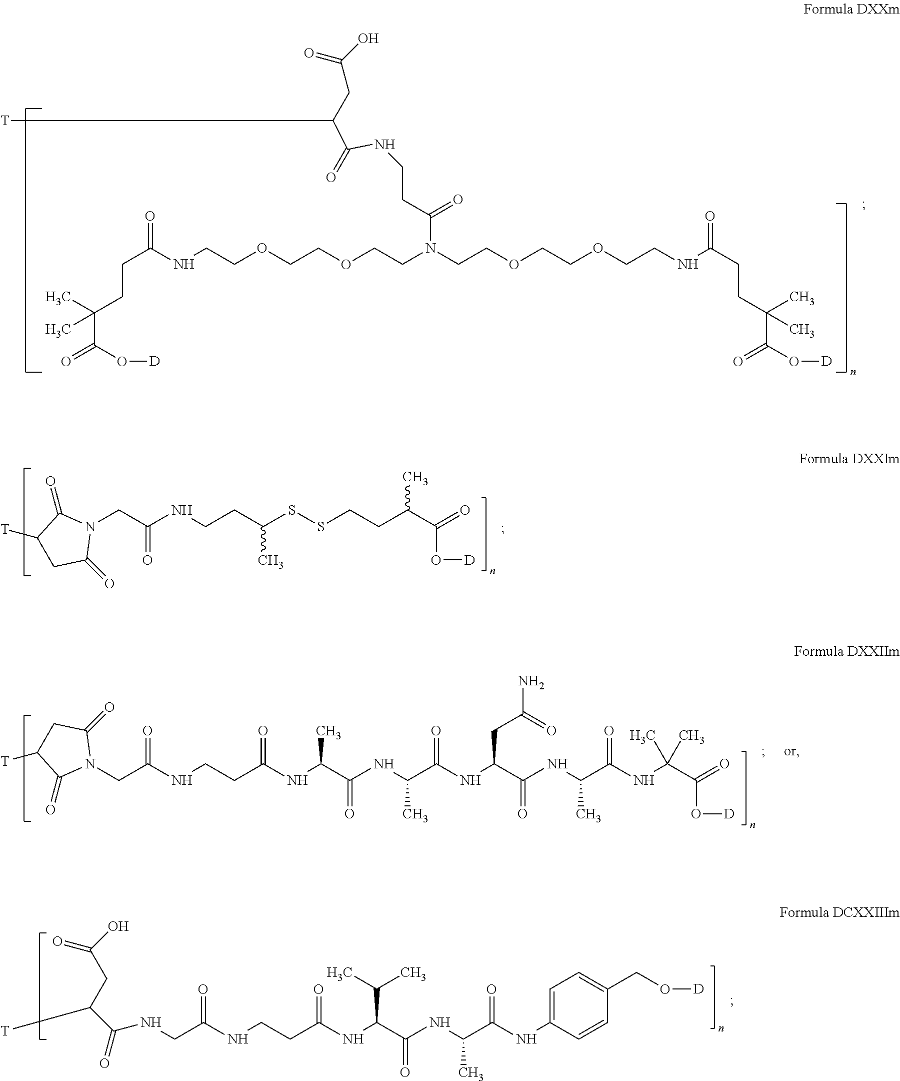
wherein O-D represents the payload molecule, wherein O is an oxygen atom of said payload molecule;
T is a targeting unit capable of binding a target molecule, cell and/or tissue; and,
n is at least 1.

35. The conjugate according to claim 21, wherein:
the targeting unit comprises a cancer-targeting antibody selected from the group of bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, epratuzumab, 2G12, lintuzumab, nimotuzumab and ibritumomab tiuxetan, or an antibody selected from the group of an anti-EGFR1 antibody, an epidermal growth factor receptor 2 (HER2/neu) antibody, an anti-CD22 antibody, an anti-CD30 antibody, an anti-CD33 antibody, an anti-Lewis y antibody, an anti-CD20 antibody, an anti-CD3 antibody, an anti-PSMA antibody, an anti-TROP2 antibody and an anti-AXL antibody; or
the targeting unit comprises an immune receptor-targeting antibody selected from the group of nivolumab, pembrolizumab, ipilimumab, atezolizumab, avelumab, durvalumab, BMS-986016, LAG525, MBG453, OMP-31M32, JNJ-61610588, enoblituzumab (MGA271), MGD009, 8H9, MEDI9447, M7824, metelimumab, fresolimumab, IMCTR1 (LY3022859), lerdelimumab (CAT-152), LY2382770, lirilumab, IPH4102, 9B12, MOXR 0916, PF-04518600 (PF-8600), MED16383, MED10562, MED16469, INCAGN01949, GSK3174998, TRX-518, BMS-986156, AMG 228, MEDI1873, MK4166, INCAGN01876, GWN323, JTX-2011, GSK3359609, MEDI-570, utomilumab (PF-05082566), urelumab, ARGX-110, BMS-936561 (MDX-1203), varlilumab, CP870893, APX005M, ADC-1013, lucatumumab, Chi Lob 7/4, dacetuzumab, SEA-CD40, RO7009789, MEDI9197; or
the targeting unit comprises a molecule selected from the group of an immune checkpoint inhibitor, an anti-immune checkpoint molecule, anti-PD-1, anti-PD-L1 antibody, anti-CTLA-4 antibody, a cancer-targeting molecule, or a targeting unit capable of binding an immune checkpoint molecule, the immune checkpoint molecule being selected from the group of: lymphocyte activation gene-3 (LAG-3, CD223), T cell immunoglobulin-3 (TIM-3), poly-N-acetyllactosamine, T (Thomsen-Friedenreich antigen), Globo H, Lewis c (type 1 N-acetyllactosamine), galectin-1, galectin-2, galectin-3, galectin-4, galectin-5, galectin-6, galectin-7, galectin-8, galectin-9, galectin-10, galectin-11, galectin-12, galectin-13, galectin-14, galectin-15, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-13, Siglec-14, Siglec-15, Siglec-16, Siglec-17, phosphatidyl serine, CEACAM-1, T cell immunoglobulin and ITIM domain (TIGIT), CD155 (poliovirus receptor-PVR), CD112 (PVRL2, nectin-2), V-domain Ig suppressor of T cell activation (VISTA, also known as programmed death-1 homolog, PD-1H), B7 homolog 3 (B7-H3, CD276), adenosine A2a receptor (A2aR), CD73, B and T cell lymphocyte attenuator (BTLA, CD272), herpes virus entry mediator (HVEM), transforming growth factor (TGF)-β, killer immunoglobulin-like receptor (KIR, CD158), KIR2DL1/2L3, KIR3DL2, phosphoinositide 3-kinase gamma (PI3K7), CD47, OX40 (CD134), Glucocorticoid-induced TNF receptor family-related protein (GITR), GITRL, Inducible co-stimulator (ICOS), 4-1BB (CD137), CD27, CD70, CD40, CD154, indoleamine-2,3-dioxygenase (IDO), toll-like receptors (TLRs), TLR1, TLR2, TLR3, TLR4, TLR5, TLR6, TLR7, TLR8, TLR9, interleukin 12 (IL-12), IL-2, IL-2R, CD122 (IL-2Rβ), CD132 (Υ_{c}), CD25 (IL-2Rα), and arginase.

36. The conjugate according to claim 21, wherein n is in the range of 1 to about 20, or 1 to about 15, or 1 to about 10, or 2 to 10, or 2 to 6, or 2 to 5, or 2 to 4, or 3 to about 20, or 3 to about 15, or 3 to about 10, or 3 to about 9, or 3 to about 8, or 3 to about 7, or 3 to about 6, or 3 to 5, or 3 to 4, or 4 to about 20, or 4 to about 15, or 4 to about 10, or 4 to about 9, or 4 to about 8, or 4 to about 7, or 4 to about 6, or 4 to 5; or about 7-9; or about 8, or 1, 2, 3, 4, 5, 6, 7, 8, 9, 10, 11, 12, 13, 14, 15, 16, 17, 18, 19, or 20; or in the range of 1 to about 1000, or 1 to about 2000, or 1 to about 400, or 1 to about 200, or 1 to about 100; or 100 to about 1000, or 200 to about 1000, or 400 to about 1000, or 600 to about 1000, or 800 to about 1000; 100 to about 800, or 200 to about 600, or 300 to about 500; or 20 to about 200, or 30 to about 150, or 40 to about 120, or 60 to about 100; over 8, over 16, over 20, over 40, over 60, over 80, over 100, over 120, over 150, over 200, over 300, over 400, over 500, over 600, over 800, or over 1000; or n is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 52, 54, 56, 58, 60, 62, 63, 64, 66, 68, 70, 72, 74, 76, 78, 80, 82, 84, 86, 88, 90, 92, 94, 96, 98, 100, 110, 120, 130, 140, 150, 160, 170, 180, 190, 200, 220, 240, 260, 280, 300, 350, 400, 450, 500, 550, 600, 650, 700, 750, 800, 850, 900, 950, 1000, 1100, 1200, 1300, 1400, 1500, 1600, 1700, 1800, 1900, 2000, or greater than 2000.

37. A pharmaceutical composition comprising the conjugate according to claim 21.

38. A method of treating cancer comprising administering the pharmaceutical composition according to claim 37 to a human.

39. The method according to claim 38, wherein the cancer is selected from the group of leukemia, lymphoma, breast cancer, prostate cancer, ovarian cancer, colorectal cancer, gastric cancer, squamous cancer, small-cell lung cancer, head-and-neck cancer, multidrug resistant cancer, glioma, melanoma, and testicular cancer.
